(19) United States
(12) Patent Application Publication
(10) Pub. No.: US 2024/0083892 A1
(43) Pub. Date: Mar. 14, 2024

(54) FUSED HETEROCYCLIC RINGS AS RIPK1 INHIBITORS

(71) Applicant: BISICHEM CO., LTD., Seongnam-si (KR)

(72) Inventors: Sangbae HAN, Seongnam-si (KR); Youngeun KWON, Seongnam-si (KR); Jiae MIN, Seongnam-si (KR); Sungmin CHOI, Seongnam-si (KR); Namhee KIM, Seongnam-si (KR); Minha KIM, Seongnam-si (KR); SeEun JEON, Seongnam-si (KR); Hongjun KANG, Seongnam-si (KR); HeeJin JEONG, Seongnam-si (KR); Sunjoo KIM, Seongnam-si (KR); KiTae PARK, Seongnam-si (KR); Jinyoung KIM, Seongnam-si (KR); Inho YANG, Seongnam-si (KR); Cheolhwan YOON, Seongnam-si (KR); Cheolkyu HAN, Seongnam-si (KR); Jeongbeob SEO, Seongnam-si (KR)

(21) Appl. No.: 17/947,527

(22) Filed: Sep. 19, 2022

Related U.S. Application Data
(60) Provisional application No. 63/245,282, filed on Sep. 17, 2021.

(51) Int. Cl. C07D 413/14; C07D 413/12; C07D 491/107

(52) U.S. Cl. CPC C07D 413/14; C07D 413/12; C07D 491/107

(57) ABSTRACT

The invention provides novel substituted heterocyclic rings represented by Formula I, or a pharmaceutically acceptable salt, solvate, polymorph, ester, tautomer or prodrug thereof, and a composition comprising these compounds. The compounds provided can be used as inhibitors of RIPK1 and the therapeutic methods.

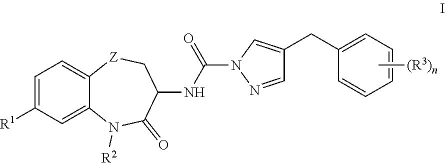

### CROSS-REFERENCE TO RELATED APPLICATION

This application claims priority from Provisional Application No. 63/245,282 filed on Sep. 17, 2021, which is incorporated herein by reference in its entirety.

### REFERENCE TO ELECTRONIC SEQUENCE LISTING

The application contains a Sequence Listing which has been submitted electronically in .XML format and is hereby incorporated by reference in its entirety. Said .XML copy, created on May 31, 2023, is named “LEE-P10003.xml” and is 11,088 bytes in size. The sequence listing contained in this .XML file is part of the specification and is hereby incorporated by reference herein in its entirety.

### TECHNICAL FIELD

This invention relates to a series of substituted heterocyclic compounds which are inhibitors of receptor-interacting protein-1 (RIP1) kinase-mediated disease or disorder and use the therapeutics.

### RELATED ART

Receptor-interacting protein-1 (RIP1) kinase is a serine/threonine protein kinase, referred to as RIPK1, RIP1 or RIP. RIP1 kinase has a crucial role whether the cell live or die. RIP1 is involved in the apoptosis and non-apoptotic cell death; necroptosis [1]. The intracellular domains of TNF receptor1 (TNFR1), FAS and TRAIL receptor 2 (TRAILR2) together include death domain (DD), they were stimulated by ligands tumor necrosis factor alpha (TNFα), Fas ligand (FASL) and TRAIL which recruit RIP1 and binding of their DD to that of RIP1. Stimulation of TNFR1 by TNFα leads to the formation of the complex I which leads to the activation of NF-kB has an important role in modulating the RIP1 of activation and activates an important cell survival program [2]. RIP1 activation can lead to cell death pathway by the formation of a RIP1-TNF receptor associated death domain protein (TRADD)-FAS-associated DD protein (FADD)—caspase 8 complexes (complex IIa), which stimulates caspase activation and leads to RIPK1-dependent apoptosis (RDA). [3-9]. If caspase-8 activity is blocked, the recruited protein receptor-interacting serine/threonine-protein kinase 3 (RIPK3) kinase which drives necroptosis by driving formation of a RIP1-RIP3-mixed lineage kinase domain-like (MLKL) complex (complex IIb), which drives the cell lysis and disruption of cell membrane [10-11].

Necroptosis and RIP1 have been serve a crucial checkpoint during embryonic development. The activation of necroptosis and RIP1 may represent an important pathological mechanism and implicated in many human diseases by mediating cell death and inflammation. Necroptosis may also has been related to disordered of pathogenesis of the central nervous system (CNS) diseases, atherosclerosis, Huntington's disease, colitis, steatohepatitis, acute hepatitis, stroke, myocardial infarction, the intestinal epithelium and skin. Therefore, necroptosis inhibitors are a crucial role for clinical drug development. [12-14]

Necroptosis can be inhibited by inactivating RIP1 kinases or RIP3 kinase. The first and often used inhibitor of necroptosis is RIP1-inhibitor necrostatin-1 (Nec-1). Nec-1 demonstrated efficiency in vitro and in vivo. Nec-1 ameliorated renal and brain ischemia/reperfusion injury, ConA-induced hepatitis, DSS-induced colitis and decreased the symptoms of Huntington's disease in a murine study [15-19].

The novel compounds of this invention inhibit RIP1 kinase activity and are, therefore, expected to be-useful in the treatment of disease and/or condition associated with inflammation and/or necroptotic cell death [20].

In recent, RIP1 kinase inhibitors differ structurally from necrostatin class of compounds [21-22].

References cited above, each of which is hereby incorporated by reference in its entirety:
1. Degterev, A., Hitomi, J., Germscheid, M., Ch'en, I., Korkina, O., Teng, X., Abbott, D., Cuny, G., Yuan, C., Wagner, G., Hedrick, S., Gerber, S., Lugovskoy, A. and Yuan, J. Identification of RIP1 kinase as a specific cellular target of necrostatins. Nat Chem Biol. 4, 313-321 (2008).
2. Ofengeim, D. and Yuan, J. Regulation of RIP1 kinase signalling at the crossroads of inflammation and cell death. Nat. Rev. Mol. Cell Biol. 14, 727-736 (2013).
3. Shan, B., Pan, H., Najafov, A. and Yuan, J. Necroptosis in development and diseases. Genes Dev. 32, 327-340 (2018).
4. Vanden Berghe, T., Linkermann, A., Jouan-Lanhouet, S., Walczak, H. and Vandenabeele, P. Regulated necrosis: the expanding network of non-apoptotic cell death pathways. Nature reviews. Molecular cell biology. 15, 135-147 (2014).
5. Newton, K. RIPK1 and RIPK3: critical regulators of inflammation and cell death. Trends in cell biology. 25, 347-353 (2015).
6. de Almagro, M. C. and Vucic, D. Necroptosis: Pathway diversity and characteristics. Semin Cell Dev Biol. 39, 56-62 (2015).
7. O'Donnell, M. A., Legarda-Addison, D., Skountzos, P., Yeh, W. C. and Ting, A. T. Ubiquitination of RIP1 regulates an NF-kappaB-independent cell-death switch in TNF signaling. Curr Biol. 17, 418-424 (2007).
8. Feoktistova, M., Geserick, P., Kellert, B., Dimitrova, D. P., Langlais, C., Hupe, M., Cain, K., MacFarlane, M., Hacker, G. and Leverkus, M. cIAPs block Ripoptosome formation, a RIP1/caspase-8 containing intracellular cell death complex differentially regulated by cFLIP isoforms. Molecular cell. 43, 449-463 (2011).
9. Bertrand, M. J., Milutinovic, S., Dickson, K. M., Ho, W. C, Boudreault, A., Durkin, J., Gillard, J. W., Jaquith, J. B., Morris, S. J. and Barker, P. A. cIAP1 and cIAP2 facilitate cancer cell survival by functioning as E3 ligases that promote RIP1 ubiquitination. Mol Cell. 30, 689-700 (2008).
10. Cho, Y. S., Challa, S., Moquin, D., Genga, R., Ray, T. D., Guildford, M. and Chan, F. K. Phosphorylation-driven assembly of the RIP1-RIP3 complex regulates programmed necrosis and virus-induced inflammation. Cell. 137, 1112-1123 (2009).
11. Sun, L., Wang, H., Wang, Z., He, S., Chen, S., Liao, D., Wang, L., Yan, J., Liu, W., Lei, X. and Wang, X. Mixed lineage kinase domain-like protein mediates necrosis signaling downstream of RIP3 kinase. Cell. 148, 213-227 (2012).
12. Zhao, J., Jitkaew, S., Cai, Z., Choksi, S., Li, Q., Luo, J. and Liu, Z. G. Mixed lineage kinase domain-like is a key receptor interacting protein 3 downstream component of TNF-induced necrosis. Proceedings of the National Academy of Sciences of the United States of America. 109, 5322-5327 (2012).
13. Sun, L., Wang, H., Wang, Z., He, S., Chen, S., Liao, D., Wang, L., Yan, J., Liu, W., Lei, X. and Wang, X. Mixed Lineage Kinase Domain-like Protein Mediates Necrosis Signaling Downstream of RIP3 Kinase. Cell. 148, 213-227 (2012).
14. Linkermann, A. and Green, D. R. Necroptosis. The New England journal of medicine. 370, 455-465 (2014).
15. Degterev, A., Huang, Z., Boyce, M., Li, Y., Jagtap, P., Mizushima, N. Cuny, G. D., Mitchison, T. J., Moskowitz, M. A. and Yuan, J. Chemical Inhibitor of Nonapoptotic Cell Death with Therapeutic Potential for Ischemic Brain Injury. Nat. Chem. Biol. 1, 112-119 (2005).
16. Linkermann, A., Brasen, J. H., Himmerkus, N., Liu, S., Huber, T. B., Kunzendorf, U. and Krautwald, S. Rip1 (Receptor-Interacting Protein Kinase 1) Mediates Necroptosis and Contributes to Renal Ischemia/Reperfusion Injury. Kidney Int. 81, 751-761 (2012).
17. Jouan-Lanhouet, S., Arshad, M. I., Piquet-Pellorce, C., Martin-Chouly, C., Le Moigne-Muller, G., Van Herreweghe, F., Takahashi, N., Sergent, O., Lagadic-Gossmann, D. and Vandenabeele, P. TRAIL Induces Necroptosis Involving RIPK1/RIPK3-Dependent PARP-1 Activation. Cell Death Differ. 19, 2003-2014 (2012).
18. Gunther, C., Martini, E., Wittkopf, N., Amann, K., Weigmann, B., Neumann, H., Waldner, M. J., Hedrick, S. M., Tenzer, S. and Neurath, M. F. Caspase-8 Regulates TNF-Alpha-Induced Epithelial Necroptosis and Terminal Ileitis. Nature. 477, 335-339 (2011).
19. Zhu, S., Zhang, Y., Bai, G. and Li, H. Necrostatin-1 Ameliorates Symptoms in R6/2 Transgenic Mouse Model of Huntington's Disease. Cell. Death Dis. 2, e115 (2011).
20. Newton, K., Dugger, D. L., Wickliffe, K. E., Kapoor, N., de Almagro, M. C, Vucic, D., Komuves, L., Ferrando, R. E., French, D. M., Webster, J., Roose-Girma, M., Warming, S. and Dixit, V. M. Activity of protein kinase RIPK3 determines whether cells die by necroptosis or apoptosis. Science. 343, 1357-1360 (2014).
21. Harris, P. A., Bandyopadhyay, D., Berger, S. B., Campobasso, N., Capriotti, C. A., Cox, J. A., Dare, L., Finger, J. N., Hoffman, S. J., Kahler, K. M., Lehr, R., Lich, J. D., Nagilla, R., Nolte, R. T., Ouellette, M. T., Pao, C. S., Schaeffer, M. C, Smallwood, A., Sun, H. H., Swift, B. A., Totoritis, R. D., Ward, P., Marquis, R. W., Bertin, J. and Gough, P. J. Discovery of Small Molecule RIP1 Kinase Inhibitors for the Treatment of Pathologies Associated with Necroptosis. ACS medicinal chemistry letters. 4, 1238-1243 (2013).
22. Najjar, M., Suebsuwong, C, Ray, S. S., Thapa, R. J., Maki, J. L., Nogusa, S., Shah, S., Saleh, D., Gough, P. J., Bertin, J., Yuan, J., Balachandran, S., Cuny, G. D. and Degterev, A. Structure Guided Design of Potent and Selective Ponatinib-Based Hybrid Inhibitors for RIPK1. Cell Rep. 24, 1850-1860 (2015).

### SUMMARY

This invention provides a compound of formula I, or a pharmaceutically acceptable salt, solvate, polymorph, ester, tautomer or prodrug thereof:

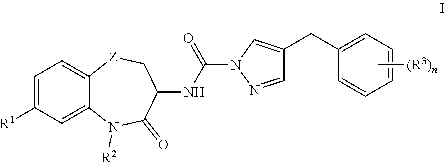

wherein
R¹ is —O[(C₁-C₆)alkyl]NR⁴R⁵, —O(C₁-C₆)alkyl, —O[gem-dimethylhydroxy(C₁-C₆)alkyl], —O(C₁-C₆)alkyl[(C₁-C₆)alkoxy], —O(C₁-C₆)alkyl[(C₃-C₆)cycloalkyl], —O(C₁-C₆)alkyl[(C₃-C₆)cycloalkyl]hydroxy, —O(C₃-C₆)cycloalkyl, —O(C₃-C₆)hydroxycycloalkyl, —O(C₁-C₆)alkyl-(hetAr² or hetAr³), —O(C₁-C₆)alkyl-(hetCyc¹ or hetCyc²), —C(O)NR⁴R⁵, —OC(O)NR⁴R⁵, —O[(C₁-C₃)alkyl]C(O)NR⁴R⁵, or

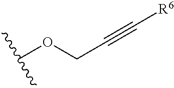

R² is H, CD₃, or optionally substituted by C₁-C₆ alkyl;
each R³ is independently H, methyl, CF₃, halogen, or cyano;
n is 1, 2 or 3;
Z is CH₂, NR², O, or S;
R⁴ is H or C₁-C₆ alkyl;
R⁵ is H, —(C₁-C₆)alkyl, —(C₁-C₆)fluoroalkyl, —(C₁-C₆)difluoroalkyl, —(C₁-C₆)trifluoroalkyl, -gem-dimethyl(C₁-C₆)hydroxy, —(C₁-C₆)hydroxyalkyl, —(C₂-C₆)dihydroxyalkyl, [(C₁-C₆)alkoxy](C₁-C₆)alkyl-, [(C₁-C₆)alkoxy]-[(C₁-C₆)alkoxy]-(C₁-C₆)alkyl-, —O(C₁-C₆)alkyl, —O(C₁-C₆)hydroxyalkyl, —O(C₁-C₆)alkyl[(C₁-C₆)alkoxy], —O(C₁-C₃)alkyl[(C₃-C₆)cycloalkyl], Cyc¹, Ar¹, —CH₂Ar¹, hetCyc¹, hetAr², hetAr³, hetCyc²(C₁-C₂)alkyl- or hetCyc³(C₁-C₂)alkyl-;
or NR⁴R⁵ forms a 4-6 membered heterocyclic ring having a ring nitrogen atom and optionally having a second ring heteroatom selected from N and O, wherein said ring is optionally substituted with one or more substituents independently selected from (C₁-C₆)alkyl, OH, alkoxy, and (C₁-C₆)hydroxyalkyl;
R⁶ is H, —(C₁-C₆)alkyl, —(C₃-C₆)cycloalkyl, -gem-dimethylhydroxy(C₁-C₆)alkyl, —(C₃-C₆)hydroxycycloalkyl, hetAr²;
hetAr¹ is a 5-membered heteroaryl ring having 2-3 ring heteroatoms, wherein at least 1 of said ring heteroatoms is N and said ring is optionally substituted with a substituent selected from (C₁-C₆)alkyl, NH₂, (C₁-C₆ hydroxyalkyl)NH—, (HO)₂P(═O)OCH₂—, (C₁-C₆)hydroxyalkyl, Cyc¹, and (C₁-C₆ alkyl)COOH;
Cyc¹ is a 3-6 membered cycloalkyl ring which is optionally substituted with one or more substituents independently selected from —(C₁-C₄ alkyl), OH, OCH₃, COOH, —(C₁-C₄ alkyl)OH, halogen and CF₃;
hetCyc¹ is a carbon-linked 4-6 membered heterocyclic ring optionally substituted with a substituent selected from (C₁-C₆)alkyl;
hetCyc² is a 5-6 membered heterocyclic ring having a ring nitrogen atom and optionally having a second ring heteroatom selected from N and O, wherein said ring is optionally substituted with a substituent selected from (C₁-C₆)alkyl, OH, (C₁-C₆)alkoxy, halogen and oxo;
hetCyc³ is a bridged 8-membered heterocyclic ring having a ring nitrogen atom and optionally having a ring oxygen atom;
Ar¹ is phenyl optionally substituted with one or more substituents independently selected from (C₁-C₆)alkoxy, halogen, (C₁-C₆)alkyl and CF₃;
hetAr² is pyridyl optionally substituted with one or more substituents independently selected from halogen, CF₃, (C₁-C₆)alkyl and (C₁-C₆)alkoxy;
hetAr³ is a 5-membered heteroaryl having 2-3 ring heteroatoms independently selected from N, O and S and optionally substituted with (C₁-C₆)alkyl and OH;
Compounds of Formula I further include the absolute configuration compounds of Formula IIa and IIb,

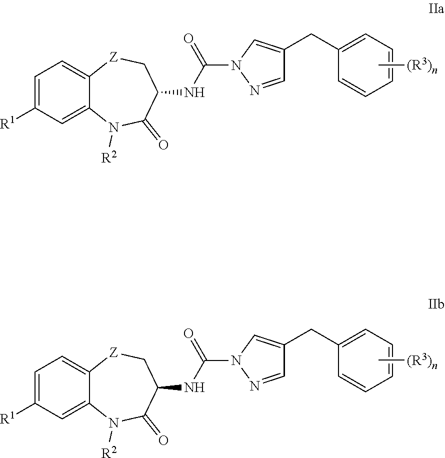

or pharmaceutically acceptable salt, solvate, polymorph, ester, tautomer or prodrug thereof.

Wherein
R¹ is —O[(C₁-C₆)alkyl]NR⁴R⁵, —O(C₁-C₆)alkyl, —O[gem-dimethylhydroxy(C₁-C₆)alkyl], —O(C₁-C₆)alkyl[(C₁-C₆)alkoxy], —O(C₁-C₆)alkyl[(C₃-C₆)cycloalkyl], —O(C₁-C₆)alkyl[(C₃-C₆)cycloalkyl]hydroxy, —O(C₃-C₆)cycloalkyl, —O(C₃-C₆)hydroxycycloalkyl, —O(C₁-C₆)alkyl-(hetAr² or hetAr³), —O(C₁-C₆)alkyl-(hetCyc¹ or hetCyc²), —C(O)NR⁴R⁵, —OC(O)NR⁴R⁵, —O[(C₁-C₃)alkyl]C(O)NR⁴R⁵, or

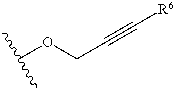

R² is H, CD₃, or optionally substituted by C₁-C₆ alkyl;
each R³ is independently H, methyl, CF₃, halogen, or cyano;
n is 1, 2 or 3;
Z is CH₂, NR², O, or S;
R⁴ is H or C₁-C₆ alkyl;
R⁵ is H, —(C₁-C₆)alkyl, —(C₁-C₆)fluoroalkyl, —(C₁-C₆)difluoroalkyl, —(C₁-C₆)trifluoroalkyl, -gem-dimethyl(C₁-C₆)hydroxy, —(C₁-C₆)hydroxyalkyl, —(C₂-C₆)dihydroxyalkyl, [(C₁-C₆)alkoxy](C₁-C₆)alkyl-, [(C₁-C₆)alkoxy]-[(C₁-C₆)alkoxy]-(C₁-C₆)alkyl-, —O(C₁-C₆)alkyl, —O(C₁-C₆)hydroxyalkyl, —O(C₁-C₆)alkyl[(C₁-C₆)alkoxy], —O(C₁-C₃)alkyl[(C₃-C₆)cycloalkyl], Cyc¹, Ar¹, —CH₂Ar¹, hetCyc¹, hetAr², hetAr³, hetCyc²(C₁-C₂)alkyl- or hetCyc³(C₁-C₂)alkyl-;
or NR⁴R⁵ forms a 4-6 membered heterocyclic ring having a ring nitrogen atom and optionally having a second ring heteroatom selected from N and O, wherein said ring is optionally substituted with one or more substituents independently selected from (C₁-C₆)alkyl, OH, alkoxy, and (C₁-C₆)hydroxyalkyl;
R⁶ is H, —(C₁-C₆)alkyl, —(C₃-C₆)cycloalkyl, -gem-dimethylhydroxy(C₁-C₆)alkyl, —(C₃-C₆)hydroxycycloalkyl, hetAr²;

In certain embodiments, the present invention provides compounds of Formula III,

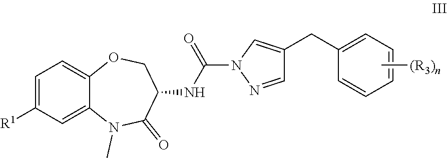

or pharmaceutically acceptable salt, solvate, polymorph, ester, tautomer or prodrug thereof.

Wherein
R¹ is —O[(C₁-C₆)alkyl]NR⁴R⁵, —O(C₁-C₆)alkyl, —O[gem-dimethylhydroxy(C₁-C₆)alkyl], —O(C₁-C₆)alkyl[(C₁-C₆)alkoxy], —O(C₁-C₆)alkyl[(C₃-C₆)cycloalkyl], —O(C₁-C₆)alkyl[(C₃-C₆)cycloalkyl]hydroxy, —O(C₃-C₆)cycloalkyl, —O(C₃-C₆)hydroxycycloalkyl, —O(C₁-C₆)alkyl-(hetAr² or hetAr³), —O(C₁-C₆)alkyl-(hetCyc¹ or hetCyc²), —C(O)NR⁴R⁵, —OC(O)NR⁴R⁵, —O[(C₁-C₃)alkyl]C(O)NR⁴R⁵, or

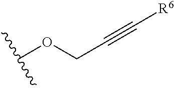

each R³ is independently H, methyl, CF₃, halogen, or cyano;
n is 1, 2 or 3;
R⁴ is H or C₁-C₆ alkyl;
R⁵ is H, —(C₁-C₆)alkyl, —(C₁-C₆)fluoroalkyl, —(C₁-C₆)difluoroalkyl, —(C₁-C₆)trifluoroalkyl, -gem-dimethyl(C₁-C₆)hydroxy, —(C₁-C₆)hydroxyalkyl, —(C₂-C₆)dihydroxyalkyl, [(C₁-C₆)alkoxy](C₁-C₆)alkyl-, [(C₁-C₆)alkoxy]-[(C₁-C₆)alkoxy]-(C₁-C₆)alkyl-, —O(C₁-C₆)alkyl, —O(C₁-C₆)hydroxyalkyl, —O(C₁-C₆)alkyl[(C₁-C₆)alkoxy], —O(C₁-C₃)alkyl[(C₃-C₆)cycloalkyl], Cyc¹, Ar¹, —CH₂Ar¹, hetCyc¹, hetAr², hetAr³, hetCyc²(C₁-C₂)alkyl- or hetCyc³(C₁-C₂)alkyl-;
or NR⁴R⁵ forms a 4-6 membered heterocyclic ring having a ring nitrogen atom and optionally having a second ring heteroatom selected from N and O, wherein said ring is optionally substituted with one or more substituents independently selected from (C₁-C₆)alkyl, OH, alkoxy, and (C₁-C₆)hydroxyalkyl;
R⁶ is H, —(C₁-C₆)alkyl, —(C₃-C₆)cycloalkyl, -gem-dimethylhydroxy(C₁-C₆)alkyl, —(C₃-C₆)hydroxycycloalkyl, hetAr²;

In certain embodiments, the present invention is directed to a pharmaceutical composition comprising an effective amount of a compound of formula I or a pharmaceutically acceptable salt, solvate, polymorph, ester, tautomer or prodrug thereof. In some embodiments, the pharmaceutical composition further comprises a pharmaceutically acceptable carrier, adjuvants and/or excipients.

In certain embodiments, such a composition may contain at least one of preservatives, agents for delaying absorption, fillers, binders, adsorbents, buffers, disintegrating agents, solubilizing agents, and other carriers, adjuvants and/or excipients as inert ingredients. The composition may be formulated with a method well-known in the art.

In certain embodiments, the present invention is directed to a method of treating a disease in an individual suffering from said disease comprising administering to said individual a therapeutically effective amount of a composition comprising a compound of formula I or a pharmaceutically acceptable salt, solvate, polymorph, ester, tautomer or prodrug thereof.

In certain embodiments, the present invention is directed to a method of treating a disorder in a mammal, comprising administering to said mammal a therapeutically effective amount of a compound of formula I or a pharmaceutically acceptable salt, solvate, polymorph, ester, tautomer or pro-drug thereof.

In certain embodiments, the present invention is directed to a method of treating a disorder in a human, comprising administering to said human a therapeutically effective amount of a compound of formula I or a pharmaceutically acceptable salt, solvate, polymorph, ester, tautomer or pro-drug thereof.

In certain embodiments, RIP1 kinase-mediated diseases or disorders are described herein and inflammatory or immune-regulatory disease or disorders include inflammatory bowel disease (including Crohn's disease and ulcerative colitis), psoriasis, systemic lupus erythematosus (SLE), retinal detachment, retinitis pigmentosa, arthritis (including rheumatoid arthritis, spondylarthritis, gout, osteoarthritis, and systemic onset juvenile idiopathic arthritis (SoJIA)), graft-versus-host diseases brought about by transplantation, nonalcoholic steatohepatitis (NASH), ischemia reperfusion, multiple sclerosis, tumor necrosis factor receptor-associated periodic syndrome, multiple organ dysfunction syndrome (MODS), thermal injury/burn, systemic inflammatory response syndrome (SIRS), radiation injury, radiotherapy, chemotherapy, pneumonias, hemorrhagic shock, trauma (including multiple trauma), traumatic brain injury, acute pancreatitis, critical illness (in general), sepsis, septic shock, Stevens-Johnson syndrome, toxic epidermal necrolysis, stroke, heat stroke, stroke-associated pneumonia, Multi-Organ Dysfunction Syndrome (MODS), Acute Respiratory Distress Syndrome (ARDS), intestinal obstruction, liver cirrhosis, surgery, major abdominal operations, abdominal aortic aneurysm repair, large bowel resections, ischemia reperfusion injury (including ischemia reperfusion injury of solid organs, (gut, brain, liver, kidney), and limb ischemia), bowel ischemia (small intestine and large intestine), cardiac surgery requiring cardio-pulmonary bypass, autoimmune hepatitis, autoimmune hepatobiliary diseases, autoimmune ITP, Parkinson's Disease, Lewy body dementia, multiple system atrophy, Parkinson-plus syndromes, tauopathies, Alzheimer's Disease, Frontotemporal dementia, amyotrophic lateral sclerosis, spinal muscular atrophy, primary lateral sclerosis, Huntington's disease, ischemia, stroke, intracranial hemorrhage, cerebral hemorrhage, muscular dystrophy, progressive muscular atrophy, progressive muscular atrophy, pseudobulbar palsy, spinal muscular atrophy, inherited muscular atrophy, peripheral neuropathies, progressive supranuclear palsy, corticobasal degeneration, demyelinating disease, allergic disease, asthma, atopic dermatitis, type I diabetes, Wegener's granulomatosis, Behcet's disease, and interleukin-1 converting enzyme associated fever syndrome.

In certain embodiments, the present invention is directed to a method of treating a pancreatic cancer, metastatic adenocarcinoma of the pancreas, pancreatic ductal adenocarcinoma, mesothelioma, melanoma, colorectal cancer, acute myeloid leukemia, metastasis, glioblastoma, breast cancer, gallbladder cancer, clear cell renal carcinoma, non-small cell lung carcinoma, and radiation induced necrosis certain the RIP1 kinase-mediated disease or disorder in a mammal, including a human, comprising administering to said mammal a therapeutically effective amount of a compound of formula I, or a pharmaceutically acceptable salt, ester, prodrug, solvate, such as hydrate, polymorph or tautomer thereof.

In certain embodiments, the present invention is directed to a method of treating a disorder or condition which is modulated by the RIP1 kinase in a mammal, including a human, comprising administering to said mammal an amount of the compound of formula I, or a pharmaceutically acceptable salt, ester, prodrug, solvate, such as hydrate, polymorph or tautomer thereof, effective to modulate said cascade. The appropriate dosage for a particular patient can be determined, according to known methods, by those skilled in the art.

In certain embodiments, the present invention is directed to use of compound of formula I or a pharmaceutically acceptable salt, ester, prodrug, solvate, such as hydrate, polymorph or tautomer thereof in the preparation of a pharmaceutical composition. The pharmaceutical composition can be used for treating a disorder or condition which is modulated by the RIP1 kinase in a mammal, including a human.

In certain embodiments, the present invention is directed to a pharmaceutical composition comprising a compound of formula I or a pharmaceutically acceptable salt, solvate, polymorph, ester, tautomer or prodrug thereof. In some embodiments, the pharmaceutical composition is in a form suitable for oral administration. In further or additional embodiments, the pharmaceutical composition is in the form of a tablet, capsule, pill, powder, sustained release formulation, solution and suspension. In some embodiments, the pharmaceutical composition is in a form suitable for parenteral injection, such as a sterile solution, suspension or emulsion; for topical administration as an ointment or cream or for rectal administration as a suppository. In further or additional embodiments, the pharmaceutical composition is in unit dosage forms suitable for single administration of precise dosages. In further or additional embodiments, the amount of compound of formula I is in the range of about 0.001 to about 1000 mg/kg body weight/day. In further or additional embodiments, the amount of compound of formula I is in the range of about 0.5 to about 50 mg/kg body weight/day.

In certain embodiments, the present invention is directed to a process for preparing a compound of formula I or a pharmaceutically acceptable salt, solvate, polymorph, ester, tautomer or prodrug thereof.

### DETAILED DESCRIPTION

The novel features of the invention are set forth with particularity in the appended claims. A better understanding of the features and advantages of the present invention will be obtained by reference to the following detailed description that sets forth illustrative embodiments, in which the principles of the invention are utilized.

While preferred embodiments of the present invention have been shown and described herein such embodiments are provided by way of example only. It should be understood that various alternatives to the embodiments of the invention described herein may be employed in practicing the invention. Those ordinary skilled in the art will appreciate that numerous variations, changes, and substitutions are possible without departing from the invention. It is intended that the following claims define the scope of aspects of the invention and that methods and structures within the scope of these claims and their equivalents be covered thereby.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

### Certain Chemical Terminology

Unless defined otherwise, all technical and scientific terms used herein have the same meaning as is commonly understood by one of skill in the art to which the claimed subject matter belongs. All patents, patent applications, published materials referred to throughout the entire disclosure herein, unless noted otherwise, are incorporated by reference in their entirety. In the event that there is a plurality of definitions for terms herein, those in this section prevail. Where reference is made to a URL or other such identifier or address, it is understood that such identifiers can change and particular information on the internet can come and go, but equivalent information can be found by searching the internet or other appropriate reference source. Reference thereto evidences the availability and public dissemination of such information.

It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a”, “an” and “the” include plural referents unless the context clearly dictates otherwise. It should also be noted that use of “or” means “and/or” unless stated otherwise. Furthermore, use of the term “including” as well as other forms, such as “include”, “includes”, and “included” is not limiting. Likewise, use of the term “comprising” as well as other forms, such as “comprise”, “comprises”, and “comprised” is not limiting.

Definition of standard chemistry terms may be found in reference works, including Carey and Sundberg “ADVANCED ORGANIC CHEMISTRY 4^{TH} ED.” Vols. A (2000) and B (2001), Plenum Press, New York. Unless otherwise indicated, conventional methods of mass spectroscopy, NMR, HPLC, IR and UV/Vis spectroscopy and pharmacology, within the skill of the art are employed. Unless specific definitions are provided, the nomenclature employed in connection with, and the laboratory procedures and techniques of, analytical chemistry, synthetic organic chemistry, and medicinal and pharmaceutical chemistry described herein are those known in the art. Standard techniques can be used for chemical syntheses, chemical analyses, pharmaceutical preparation, formulation, and delivery, and treatment of patients. Reactions and purification techniques can be performed e.g., using kits of manufacturer's specifications or as commonly accomplished in the art or as described herein. The foregoing techniques and procedures can be generally performed of conventional methods well known in the art and as described in various general and more specific references that are cited and discussed throughout the present specification. Throughout the specification, groups and substituents thereof can be chosen by one skilled in the field to provide stable moieties and compounds.

Unless otherwise noted, the use of general chemical terms, such as though not limited to “alkyl,” “amine,” “aryl,” are equivalent to their optionally substituted forms. For example, “alkyl,” as used herein, includes optionally substituted alkyl.

The term “optional” or “optionally” means that the subsequently described event or circumstance may or may not occur, and that the description includes instances where said event or circumstance occurs and instances in which it does not. For example, “optionally substituted alkyl” means either “alkyl” or “substituted alkyl” as defined below. Further, an optionally substituted group may be un-substituted (e.g., CH₂CH₃), fully substituted (e.g., CF₂CF₃), mono-substituted (e.g., CH₂CH₂F) or substituted at a level anywhere in-between fully substituted and mono-substituted (e.g., CH₂CHF₂, CF₂CH₃, CFHCHF₂, etc.). It will be understood by those skilled in the art with respect to any group containing one or more substituents that such groups are not intended to introduce any substitution or substitution patterns (e.g., substituted alkyl includes optionally substituted cycloalkyl groups, which in turn are defined as including optionally substituted alkyl groups, potentially ad infinitum) that are sterically impractical and/or synthetically non-feasible. Thus, any substituents described should generally be understood as having a maximum molecular weight of about 1,000 daltons, and more typically, up to about 500 daltons (except in those instances where macromolecular substituents are clearly intended, e.g., polypeptides, polysaccharides, polyethylene glycols, DNA, RNA and the like).

As used herein, C₁-Cn, includes C₁-C₂, C₁-C₃, . . . C₁-Cn. By way of example only, a group designated as “C₁-C₄” indicates that there are one to four carbon atoms in the moiety, i.e. groups containing 1 carbon atom, 2 carbon atoms, 3 carbon atoms or 4 carbon atoms, as well as the ranges C₁-C₂ and C₁-C₃. Thus, by way of example only, “C₁-C₄ alkyl” indicates that there are one to four carbon atoms in the alkyl group, i.e., the alkyl group is selected from among methyl, ethyl, propyl, iso-propyl, n-butyl, isobutyl, sec-butyl, and t-butyl. Whenever it appears herein, a numerical range such as “1 to 10” refers to each integer in the given range; e.g., “1 to 10 carbon atoms” means that the group may have 1 carbon atom, 2 carbon atoms, 3 carbon atoms, 4 carbon atoms, 5 carbon atoms, 6 carbon atoms, 7 carbon atoms, 8 carbon atoms, 9 carbon atoms, or 10 carbon atoms.

The terms “heteroatom” or “hetero” as used herein, alone or in combination, refer to an atom other than carbon and hydrogen. Heteroatoms are independently selected from among oxygen, nitrogen, sulfur, phosphorous, silicon, selenium and tin but are not limited to these atoms. In embodiments in which two or more heteroatoms are present, the two or more heteroatoms can be the same as each another, or some or all of the two or more heteroatoms can each be different from the others.

The term “alkyl” as used herein, alone or in combination, refers to an optionally substituted straight-chain, or optionally substituted branched-chain saturated hydrocarbon monoradical having from one to about ten carbon atoms, more preferably one to six carbon atoms. Examples include, but are not limited to methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl and hexyl, and longer alkyl groups, such as heptyl, octyl and the like. Whenever it appears herein, a numerical range such as “C₁-C₆ alkyl” or “C_{1_6} alkyl”, means that the alkyl group may consist of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated.

The term “aliphatic” as used herein, alone or in combination, refers to an optionally substituted, straight-chain or branched-chain, non-cyclic, saturated, partially unsaturated, or fully unsaturated nonaromatic hydrocarbon. Thus, the term collectively includes alkyl, alkenyl and alkynyl groups.

The terms “cycle”, “cyclic”, “ring” and “membered ring” as used herein, alone or in combination, refer to any covalently closed structure, including alicyclic, heterocyclic, aromatic, heteroaromatic and polycyclic fused or non-fused ring systems as described herein. Rings can be optionally substituted. Rings can form part of a fused ring system. The term “membered” is meant to denote the number of skeletal atoms that constitute the ring. Thus, by way of example only, cyclohexane, pyridine, pyran and pyrimidine are six-membered rings and cyclopentane, pyrrole, tetrahydrofuran and thiophene are five-membered rings.

The term “cycloalkyl” as used herein, alone or in combination, refers to an optionally substituted, saturated, hydrocarbon monoradical ring, containing from three to about fifteen ring carbon atoms or from three to about ten ring carbon atoms, though may include additional, non-ring carbon atoms as substituents (e.g. methylcyclopropyl).

A non-limiting example of “cycloalkyl” includes azinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexyl, 3-azabicyclo [4.1.0]heptyl, 3H-indolyl and quinolizinyl and the like. The terms also include all ring forms of the carbohydrates, including but not limited to the monosaccharides, the disaccharides and the oligosaccharides.

The term “aromatic” as used herein, refers to a planar, cyclic or polycyclic, ring moiety having a delocalized at-electron system containing 4n+2 n electrons, where n is an integer. Aromatic rings can be formed by five, six, seven, eight, nine, or more than nine atoms. Aromatics can be optionally substituted and can be monocyclic or fused-ring polycyclic. The term aromatic encompasses both all carbon containing rings (e.g., phenyl) and those rings containing one or more heteroatoms (e.g., pyridine).

### Certain Pharmaceutical Terminology

The term “Necroptosis assay for RIP1 activity” as used herein refers to a compound that exhibits an IC₅₀, with respect to RIP1 kinase activity, of no more than about 100 □M or not more than about 50 □M, as measured in the kinase assay described generally herein. “IC₅₀” is that concentration of inhibitor which reduces the activity of an enzyme to half-maximal level. Compounds described herein have been discovered to exhibit inhibition against RIPK1. Compounds of the present invention preferably exhibit an IC₅₀ with respect to RIPK1 of no more than about 10 □M, more preferably, no more than about 5 □M, even more preferably not more than about 1 □M, and most preferably, not more than about 200 nM, as measured in necroptosis assay described herein.

The term “selective,” “selectively,” or “selectivity” as used herein refers to a compound of this invention having a lower IC₅₀ value for the enzyme as compared to any other enzymes (e.g., at least 2, 5, 10 or more-fold lower).

The term “subject”, “patient” or “individual” as used herein in reference to individuals suffering from a disorder, a condition, and the like, encompasses mammals and non-mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods and compositions provided herein, the mammal is a human.

The terms “treat,” “treating” or “treatment,” and other grammatical equivalents as used herein, include alleviating, abating or ameliorating a disease or condition symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition, and are intended to include prophylaxis. The terms further include achieving a therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made.

The terms “effective amount”, “therapeutically effective amount” or “pharmaceutically effective amount” as used herein, refer to a sufficient amount of at least one agent or compound being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in a disease. An appropriate “effective” amount in any individual case may be determined using techniques, such as a dose escalation study.

The terms “administer,” “administering”, “administration,” and the like, as used herein, refer to the methods that may be used to enable delivery of compounds or compositions to the desired site of biological action. These methods include, but are not limited to oral routes, intraduodenal routes, parenteral injection (including intravenous, subcutaneous, intraperitoneal, intramuscular, intravascular or infusion), topical and rectal administration. Those of skill in the art are familiar with administration techniques that can be employed with the compounds and methods described herein, e.g., as discussed in Goodman and Gilman, The Pharmacological Basis of Therapeutics, current ed.; Pergamon; and Remington's, Pharmaceutical Sciences (current edition), Mack Publishing Co., Easton, Pa. In preferred embodiments, the compounds and compositions described herein are administered orally.

The term “acceptable” as used herein, with respect to a formulation, composition or ingredient, means having no persistent detrimental effect on the general health of the subject being treated.

The term “pharmaceutically acceptable” as used herein, refers to a material, such as a carrier or diluent, which does not abrogate the biological activity or properties of the compounds described herein, and is relatively nontoxic, i.e., the material may be administered to an individual without causing undesirable biological effects or interacting in a deleterious manner with any of the components of the composition in which it is contained.

The term “pharmaceutical composition,” as used herein, refers to a biologically active compound, optionally mixed with at least one pharmaceutically acceptable chemical component, such as, though not limited to carriers, stabilizers, diluents, dispersing agents, suspending agents, thickening agents, and/or excipients.

The term “carrier” as used herein, refers to relatively nontoxic chemical compounds or agents that facilitate the incorporation of a compound into cells or tissues.

The term “agonist,” as used herein, refers to a molecule such as a compound, a drug, an enzyme activator or a hormone modulator which enhances the activity of another molecule or the activity of a receptor site.

The term “antagonist,” as used herein, refers to a molecule such as a compound, a drug, an enzyme inhibitor, or a hormone modulator, which diminishes, or prevents the action of another molecule or the activity of a receptor site.

The term “modulate,” as used herein, means to interact with a target either directly or indirectly so as to alter the activity of the target, including, by way of example only, to enhance the activity of the target, to inhibit the activity of the target, to limit the activity of the target, or to extend the activity of the target.

The term “modulator,” as used herein, refers to a molecule that interacts with a target either directly or indirectly. The interactions include, but are not limited to, the interactions of an agonist and an antagonist.

The term “pharmaceutically acceptable salt” as used herein, refers to salts that retain the biological effectiveness of the free acids and bases of the specified compound and that are not biologically or otherwise undesirable. Compounds described herein may possess acidic or basic groups and therefore may react with any of a number of inorganic or organic bases, and inorganic and organic acids, to form a pharmaceutically acceptable salt. These salts can be prepared in situ during the final isolation and purification of the compounds of the invention, or by separately reacting a purified compound in its free base form with a suitable organic or inorganic acid, and isolating the salt thus formed. Examples of pharmaceutically acceptable salts include those salts prepared by reaction of the compounds described herein with a mineral or organic acid or an inorganic base, such salts including, acetate, acrylate, adipate, alginate, aspartate, benzoate, benzenesulfonate, bisulfate, bisulfite, bromide, butyrate, butyn-1,4-dioate, camphorate, camphorsulfonate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, hydroxybutyrate, hydrochloride, hydrobromide, hydroiodide, 2-hydroxyethanesulfonate, iodide, isobutyrate, lactate, maleate, malonate, methanesulfonate, mandelate. metaphosphate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, suberate, sebacate, sulfonate, tartrate, thiocyanate, tosylate, undecanoate, and xylene sulfonate. Other acids, such as oxalic, while not in themselves pharmaceutically acceptable, may be employed in the preparation of salts useful as intermediates in obtaining the compounds of the invention and their pharmaceutically acceptable acid addition salts (See examples at Berge et al., J. Pharm. Sci. 1977, 66, 1-19.). Further, those compounds described herein which may comprise a free acid group may react with a suitable base, such as the hydroxide, carbonate or bicarbonate of a pharmaceutically acceptable metal cation, with ammonia, or with a pharmaceutically acceptable organic primary, secondary or tertiary amine. Representative alkali or alkaline earth salts include the lithium, sodium, potassium, calcium, magnesium, and aluminum salts and the like. Illustrative examples of bases include sodium hydroxide, potassium hydroxide, choline hydroxide, sodium carbonate, and the like. Representative organic amines useful for the formation of base addition salts include ethylamine, diethylamine, ethylenediamine, ethanolamine, diethanolamine, piperazine and the like. It should be understood that the compounds described herein also include the quaternization of any basic nitrogen-containing groups they may contain. Water or oil-soluble or dispersible products may be obtained by such quaternization. See, for example, Berge et al., supra.

The term “solvate” as used herein refers to a combination of a compound of this invention with a solvent molecule formed by solvation. In some situations, the solvate refers to a hydrate, i.e., the solvent molecule is a water molecule, the combination of a compound of this invention and water forms a hydrate.

The term “polymorph” or “polymorphism” as used herein refers to a compound of this invention present in different crystal lattice forms.

The term “ester” as used herein refers to a derivative of a compound of this invention derived from an oxoacid group and a hydroxyl group, either one of which can be present at the compound of this invention.

The term “tautomer” as used herein refers to an isomer readily interconverted from a compound of this invention by e.g., migration of a hydrogen atom or proton.

The term “pharmaceutically acceptable derivative or prodrug” as used herein, refers to any pharmaceutically acceptable salt, ester, salt of an ester or other derivative of a compound of this invention, which, upon administration to a recipient, is capable of providing, either directly or indirectly, a compound of this invention or a pharmaceutically active metabolite or residue thereof. Particularly favored derivatives or prodrugs are those that increase the bioavailability of the compounds of this invention when such compounds are administered to a patient (e.g., by allowing orally administered compound to be more readily absorbed into blood) or which enhance delivery of the parent compound to a biological compartment (e.g., the brain or lymphatic system).

Pharmaceutically acceptable prodrugs of the compounds described herein include, but are not limited to, esters, carbonates, thiocarbonates, N-acyl derivatives, N-acyloxyalkyl derivatives, quaternary derivatives of tertiary amines, N-Mannich bases, Schiff bases, amino acid conjugates, phosphate esters, metal salts and sulfonate esters. Various forms of prodrugs are well known in the art. See for example Design of Prodrugs, Bundgaard, A. Ed., Elseview, 1985 and Method in Enzymology, Widder, K. et al., Ed.; Academic, 1985, vol. 42, p. 309-396; Bundgaard, H. “Design and Application of Prodrugs” in A Textbook of Drug Design and Development, Krosgaard-Larsen and H. Bundgaard, Ed., 1991, Chapter 5, p. 113-191; and Bundgaard, H., Advanced Drug Delivery Review, 1992, 8, 1-38, each of which is incorporated herein by reference. The prodrugs described herein include, but are not limited to, the following groups and combinations of these groups; amine derived prodrugs: Hydroxy prodrugs include, but are not limited to acyloxyalkyl esters, alkoxycarbonyloxyalkyl esters, alkyl esters, aryl esters and disulfide containing esters.

The terms “enhance” or “enhancing,” as used herein, means to increase or prolong either in potency or duration of a desired effect. Thus, in regard to enhancing the effect of therapeutic agents, the term “enhancing” refers to the ability to increase or prolong, either in potency or duration, the effect of other therapeutic agents on a system.

An “enhancing-effective amount,” as used herein, refers to an amount adequate to enhance the effect of another therapeutic agent in a desired system.

The terms “pharmaceutical combination”, “administering an additional therapy”, “administering an additional therapeutic agent” and the like, as used herein, refer to a pharmaceutical therapy resulting from mixing or combining more than one active ingredient and includes both fixed and non-fixed combinations of the active ingredients. The term “fixed combination” means that at least one of the compounds described herein, and at least one co-agent, are both administered to a patient simultaneously in the form of a single entity or dosage. The term “non-fixed combination” means that at least one of the compounds described herein, and at least one co-agent, are administered to a patient as separate entities either simultaneously, concurrently or sequentially with variable intervening time limits, wherein such administration provides effective levels of the two or more compounds in the body of the patient. These also apply to cocktail therapies, e.g. the administration of three or more active ingredients.

The terms “co-administration”, “administered in combination with” and their grammatical equivalents or the like, as used herein, are meant to encompass administration of the selected therapeutic agents to a single patient, and are intended to include treatment regimens in which the agents are administered by the same or different route of administration or at the same or different times. In some embodiments the compounds described herein will be co-administered with other agents. These terms encompass administration of two or more agents to an animal so that both agents and/or their metabolites are present in the animal at the same time. They include simultaneous administration in separate compositions, administration at different times in separate compositions, and/or administration in a composition in which both agents are present. Thus, in some embodiments, the compounds of the invention and the other agent (s) are administered in a single composition.

The term “metabolite,” as used herein, refers to a derivative of a compound which is formed when the compound is metabolized.

The term “active metabolite,” as used herein, refers to a biologically active derivative of a compound that is formed when the compound is metabolized.

The term “metabolized,” as used herein, refers to the sum of the processes (including, but not limited to, hydrolysis reactions and reactions catalyzed by enzymes) by which a particular substance is changed by an organism. Thus, enzymes may produce specific structural alterations to a compound. For example, cytochrome P450 catalyzes a variety of oxidative and reductive reactions while uridine diphosphate glucuronyl transferases catalyze the transfer of an activated glucuronic-acid molecule to aromatic alcohols, aliphatic alcohols, carboxylic acids, amines and free sulfhydryl groups. Further information on metabolism may be obtained from The Pharmacological Basis of Therapeutics, 9th Edition, McGraw-Hill (1996).

### EXPERIMENTAL PART

NMR spectra were recorded in CDCl₃ solution in 5-mm o.d. tubes (Norell, Inc. 507-HP) at 30° C. and were collected on JEOL at 400 MHz for ¹H. The chemical shifts (δ) are relative to tetramethylsilane (TMS=0.00 ppm) and expressed in ppm. LC/MS was taken on Ion-trap Mass Spectrometer on ISQ EM, Thermo Fisher Vanquish Flex (Column: hypersil Gold (C18, Ø2.1×50 mm, 1.9 μm, 120 Å, 30° C.) operating in ESI(+) ionization mode; flow rate=0.5 mL/min. Mobile phase=0.01% heptafluorobutyric acid (HFBA) and 1.0% isopropyl alcohol (IPA) in water or CH₃CN.

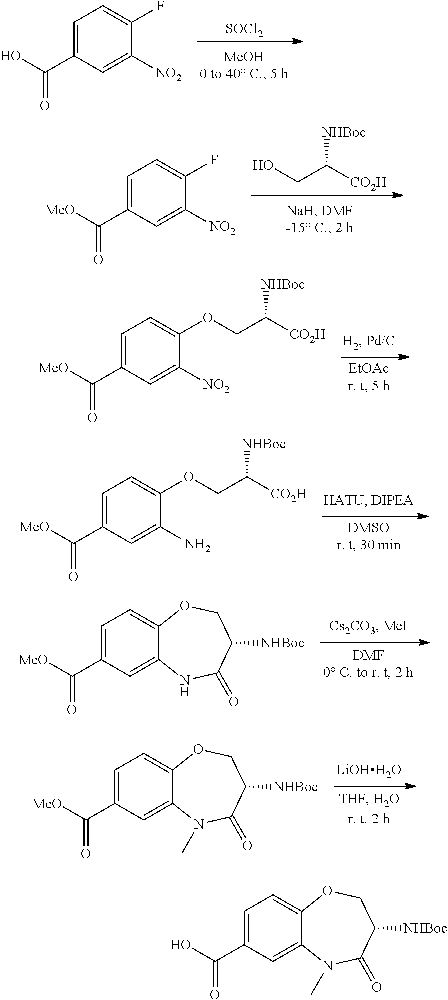

### Intermediate 1:

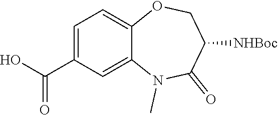 [SMILES: CN1C(=O)[C@@H](NC(=O)OC(C)(C)C)COc2ccc(C(=O)O)cc21]

### Step A: methyl 4-fluoro-3-nitrobenzoate

To a solution of 4-fluoro-3-nitrobenzoic acid (3.00 g, 16.2 mmol) in MeOH (16 mL) was dropwise added SOCl₂ (3.55 mL, 48.6 mmol) at 0° C. The reaction mixture was stirred at 40° C. for 5 hours. After concentration in vacuo, the residue was partitioned between EtOAc and water. The separated organic layer was washed with saturated aq. NaHCO₃, dried over Na₂SO₄, filtered, and concentrated in vacuo to afford methyl 4-fluoro-3-nitrobenzoate (3.20 g, 99%) as yellow green oil, which was used for the next step without purification. ¹H-NMR (400 MHz, CDCl₃): δ 8.75 (1H, dd, J=7.2, 2.4 Hz), 8.33 (1H, ddd, J=8.8, 4.0, 2.4 Hz), 7.39 (1H, dd, J=10.4, 8.8 Hz), 3.98 (3H, s).

### Step B: N-(tert-butoxycarbonyl)-O-(4-(methoxycarbonyl)-2-nitrophenyl)-L-serine

To a suspension of NaH (55 wt %, 0.300 g, 7.53 mmol) in DMF (5.0 mL) was slowly added a solution of (tert-butoxycarbonyl)-L-serine (0.773 g, 3.39 mmol) in DMF (5.0 mL) at −15° C. The mixture was stirred at −15° C. for 1 hour. After addition of a solution of methyl 4-fluoro-3-nitrobenzoate (0.500 g, 2.51 mmol) in DMF (2.5 mL) at −15° C., the reaction mixture was stirred at −15° C. for further 1 hour. After carefully quenched with 1 M aq. HCl until pH 3-4 at −15° C., the mixture was extracted with EtOAc twice. The combined organic layers were washed with water and brine, dried over Na₂SO₄, filtered, and concentrated in vacuo to afford N-(tert-butoxycarbonyl)-O-(4-(methoxycarbonyl)-2-nitrophenyl)-L-serine (1.42 g, crude) as yellow oil, which was used for the next step without further purification. ¹H-NMR (400 MHz, CDCl₃): δ 8.52 (1H, d, J=2.4 Hz), 8.21 (1H, dd, J=8.8, 2.0 Hz), 7.13 (1H, d, J=9.2 Hz), 5.64 (1H, d, J=8.0 Hz), 4.75-4.73 (1H, m), 4.68 (1H, dd, J=9.6, 2.4 Hz), 4.48 (1H, dd, J=8.8, 2.8 Hz), 3.94 (3H, s), 1.46 (9H, s).

### Step C: O-(2-amino-4-(methoxycarbonyl)phenyl)-N-(tert-butoxycarbonyl)-L-serine

A suspension of N-(tert-butoxycarbonyl)-O-(4-(methoxycarbonyl)-2-nitrophenyl)-L-serine (965 mg, 2.51 mmol) and Pd/C (10 wt %, 91.0 mg, 0.0850 mmol) in EtOAc (8.4 mL) was stirred at room temperature for 5 hours under H₂ atmosphere (1 atm). The reaction mixture was filtered through a Celite pad and washed with EtOAc. The filtrate was concentrated in vacuo to afford O-(2-amino-4-(methoxycarbonyl)phenyl)-N-(tert-butoxycarbonyl)-L-serine (1.32 g, crude) as red oil, which was used for the next step without purification. LC-MS: m/z=355.1 [M+H]⁺.

### Step D: methyl (S)-3-((tert-butoxycarbonyl)amino)-4-oxo-2,3,4,5-tetrahydrobenzo[b][1,4]oxazepine-7-carboxylate

To a solution of O-(2-amino-4-(methoxycarbonyl)phenyl)-N-(tert-butoxycarbonyl)-L-serine (0.100 g, 0.282 mmol) and DiPEA (0.0540 mL, 0.310 mmol) in DMSO (1.2 mL) was added HATU (0.118 g, 0.310 mmol) at room temperature. The reaction mixture was stirred at room temperature for 30 min. After addition of water, the mixture was stirred at room temperature for further 30 min. A precipitated solid was collected by filtration, washed with water, and dried under vacuum to afford methyl (S)-3-((tert-butoxycarbonyl)amino)-4-oxo-2,3,4,5-tetrahydrobenzo[b][1,4] oxazepine-7-carboxylate (47.0 mg, 49% for 3 steps) as beige solid. ¹H-NMR (400 MHz, CDCl₃): δ 7.81 (2H, dd, J=8.5, 2.1 Hz), 7.70 (1H, d, J=1.6 Hz), 7.15 (1H, d, J=8.4 Hz), 5.58 (1H, d, J=5.2 Hz), 4.69-4.63 (2H, m), 4.28 (1H, dd, J=12.0, 11.2 Hz), 3.92 (3H, s), 1.43 (9H, s).

### Step E: methyl (S)-3-((tert-butoxycarbonyl)amino)-5-methyl-4-oxo-2,3,4,5-tetrahydrobenzo [b][1,4]oxazepine-7-carboxylate

To a solution of methyl (S)-3-((tert-butoxycarbonyl)amino)-4-oxo-2,3,4,5-tetrahydrobenzo[b][1,4]oxazepine-7-carboxylate (570 mg, 1.69 mmol) and Cs₂CO₃ (880 mg, 2.70 mmol) in DMF (5.6 mL) was dropwise added Mel (0.148 mL, 2.36 mmol) at room temperature. The reaction mixture was stirred at room temperature for 2 hours. After quenched with water, the mixture was extracted with EtOAc twice. The combined organic layers were washed with brine, dried over Na₂SO₄, filtered, and concentrated in vacuo. The residue was purified by column chromatography on SiO₂ (Hexanes:EtOAc=2:1) to afford methyl (S)-3-((tert-butoxycarbonyl)amino)-5-methyl-4-oxo-2,3,4,5-tetrahydrobenzo[b][1,4]oxazepine-7-carboxylate (380 mg, 64%) as yellow oil. ¹H-NMR (400 MHz, CDCl₃): δ 7.90-7.87 (2H, m), 7.20 (1H, d, J=8.4 Hz), 5.61 (1H, d, J=6.4 Hz), 4.66 (1H, dt, J=10.8, 6.8 Hz), 4.62-4.58 (1H, m), 4.25 (1H, dd, J=10.8, 9.6 Hz), 3.93 (3H, s), 3.44 (3H, s), 1.40 (9H, s).

### Step F: (S)-3-((tert-butoxycarbonyl)amino)-5-methyl-4-oxo-2,3,4,5-tetrahydrobenzo[b][1,4]oxazepine-7-carboxylic acid

To a solution of methyl (S)-3-((tert-butoxycarbonyl)amino)-5-methyl-4-oxo-2,3,4,5-tetrahydrobenzo[b][1,4]oxazepine-7-carboxylate (380 mg, 1.09 mmol) in THF (12 mL) and water (3.8 mL) was added a solution of LiOH—H₂O (68.0 mg, 1.63 mmol) in water (0.76 mL) at room temperature. The reaction mixture was stirred at room temperature for 2 hours. After dilution with icy water, the mixture was washed with EtOAc. The separated aqueous layer was acidified with 1 M aq. HCl until pH 3, and then extracted with EtOAc twice. The combined organic layers were washed with brine, dried over Na₂SO₄, filtered, and concentrated in vacuo to afford (S)-3-((tert-butoxycarbonyl)amino)-5-methyl-4-oxo-2,3,4,5-tetrahydrobenzo[b][1,4]-oxazepine-7-carboxylic acid (360 mg, 99%) as yellow solid. ¹H-NMR (400 MHz, CDCl₃): δ 7.91 (1H, d, J=1.2 Hz), 7.89 (1H, dd, J=8.4, 2.0 Hz), 7.23 (1H, s), 5.73 (1H, d, J=7.2 Hz), 4.73 (1H, dt, J=11.6, 7.2 Hz), 4.63 (1H, dd, J=9.6, 7.2 Hz), 4.29 (1H, dd, J=11.2, 9.6 Hz), 3.45 (3H, s), 2.35 (2H, s), 1.41 (9H, s).

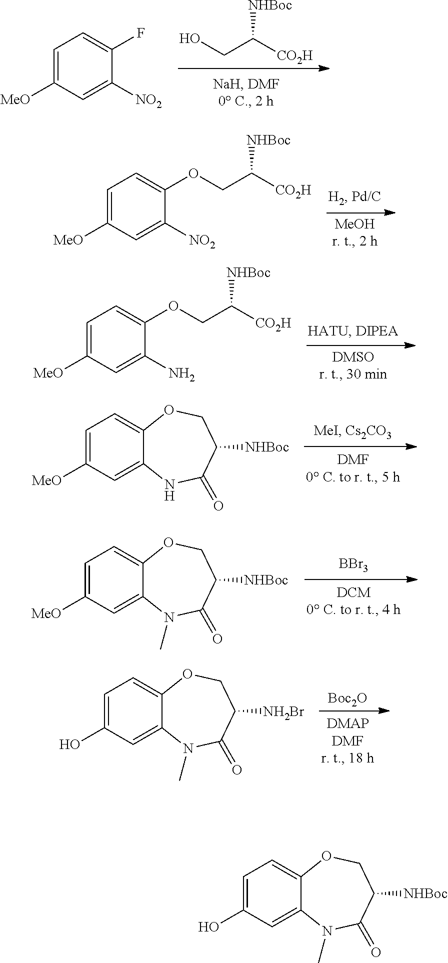

### Intermediate 2: tert-butyl (S)-(7-hydroxy-5-methyl-4-oxo-2,3,4,5-tetrahydrobenzo[b][1,4]oxazepin-3-yl)carbamate

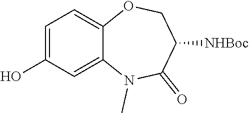 [SMILES: CN1C(=O)[C@@H](NC(=O)OC(C)(C)C)COc2ccc(O)cc21]

### Step A: (S)-2-(tert-butoxycarbonylamino)-3-(4-methoxy-2-nitrophenoxy)propanoic acid

To a suspension of NaH (55 wt %, 460 mg, 10.5 mmol) in dry DMF (20 mL) was slowly added a solution of N-Boc-L-serine (1.00 g, 4.87 mmol) in dry DMF (5.0 mL) at 0° C. The mixture was stirred at room temperature for 30 minutes and cooled to 0° C. After addition of a solution of 1-fluoro-4-methoxy-2-nitrobenzene (900 mg, 5.26 mmol) in dry DMF (5.0 mL) at 0° C., the reaction mixture was stirred at 0° C. for 2 hours. After quenched with 0.5 M aq. HCl, the mixture was extracted with EtOAc, washed with brine, dried over Na₂SO₄, filtered, and concentrated in vacuo. The residue was purified by column chromatography on SiO₂ (Hexanes:EtOAc=4:1 to 1:1) to afford (S)-2-(tert-butoxycarbonylamino)-3-(4-methoxy-2-nitrophenoxy)propanoic acid (900 mg, 48%) as a yellow oil. LC-MS: m/z=257.01 [M+H]⁺.

### Step B: (S)-3-(2-amino-4-methoxyphenoxy)-2-(tert-butoxycarbonylamino)propanoic acid

A suspension of (S)-2-(tert-butoxycarbonylamino)-3-(4-methoxy-2-nitrophenoxy)propanoic acid (350 mg, 0.982 mmol) and Pd/C (5 wt %, 50 mg) in MeOH (10 mL) was stirred at room temperature for 2 hours under H₂ atmosphere (1 atm). After filtration through a Celite pad while washing with MeOH, the filtrate was concentrated in vacuo to afford (S)-3-(2-amino-4-methoxyphenoxy)-2-(tert-butoxycarbonylamino)propanoic acid (200 mg, 62%) as a black solid. LC-MS: m/z=326.89 [M+H]⁺.

### Step C: (S)-tert-butyl 7-methoxy-4-oxo-2,3,4,5-tetrahydrobenzo[b][1,4]oxazepin-3-ylcarbamate

To solution of (S)-3-(2-amino-4-methoxyphenoxy)-2-(tert-butoxycarbonylamino)propanoic acid (320 mg, 0.981 mmol) in DMSO (3.0 mL) was added DIPEA (514 μL, 2.94 mmol) followed by HATU (373 mg, 0.981 mmol) at 0° C. The reaction mixture was stirred at room temperature for 30 minutes. After quenched with ice-water, the mixture was extracted with EtOAc, dried over Na₂SO₄, filtered, and concentrated in vacuo. The residue was purified by column chromatography on SiO₂ (Hexanes:EtOAc=2:1) to afford (S)-tert-butyl 7-methoxy-4-oxo-2,3,4,5-tetrahydrobenzo[b][1,4]oxazepin-3-ylcarbamate (200 mg, 66%) as a white solid. ¹H-NMR (400 MHz, CDCl₃): □□7.17 (1H, brs), 6.90 (1H, d, J=8.8 Hz), 6.68-6.64 (2H, m), 5.48 (1H, brs), 4.69-4.61 (2H, m), 4.21 (1H, t, J=9.6 Hz), 3.79 (3H, s), 1.42 (9H, s).

### Step D: tert-butyl (S)-(7-methoxy-5-methyl-4-oxo-2,3,4,5-tetrahydrobenzo[b][1,4]oxazepin-3-yl)carbamate

To a solution of (S)-tert-butyl 7-methoxy-4-oxo-2,3,4,5-tetrahydrobenzo[b][1,4]oxazepin-3-ylcarbamate (200 mg, 0.649 mmol) in DMF (5.0 mL) was added Cs₂CO₃ (254 mg, 0.778 mmol) followed by a solution of Mel (48.7 μL, 0.778 mmol) in DMF (1.0 mL) at 0° C. The reaction mixture was stirred for 4 hours at 0° C. and then at room temperature for further 1 hour. After quenched with ice-water, the mixture was extracted with EtOAc, dried over Na₂SO₄, filtered, and concentrated in vacuo. The residue was purified by column chromatography on SiO₂ (Hexanes:EtOAc=3:1) to afford tert-butyl (S)-(7-methoxy-5-methyl-4-oxo-2,3,4,5-tetrahydrobenzo[b][1,4]oxazepin-3-yl)carbamate (150 mg, 72%) as a colorless oil. LC-MS: m/z=266.87 [M-tBu+H]⁺.

### Step E: (S)-3-amino-7-hydroxy-5-methyl-2,3-dihydrobenzo[b][1,4]oxazepin-4(5H)-one

To a solution of tert-butyl (S)-(7-methoxy-5-methyl-4-oxo-2,3,4,5-tetrahydrobenzo[b][1,4]oxazepin-3-yl)carbamate (1.90 g, 5.89 mmol) in DCM (19 mL) was added BBr₃ (18.0 mL, 17.7 mmol) at 0° C. The reaction mixture was stirred at room temperature for 4 hours. A precipitated solid was collected by filtration, washed with EtO₂ and dried under vacuum to afford (S)-3-amino-7-hydroxy-5-methyl-2,3-dihydrobenzo[b][1,4]oxazepin-4(5H)-one (1.70 g, 100%) as a white solid. ¹H-NMR (400 MHz, DMSO-d₆): δ 9.49 (1H, s), 6.90 (1H, d, J=8.4 Hz), 6.68 (1H, d, J=2.8 Hz), 6.54 (1H, dd, J=9.0, 2.6 Hz), 4.13 (1H, dd, J=9.8, 7.3 Hz), 3.85-3.80 (1H, m), 3.51 (1H, dd, J=11.6, 8.0 Hz), 3.19 (3H, s).

### Step F: tert-butyl (S)-(7-hydroxy-5-methyl-4-oxo-2,3,4,5-tetrahydrobenzo[b][1,4]oxazepin-3-yl)carbamate

To a solution of (S)-3-amino-7-hydroxy-5-methyl-2,3-dihydrobenzo[b][1,4]oxazepin-4(5H)-one (300 mg, 1.44 mmol) in DMF (4.8 mL) was added (Boc)₂O (629 mg, 2.88 mmol) and DMAP (35.0 mg, 0.288 mmol) at room temperature. The reaction mixture was stirred for 18 hours at room temperature. After quenched with water, the mixture was extracted with EtOAc, washed with water and brine, dried over Na₂SO₄, filtered, and concentrated in vacuo. The residue was purified by column chromatography on SiO₂ (Hexanes:EtOAc=3:1 to 1:1) to afford tert-butyl (S)-(7-hydroxy-5-methyl-4-oxo-2,3,4,5-tetrahydrobenzo[b][1,4]oxazepin-3-yl)carbamate (205 mg, 46%) as a white solid. ¹H-NMR (400 MHz, DMSO-d₆): δ 9.59 (1H, s), 7.08 (1H, d, J=8.8 Hz), 6.94 (1H, d, J=8.8 Hz), 6.74 (1H, d, J=2.8 Hz), 6.58 (1H, dd, J=8.2, 3.0 Hz), 4.33-4.27 (1H, m), 4.18-4.12 (2H, m), 3.14 (3H, s), 1.30 (9H, s).

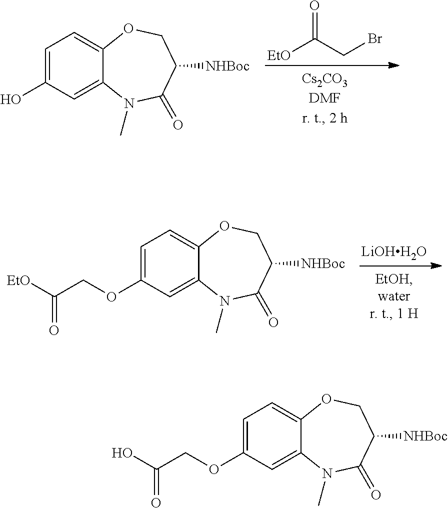

### Intermediate 3: (S)-2-((3-((tert-butoxycarbonyl)amino)-5-methyl-4-oxo-2,3,4,5-tetrahydrobenzo[b][1,4]oxazepin-7-yl)oxy)acetic acid

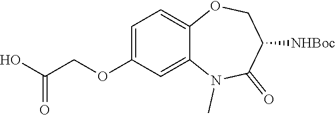 [SMILES: CN1C(=O)[C@@H](NC(=O)OC(C)(C)C)COc2ccc(OCC(=O)O)cc21]

### Step A: ethyl (S)-2-((3-((tert-butoxycarbonyl)amino)-5-methyl-4-oxo-2,3,4,5-tetrahydrobenzo[b][1,4]oxazepin-7-yl)oxy)acetate

To a solution of tert-butyl (S)-(7-hydroxy-5-methyl-4-oxo-2,3,4,5-tetrahydrobenzo[b][1,4]oxazepin-3-yl)carbamate (Intermediate 2, 0.200 g, 0.649 mmol) in DMF (6.5 mL) was added ethyl bromoacetate (0.0940 mL, 0.843 mmol) and Cs₂CO₃ (0.634 g, 1.95 mmol) at room temperature. The reaction mixture was stirred at room temperature for 2 hours. After quenched with water, the mixture was extracted with EtOAc twice. The combined organic layers were washed with brine, dried over Na₂SO₄, filtered, and concentrated in vacuo. The residue was purified by column chromatography on SiO₂ (Hexanes:EtOAc=2:1 to 1:1) to afford ethyl (S)-2-((3-((tert-butoxycarbonyl)amino)-5-methyl-4-oxo-2,3,4,5-tetrahydrobenzo[b][1,4]oxazepin-7-yl)oxy)acetate (0.232 g, 91%) as white solid. ¹H-NMR (400 MHz, CDCl₃): δ 7.07 (1H, d, J=8.8 Hz), 6.81 (1H, d, J=2.8 Hz), 6.70 (1H, dd, J=8.8, 2.2 Hz), 5.54 (1H, d, J=7.2 Hz), 4.65 (1H, dt, J=11.6, 7.2 Hz), 4.61 (2H, s), 4.53 (1H, dd, J=9.6, 7.6 Hz), 4.29 (2H, q, J=7.2 Hz), 4.11 (1H, dd, J=10.8, 10.0 Hz), 3.38 (3H, s), 1.40 (9H, s), 1.32 (3H, t, J=7.2 Hz).

### Step B: (S)-2-((3-((tert-butoxycarbonyl)amino)-5-methyl-4-oxo-2,3,4,5-tetrahydrobenzo[b][1,4]oxazepin-7-yl)oxy)acetic acid

To a solution of ethyl (S)-2-((3-((tert-butoxycarbonyl)amino)-5-methyl-4-oxo-2,3,4,5-tetrahydrobenzo[b][1,4]oxazepin-7-yl)oxy)acetate (0.232 g, 0.588 mmol) in EtOH (4.4 mL) and water (1.5 mL) was added LiOH hydrate (0.247 g, 5.88 mmol) at 0° C. The reaction mixture was stirred at room temperature for 1 hour. After quenched with water, the mixture was washed with EtOAc. The separated aqueous layer was acidified with 1 M aq. HCl until pH 3, and then extracted with EtOAc twice. The combined organic layers were washed with brine, dried over Na₂SO₄, filtered, and concentrated in vacuo to afford (S)-2-((3-((tert-butoxycarbonyl)amino)-5-methyl-4-oxo-2,3,4,5-tetrahydrobenzo[b][1,4]oxazepin-7-yl)oxy)acetic acid (0.209 g, 97%) as a white solid. ¹H-NMR (400 MHz, CDCl₃): δ 7.09 (1H, d, J=8.4 Hz), 6.82 (1H, d, J=2.8 Hz), 6.72 (1H, dd, J=8.8, 2.8 Hz), 5.60 (1H, d, J=7.2 Hz), 4.68 (1H, dt, J=11.6, 7.6 Hz), 4.63 (2H, s), 4.53 (1H, dd, J=9.6, 7.2 Hz), 4.13 (1H, dd, J=11.2, 9.2 Hz), 3.38 (3H, s), 1.40 (9H, s).

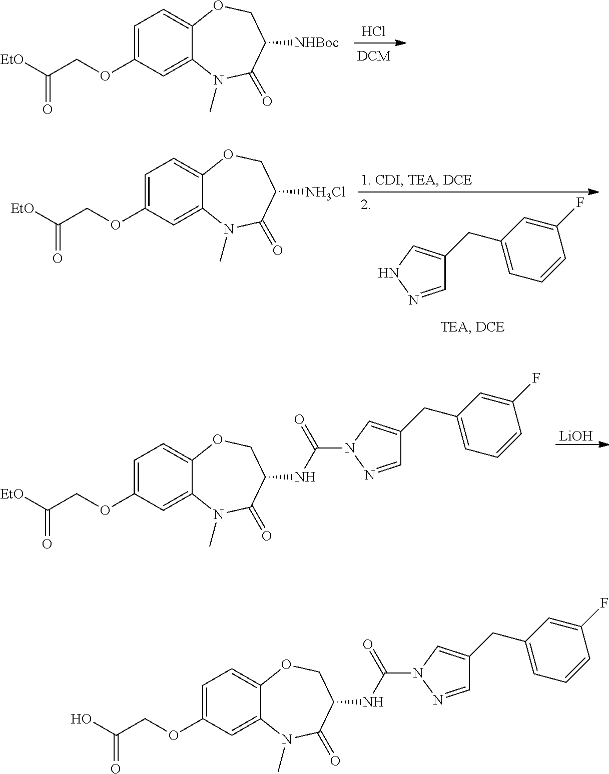

### Intermediate 4. (S)-2-((3-(4-(3-fluorobenzyl)-1H-pyrazole-1-carboxamido)-5-methyl-4-oxo-2,3,4,5-tetrahydrobenzo[b][1,4]oxazepin-7-yl)oxy)acetic acid

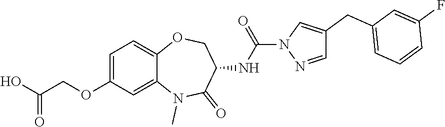 [SMILES: CN1C(=O)[C@@H](NC(=O)n2cc(Cc3cccc(F)c3)cn2)COc2ccc(OCC(=O)O)cc21]

### Step A: ethyl (S)-2-((3-amino-5-methyl-4-oxo-2,3,4,5-tetrahydrobenzo[b][1,4]oxazepin-7-yl)oxy)acetate hydrochloride

To a solution of ethyl (S)-24(3-((tert-butoxycarbonyl)amino)-5-methyl-4-oxo-2,3,4,5-tetrahydrobenzo[b][1,4]oxazepin-7-yl)oxy)acetate (Step A in preparation of Intermediate 3, 0.100 g, 0.254 mmol) in DCM (2.5 mL) was added HCl (4 M solution in dioxane, 1.90 mL, 7.61 mmol) at 0° C. The reaction mixture was stirred at room temperature for 18 hours and concentrated in vacuo to afford ethyl (S)-24(3-amino-5-methyl-4-oxo-2,3,4,5-tetrahydrobenzo[b][1,4]oxazepin-7-yl)oxy)acetate hydrochloride (84.0 mg, 100%) as a yellow oil, which was used for the next step without further purification. LC-MS: m/z=295.0 [M+H]⁺.

### Step B: ethyl (S)-24(3-(4-(3-fluorobenzyl)-1H-pyrazole-1-carboxamido)-5-methyl-4-oxo-2,3,4,5-tetrahydrobenzo[b][1,4]oxazepin-7-yl)oxy)acetate

To a solution of ethyl (S)-24(3-amino-5-methyl-4-oxo-2,3,4,5-tetrahydrobenzo[b][1,4]oxazepin-7-yl)oxy)acetate hydrochloride (84.0 mg, 0.254 mmol) in DCE (2.5 mL) was added CDI (49.0 mg, 0.305 mmol) followed by TEA (0.0880 ml, 0.635 mmol) at 0° C. The reaction mixture was stirred at room temperature for 1 hour. After quenched with water, the mixture was extracted with DCM twice. The combined organic layers were washed with brine, dried over Na₂SO₄, filtered, and concentrated in vacuo.

To a solution of the residue in DCE (2.5 mL) was added 4-(3-fluorobenzyl)-1H-pyrazole hydrochloride (Intermediate 6, 65.0 mg, 0.305 mmol) followed by TEA (0.0880 mL, 0.635 mmol) at 0° C., the reaction mixture was stirred at 45° C. for 18 hours. After quenched with water, the mixture was extracted with DCM twice. The combined organic layers were washed with brine, dried over Na₂SO₄, filtered, and concentrated in vacuo. The residue was purified by column chromatography on SiO₂ (Hexanes:EtOAc=1:1) to afford ethyl (S)-2-((3-(4-(3-fluorobenzyl)-1H-pyrazole-1-carboxamido)-5-methyl-4-oxo-2,3,4,5-tetrahydrobenzo[b][1,4]oxazepin-7-yl)oxy)acetate (0.101 g, 80%) as pale-yellow foam. ¹H-NMR (400 MHz, CDCl₃): δ 8.00 (1H, d, J=7.2 Hz), 7.89 (1H, d, J=0.8 Hz), 7.47 (1H, s), 7.28-7.22 (1H, m), 7.12 (1H, d, J=8.4 Hz), 6.96-6.85 (3H, m), 6.84 (1H, d, J=2.8 Hz), 6.74 (1H, dd, J=9.2, 3.2 Hz), 4.90 (1H, dt, J=11.2, 7.6 Hz), 4.67 (1H, dd, J=9.6, 7.6 Hz), 4.63 (2H, s), 4.29 (2H, q, J=7.2 Hz), 4.26-4.22 (1H, m), 3.81 (2H, s), 3.41 (3H, s), 1.32 (3H, t, J=7.2 Hz).

### Step C: (S)-2-((3-(4-(3-fluorobenzyl)-1H-pyrazole-1-carboxamido)-5-methyl-4-oxo-2,3,4,5-tetrahydrobenzo[b][1,4]oxazepin-7-yl)oxy)acetic acid

To a solution of ethyl (S)-2-((3-(4-(3-fluorobenzyl)-1H-pyrazole-1-carboxamido)-5-methyl-4-oxo-2,3,4,5-tetrahydrobenzo[b][1,4]oxazepin-7-yl)oxy)acetate (0.100 g, 0.201 mmol) in THF (0.29 mL), EtOH (1.2 mL) and water (0.58 mL) was added LiOH hydrate (42.0 mg, 1.01 mmol) at 0° C. The reaction mixture was stirred at 0° C. for 10 minutes. After dilution with water, the mixture was washed with EtOAc. The separated aqueous layer was acidified with 1 M aq. HCl solution until pH 3 and then extracted with EtOAc twice. The combined organic layers were washed with brine, dried over Na₂SO₄, filtered, and concentrated in vacuo to afford (S)-2-((3-(4-(3-fluorobenzyl)-1H-pyrazole-1-carboxamido)-5-methyl-4-oxo-2,3,4,5-tetrahydrobenzo[b][1,4]oxazepin-7-yl)oxy)acetic acid (37.0 mg, 39%) as a white foam. ¹H-NMR (400 MHz, CDCl₃): δ 8.02 (1H, d, J=7.2 Hz), 7.89 (1H, s), 7.47 (1H, s), 7.26-7.22 (1H, m), 7.13 (1H, d, J=8.8 Hz), 6.96-6.85 (4H, m), 6.76 (1H, dd, J=8.8, 2.8 Hz), 4.92 (1H, dt, J=11.2, 7.6 Hz), 4.65 (3H, m), 4.30 (1H, t, J=10.4 Hz), 3.81 (2H, s), 3.41 (3H, s)

### General Synthetic Scheme for Pyrazole Intermediates

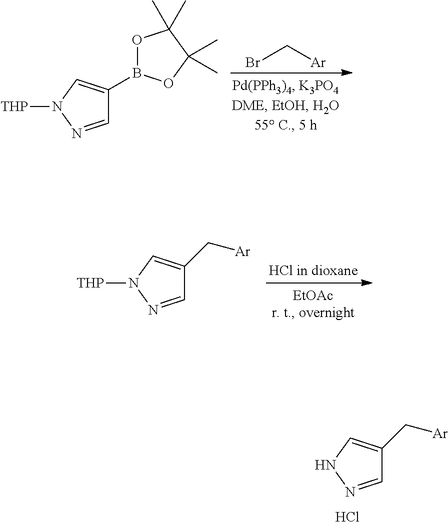

### Intermediate 5: 4-(2-fluorobenzyl)-1H-pyrazole hydrochloride

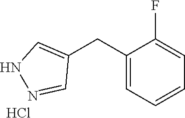 [SMILES: Fc1ccccc1Cc1cn[nH]c1 ; Cl]

### Step A: 4-(2-fluorobenzyl]-1-(oxan-2-yl)pyrazole

To a solution of 1-(oxan-2-yl)-4-(4,4,5,5-tetramethyl-1,3,2-dioxaborolan-2-yl)pyrazole (3.00 g, 10.8 mmol) and 1-(bromomethyl)-2-fluorobenzene (2.65 g, 14.0 mmol) in DME (36 mL), EtOH (9.0 mL) and H₂O (9.0 mL) was added Pd(PPh₃)₄ (0.250 g, 0.216 mmol) and K₃PO₄ (6.87 g, 32.4 mmol) at room temperature. The reaction mixture was stirred at 60° C. for 4 hours under N₂ atmosphere. After dilution with water at room temperature, the mixture was extracted with EtOAc twice. The combined organic layers were washed with brine, dried over Na₂SO₄, filtered, and concentrated in vacuo. The residue was purified by column chromatography on SiO₂ (pet. Ether:EtOAc=5:1) to afford 4-(2-fluorobenzyl)-1-(tetrahydro-2H-pyran-2-yl)-1H-pyrazole (1.73 g, 61%) as a yellow solid. LC-MS (ESI) m/z=261.1 [M+H]⁺

### Step B: 4-(2-fluorobenzyl)-1H-pyrazole hydrochloride

To a solution of 4-(2-fluorobenzyl)-1-(tetrahydro-2H-pyran-2-yl)-1H-pyrazole (1.73 g, 6.65 mmol) in EtOAc (23 mL) was added HCl (4 M in 1,4-dioxane, 16.6 mL, 66.5 mmol) at 0° C. The reaction mixture was stirred at room temperature overnight. A precipitated solid was collected by filtration, washed with EtOAc, and dried under vacuum to afford 4-(2-fluorobenzyl)-1H-pyrazole hydrochloride (0.950 g, 66%) as an off-white solid. ¹H-NMR (400 MHz, DMSO-d₆): δ 7.94 (1H, s), 7.75 (1H, s), 7.27 (2H, dddd, J=10.2, 7.3, 5.6, 2.5 Hz), 7.20-7.09 (2H, m), 3.85 (2H, s). LC-MS (ESI) m/z: [M+H]+=177.0

### Intermediate 6: 4-(3-fluorobenzyl)-1H-pyrazole hydrochloride

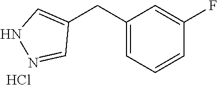 [SMILES: Fc1cccc(Cc2cn[nH]c2)c1 ; Cl]

The title compound was prepared in a similar fashion to Intermediate 6 from 1-(bromomethyl)-3-fluorobenzene in 2 steps (53%) as a white solid. ¹H-NMR (400 MHz, DMSO-d₆): δ 11.78 (2H, brs), 7.88 (2H, d, J=2.6 Hz), 7.33 (1H, td, J=8.0, 6.2 Hz), 7.12-6.92 (3H, m), 3.86 (2H, s). LC-MS (ESI) m/z=177.0 [M+H]⁺

### Intermediate 7: 4-(4-fluorobenzyl)-1H-pyrazole hydrochloride

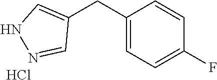 [SMILES: Fc1ccc(Cc2cn[nH]c2)cc1 ; Cl]

The title compound was prepared in a similar fashion to Intermediate 6 from 1-(bromomethyl)-4-fluorobenzene in 2 steps (62%) as a light brown solid. ¹H-NMR (400 MHz, DMSO-d₆): δ 11.52 (3H, s), 7.97-7.90 (2H, m), 7.31-7.24 (2H, m), 7.16-7.07 (2H, m), 3.84 (2H, s). LC-MS (ESI) m/z=177.0 [M+H]⁺

### Intermediate 8: 4-(2,3-difluorobenzyl)-1H-pyrazole hydrochloride

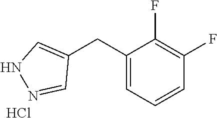 [SMILES: Fc1cccc(Cc2cn[nH]c2)c1F ; Cl]

The title compound was prepared in a similar fashion to Intermediate 6 from 1-(bromomethyl)-2,3-difluorobenzene in 2 steps (59%) as an off-white solid. ¹H-NMR (400 MHz, DMSO-d₆): δ 9.16 (3H, s), 7.73 (2H, s), 7.28 (1H, dtd, J=10.5, 7.9, 2.1 Hz), 7.19-7.04 (2H, m), 3.89 (2H, d, J=1.6 Hz). LC-MS (ESI) m/z=195 [M+H]⁺

### Intermediate 9: 4-(3,4-difluorobenzyl)-1H-pyrazole hydrochloride

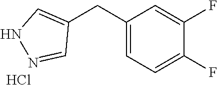 [SMILES: Fc1ccc(Cc2cn[nH]c2)cc1F ; Cl]

The title compound was prepared in a similar fashion to Intermediate 6 from 1-(bromomethyl)-3,4-difluorobenzene in 2 steps (63%) as an off-white solid. ¹H-NMR (400 MHz, DMSO-d₆): δ 7.62-7.55 (2H, m), 7.37-7.21 (2H, m), 7.05 (1H, ddt, J=8.3, 4.0, 1.7 Hz), 3.80 (2H, s). LC-MS (ESI) m/z=195.0 [M+H]⁺.

### Intermediate 10: 4-(3,5-difluorobenzyl)-1H-pyrazole hydrochloride

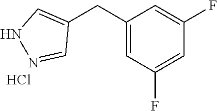 [SMILES: Cl ; Fc1cc(F)cc(Cc2cn[nH]c2)c1]

The title compound was prepared in a similar fashion to Intermediate 6 from 1-(bromomethyl)-3,5-difluorobenzene in 2 steps (66%) as a light-yellow solid. ¹H-NMR (400 MHz, DMSO-d₆): δ 9.53 (3H, s), 7.84-7.65 (2H, m), 7.03 (1H, tt, J=9.5, 2.5 Hz), 6.96 (2H, qd, J=5.9, 5.1, 3.2 Hz), 3.85 (2H, d, J=1.9 Hz). LC-MS (ESI) m/z=195.0 [M+H]⁺.

### Intermediate 11: 4-(2,4-difluorobenzyl)-1H-pyrazole hydrochloride

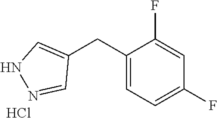 [SMILES: Fc1ccc(Cc2cn[nH]c2)c(F)c1 ; Cl]

The title compound was prepared in a similar fashion to Intermediate 6 from 1-(bromomethyl)-2,4-difluorobenzene in 2 steps (52%) as an off-white solid. ¹H-NMR (400 MHz, DMSO-d₆): δ 9.99 (3H, s), 7.69-7.62 (2H, m), 7.32 (1H, td, J=8.8, 6.6 Hz), 7.19 (1H, ddd, J=10.5, 9.4, 2.6 Hz), 7.02 (1H, tdd, J=8.6, 2.6, 1.1 Hz), 3.81 (2H, s). LC-MS (ESI) m/z=195.0 [M+H]⁺.

### Intermediate 12: 4-(2,6-difluorobenzyl)-1H-pyrazole hydrochloride

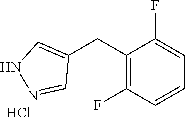 [SMILES: Fc1cccc(F)c1Cc1cn[nH]c1 ; Cl]

The title compound was prepared in a similar fashion to Intermediate 6 from 2-(bromomethyl)-1,3-difluorobenzene in 2 steps (52%) as an off-white solid. ¹H-NMR (400 MHz, DMSO-d₆): δ 7.52 (1H, ddd, J=26.2, 9.3, 4.8 Hz), 7.33 (1H, dddd, J=15.1, 8.4, 6.7, 1.7 Hz), 7.07 (4H, q, J=7.0, 6.4 Hz), 3.81 (2H, d, J=3.9 Hz). LC-MS (ESI) m/z=195.4 [M+H]⁺.

### Intermediate 13: 4-(3-(trifluoromethyl)benzyl)-1H-pyrazole hydrochloride

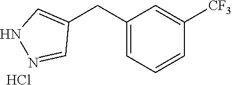 [SMILES: FC(F)(F)c1cccc(Cc2cn[nH]c2)c1 ; Cl]

The title compound was prepared in a similar fashion to Intermediate 6 from 1-(bromomethyl)-3-(trifluoromethyl)benzene in 2 steps (70%) as a pale-yellow solid. ¹H-NMR (400 MHz, DMSO-d₆): δ 12.08 (2H, brs), 7.93 (2H, s), 7.61 (1H, d, J=2.0 Hz), 7.59-7.51 (3H, m), 3.96 (2H, s). LC-MS (ESI) m/z=227.0 [M+H]⁺.

### Intermediate 14: 4-(3-chlorobenzyl)-1H-pyrazole hydrochloride

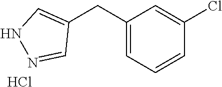 [SMILES: Clc1cccc(Cc2cn[nH]c2)c1 ; Cl]

The title compound was prepared in a similar fashion to Intermediate 6 from 1-(bromomethyl)-3-chlorobenzene in 2 steps (63%) as a light brown solid. ¹H-NMR (400 MHz, DMSO-d₆): δ 13.01 (2H, brs), 7.95-7.79 (2H, m), 7.36-7.28 (2H, m), 7.25 (1H, dt, J=7.9, 1.5 Hz), 7.21 (1H, dt, J=7.3, 1.4 Hz), 3.87-3.83 (2H, m). LC-MS (ESI) m/z=193.0 [M+H]⁺.

### Intermediate 15: 3-((1H-pyrazol-4-yl)methyl)benzonitrile

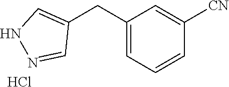 [SMILES: N#Cc1cccc(Cc2cn[nH]c2)c1 ; Cl]

The title compound was prepared in a similar fashion to Intermediate 6 from 3-(bromomethyl)-benzonitrile in 2 steps (66%) as a light brown solid. ¹H NMR (400 MHz, DMSO-d₆): δ 12.67 (2H, brs), 7.85 (2H, d, J=11.8 Hz), 7.72 (1H, d, J=1.9 Hz), 7.67 (1H, dd, J=7.6, 1.5 Hz), 7.63-7.56 (1H, m), 7.51 (1H, t, J=7.7 Hz), 3.91 (2H, s). LC-MS (ESI) m/z=184.1 [M+H]⁺.

### General Synthetic Scheme for Amide Analogues

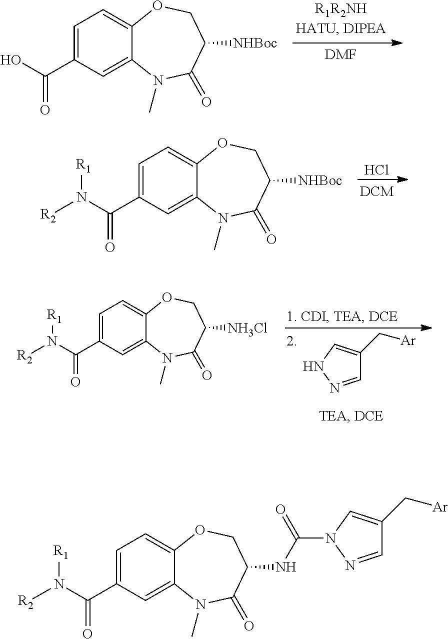

### Example 1: (S)-4-(3-fluorobenzyl)-N-(5-methyl-4-oxo-7-(piperidine-1-carbonyl)-2,3,4,5-tetrahydrobenzo[b][1,4]oxazepin-3-yl)-1H-pyrazole-1-carboxamide

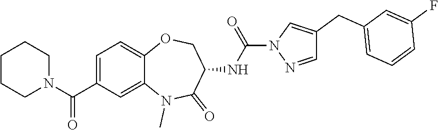 [SMILES: CN1C(=O)[C@@H](NC(=O)n2cc(Cc3cccc(F)c3)cn2)COc2ccc(C(=O)N3CCCCC3)cc21]

### Step A: tert-butyl (S)-(5-methyl-4-oxo-7-(piperidine-1-carbonyl)-2,3,4,5-tetrahydrobenzo[b][1,4]oxazepin-3-yl)carbamate

To a solution of (S)-3-((tert-butoxycarbonyl)amino)-5-methyl-4-oxo-2,3,4,5-tetrahydrobenzo[b][1,4]oxazepine-7-carboxylic acid (Intermediate 1, 0.100 g, 0.297 mmol) and piperidine (0.0350 mL, 0.357 mmol) in DMF (1.5 mL) was added DIPEA (0.156 mL, 0.892 mmol) followed by HATU (0.170 g, 0.446 mmol) at 0° C. The reaction mixture was stirred at 0° C. for 30 minutes. After quenched with water, the reaction mixture was extracted with EtOAc twice. The combined organic layers were washed with brine, dried over Na₂SO₄, filtered, and concentrated in vacuo. The residue was purified by column chromatography on SiO₂ (DCM:EtOAc=1:1) to afford tert-butyl (S)-(5-methyl-4-oxo-7-(piperidine-1-carbonyl)-2,3,4,5-tetrahydrobenzo[b]-[1,4]oxazepin-3-yl)carbamate (32.0 mg, 27%) as a white solid ¹H-NMR (400 MHz, MeOH-d₄): δ 7.45 (1H, d, J=2.0 Hz), 7.32-7.25 (2H, m), 4.57 (1H, dd, J=11.6, 7.6 Hz), 4.44 (1H, dd, J=9.6, 7.6 Hz), 4.30 (1H, dd, J=11.6, 9.6 Hz), 3.79-3.61 (2H, br s), 3.53-3.41 (2H, br s), 3.39 (3H, s), 1.78-1.51 (6H, m), 1.40 (9H, s).

### Step B: (S)-3-amino-5-methyl-7-(piperidine-1-carbonyl)-2,3-dihydrobenzo[b]-[1,4]oxazepin-4(5H)-one hydrochloride

To a solution of tert-butyl (S)-(5-methyl-4-oxo-7-(piperidine-1-carbonyl)-2,3,4,5-tetrahydrobenzo[b][1,4]oxazepin-3-yl)carbamate (32.0 mg, 0.0790 mmol) in DCM (0.80 mL) was added HCl (4 M solution in dioxane, 0.198 mL, 0.793 mmol) at 0° C. The reaction mixture was stirred at room temperature for 20 hours and concentrated in vacuo to afford (S)-3-amino-5-methyl-7-(piperidine-1-carbonyl)-2,3-dihydrobenzo[b][1,4]oxazepin-4(5H)-one HCl (28.0 mg) as a colorless oil, which was used for the next reaction without further purification. ¹H-NMR (400 MHz, MeOH-d₄): δ 7.49 (1H, s), 7.33 (2H, s), 4.67 (1H, dd, J=9.6, 7.6 Hz), 4.52 (1H, dd, J=11.2, 9.6 Hz), 4.43 (1H, dd, J=10.8, 7.2 Hz), 3.75-3.64 (2H, m), 3.50-3.37 (2H, m), 3.44 (3H, s), 1.73-1.57 (6H, m).

### Step C: (S)-4-(3-fluorobenzyl)-N-(5-methyl-4-oxo-7-(piperidine-1-carbonyl)-2,3,4,5-tetrahydrobenzo[b][1,4]oxazepin-3-yl)-1H-pyrazole-1-carboxamide

To a solution of (S)-3-amino-5-methyl-7-(piperidine-1-carbonyl)-2,3-dihydrobenzo[b][1,4]oxazepin-4(5H)-one hydrochloride (27.0 mg, 0.0790 mmol) in DCE (0.80 mL) was added CDI (26.0 mg, 0.159 mmol) followed by TEA (0.0280 mL, 0.199 mmol) at 0° C. The reaction mixture was stirred at 0° C. for 1 hour. After quenched with water, the mixture was extracted with DCM twice. The combined organic layers were washed with brine, dried over Na₂SO₄, filtered, and concentrated in vacuo.

To a solution of the residue in DCE (0.80 mL) was added 4-(3-fluorobenzyl)-1H-pyrazole hydrochloride (Intermediate 6, 20.0 mg, 0.0950 mmol) followed by TEA (0.0280 mL, 0.199 mmol) at 0° C. The reaction mixture was stirred at 40° C. for 3 hours. After quenched with water, the reaction mixture was extracted with DCM twice. The combined organic layers were washed with brine, dried over Na₂SO₄, filtered, and concentrated in vacuo. The residue was purified by prep-TLC on SiO₂ (DCM:EtOAc=1:1) afford (S)-4-(3-fluorobenzyl)-N-(5-methyl-4-oxo-7-(piperidine-1-carbonyl)-2,3,4,5-tetrahydrobenzo[b][1,4]oxazepin-3-yl)-1H-pyrazole-1-carboxamide (17.0 mg, 42%) as a yellow foam. ¹H-NMR (400 MHz, CDCl₃): δ 8.02 (1H, d, J=7.2 Hz), 7.88 (1H, d, J=0.8 Hz), 7.48 (1H, s), 7.33 (1H, d, J=1.6 Hz), 7.28-7.19 (3H, m), 6.96-6.85 (3H, m), 4.94 (1H, dt, J=11.2, 7.2 Hz), 4.73 (1H, dd, J=9.6, 7.6 Hz), 4.34 (1H, dd, J=11.2, 9.6 Hz), 3.81 (2H, s), 3.80-3.59 (2H, m), 3.51-3.36 (2H, m), 3.44 (3H, s), 1.75-1.58 (6H, m). LC-MS: m/z=506.10 [M+H]⁺.

### Example 2: (S)—N-(7-(4,4-dimethylpiperidine-1-carbonyl)-5-methyl-4-oxo-2,3,4,5-tetrahydrobenzo[b][1,4]oxazepin-3-yl)-4-(3-fluorobenzyl)-1H-pyrazole-1-carboxamide

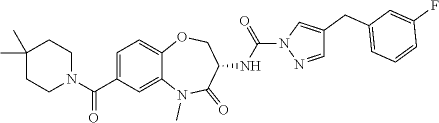 [SMILES: CN1C(=O)[C@@H](NC(=O)n2cc(Cc3cccc(F)c3)cn2)COc2ccc(C(=O)N3CCC(C)(C)CC3)cc21]

### Step A: tert-butyl (S)-(7-(4,4-dimethylpiperidine-1-carbonyl)-5-methyl-4-oxo-2,3,4,5-tetrahydrobenzo[b][1,4]oxazepin-3-yl)carbamate

The title compound was prepared in a similar fashion to Example 1 (Step A) with Intermediate 1 and 4,4-dimethylpiperidine hydrochloride. The crude product was purified by column chromatography on SiO₂ (Hexanes:EtOAc=2:1 to 1:1) to afford tert-butyl (S)-(7-(4,4-dimethylpiperidine-1-carbonyl)-5-methyl-4-oxo-2,3,4,5-tetrahydrobenzo[b][1,4]oxazepin-3-yl)carbamate (94%) as colorless oil. ¹H-NMR (400 MHz, CDCl₃) δ 7.30 (1H, d, J=1.6 Hz), 7.21 (1H, dd, J=8.0, 2.4 Hz), 7.14 (1H, d, J=8.4 Hz), 5.53 (1H, d, J=6.4 Hz), 4.68 (1H, dt, J=11.2, 7.6 Hz), 4.61 (1H, dd, J=9.2, 7.6 Hz), 4.20 (1H, dd, J=11.2, 9.6 Hz), 3.78-3.65 (2H, m), 3.49-3.32 (2H, m), 3.41 (3H, s), 1.52-1.47 (2H, m), 1.40 (9H, s), 1.35-1.30 (2H, m), 1.02 (6H, s).

### Step B: (S)-3-amino-7-(4,4-dimethylpiperidine-1-carbonyl)-5-methyl-2,3-dihydrobenzo[b][1,4]oxazepin-4(5H)-one hydrochloride

The title compound was prepared in a similar fashion to Example 1 (Step B) with tert-butyl (S)-(7-(4,4-dimethylpiperidine-1-carbonyl)-5-methyl-4-oxo-2,3,4,5-tetrahydrobenzo[b][1,4]oxazepin-3-yl)carbamate. ¹H-NMR (400 MHz, MeOH-d₄): δ 7.50 (1H, d, J=1.2 Hz), 7.33 (2H, d, J=1.6 Hz), 4.66 (1H, dd, J=9.6, 7.6 Hz), 4.52 (1H, dd, J=10.8, 9.6 Hz), 4.42 (1H, dd, J=10.8, 7.2 Hz), 3.81-3.69 (2H, m), 3.52-3.38 (2H, m), 3.44 (3H, s), 1.48 (2H, m), 1.36 (2H, m) 1.03 (6H, s).

### Step C: (S)—N-(7-(4,4-dimethylpiperidine-1-carbonyl)-5-methyl-4-oxo-2,3,4,5-tetrahydrobenzo[b][1,4]oxazepin-3-yl)-4-(3-fluorobenzyl)-1H-pyrazole-1-carboxamide

The title compound was prepared in a similar fashion to Example 1 (Step C) with (S)-3-amino-7-(4,4-dimethylpiperidine-1-carbonyl)-5-methyl-2,3-dihydrobenzo[b][1,4]oxazepin-4(5H)-one hydrochloride and 4-(3-fluorobenzyl)-1H-pyrazole hydrochloride (Intermediate 6). The crude product was purified by prep-TLC on SiO₂ (DCM:EtOAc=5:1) to afford (S)—N-(7-(4,4-dimethylpiperidine-1-carbonyl)-5-methyl-4-oxo-2,3,4,5-tetrahydrobenzo[b][1,4]oxazepin-3-yl)-4-(3-fluorobenzyl)-1H-pyrazole-1-carboxamide (44% for 2 steps) as a white foam. ¹H-NMR (400 MHz, CDCl₃): δ 8.01 (1H, d, J=7.2 Hz), 7.88 (1H, s), 7.48 (1H, s), 7.33 (1H, d, J=2.0 Hz), 7.28-7.19 (3H, m), 6.97-6.85 (3H, m), 4.93 (1H, dt, J=11.2, 7.2 Hz), 4.73 (1H, dd, J=9.6, 7.6 Hz), 4.34 (1H, dd, J=11.2, 9.6 Hz), 3.82 (2H, s), 3.79-3.63 (2H, m), 3.53-3.37 (2H, m), 3.45 (3H, s), 1.54-1.42 (2H, m), 1.41-1.29 (2H, m), 1.02 (6H, s). LC-MS: m/z=534.20 [M+H]⁺.

### Example 3: ((S)—N-(7-(4,4-difluoropiperidine-1-carbonyl)-5-methyl-4-oxo-2,3,4,5-tetrahydrobenzo[b][1,4]oxazepin-3-yl)-4-(3-fluorobenzyl)-1H-pyrazole-1-carboxamide

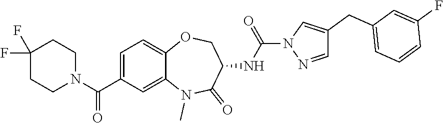 [SMILES: CN1C(=O)[C@@H](NC(=O)n2cc(Cc3cccc(F)c3)cn2)COc2ccc(C(=O)N3CCC(F)(F)CC3)cc21]

### Step A: tert-butyl (S)-(7-(4,4-difluoropiperidine-1-carbonyl)-5-methyl-4-oxo-2,3,4,5-tetrahydrobenzo[b][1,4]oxazepin-3-yl)carbamate

The title compound was prepared in a similar fashion to Example 1 (Step A) with Intermediate 1 and 4,4-difluoropiperidine hydrochloride. The crude product was purified by column chromatography on SiO₂ (DCM:EtOAc=3:1) to afford tert-butyl (S)-(7-(4,4-difluoropiperidine-1-carbonyl)-5-methyl-4-oxo-2,3,4,5-tetrahydrobenzo[b][1,4]oxazepin-3-yl)carbamate (98%) as a white foam. ¹H-NMR (400 MHz, CDCl₃): δ 7.33 (1H, d, J=1.6 Hz), 7.23 (1H, dd, J=8.0, 1.6 Hz), 7.18 (1H, d, J=8.0 Hz), 5.56 (1H, d, J=6.8 Hz), 4.69 (1H, dt, J=11.6, 6.8 Hz), 4.60 (1H, dd, J=9.6, 6.8 Hz), 4.22 (1H, dd, J=11.6, 9.6 Hz), 4.05-3.49 (4H, m), 3.42 (3H, s), 2.22-1.90 (4H, m), 1.40 (9H, s).

### Step B: (S)-3-amino-7-(4,4-difluoropiperidine-1-carbonyl)-5-methyl-2,3-dihydrobenzo[b][1,4]oxazepin-4(5H)-one hydrochloride

The title compound was prepared in a similar fashion to Example 1 (Step B) with tert-butyl (S)-(7-(4,4-difluoropiperidine-1-carbonyl)-5-methyl-4-oxo-2,3,4,5-tetrahydrobenzo[b][1,4]oxazepin-3-yl)carbamate. ¹H-NMR (400 MHz, MeOH-d₄): δ 7.56 (1H, d, J=2.0 Hz), 7.40 (1H, dd, J=8.4, 2.0 Hz), 7.33 (1H, d, J=8.4 Hz), 4.66 (1H, dd, J=9.6, 7.2 Hz), 4.52 (1H, dd, J=11.2, 9.6 Hz), 4.42 (1H, dd, J=10.8, 7.2 Hz), 3.95-3.76 (2H, m), 3.75-3.53 (2H, m), 3.45 (3H, s), 2.07-1.99 (4H, m).

### Step C: ((S)—N-(7-(4,4-difluoropiperidine-1-carbonyl)-5-methyl-4-oxo-2,3,4,5-tetrahydrobenzo[b][1,4]oxazepin-3-yl)-4-(3-fluorobenzyl)-1H-pyrazole-1-carboxamide

The title compound was prepared in a similar fashion to Example 1 with (S)-3-amino-7-(4,4-difluoropiperidine-1-carbonyl)-5-methyl-2,3-dihydrobenzo[b][1,4]oxazepin-4(5H)-one hydrochloride and 4-(3-fluorobenzyl)-1H-pyrazole hydrochloride (Intermediate 6). The crude product was purified by column chromatography on SiO₂ (DCM:EtOAc=8:1) to afford (S)—N-(7-(4,4-difluoropiperidine-1-carbonyl)-5-methyl-4-oxo-2,3,4,5-tetrahydrobenzo[b][1,4]oxazepin-3-yl)-4-(3-fluorobenzyl)-1H-pyrazole-1-carboxamide (52% for 2 steps) as a white foam. ¹H-NMR (400 MHz, CDCl₃): δ 8.01 (1H, d, J=6.8 Hz), 7.88 (1H, s), 7.48 (1H, s), 7.37 (1H, d, J=1.6 Hz), 7.29-7.22, (3H, m), 6.97-6.85 (3H, m), 4.94 (1H, dt, J=11.6, 7.2 Hz), 4.73 (1H, dd, J=9.8, 7.2 Hz), 4.36 (1H, dd, J=11.6, 10.0 Hz), 4.02-3.55 (4H, m), 3.82 (2H, s), 3.45 (3H, s), 2.22-1.91 (4H, m). LC-MS: m/z=542.1 [M+H]⁺.

### Example 4: (S)-4-(3-fluorobenzyl)-N-(7-(4-hydroxypiperidine-1-carbonyl)-5-methyl-4-oxo-2,3,4,5-tetrahydrobenzo[b][1,4]oxazepin-3-yl)-1H-pyrazole-1-carboxamide

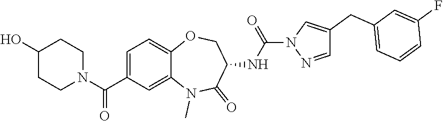 [SMILES: CN1C(=O)[C@@H](NC(=O)n2cc(Cc3cccc(F)c3)cn2)COc2ccc(C(=O)N3CCC(O)CC3)cc21]

### Step A: tert-butyl-(S)-(7-(4-hydroxypiperidine-1-carbonyl)-5-methyl-4-oxo-2,3,4,5-tetrahydrobenzo[b][1,4]oxazepin-3-yl)carbamate

The title compound was prepared in a similar fashion to Example 1 (Step A) with Intermediate 1 and piperidin-4-ol. The crude product was purified by column chromatography on SiO₂ (DCM:MeOH=20:1) to give tert-butyl-(S)-(7-(4-hydroxypiperidine-1-carbonyl)-5-methyl-4-oxo-2,3,4,5-tetrahydrobenzo[b][1,4]oxazepin-3-yl)carbamate (80%) as a white foam. ¹H-NMR (400 MHz, CDCl₃): δ 7.30 (1H, d, J=1.8 Hz), 7.22 (1H, dd, J=8.2, 1.8 Hz), 7.15 (1H, d, J=7.8 Hz), 5.53 (1H, d, J=6.9 Hz), 4.71-4.65 (1H. m), 4.60 (1H, dd, J=9.6, 7.3 Hz), 4.20 (1H, dd, J=11.0, 9.6 Hz), 4.02 (1H, td, J=7.9, 3.8 Hz), 3.41 (3H, s), 3.35-3.20 (2H, m), 2.02-1.84 (2H, m), 1.48 (4H, m), 1.40 (9H, s).

### Step B: (S)-3-amino-7-(4-hydroxypiperidine-1-carbonyl)-5-methyl-2,3-dihydrobenzo[b][1,4]oxazepin-4(5H)-one hydrochloride

The title compound was prepared in a similar fashion to Example 1 (Step B) with tert-butyl-(S)-(7-(4-hydroxypiperidine-1-carbonyl)-5-methyl-4-oxo-2,3,4,5-tetrahydrobenzo[b][1,4]oxazepin-3-yl)carbamate. LC-MS: m/z=320.10 [M+H]⁺.

### Step C: (S)-4-(3-fluorobenzyl)-N-(7-(4-hydroxypiperidine-1-carbonyl)-5-methyl-4-oxo-2,3,4,5-tetrahydrobenzo[b][1,4]oxazepin-3-yl)-1H-pyrazole-1-carboxamide

The title compound was prepared in a similar fashion to Example 1 (Step C) with (S)-3-amino-7-(4-hydroxypiperidine-1-carbonyl)-5-methyl-2,3-dihydrobenzo[b][1,4]oxazepin-4(5H)-one hydrochloride and 4-(3-fluorobenzyl)-1H-pyrazole hydrochloride (Intermediate 6). The crude product was purified by column chromatography on SiO₂ (DCM:EtOAc=10:1) to give (S)-4-(3-fluorobenzyl)-N-(7-(4-hydroxypiperidine-1-carbonyl)-5-methyl-4-oxo-2,3,4,5tetrahydrobenzo[b][1,4]oxazepin-3-yl)-1H-pyrazole-1-carboxamide (2% for 2 steps) as a white foam. ¹H-NMR (400 MHz, CDCl₃): δ 8.01 (1H, d, J=7.3 Hz), 7.88 (1H, s), 7.48 (1H, s), 7.34 (3H, d, J=1.8 Hz), 7.24 (3H, d, J=7.8 Hz), 7.00-6.85 (3H, m), 4.97-4.91 (1H, m), 4.73 (1H, dd, J=9.6, 7.3 Hz), 4.35 (1H, dd, J=11.2, 9.8 Hz), 4.12-4.25 (1H, m), 4.02 (1H, m), 3.82 (2H, s), 3.44 (3H, s), 3.37-3.39 (1H, m), 2.01-1.91 (2H, m), 1.34-1.28 (2H, m), 0.89-0.82 (2H, m). LC-MS: m/z=522.10 [M+H]⁺

### Example 5: (S)-4-(3-fluorobenzyl)-N-(7-(4-hydroxy-4-methylpiperidine-1-carbonyl)-5-methyl-4-oxo-2,3,4,5-tetrahydrobenzo[b][1,4]oxazepin-3-yl)-1H-pyrazole-1-carboxamide

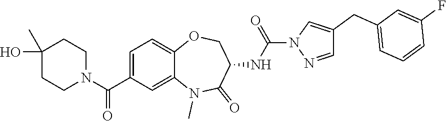 [SMILES: CN1C(=O)[C@@H](NC(=O)n2cc(Cc3cccc(F)c3)cn2)COc2ccc(C(=O)N3CCC(C)(O)CC3)cc21]

### Step A: tert-butyl (S)-(7-(4-hydroxy-4-methylpiperidine-1-carbonyl)-5-methyl-4-oxo-2,3,4,5-tetrahydrobenzo[b][1,4]oxazepin-3-yl)carbamate

The title compound was prepared in a similar fashion to Example 1 (Step A) with Intermediate 1 and 4-methylpiperidin-4-ol. The crude product was purified by column chromatography on SiO₂ (EtOAc:MeOH=9:1) to afford tert-butyl (S)-(7-(4-hydroxy-4-methylpiperidine-1-carbonyl)-5-methyl-4-oxo-2,3,4,5-tetrahydrobenzo[b][1,4]oxazepin-3-yl)carbamate (89%) as a colorless oil. ¹H-NMR (400 MHz, CDCl₃): δ 7.31 (1H, s), 7.22 (1H, dd, J=8.0, 2.0 Hz), 7.15 (1H, d, J=8.0 Hz), 5.55 (1H, d, J=6.8 Hz), 4.71-4.65 (1H, dt, 11.6, 6.8 Hz), 4.61 (1H, dd, J=9.6, 7.6 Hz), 4.20 (1H, dd, J=11.2, 9.6 Hz), 3.65-3.25 (3H, br), 3.41 (3H, s), 1.78-1.50 (5H, br), 1.40 (9H, s), 1.32 (3H, s).

### Step B: (S)-3-amino-7-(4-hydroxy-4-methylpiperidine-1-carbonyl)-5-methyl-2,3-dihydrobenzo[b][1,4]oxazepin-4(5H)-one hydrochloride

The title compound was prepared in a similar fashion to Example 1 (Step B) with tert-butyl (S)-(7-(4-hydroxy-4-methylpiperidine-1-carbonyl)-5-methyl-4-oxo-2,3,4,5tetrahydrobenzo[b][1,4]oxazepin-3-yl)carbamate. LC-MS: m/z=334.10 [M+H]⁺.

### Step C: (S)-4-(3-fluorobenzyl)-N-(7-(4-hydroxy-4-methylpiperidine-1-carbonyl)-5-methyl-4-oxo-2,3,4,5-tetrahydrobenzo[b][1,4]oxazepin-3-yl)-1H-pyrazole-1-carboxamide

The title compound was prepared in a similar fashion to Example 1 (Step C) with (S)-3-amino-7-(4-hydroxy-4-methylpiperidine-1-carbonyl)-5-methyl-2,3-dihydrobenzo[b][1,4]oxazepin-4(5H)-one hydrochloride and 4-(3-fluorobenzyl)-1H-pyrazole hydrochloride (Intermediate 6). The crude product was purified by column chromatography on SiO₂ (EtOAc:MeOH=30:1) to afford (S)-4-(3-fluorobenzyl)-N-(7-(4-hydroxy-4-methylpiperidine-1-carbonyl)-5-methyl-4-oxo-2,3,4,5-tetrahydrobenzo[b][1,4] oxazepin-3-yl)-1H-pyrazole-1-carboxamide (67% for 2 steps) as pale yellow foam. ¹H-NMR (400 MHz, CDCl₃): δ 8.02 (1H, d, J=7.2 Hz), 7.88 (1H, s), 7.48 (1H, s), 7.34 (1H, d, J=1.6 Hz), 7.28-7.24 (2H, m), 7.21 (1H, d, J=8.2 Hz), 6.97-6.85 (3H, m), 4.94 (1H, dt, 11.6, 7.2 Hz), 4.73 (1H, dd, J=9.6, 7.6 Hz), 4.35 (1H, dd, J=11.2, 9.6 Hz), 3.81 (2H, s), 3.68-3.27 (3H, m), 3.44 (3H, s), 1.80-1.48 (5H, m), 1.32 (3H, s). LC-MS: m/z=536.20 [M+H]⁺.

### Example 6: (S)-4-(3-fluorobenzyl)-N-(7-(4-(2-hydroxypropan-2-yl)piperidine-1-carbonyl)-5-methyl-4-oxo-2,3,4,5-tetrahydrobenzo[b][1,4]oxazepin-3-yl)-1H-pyrazole-1-carboxamide

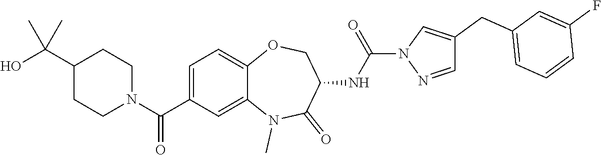 [SMILES: CN1C(=O)[C@@H](NC(=O)n2cc(Cc3cccc(F)c3)cn2)COc2ccc(C(=O)N3CCC(C(C)(C)O)CC3)cc21]

### Step A: (S)-(7-(4-(2-hydroxypropan-2-yl)piperidine-1-carbonyl)-5-methyl-4-oxo-2,3,4,5-tetrahydrobenzo[b][1,4]oxazepin-3-yl)carbamate

The title compound was prepared in a similar fashion to Example 1 (Step A) with Intermediate 1 and 2-(piperidin-4-yl)propan-2-ol. The crude product was purified by column chromatography on SiO₂ (DCM:MeOH=25:1) to afford tert-butyl (S)-(7-(4-(2-hydroxypropan-2-yl)piperidine-1-carbonyl)-5-methyl-4-oxo-2,3,4,5-tetrahydrobenzo[b][1,4]oxazepin-3-yl)carbamate (94%) as a yellow foam. ¹H-NMR (400 MHz, CDCl₃): δ 7.31 (1H, s), 7.22 (1H, d, J=7.6 Hz), 7.15 (1H, d, J=8.4 Hz), 5.53 (1H, d, J=6.8 Hz), 4.91-4.73 (1H, br s), 4.68 (1H, dt, J=11.6, 6.8 Hz), 4.61 (1H, dd, J=9.6, 7.2 Hz), 4.20 (1H, dd, J=11.2, 9.6 Hz), 3.98-3.82 (1H, br s), 3.41 (3H, s), 3.13-2.93 (1H, br s), 2.78-2.63 (1H, br s), 1.98-1.74 (2H, br s), 1.61-1.54 (1H, m), 1.40 (9H, s), 1.23-1.37 (2H, m), 1.22 (6H, s)

### Step B: (S)-3-amino-7-(4-(2-hydroxypropan-2-yl)piperidine-1-carbonyl)-5-methyl-2,3-dihydrobenzo[b][1,4]oxazepin-4(5H)-one hydrochloride

The title compound was prepared in a similar fashion to Example 1 (Step B) with tert-butyl (S)-(7-(4-(2-hydroxypropan-2-yl)piperidine-1-carbonyl)-5-methyl-4-oxo-2,3,4,5-tetrahydrobenzo[b][1,4]oxazepin-3-yl)carbamate. ¹H-NMR (400 MHz, MeOH-d₄): δ 7.51 (1H, d, J=3.6 Hz), 7.34 (2H, d, J=2.0 Hz), 4.77-4.71 (1H, br s), 4.67 (1H, dd, J=9.6, 7.2 Hz), 4.52 (1H, dd, J=11.2, 9.6 Hz), 4.43 (1H, dd, J=11.2, 7.2 Hz), 3.89-3.77 (1H, br s), 3.45 (3H, s), 3.20-3.07 (1H, br s), 2.80 (1H, m), 2.08-1.63 (3H, m), 1.58 (3H, s), 1.48-1.28 (2H, m), 1.17 (3H, s).

### Step C: (S)-4-(3-fluorobenzyl)-N-(7-(4-(2-hydroxypropan-2-yl)piperidine-1-carbonyl)-5-methyl-4-oxo-2,3,4,5-tetrahydrobenzo[b][1,4]oxazepin-3-yl)-1H-pyrazole-1-carboxamide

The title compound was prepared in a similar fashion to Example 1 (Step C) with (S)-3-amino-7-(4-(2-hydroxypropan-2-yl)piperidine-1-carbonyl)-5-methyl-2,3-dihydrobenzo[b][1,4]oxazepin-4(5H)-one hydrochloride and 4-(3-fluorobenzyl)-1H-pyrazole hydrochloride (Intermediate 6). The crude product was purified by column chromatography on SiO₂ (Hexanes:EtOAc=1:3 to 1:5) to afford (S)-4-(3-fluorobenzyl)-N-(7-(4-(2-hydroxypropan-2-yl)piperidine-1-carbonyl)-5-methyl-4-oxo-2,3,4,5-tetrahydrobenzo[b][1,4]oxazepin-3-yl)-1H-pyrazole-1-carboxamide (25% for 2 steps) as a white foam. ¹H-NMR (400 MHz, CDCl₃) δ 8.02 (1H, d, J=6.8 Hz), 7.89 (1H, s), 7.48 (1H, s), 7.35 (1H, d, J=1.2 Hz), 7.31-7.20 (3H, m), 6.97-6.85 (3H, m), 4.94 (1H, dt, J=11.2, 7.2 Hz), 4.88-4.80 (1H, br s), 4.73 (1H, dd, J=9.6, 7.2 Hz), 4.35 (1H, dd, J=11.2, 9.6 Hz), 3.97-3.85 (1H, br s), 3.81 (2H, s), 3.45 (3H, s), 3.15-2.92 (1H, br s), 2.83-2.63 (1H, br s), 2.00-1.74 (2H, m), 1.58 (1H, tt, J=12.0, 3.2 Hz), 1.40-1.24 (2H, m), 1.21 (6H, s). LC-MS: m/z=564.1 [M+H]⁺.

### Example 7: (S)-4-(3-fluorobenzyl)-N-(5-methyl-7-(4-methylpiperazine-1-carbonyl)-4-oxo-2,3,4,5-tetrahydrobenzo[b][1,4]oxazepin-3-yl)-1H-pyrazole-1-carboxamide

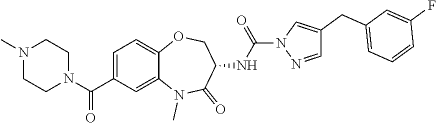 [SMILES: CN1CCN(C(=O)c2ccc3c(c2)N(C)C(=O)[C@@H](NC(=O)n2cc(Cc4cccc(F)c4)cn2)CO3)CC1]

### Step A: tert-butyl (S)-(5-methyl-7-(4-methylpiperazine-1-carbonyl)-4-oxo-2,3,4,5-tetrahydrobenzo[b][1,4]oxazepin-3-yl)carbamate

The title compound was prepared in a similar fashion to Example 1 (Step A) with Intermediate 1 and 1-methylpiperazine. The crude product was purified by column chromatography on SiO₂ (pet.Ether:EtOAc=1:1) to afford tert-butyl (S)-(5-methyl-7-(4-methylpiperazine-1-carbonyl)-4-oxo-2,3,4,5-tetrahydrobenzo[b][1,4]oxazepin-3-yl)carbamate (92%) as colorless oil. ¹H-NMR (400 MHz, CDCl₃): δ 7.32 (1H, d, J=1.6 Hz), 7.23 (1H, dd, J=8.4, 2.0 Hz), 7.16 (1H, d, J=8.4 Hz), 5.56 (1H, d, J=6.8 Hz), 4.68 (1H, dt, J=11.2, 7.2 Hz), 4.60 (1H, dd, J=9.2, 7.6 Hz), 4.21 (1H, dd, J=10.8, 9.6 Hz), 3.81 (2H, brs), 3.50 (2H, brs), 3.41 (3H, s), 2.49 (4H, brs), 2.37 (3H, s), 1.40 (9H, s).

### Step B: (S)-3-amino-5-methyl-7-(4-methylpiperazine-1-carbonyl)-2,3-dihydrobenzo[b][1,4]oxazepin-4(5H)-one hydrochloride

The title compound was prepared in a similar fashion to Example 1 (Step B) with tert-butyl (S)-(5-methyl-7-(4-methylpiperazine-1-carbonyl)-4-oxo-2,3,4,5-tetrahydrobenzo[b][1,4]oxazepin-3-yl)carbamate. LC-MS: m/z=319.1 [M+H]⁺.

### Step C: (S)-4-(3-fluorobenzyl)-N-(5-methyl-7-(4-methylpiperazine-1-carbonyl)-4-oxo-2,3,4,5-tetrahydrobenzo[b][1,4]oxazepin-3-yl)-1H-pyrazole-1-carboxamide

The title compound was prepared in a similar fashion to Example 1 (Step c) with (S)-3-amino-5-methyl-7-(4-methylpiperazine-1-carbonyl)-2,3-dihydrobenzo[b][1,4]oxazepin-4(5H)-one hydrochloride and 4-(3-fluorobenzyl)-1H-pyrazole hydrochloride (Intermediate 6). The crude product was purified by prep-TLC on SiO₂ (EtOAc:MeOH=3:1) to afford (S)-4-(3-fluorobenzyl)-N-(5-methyl-7-(4-methylpiperazine-1-carbonyl)-4-oxo-2,3,4,5-tetrahydrobenzo[b][1,4]oxazepin-3-yl)-1H-pyrazole-1-carboxamide (6% for 2 steps) as a white solid. ¹H-NMR (400 MHz, CDCl₃): δ 8.01 (1H, d, J=7.2 Hz), 7.88 (1H, d, J=0.8 Hz), 7.48 (1H, s), 7.35 (1H, d, J=1.6 Hz), 7.28-7.21 (3H, m), 6.97-6.85 (3H, m), 4.94 (1H, dt, J=11.6, 7.2 Hz), 4.72 (1H, dd, J=9.6, 7.6 Hz), 4.35 (1H, dd, J=11.6, 10.0 Hz), 3.82 (2H, s), 3.62-3.48 (4H, br), 3.45 (3H, s), 2.51-2.44 (4H, m), 2.34 (3H, s). LC-MS: m/z=521.2 [M+H]⁺.

### Example 8: (S)-4-(3-fluorobenzyl)-N-(5-methyl-7-(4-methylpiperazine-1-carbonyl)-4-oxo-2,3,4,5-tetrahydrobenzo[b][1,4]oxazepin-3-yl)-1H-pyrazole-1-carboxamide

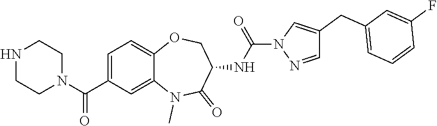 [SMILES: CN1C(=O)[C@@H](NC(=O)n2cc(Cc3cccc(F)c3)cn2)COc2ccc(C(=O)N3CCNCC3)cc21]

### Step A: benzyl (S)-4-(3-((tert-butoxycarbonyl)amino)-5-methyl-4-oxo-2,3,4,5-tetrahydrobenzo[b][1,4]oxazepine-7-carbonyl)piperazine-1-carboxylate

The title compound was prepared in a similar fashion to Example 1 (Step A) with Intermediate 1 and benzyl piperazine-1-carboxylate. The crude product was purified by column chromatography on SiO₂ (pet.Ether:EtOAc=1:1) to afford benzyl (S)-4-(3-((tert-butoxycarbonyl)amino)-5-methyl-4-oxo-2,3,4,5-tetrahydrobenzo[b][1,4]oxazepine-7-carbonyl)piperazine-1-carboxylate (71%) as white solid. ¹H-NMR (400 MHz, CDCl₃): δ 7.39-7.34 (5H, m), 7.32 (1H, d, J=1.6 Hz), 7.22 (1H, dd, J=8.4, 2.0 Hz), 7.17 (1H, d, J=8.0 Hz), 5.55 (1H, d, J=7.2 Hz), 5.16 (2H, s), 4.68 (1H, dt, J=11.2, 7.2 Hz), 4.59 (1H, dd, J=9.6, 7.2 Hz), 4.21 (1H, dd, J=11.6, 9.6 Hz), 3.85-3.43 (8H, m), 3.41 (3H, s), 1.40 (9H, s).

### Step B: tert-butyl (S)-(5-methyl-7-(4-methylpiperazine-1-carbonyl)-4-oxo-2,3,4,5-tetrahydrobenzo[b][1,4]oxazepin-3-yl)carbamate

The title compound was prepared in a similar fashion to Example 1 (Step B) with benzyl (S)-4-(3-((tert-butoxycarbonyl)amino)-5-methyl-4-oxo-2,3,4,5-tetrahydrobenzo[b][1,4]oxazepine-7-carbonyl)piperazine-1-carboxylate. LC-MS: m/z=439.10 [M+H]⁺.

### Step C: benzyl (S)-4-(3-(4-(3-fluorobenzyl)-1H-pyrazole-1-carboxamido)-5-methyl-4-oxo-2,3,4,5-tetrahydrobenzo[b][1,4]oxazepine-7-carbonyl)piperazine-1-carboxylate

The title compound was prepared in a similar fashion to Example 1 (Step C) with benzyl (S)-4-(3-amino-5-methyl-4-oxo-2,3,4,5-tetrahydrobenzo[b][1,4]oxazepine-7-carbonyl)piperazine-1-carboxylate hydrochloride and 4-(3-fluorobenzyl)-1H-pyrazole hydrochloride (Intermediate 6). The crude product was purified by column chromatography on SiO₂ (Pet.Ether:EtOAc=1:3 to 1:5) to afford benzyl (S)-4-(3-(4-(3-fluorobenzyl)-1H-pyrazole-1-carboxamido)-5-methyl-4-oxo-2,3,4,5-tetrahydrobenzo[b][1,4]oxazepine-7-carbonyl)piperazine-1-carboxylate (57% for 2 steps) as a colorless oil. ¹H-NMR (400 MHz, CDCl₃): δ 8.00 (1H, d, J=7.2 Hz), 7.88 (1H, s), 7.48 (1H, s), 7.41-7.31 (5H, m), 7.28-7.22 (3H, m), 6.97-6.86 (3H, m), 5.16 (2H, s), 4.94 (1H, dt, J=11.2, 7.2 Hz), 4.72 (1H, dd, J=10.0, 7.2 Hz), 4.36 (1H, dd, J=11.2, 10.0 Hz), 3.90-3.48 (8H, br), 3.82 (2H, s), 3.44 (3H, s),

### Step D: (S)-4-(3-fluorobenzyl)-N-(5-methyl-7-(4-methylpiperazine-1-carbonyl)-4-oxo-2,3,4,5-tetrahydrobenzo[b][1,4]oxazepin-3-yl)-1H-pyrazole-1-carboxamide

A suspension of benzyl (S)-4-(3-(4-(3-fluorobenzyl)-1H-pyrazole-1-carboxamido)-5-methyl-4-oxo-2,3,4,5-tetrahydrobenzo[b][1,4]oxazepine-7-carbonyl)piperazine-1-carboxylate (38.0 mg, 0.0590 mmol) and 10% Pd/C (6.31 mg, 5.93 μmol) in THF (0.30 mL) and MeOH (0.30 mL) was stirred at room temperature for 18 hours under H₂ atmosphere (1 atm). The reaction mixture was filtered through a Celite pad and washed with MeOH. The filtrate was concentrated in vacuo. The residue was purified by column chromatography on SiO₂ (DCM:MeOH=10:1 to 8:1) to afford (S)-4-(3-fluorobenzyl)-N-(5-methyl-4-oxo-7-(piperazine-1-carbonyl)-2,3,4,5-tetrahydrobenzo[b][1,4]oxazepin-3-yl)-1H-pyrazole-1-carboxamide (6.00 mg, 20%) as a white solid. ¹H-NMR (400 MHz, CDCl₃): δ 7.99 (1H, d, J=6.8 Hz), 7.87 (1H, s), 7.46 (1H, s), 7.33 (1H, d, J=1.6 Hz), 7.26-7.19 (3H, m), 6.95-6.83 (3H, m), 4.92 (1H, dt, J=11.6, 7.2 Hz), 4.71 (1H, dd, J=9.6, 7.2 Hz), 4.33 (1H, dd, J=11.2, 10.0 Hz), 3.86-3.37 (4H, brs), 3.80 (2H, s), 3.43 (3H, s), 3.02-2.78 (4H, brs). LC-MS: m/z=507.0 [M+H]⁺.

### Example 9: (S)-4-(3-fluorobenzyl)-N-(5-methyl-7-(morpholine-4-carbonyl)-4-oxo-2,3,4,5-tetrahydrobenzo[b][1,4]oxazepin-3-yl)-1H-pyrazole-1-carboxamide

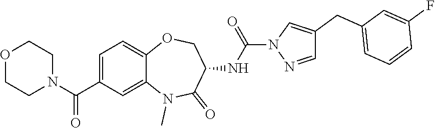 [SMILES: CN1C(=O)[C@@H](NC(=O)n2cc(Cc3cccc(F)c3)cn2)COc2ccc(C(=O)N3CCOCC3)cc21]

### Step A: (S)-3-((tert-butoxycarbonyl)amino)-5-methyl-4-oxo-2,3,4,5-tetrahydrobenzo[b][1,4]oxazepine-7-carboxylic acid

The title compound was prepared in a similar fashion to Example 1 (Step A) with Intermediate 1 and morpholine. The crude product was purified by column chromatography on SiO₂ (pet.Ether:EtOAc=1:2 to 1:3) to afford tert-butyl (S)-(5-methyl-7-(morpholine-4-carbonyl)-4-oxo-2,3,4,5-tetrahydrobenzo[b][1,4]oxazepin-3-yl)carbamate (91%) as a pale yellow solid. ¹H-NMR (400 MHz, CDCl₃): δ 7.33 (1H, d, J=1.6 Hz), 7.22 (1H, dd, J=8.4, 1.6 Hz), 7.16 (1H, d, J=8.4 Hz), 5.56 (1H, d, J=7.6 Hz), 4.68 (1H, dt, J=11.2, 7.2 Hz), 4.59 (1H, dd, J=9.6, 7.2 Hz), 4.21 (1H, dd, J=11.2, 9.6 Hz), 3.73 (8H, brs), 3.42 (3H, s), 1.40 (9H, s).

### Step B: (S)-3-((tert-butoxycarbonyl)amino)-5-methyl-4-oxo-2,3,4,5-tetrahydrobenzo[b][1,4]oxazepine-7-carboxylic acid

The title compound was prepared in a similar fashion to Example 1 (Step B) with tert-butyl (S)-(5-methyl-7-(morpholine-4-carbonyl)-4-oxo-2,3,4,5-tetrahydrobenzo[b][1,4]oxazepin-3-yl)carbamate. ¹H-NMR (400 MHz, DMSO-d₆): δ 8.60 (2H, s), 7.57 (1H, s), 7.33 (2H, s), 4.62 (1H, dd, J=9.6, 7.6 Hz), 4.48 (1H, dd, J=10.8, 10.4 Hz), 4.40 (1H, dd, J=10.4, 7.2 Hz), 3.63 (4H, brs), 3.41 (4H, brs), 3.36 (3H, s).

### Step C: (S)-4-(3-fluorobenzyl)-N-(5-methyl-7-(morpholine-4-carbonyl)-4-oxo-2,3,4,5-tetrahydrobenzo[b][1,4]oxazepin-3-yl)-1H-pyrazole-1-carboxamide

The title compound was prepared in a similar fashion to Example 1 (Step C) with (S)-3-amino-5-methyl-7-(morpholine-4-carbonyl)-2,3-dihydrobenzo[b][1,4]oxazepin-4(5H)-one hydrochloride and 4-(3-fluorobenzyl)-1H-pyrazole hydrochloride (Intermediate 6). The crude product was purified by column chromatography on SiO₂ (Pet.Ether:EtOAc=1:1) to afford (S)-4-(3-fluorobenzyl)-N-(5-methyl-7-(morpholine-4-carbonyl)-4-oxo-2,3,4,5-tetrahydrobenzo[b][1,4]oxazepin-3-yl)-1H-pyrazole-1-carboxamide (56% for 2 steps) as a white foam. ¹H-NMR (400 MHz, CDCl₃): δ 8.01 (1H, d, J=7.2 Hz), 7.88 (1H, d, J=0.8 Hz,), 7.48 (1H, s), 7.37 (1H, d, J=1.6 Hz), 7.28-7.22 (3H, m), 6.97-6.85 (3H, m), 4.94 (1H, dt, J=11.6, 7.2 Hz), 4.72 (1H, dd, J=9.6, 7.2 Hz), 4.36 (1H, dd, J=11.6, 9.6 Hz), 4.00-3.49 (8H, brs), 3.82 (2H, s), 3.45 (3H, s). LC-MS: m/z=508.1 [M+H]⁺.

### Example 10: 4-(3-fluorobenzyl)-N-((3S)-7-(3-hydroxypyrrolidine-1-carbonyl)-5-methyl-4-oxo-2,3,4,5-tetrahydrobenzo[b][1,4]oxazepin-3-yl)-1H-pyrazole-1-carboxamide

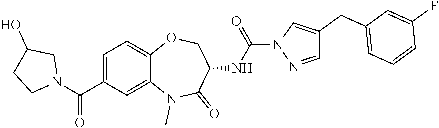 [SMILES: CN1C(=O)[C@@H](NC(=O)n2cc(Cc3cccc(F)c3)cn2)COc2ccc(C(=O)N3CCC(O)C3)cc21]

### Step A: tert-butyl ((3S)-7-(3-hydroxypyrrolidine-1-carbonyl)-5-methyl-4-oxo-2,3,4,5-tetrahydrobenzo[b][1,4]oxazepin-3-yl)carbamate

The title compound was prepared in a similar fashion to Example 1 (Step A) with Intermediate 1 and pyrrolidin-3-ol. The crude product was purified by column chromatography on SiO₂ (DCM:MeOH=10:1) to afford tert-butyl ((3S)-7-(3-hydroxypyrrolidine-1-carbonyl)-5-methyl-4-oxo-2,3,4,5-tetrahydrobenzo[b][1,4]oxazepin-3-yl)carbamate (93%) as a colorless oil. ¹H-NMR (400 MHz, CDCl₃): δ 7.45-7.32 (2H, m), 7.16 (1H, dd, J=8.0, 4.0 Hz,), 5.56 (1H, d, J=6.4 Hz), 4.69-4.63 (1H, m), 4.61-4.57 (1H, m), 4.46 and 4.56 (1H, s), 4.21 (1H, dd, J=11.2, 9.6 Hz), 3.85-3.74 (2H, m), 3.67-3.60 (1H, m), 3.56-3.47 (1H, m), 3.42-3.40 (3H, m), 2.12-1.94 (2H, m), 1.37-1.43 (9H, s).

### Step B: (3S)-3-amino-7-(3-hydroxypyrrolidine-1-carbonyl)-5-methyl-2,3-dihydrobenzo[b][1,4]oxazepin-4(5H)-one hydrochloride

The title compound was prepared in a similar fashion to Example 1 (Step B) with tert-butyl ((3S)-7-(3-hydroxypyrrolidine-1-carbonyl)-5-methyl-4-oxo-2,3,4,5-tetrahydrobenzo[b][1,4]oxazepin-3-yl)carbamate. ¹H-NMR (400 MHz, MeOH-d₄): δ 7.63 (1H, s), 7.48 (1H, d, J=6.4 Hz), 7.34 (1H, d, J=7.2 Hz), 4.72-4.68 (1H, m), 4.55 (1H, t, J=10.5 Hz), 4.43-4.35 (1H, m), 4.54 and 4.42 (1H, brs), 3.83-3.69 (2H, m), 3.61-3.50 (1H, m), 3.46 (3H, d, J=5.6 Hz), 3.39 (1H, m), 2.13-1.98 (2H, m).

### Step C: 4-(3-fluorobenzyl)-N-((3S)-7-(3-hydroxypyrrolidine-1-carbonyl)-5-methyl-4-oxo-2,3,4,5-tetrahydrobenzo[b][1,4]oxazepin-3-yl)-1H-pyrazole-1-carboxamide

The title compound was prepared in a similar fashion to Example 1 (Step C) with (3S)-3-amino-7-(3-hydroxypyrrolidine-1-carbonyl)-5-methyl-2,3-dihydrobenzo[b][1,4]oxazepin-4(5H)-one hydrochloride and 4-(3-fluorobenzyl)-1H-pyrazole hydrochloride (Intermediate 6). The crude product was purified by column chromatography on SiO₂ (EtOAc:MeOH=30:1) to afford (S)-4-(3-fluorobenzyl)-N-(7-(4-hydroxy-4-methylpiperidine-1-carbonyl)-5-methyl-4-oxo-2,3,4,5-tetrahydrobenzo[b][1,4]oxazepin-3-yl)-1H-pyrazole-1-carboxamide (67% for 2 steps) as a yellow foam. ¹H-NMR (400 MHz, CDCl₃): δ 8.01 (1H, d, J=7.6 Hz), 7.88 (1H, s), 7.49-7.37 (3H, m), 7.28-7.21 (2H, m), 6.97-6.85 (3H, m), 4.95-4.89 (1H, dt, J=11.6, 7.2 Hz), 4.72 (1H, dd, J=9.6, 7.2 Hz), 4.63 and 4.52 (1H, brs), 4.35 (1H, dd, J=11.2, 10.0 Hz), 3.86-3.74 (2H, m), 3.81 (2H, s), 3.69-3.52 (2H, m), 3.45 (3H, d, J=6.4 Hz), 2.07-2.01 (2H, m). LC-MS: m/z=508.1 [M+H]⁺.

### Example 11: (S)-3-(4-(3-fluorobenzyl)-1H-pyrazole-1-carboxamido)-N,5-dimethyl-4-oxo-N-(pyridin-4-yl)-2,3,4,5-tetrahydrobenzo[b][1,4]oxazepine-7-carboxamide

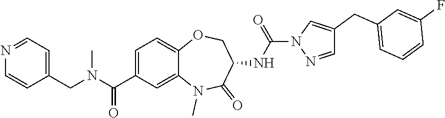 [SMILES: CN(Cc1ccncc1)C(=O)c1ccc2c(c1)N(C)C(=O)[C@@H](NC(=O)n1cc(Cc3cccc(F)c3)cn1)CO2]

### Step A: tert-butyl (S)-(5-methyl-7-(methyl(pyridin-4-ylmethyl)carbamoyl)-4-oxo-2,3,4,5-tetrahydrobenzo[b][1,4]oxazepin-3-yl)carbamate

The title compound was prepared in a similar fashion to Example 1 (Step A) with Intermediate 1 and N-methyl-1-(pyridin-4-yl)methanamine. The crude product was purified by column chromatography on SiO₂ (DCM:MeOH=30:1) to afford tert-butyl (S)-(5-methyl-7-(methyl(pyridin-4-ylmethyl)carbamoyl)-4-oxo-2,3,4,5-tetrahydrobenzo[b][1,4]oxazepin-3-yl)carbamate (92%) as a white foam. ¹H-NMR (400 MHz, CDCl₃): δ 8.64 (2H, d, J=3.6 Hz), 7.47-7.06 (5H, m), 5.62 (1H, d, J=6.8 Hz), 4.84-4.59 (4H, m), 4.24-4.19 (1H, m), 3.43-3.32 (3H, m), 3.02-2.96 (3H, m), 1.40 (9H, s).

### Step B: (S)-3-amino-N,5-dimethyl-4-oxo-N-(pyridin-4-yl)-2,3,4,5-tetrahydrobenzo[b][1,4]oxazepine-7-carboxamide hydrochloride

The title compound was prepared in a similar fashion to Example 1 with tert-butyl (S)-(5-methyl-7-(methyl(pyridin-4-yl)carbamoyl)-4-oxo-2,3,4,5-tetrahydrobenzo[b][1,4]oxazepin-3-yl)carbamate. ¹H-NMR (400 MHz, MeOH-d₄): δ 8.86 (2H, d, J=6.0 Hz), 8.11 (2H, d, J=4.8 Hz), 7.71 (1H, s), 7.55 (1H, d, J=8.0 Hz), 7.39 (1H, d, J=8.0 Hz), 5.06 (2H, s), 4.72-4.66 (1H, m), 4.61-4.52 (1H, m), 4.48-4.42 (1H, m), 3.48 (3H, s), 3.19 (3H, s).

### Step C: (S)-3-(4-(3-fluorobenzyl)-1H-pyrazole-1-carboxamido)-N,5-dimethyl-4-oxo-N-(pyridin-4-yl)-2,3,4,5-tetrahydrobenzo[b][1,4]oxazepine-7-carboxamide

The title compound was prepared in a similar fashion to Example 1 with (S)-3-amino-N,5-dimethyl-4-oxo-N-(pyridin-4-yl)-2,3,4,5-tetrahydrobenzo[b][1,4]oxazepine-7-carboxamide hydrochloride and 4-(3-fluorobenzyl)-1H-pyrazole hydrochloride (Intermediate 6). The crude product was purified by column chromatography on SiO₂ (Hexanes:EtOAc=1:5) to afford (S)-3-(4-(3-fluorobenzyl)-1H-pyrazole-1-carboxamido)-N,5-dimethyl-4-oxo-N-(pyridin-4-yl)-2,3,4,5-tetrahydrobenzo[b][1,4]oxazepine-7-carboxamide (49% for 2 steps) as a white foam. ¹H-NMR (400 MHz, CDCl₃): δ 8.64 (2H, d, J=5.2 Hz), 7.99 (1H, d, J=6.8 Hz), 7.87 (1H, s), 7.47 (1H, s), 7.44-7.12 (6H, m), 6.96-6.85 (3H, m), 4.91 (1H, s), 4.75-4.72 (3H, m), 4.38-4.33 (1H, m), 3.81 (2H, s), 3.51-3.30 (3H, m), 3.13-2.99 (3H, m). LC-MS: m/z=543.2 [M+H]⁺.

### Example 12: (S)-3-(4-(3-fluorobenzyl)-1H-pyrazole-1-carboxamido)-N,5-dimethyl-4-oxo-N-(pyridin-4-yl)-2,3,4,5-tetrahydrobenzo[b][1,4]oxazepine-7-carboxamide

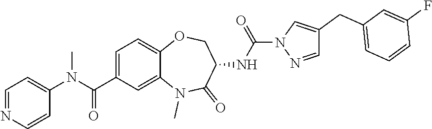 [SMILES: CN(C(=O)c1ccc2c(c1)N(C)C(=O)[C@@H](NC(=O)n1cc(Cc3cccc(F)c3)cn1)CO2)c1ccncc1]

### Step A: tert-butyl (S)-(5-methyl-7-(methyl(pyridin-4-yl)carbamoyl)-4-oxo-2,3,4,5-tetrahydrobenzo[b][1,4]oxazepin-3-yl)carbamate

The title compound was prepared in a similar fashion to Example 1 (Step A) with Intermediate 1 and N-methylpyridin-4-amine. The crude product was purified by column chromatography on SiO₂ (EtOAc:MeOH=20:1) to afford tert-butyl (S)-(5-methyl-7-(methyl(pyridin-4-yl)carbamoyl)-4-oxo-2,3,4,5-tetrahydrobenzo[b][1,4]oxazepin-3-yl)carbamate (99%) as a white foam. ¹H-NMR (400 MHz, CDCl₃): δ 8.52 (2H, d, J=6.4 Hz), 7.24 (1H, d, J=2.0 Hz), 7.17 (1H, dd, J=8.4, 2.0 Hz), 7.02-6.96 (3H, m), 5.52 (1H, d, J=6.0 Hz), 4.62-4.53 (2H, m), 4.21-4.14 (1H, m), 3.54 (3H, s), 3.19 (3H, s), 1.40 (9H, s).

### Step B: (S)-3-amino-N,5-dimethyl-4-oxo-N-(pyridin-4-yl)-2,3,4,5-tetrahydrobenzo[b][1,4]oxazepine-7-carboxamide hydrochloride

The title compound was prepared in a similar fashion to Example 1 (Step B) with tert-butyl (S)-(5-methyl-7-(methyl(pyridin-4-yl)carbamoyl)-4-oxo-2,3,4,5-tetrahydrobenzo[b][1,4]oxazepin-3-yl)carbamate. ¹H-NMR (400 MHz, MeOH-d₄): δ 8.66 (2H, d, J=7.2 Hz), 7.94 (2H, d, J=7.2 Hz), 7.80 (1H, d, J=1.6 Hz), 7.53 (1H, dd, J=8.8, 1.6 Hz), 7.33 (1H, d, J=8.0 Hz), 4.74 (1H, dd, J=9.2, 6.8 Hz), 4.61-4.50 (2H, m), 3.64 (3H, s), 3.43 (3H, s).

### Step C: (S)-3-(4-(3-fluorobenzyl)-1H-pyrazole-1-carboxamido)-N,5-dimethyl-4-oxo-N-(pyridin-4-yl)-2,3,4,5-tetrahydrobenzo[b][1,4]oxazepine-7-carboxamide

The title compound was prepared in a similar fashion to Example 1 with (S)-3-amino-N,5-dimethyl-4-oxo-N-(pyridin-4-yl)-2,3,4,5-tetrahydrobenzo[b][1,4]oxazepine-7-carboxamide hydrochloride and 4-(3-fluorobenzyl)-1H-pyrazole hydrochloride (Intermediate 6) The crude product was purified by column chromatography on SiO₂ (Hexanes:EtOAc=1:5) to afford (S)-3-(4-(3-fluorobenzyl)-1H-pyrazole-1-carboxamido)-N,5-dimethyl-4-oxo-N-(pyridin-4-yl)-2,3,4,5-tetrahydrobenzo[b][1,4]oxazepine-7-carboxamide (49% for 2 steps) as a white foam. ¹H-NMR (400 MHz, CDCl₃): δ 8.52 (2H, dd, J=4.8, 2.0 Hz), 7.95 (1H, d, J=7.6 Hz), 7.88 (1H, d, J=0.8 Hz), 7.46 (1H, s), 7.28-7.21 (3H, m), 7.07 (1H, d, J=8.0 Hz), 6.99 (2H, dd, J=4.8, 2.0 Hz), 6.96-6.84 (3H, m), 4.82 (1H, dt, J=11.6, 7.2 Hz), 4.67 (1H, dd, J=9.6, 7.2 Hz), 4.32 (1H, dd, J=11.2, 9.6 Hz), 3.81 (2H, s), 3.55 (3H, s), 3.21 (3H, s). LC-MS: m/z=529.10 [M+H]⁺.

### Example 13: (S)-3-(4-(3-fluorobenzyl)-1H-pyrazole-1-carboxamido)-N-(2-hydroxy-2-methylpropoxy)-5-methyl-4-oxo-2,3,4,5-tetrahydrobenzo[b][1,4]oxazepine-7-carboxamide

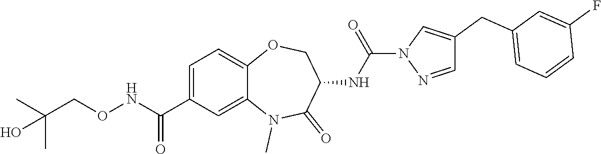 [SMILES: CN1C(=O)[C@@H](NC(=O)n2cc(Cc3cccc(F)c3)cn2)COc2ccc(C(=O)NOCC(C)(C)O)cc21]

### Step A: tert-butyl (S)-(7-((2-hydroxy-2-methylpropoxy)carbamoyl)-5-methyl-4-oxo-2,3,4,5-tetrahydrobenzo[b][1,4]oxazepin-3-yl)carbamate

The title compound was prepared in a similar fashion to Example 1 (Step A) with Intermediate 1 and 1-(aminooxy)-2-methylpropan-2-ol. The crude product was purified by column chromatography on SiO₂ (Hexanes:EtOAc=3:1) to afford tert-butyl (S)-(7-((2-hydroxy-2-methylpropoxy)carbamoyl)-5-methyl-4-oxo-2,3,4,5-tetrahydrobenzo[b][1,4]oxazepin-3-yl)carbamate (35%) as a white solid. ¹H-NMR (400 MHz, MeOH-d₄): δ 7.80 (1H, d, J=2.0 Hz), 7.68 (1H, dd, J=8.4, 2.0 Hz), 7.28 (1H, d, J=8.4 Hz), 4.55 (1H, dd, J=12.0, 7.6 Hz), 4.44 (1H, dd, J=9.6, 7.2 Hz), 4.33 (1H, dd, J=12.0, 10.0 Hz), 3.88 (2H, s), 3.62 (1H, s), 3.41 (3H, s), 1.41 (9H, s), 1.29 (6H, s).

### Step B: ((S)-3-amino-N-(2-hydroxy-2-methylpropoxy)-5-methyl-4-oxo-2,3,4,5-tetrahydrobenzo[b][1,4]oxazepine-7-carboxamide hydrochloride

The title compound was prepared in a similar fashion to Example 1 (Step B) with tert-butyl (S)-(7-((2-hydroxy-2-methylpropoxy)carbamoyl)-5-methyl-4-oxo-2,3,4,5-tetrahydrobenzo[b][1,4]oxazepin-3-yl)carbamate. LC-MS: m/z=324.1 [M+H]⁺.

### Step C: (S)-3-(4-(3-fluorobenzyl)-1H-pyrazole-1-carboxamido)-N-(2-hydroxy-2-methylpropoxy)-5-methyl-4-oxo-2,3,4,5-tetrahydrobenzo[b][1,4]oxazepine-7-carboxamide

The title compound was prepared in a similar fashion to Example 1 (Step C) with (S)-3-amino-N-(2-hydroxy-2-methylpropoxy)-5-methyl-4-oxo-2,3,4,5-tetrahydrobenzo[b][1,4]oxazepine-7-carboxamide hydrochloride and 4-(3-fluorobenzyl)-1H-pyrazole hydrochloride (Intermediate 6). The crude product was purified by column chromatography on SiO₂ (Hexanes:EtOAc=1:4) to give (S)-3-(4-(3-fluorobenzyl)-1H-pyrazole-1-carboxamido)-N-(2-hydroxy-2-methylpropoxy)-5-methyl-4-oxo-2,3,4,5-tetrahydrobenzo[b][1,4]oxazepine-7-carboxamide (6% for 2 steps) as a white foam. ¹H-NMR (400 MHz, CDCl₃): δ 7.99 (1H, d, J=7.2 Hz), 7.88 (1H, d, J=0.8 Hz), 7.47 (1H, s), 7.27-7.22 (1H, m), 7.13 (1H, dd, J=6.8, 2.8 Hz), 6.96-6.82 (5H, m), 4.91 (1H, td, J=10.8, 6.0 Hz), 4.74 (1H, d, J=2.0 Hz), 4.67 (1H, dd, J=10.0, 7.6 Hz), 4.25 (1H, dd, J=11.2, 9.6 Hz), 3.81 (2H, s), 3.42 (3H, s), 1.53 (6H, s). LC-MS: m/z=526.10 [M+H]⁺

### Example 14: (S)-4-(3-fluorobenzyl)-N-(5-methyl-4-oxo-7-(2-oxa-6-azaspiro[3.3]heptane-6-carbonyl)-2,3,4,5-tetrahydrobenzo[b][1,4]oxazepin-3-yl)-1H-pyrazole-1-carboxamide

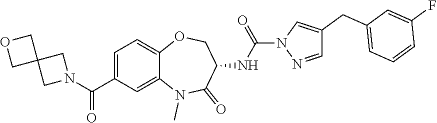 [SMILES: CN1C(=O)[C@@H](NC(=O)n2cc(Cc3cccc(F)c3)cn2)COc2ccc(C(=O)N3CC4(COC4)C3)cc21]

### Step A: methyl (S)-3-amino-5-methyl-4-oxo-2,3,4,5-tetrahydrobenzo[b][1,4]oxazepine-7-carboxylate hydrochloride

To a solution of methyl (S)-3-((tert-butoxycarbonyl)amino)-5-methyl-4-oxo-2,3,4,5-tetrahydrobenzo[b][1,4]oxazepine-7-carboxylate (Step D in Intermediate 1, 0.500 g, 1.43 mmol) in DCM (14 mL) was added HCl (4 M solution in dioxane, 3.57 mL, 14.3 mmol) at 0° C. The reaction mixture was stirred at room temperature for 20 hours. A precipitated solid was collected by filtration, washed with DCM, and dried under vacuum to afford methyl (S)-3-amino-5-methyl-4-oxo-2,3,4,5-tetrahydrobenzo[b][1,4]oxazepine-7-carboxylate hydrochloride (0.392 g, 96%) as a white solid. ¹H-NMR (400 MHz, CDCl₃): δ 8.06 (1H, d, J=1.6 Hz), 7.96 (1H, dd, J=8.4, 1.6 Hz), 7.36 (1H, d, J=8.4 Hz), 4.67 (1H, dd, J=10.0, 7.2 Hz), 4.55 (1H, dd, J=11.6, 10.0 Hz), 4.39 (1H, dd, J=11.2, 7.2 Hz), 3.93 (3H, s), 3.46 (3H, s)

### Step B: methyl (S)-3-(4-(3-fluorobenzyl)-1H-pyrazole-1-carboxamido)-5-methyl-4-oxo-2,3,4,5-tetrahydrobenzo[b][1,4]oxazepine-7-carboxylate

The title compound was prepared in a similar fashion to Example 1 (Step C) with methyl (S)-3-amino-5-methyl-4-oxo-2,3,4,5-tetrahydrobenzo[b][1,4]oxazepine-7-carboxylate hydrochloride and 4-(3-fluorobenzyl)-1H-pyrazole hydrochloride (Intermediate 6). The crude product was purified by column chromatography on SiO₂ (Hexanes:EtOAc=1:1) to afford methyl (S)-3-(4-(3-fluorobenzyl)-1H-pyrazole-1-carboxamido)-5-methyl-4-oxo-2,3,4,5-tetrahydrobenzo[b][1,4]oxazepine-7-carboxylate (93%) as a white foam. ¹H-NMR (400 MHz, CDCl₃): δ 8.00 (1H, d, J=7.2 Hz), 7.93 (2H, m), 7.88 (1H, d, J=1.2 Hz), 7.48 (1H, s), 7.28-7.23 (2H, m), 6.96-6.85 (3H, m), 4.90 (1H, dt, J=11.2, 7.2 Hz), 4.73 (1H, dd, J=9.6, 7.2 Hz), 4.38 (1H, dd, J=11.2, 9.6 Hz), 3.95 (3H, s), 3.81 (2H, s), 3.47 (3H, s)

### Step C: (S)-3-(4-(3-fluorobenzyl)-1H-pyrazole-1-carboxamido)-5-methyl-4-oxo-2,3,4,5-tetrahydrobenzo[b][1,4]oxazepine-7-carboxylic acid

To a solution of methyl (S)-3-(4-(3-fluorobenzyl)-1H-pyrazole-1-carboxamido)-5-methyl-4-oxo-2,3,4,5-tetrahydrobenzo[b][1,4]oxazepine-7-carboxylate (50.0 mg, 0.111 mmol) in THF (1.1 mL) was added 2 M aq. LiOH hydrate (0.276 mL, 0.553 mmol) at 0° C. The reaction mixture was stirred at 10° C. for 20 hours. After concentration in vacuo, the residue was diluted with water and treated with 1 M aq. HCl. A precipitated solid was collected by filtration, washed with water, and dried under vacuum to afford (S)-3-(4-(3-fluorobenzyl)-1H-pyrazole-1-carboxamido)-5-methyl-4-oxo-2,3,4,5-tetrahydrobenzo[b][1,4]-oxazepine-7-carboxylic acid (34.0 mg, 70%) as white solid. LC-MS: m/z=439.0 [M+H]⁺.

### Step D: (S)-4-(3-fluorobenzyl)-N-(5-methyl-4-oxo-7-(2-oxa-6-azaspiro[3.3]heptane-6-carbonyl)-2,3,4,5-tetrahydrobenzo[b][1,4]oxazepin-3-yl)-1H-pyrazole-1-carboxamide

To a solution of (S)-3-(4-(3-fluorobenzyl)-1H-pyrazole-1-carboxamido)-5-methyl-4-oxo-2,3,4,5-tetrahydrobenzo[b][1,4]oxazepine-7-carboxylic acid (27.0 mg, 0.0620 mmol) and 2-oxa-6-azaspiro[3.3]heptane (6.11 mg, 0.0620 mmol) in DMSO (0.62 mL) was added DIPEA (0.0320 mL, 0.185 mmol) followed by HATU (35.0 mg, 0.0920 mmol) at 0° C. The reaction mixture was stirred at 0° C. for 30 minutes. After quenched with water, the mixture was extracted with EtOAc twice. The combined organic layers were washed with brine, dried over Na₂SO₄, filtered, and concentrated in vacuo. The residue was purified by column chromatography on SiO₂ (DCM:MeOH=30:1) to afford (S)-4-(3-fluorobenzyl)-N-(5-methyl-4-oxo-7-(2-oxa-6-azaspiro[3.3]heptane-6-carbonyl)-2,3,4,5-tetrahydrobenzo[b][1,4]oxazepin-3-yl)-1H-pyrazole-1-carboxamide (21 mg, 66%) as a white foam. ¹H-NMR (400 MHz, CDCl₃): δ 8.00 (1H, d, J=6.8 Hz), 7.88 (1H, d, J=0.8 Hz), 7.61 (1H, d, J=1.6 Hz), 7.48 (1H, s), 7.42 (1H, dd, J=8.0, 2.0 Hz), 7.28-7.24 (1H, m), 7.22 (1H, d, J=8.0 Hz), 6.97-6.85 (3H, m), 4.91 (1H, dt, J=11.6, 7.6 Hz), 4.84 (4H, d, J=7.6 Hz), 4.72 (1H, dd, J=9.6, 7.6 Hz), 4.51 (2H, s), 4.37 (2H, s), 4.36 (1H, dd, J=11.6, 9.6 Hz), 3.81 (2H, s), 3.45 (3H, s). LC-MS: m/z=520.10 [M+H]⁺.

### General Synthetic Scheme for Ether Analogues

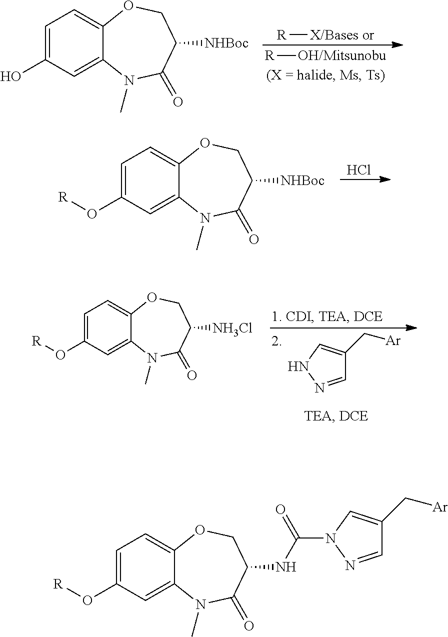

### Example 15: (S)-4-(3-fluorobenzyl)-N-(5-methyl-7-((4-methylpentyl)oxy)-4-oxo-2,3,4,5-tetrahydrobenzo[b][1,4]oxazepin-3-yl)-1H-pyrazole-1-carboxamide

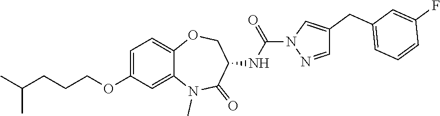 [SMILES: CC(C)CCCOc1ccc2c(c1)N(C)C(=O)[C@@H](NC(=O)n1cc(Cc3cccc(F)c3)cn1)CO2]

### Step A: tert-butyl (S)-(5-methyl-7-((4-methylpentyl)oxy)-4-oxo-2,3,4,5-tetrahydrobenzo[b][1,4]oxazepin-3-yl)carbamate

To a solution of tert-butyl (S)-(7-hydroxy-5-methyl-4-oxo-2,3,4,5-tetrahydrobenzo[b][1,4]oxazepin-3-yl)carbamate (Intermediate 2, 0.150 g, 0.486 mmol) and 4-methylpentyl methanesulfonate (0.105 g, 0.584 mmol) in DMF (4.9 mL) was added Cs₂CO₃ (0.317 g, 0.973 mmol) at 0° C. The reaction mixture was stirred at room temperature for 20 hours. After quenched with water, the mixture was extracted with EtOAc twice. The combined organic layers were washed with brine, dried over Na₂SO₄, filtered, and concentrated in vacuo. The residue was purified by column chromatography on SiO₂ (Hexanes:EtOAc=5:1) to afford tert-butyl (S)-(5-methyl-7-((4-methylpentyl)oxy)-4-oxo-2,3,4,5-tetrahydrobenzo[b][1,4]oxazepin-3-yl)carbamate (0.191 g, 100%) as a white solid. ¹H-NMR (400 MHz, CDCl₃): δ 7.04 (1H, dd, J=7.2, 0.8 Hz), 6.71-6.68 (2H, m), 5.51 (1H, d, J=7.6 Hz), 4.65 (1H, dt, J=11.2, 7.2 Hz), 4.52 (1H, dd, J=9.6, 7.2 Hz), 4.22 (1H, t, J=6.4 Hz), 4.09-4.07 (1H, m), 3.91 (2H, t, J=6.4 Hz), 3.38 (3H, s), 3.01 (2H, s), 1.82-1.72 (2H, m), 1.67-1.55 (1H, m), 1.40 (9H, s), 1.28-1.38 (2H, m), 0.93 (6H, d, J=6.4 Hz)

### Step B: (S)-3-amino-5-methyl-7-((4-methylpentyl)oxy)-2,3-dihydrobenzo[b][1,4]oxazepin-4(5H)-one hydrochloride

To a solution of tert-butyl (S)-(5-methyl-7-((4-methylpentyl)oxy)-4-oxo-2,3,4,5-tetrahydrobenzo[b][1,4]oxazepin-3-yl)carbamate (0.191 g, 0.487 mmol) in DCM (4.9 mL) was added HCl (4 M solution in dioxane, 1.22 mL, 4.87 mmol) at 0° C. The reaction mixture was stirred at room temperature for 5 hours. After concentration in vacuo, the residue was solidified from DCM and Et₂O. The solid was collected by filtration and dried under vacuum to afford (S)-3-amino-5-methyl-7-((4-methylpentyl)oxy)-2,3-dihydrobenzo[b][1,4]oxazepin-4(5H)-one hydrochloride (0.123 g, 77%) as a white solid. ¹H-NMR (400 MHz, CDCl₃): δ 7.05 (1H, d, J=8.8 Hz), 6.72 (1H, d, J=2.8 Hz), 6.69 (1H, dd, J=8.8, 2.8 Hz), 4.84-4.72 (2H, m), 4.45 (1H, dd, J=10.4, 8.0 Hz), 3.94-3.84 (2H, m), 3.26 (3H, s), 1.80-1.73 (2H, m), 1.66-1.56 (1H, m), 1.35-1.30 (2H, m), 0.93 (3H, s), 0.91 (3H, s)

### Step C: (S)-4-(3-fluorobenzyl)-N-(5-methyl-7-((4-methylpentyl)oxy)-4-oxo-2,3,4,5-tetrahydrobenzo[b][1,4]oxazepin-3-yl)-1H-pyrazole-1-carboxamide

To a solution of (S)-3-amino-5-methyl-7-((4-methylpentyl)oxy)-2,3-dihydrobenzo[b][1,4]oxazepin-4(5H)-one hydrochloride (0.123 g, 0.374 mmol) in DCE (3.7 mL) was added CDI (0.121 g, 0.748 mmol) followed by TEA (0.130 ml, 0.935 mmol) at 0° C. The reaction mixture was stirred at 0° C. for 1.5 hours. After quenched with water, the mixture was extracted with DCM twice. The combined organic layers were washed with brine, dried over Na₂SO₄, filtered, and concentrated in vacuo.

To a solution of the residue in DCE (3.7 mL) was added 4-(3-fluorobenzyl)-1H-pyrazole hydrochloride (Intermediate 6, 0.0950 g, 0.449 mmol) followed by TEA (0.130 mL, 0.935 mmol) at 0° C., the reaction mixture was stirred at 40° C. for 16 hours. After quenched with water, the mixture was extracted with DCM twice. The combined organic layers were washed with brine, dried over Na₂SO₄, filtered, and concentrated in vacuo. The residue was purified by column chromatography on SiO₂ (Hexanes:EtOAc=5:1) to afford (S)-4-(3-fluorobenzyl)-N-(5-methyl-7-((4-methylpentyl)oxy)-4-oxo-2,3,4,5-tetrahydrobenzo[b][1,4]-oxazepin-3-yl)-1H-pyrazole-1-carboxamide (0.127 g, 68%) as a colorless oil. ¹H-NMR (400 MHz, CDCl₃): δ 7.98 (1H, d, J=7.6 Hz), 7.88 (1H, d, J=0.8 Hz), 7.47 (1H, s), 7.28-7.22 (1H, m), 7.10 (1H, dd, J=6.8, 2.8 Hz), 6.96-6.85 (3H, m), 6.75 (2H, m), 4.90 (1H, dt, J=10.8, 7.6 Hz), 4.66 (1H, dd, J=10.0, 7.6 Hz), 4.24 (1H, dd, J=10.8, 9.6 Hz), 3.93 (2H, t, J=6.4 Hz), 3.81 (2H, s), 3.42 (3H, s), 1.83-1.76 (2H, m), 1.63 (1H, m), 1.38-1.32 (2H, m), 0.94 (3H, s), 0.92 (3H, s). LC-MS: m/z=495.20 [M+H]⁺.

### Example 16: (S)—N-(7-(3-cyclohexylpropoxy)-5-methyl-4-oxo-2,3,4,5-tetrahydrobenzo[b][1,4]oxazepin-3-yl)-4-(3-fluorobenzyl)-1H-pyrazole-1-carboxamide

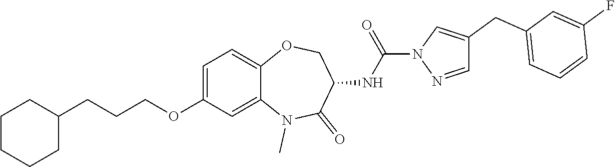 [SMILES: CN1C(=O)[C@@H](NC(=O)n2cc(Cc3cccc(F)c3)cn2)COc2ccc(OCCCC3CCCCC3)cc21]

### Step A: tert-butyl (S)-(7-(3-cyclohexylpropoxy)-5-methyl-4-oxo-2,3,4,5-tetrahydrobenzo[b][1,4]oxazepin-3-yl)carbamate

The title compound was prepared in a similar fashion to Example 15 (Step A) with Intermediate 2 and 3-cyclohexylpropyl methanesulfonate. The crude product was purified by column chromatography on SiO₂ (Hexanes:EtOAc=5:1) to afford tert-butyl (S)-(7-(3-cyclohexylpropoxy)-5-methyl-4-oxo-2,3,4,5-tetrahydrobenzo[b][1,4]-oxazepin-3-yl)carbamate (86%) as a colorless oil. ¹H-NMR (400 MHz, CDCl₃): δ 7.04 (1H, dd, J=7.2, 2 Hz), 6.70-6.68 (2H, m), 5.49 (1H, d, J=7.2 Hz), 4.65 (1H, dt, J=10.8, 7.2 Hz), 4.52 (1H, dd, J=9.6, 7.2 Hz), 4.09 (1H, dd, J=10.8, 9.6 Hz), 3.90 (2H, t, J=6.8 Hz), 3.38 (3H, s), 1.82-1.67 (7H, m), 1.40 (9H, s), 1.36-1.18 (6H, m), 0.96-0.87 (2H, m)

### Step B: (S)-3-amino-7-(3-cyclohexylpropoxy)-5-methyl-2,3-dihydrobenzo[b][1,4]oxazepin-4(5H)-one hydrochloride

The title compound was prepared in a similar fashion to Example 15 (Step B) with tert-butyl (S)-(7-(3-cyclohexylpropoxy)-5-methyl-4-oxo-2,3,4,5-tetrahydrobenzo[b][1,4]oxazepin-3-yl)carbamate. After concentration in vacuo, the residue was solidified from DCM and isopropyl ether to afford (S)-3-amino-7-(3-cyclohexylpropoxy)-5-methyl-2,3-dihydrobenzo[b][1,4]oxazepin-4(5H)-one hydrochloride (83%) as a light blue solid. ¹H-NMR (400 MHz, CDCl₃): δ 7.05 (1H, d, J=8.8 Hz), 6.72-6.67 (2H, m), 4.84-4.70 (2H, m), 4.44 (1H, dd, J=11.2, 8.8 Hz), 3.93-3.84 (2H, m), 3.27 (3H, s), 1.81-1.64 (7H, m), 1.35-1.10 (6H, m), 0.95-0.85 (2H, m)

### Step C: (S)—N-(7-(3-cyclohexylpropoxy)-5-methyl-4-oxo-2,3,4,5-tetrahydrobenzo[b][1,4]oxazepin-3-yl)-4-(3-fluorobenzyl)-1H-pyrazole-1-carboxamide

The title compound was prepared in a similar fashion to Example 15 (Step C) with (S)-3-amino-7-(3-cyclohexylpropoxy)-5-methyl-2,3-dihydrobenzo[b][1,4]oxazepin-4(5H)-one hydrochloride and 4-(3-fluorobenzyl)-1H-pyrazole hydrochloride (Intermediate 6). The crude product was purified by column chromatography on SiO₂ (Hexanes:EtOAc=5:1) to afford (S)—N-(7-(3-cyclohexylpropoxy)-5-methyl-4-oxo-2,3,4,5-tetrahydrobenzo[b][1,4]oxazepin-3-yl)-4-(3-fluorobenzyl)-1H-pyrazole-1-carboxamide (69%) as an colorless oil. ¹H-NMR (400 MHz, CDCl₃): δ 7.99 (1H, d, J=7.6 Hz), 7.88 (1H, d, J=0.8 Hz), 7.47 (1H, s), 7.28-7.22 (1H, m), 7.10 (1H, dd, J=6.4, 2.8 Hz), 6.96-6.85 (3H, m), 6.74 (2H, m), 4.90 (1H, dt, J=11.2, 7.6 Hz), 4.66 (1H, dd, J=10.4, 9.6 Hz), 4.24 (1H, dd, J=11.2, 9.6 Hz), 3.93 (2H, t, J=6.4 Hz), 3.81 (2H, s), 3.41 (3H, s), 1.83-1.65 (7H, m), 1.37-1.13 (6H, m), 0.97-0.85 (2H, m). LC-MS: m/z=535.20 [M+H]⁺.

### Example 17: (S)—N-(7-(2-(dimethylamino)ethoxy)-5-methyl-4-oxo-2,3,4,5-tetrahydrobenzo[b][1,4]oxazepin-3-yl)-4-(3-fluorobenzyl)-1H-pyrazole-1-carboxamide

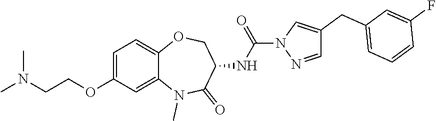 [SMILES: CN(C)CCOc1ccc2c(c1)N(C)C(=O)[C@@H](NC(=O)n1cc(Cc3cccc(F)c3)cn1)CO2]

### Step A: tert-butyl (S)-(7-(2-(dimethylamino)ethoxy)-5-methyl-4-oxo-2,3,4,5-tetrahydrobenzo[b][1,4]oxazepin-3-yl)carbamate

To a solution of tert-butyl (S)-(7-hydroxy-5-methyl-4-oxo-2,3,4,5-tetrahydrobenzo[b][1,4]oxazepin-3-yl)carbamate (Intermediate 2, 130 mg, 0.442 mmol) and 2-(dimethylamino)ethan-1-ol (0.0630 mL, 0.632 mmol) in THF (4.0 mL) was added PPh₃ (221 mg, 0.843 mmol) followed by DIAD (0.164 mL, 0.843 mmol) at 0° C. The reaction mixture was stirred at room temperature for 2 hours. After concentration in vacuo, the residue was purified by column chromatography on SiO₂ (Hexanes:EtOAc=1:1 to EtOAc:MeOH=10:1 to EtOAc:MeOH:NH₄OH=100:10:1) followed by column chromatography on NH₂—SiO₂ (Hexanes:EtOAc=3:1 to 1:1) to give tert-butyl (S)-(7-(2-(dimethylamino)ethoxy)-5-methyl-4-oxo-2,3,4,5-tetrahydrobenzo[b][1,4]oxazepin-3-yl)carbamate (40.0 mg, 25%) as a yellow oil. ¹H-NMR (400 MHz, CDCl₃): δ 7.03 (1H, d, J=8.8 Hz), 6.75-6.70 (2H, m), 5.46 (1H, d, J=7.2 Hz), 4.67-4.60 (1H, m), 4.51 (1H, dd, J=9.6, 7.2 Hz), 4.10-4.00 (3H, m), 3.36 (3H, s), 2.77-2.67 (2H, m), 2.37 (6H, s), 1.38 (9H, s).

### Step B: (S)-3-amino-7-(2-(dimethylamino)ethoxy)-5-methyl-2,3-dihydrobenzo[b][1,4]oxazepin-4(5H)-one hydrochloride

The title compound was prepared in a similar fashion to Example 15 (Step B) with tert-butyl (S)-(7-(2-(dimethylamino)ethoxy)-5-methyl-4-oxo-2,3,4,5-tetrahydrobenzo[b][1,4]oxazepin-3-yl)carbamate. After concentration in vacuo, the crude product was used for the next reaction without purification. LC-MS: m/z=280.1 [M+H]⁺.

### Step C: (S)-4-(3-fluorobenzyl)-N-(5-methyl-4-oxo-7-(3-(pyrrolidin-1-yl)prop-1-yn-1-yl)-2,3,4,5-tetrahydrobenzo[b][1,4]oxazepin-3-yl)-1H-pyrazole-1-carboxamide

The title compound was prepared in a similar fashion to Example 15 (Step C) with (S)-3-amino-7-(2-(dimethylamino)ethoxy)-5-methyl-2,3-dihydrobenzo[b][1,4]oxazepin-4(5H)-one hydrochloride and 4-(3-fluorobenzyl)-1H-pyrazole hydrochloride (Intermediate 6). The crude product was purified by column chromatography on SiO₂ (DCM:MeOH=50:1 to 20:1) to give (S)—N-(7-(2-(dimethylamino)ethoxy)-5-methyl-4-oxo-2,3,4,5-tetrahydrobenzo[b][1,4]oxazepin-3-yl)-4-(3-fluorobenzyl)-1H-pyrazole-1-carboxamide (16% for 2 steps) as a yellow oil. ¹H-NMR (400 MHz, CDCl₃): δ7.97 (1H, d, J=7.2 Hz), 7.86 (1H, s), 7.45 (1H, s), 7.29-7.21 (1H, m), 7.09 (1H, d, J=8.8 Hz), 6.98-8.84 (3H, m), 6.78-6.74 (2H, m), 4.92-4.85 (1H, m), 4.65 (1H, dd, J=9.6, 7.6 Hz), 4.23 (1H, dd, J=10.8, 10.0 Hz), 4.05 (2H, t, J=5.8 Hz), 3.80 (2H, s), 3.39 (3H, s), 2.79-2.69 (2H, m), 2.35 (6H, s). LC-MS: m/z=482.1 [M+H]⁺.

### Example 18: (S)-4-(3-fluorobenzyl)-N-(7-(2-(4-hydroxy-4-methylpiperidin-1-yl)ethoxy)-5-methyl-4-oxo-2,3,4,5-tetrahydrobenzo[b][1,4]oxazepin-3-yl)-1H-pyrazole-1-carboxamide

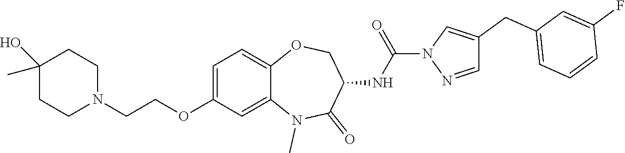 [SMILES: CN1C(=O)[C@@H](NC(=O)n2cc(Cc3cccc(F)c3)cn2)COc2ccc(OCCN3CCC(C)(O)CC3)cc21]

### Step A: tert-butyl-(S)-(7-(2-(4-hydroxy-4-methylpiperidin-1-yl)ethoxy)-5-methyl-4-oxo-2,3,4,5-tetrahydrobenzo[b][1,4]oxazepin-3-yl)carbamate

A mixture of tert-butyl (S)-(7-hydroxy-5-methyl-4-oxo-2,3,4,5-tetrahydrobenzo[b][1,4]oxazepin-3-yl)carbamate (Intermediate 2, 91.0 mg, 0.290 mmol), 1-(2-chloroethyl)-4-methylpiperidin-4-ol (100 mg, 0.560 mmol), NaI (4.00 mg, 0.030 mmol) and K₂CO₃ (123 mg, 0.890 mmol) in DMF (1.0 mL) was stirred at 80° C. for 4 hours. After quenched with water, the mixture was extracted with DCM twice. The combined organic layers were washed with water and brine, dried over Na₂SO₄, filtered, and concentrated in vacuo. The residue was purified by column chromatography on SiO₂ (DCM:MeOH=10:1) to afford tert-butyl-(S)-(7-(2-(4-hydroxy-4-methylpiperidin-1-yl)ethoxy)-5-methyl-4-oxo-2,3,4,5-tetrahydrobenzo[b][1,4]oxazepin-3-yl)carbamate (50.0 mg, 59%) as a white foam. ¹H-NMR (400 MHz, CDCl₃): δ 7.04 (1H, d, J=8.7 Hz), 6.74-6.70 (2H, m), 5.47 (1H, d, J=7.3 Hz), 4.68-4.61 (1H, m), 4.52 (1H, dd, J=9.6, 7.3 Hz), 4.09 (3H, dd, J=11.2, 9.8 Hz), 3.37 (3H, s), 2.83-2.60 (4H, m), 1.82-1.73 (2H, m), 1.65-1.59 (4H, m), 1.39 (9H, s), 1.27 (3H, s).

### Step B: of (S)-3-amino-7-(2-(4-hydroxy-4-methylpiperidin-1-yl)ethoxy)-5-methyl-2,3-dihydrobenzo[b][1,4]oxazepin-4(5H)-one hydrochloride

The title compound was prepared in a similar fashion to Example 15 (Step B) with tert-butyl-(S)-(7-(2-(4-hydroxy-4-methylpiperidin-1-yl)ethoxy)-5-methyl-4-oxo-2,3,4,5-tetrahydrobenzo[b][1,4] oxazepin-3-yl)carbamate. After concentration in vacuo, the crude product was used for the next reaction without purification. LC-MS: m/z=350.10 [M+H]⁺.

### Step C: (S)-4-(3-fluorobenzyl)-N-(7-(2-(4-hydroxy-4-methylpiperidin-1-yl)ethoxy)-5-methyl-4-oxo-2,3,4,5-tetrahydrobenzo[b][1,4]oxazepin-3-yl)-1H-pyrazole-1-carboxamide

The title compound was prepared in a similar fashion to Example 15 (Step C) with (S)-3-amino-7-(2-(4-hydroxy-4-methylpiperidin-1-yl)ethoxy)-5-methyl-2,3-dihydrobenzo[b][1,4]oxazepin-4(5H)-one hydrochloride and 4-(3-fluorobenzyl)-1H-pyrazole hydrochloride (Intermediate 6) The crude product was purified by column chromatography on SiO₂ (DCM:MeOH=20:1) to give (S)-4-(3-fluorobenzyl)-N-(7-(2-(4-hydroxy-4-methylpiperidin-1-yl)ethoxy)-5-methyl-4-oxo-2,3,4,5-tetrahydrobenzo[b][1,4] oxazepin-3-yl)-1H-pyrazole-1-carboxamide (42% for 2 steps) as a white foam. ¹H-NMR (400 MHz, CDCl₃): δ 7.98 (1H, d, J=7.3 Hz), 7.88 (1H, s), 7.47 (1H, s), 7.24-7.22 (1H, m), 7.10 (1H, dd, J=6.6, 2.5 Hz), 7.00-6.85 (3H, m), 6.76 (2H, dd, J=7.3, 2.7 Hz), 4.93-4.86 (1H, m), 4.65 (1H, dd, J=9.8, 7.5 Hz), 4.24 (1H, dd, J=11.2, 9.8 Hz), 4.10 (2H, t, J=5.9 Hz), 3.81 (2H, s), 3.41 (3H, s), 2.84 (2H, t, J=5.7 Hz), 2.71-2.69 (2H, m), 2.55-2.49 (2H, m), 1.73 (2H, m), 1.63 (2H, m), 1.26 (3H, s). LC-MS: m/z=552.20 [M+H]⁺.

### Example 19: (S)-4-(3-fluorobenzyl)-N-(7-(2-(2-hydroxyethoxy)ethoxy)-5-methyl-4-oxo-2,3,4,5-tetrahydrobenzo[b][1,4]oxazepin-3-yl)-1H-pyrazole-1-carboxamide

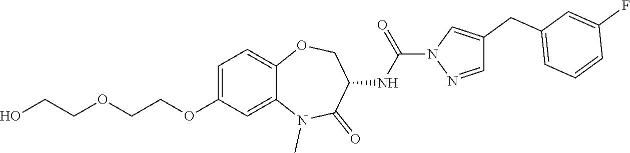 [SMILES: CN1C(=O)[C@@H](NC(=O)n2cc(Cc3cccc(F)c3)cn2)COc2ccc(OCCOCCO)cc21]

### Step A: tert-butyl (S)-(5-methyl-4-oxo-7-(4,4,5,5-tetramethyl-1,3,2-dioxaborolan-2-yl)-2,3,4,5-tetrahydrobenzo[b][1,4]oxazepin-3-yl)carbamate

The title compound was prepared in a similar fashion to Example 15 (Step A) with Intermediate 2 and 2-(2-hydroxyethoxy)ethyl 4-methylbenzenesulfonate. The crude product was purified by column chromatography on SiO₂ (Hexanes:EtOAc=1:4 to 1:9) to afford tert-butyl (S)-(7-(2-(2-hydroxyethoxy)ethoxy)-5-methyl-4-oxo-2,3,4,5-tetrahydrobenzo[b][1,4]oxazepin-3-yl)carbamate (86%) as a white solid. LC-MS: m/z=341.1 [M-tBu+H]⁺.

### Step B: (S)-3-amino-7-(2-(2-hydroxyethoxy)ethoxy)-5-methyl-2,3-dihydrobenzo[b][1,4]oxazepin-4(5H)-one hydrochloride

The title compound was prepared in a similar fashion to Example 15 (Step B) with tert-butyl (S)-(7-(1-(2-hydroxy-2-methylpropyl)-1H-pyrazol-4-yl)-5-methyl-4-oxo-2,3,4,5-tetrahydrobenzo[b][1,4] oxazepin-3-yl)carbamate. After concentration in vacuo, the crude product was used for the next reaction without purification. ¹H-NMR (400 MHz, CDCl₃): δ 7.05 (1H, dd, J=8.8, 2.0 Hz), 6.77-6.75 (1H, m), 6.72 (1H, td, J=8.8, 2.4 Hz), 4.41-4.36 (1H, m), 4.15-4.11 (2H, m), 4.08-4.02 (1H, m), 3.89-3.56 (2H, m), 3.78-3.67 (5H, m), 3.38 (3H, s).

### Step C: (S)-4-(3-fluorobenzyl)-N-(7-(2-(2-hydroxyethoxy)ethoxy)-5-methyl-4-oxo-2,3,4,5-tetrahydrobenzo[b][1,4]oxazepin-3-yl)-1H-pyrazole-1-carboxamide

The title compound was prepared in a similar fashion to Example 15 (Step C) with (S)-3-amino-7-(2-(2-hydroxyethoxy)ethoxy)-5-methyl-2,3-dihydrobenzo[b][1,4]oxazepin-4(5H)-one hydrochloride and 4-(3-fluorobenzyl)-1H-pyrazole hydrochloride (Intermediate 6). The crude product was purified by column chromatography on SiO₂ (Hexanes:EtOAc=1:4) to give (S)-4-(3-fluorobenzyl)-N-(7-(2-(2-hydroxyethoxy)ethoxy)-5-methyl-4-oxo-2,3,4,5-tetrahydrobenzo[b][1,4]oxazepin-3-yl)-1H-pyrazole-1-carboxamide (17% for 2 steps) as a white foam. ¹H-NMR (400 MHz, CDCl₃): δ 7.97 (1H, d, J=7.6 Hz), 7.88 (1H, s), 7.47 (1H, s), 7.27-7.22 (1H, m), 7.11 (1H, d, J=8.4 Hz), 6.96-6.93 (1H, m), 6.91-6.84 (2H, m), 6.80-6.76 (2H, m), 4.90 (1H, td, J=11.2, 7.0 Hz), 4.65 (1H, dd, J=9.6, 7.6 Hz), 4.25 (1H, dd, J=11.2, 10.4 Hz), 4.14 (2H, s), 3.90-3.87 (2H, m), 3.81-3.77 (4H, m), 3.70-3.68 (2H, m), 3.41 (3H, s), 2.35 (6H, s). LC-MS: m/z=499.0 [M+H]⁺.

### Example 20: (S)-4-(3-fluorobenzyl)-N-(7-(2-hydroxy-2-methylpropoxy)-5-methyl-4-oxo-2,3,4,5-tetrahydrobenzo[b][1,4]oxazepin-3-yl)-1H-pyrazole-1-carboxamide

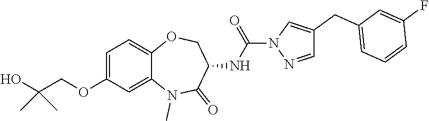 [SMILES: CN1C(=O)[C@@H](NC(=O)n2cc(Cc3cccc(F)c3)cn2)COc2ccc(OCC(C)(C)O)cc21]

### Step A: tert-butyl (S)-(7-(2-hydroxy-2-methylpropoxy)-5-methyl-4-oxo-2,3,4,5-tetrahydrobenzo[b][1,4]oxazepin-3-yl)carbamate

To a solution of tert-butyl (S)-(7-hydroxy-5-methyl-4-oxo-2,3,4,5-tetrahydrobenzo[b][1,4]oxazepin-3-yl)carbamate (Intermediate 2, 130 mg, 0.422 mmol) in DMF (4.0 mL) was added 2,2-dimethyloxirane (3.74 mL, 4.22 mmol) followed by Cs₂CO₃ (412 mg, 1.27 mmol) at room temperature. The reaction mixture was stirred at 60° C. for 24 hours. After quenched with water, the mixture was extracted with EtOAc twice. The combined organic layers were dried over Na₂SO₄, filtered, and concentrated in vacuo. The residue was purified by column chromatography on SiO₂ (Hexanes:EtOAc=2:1) to afford tert-butyl (S)-(7-(2-hydroxy-2-methylpropoxy)-5-methyl-4-oxo-2,3,4,5-tetrahydrobenzo[b][1,4]oxazepin-3-yl)carbamate (40.0 mg, 24%) as a white solid. ¹H-NMR (400 MHz, CDCl₃): δ 7.05 (1H, d, J=7.6 Hz), 6.74-6.70 (2H, m), 5.45 (1H, d, J=7.6 Hz), 4.66-4.60 (1H, m), 4.51 (1H, dd, J=9.6, 7.2 Hz), 4.13-4.06 (1H, m), 3.75 (2H, s), 3.38 (3H, s), 2.13 (1H, s), 1.38 (9H, s), 1.36 (6H, s).

### Step B: (S)-3-amino-7-(2-hydroxy-2-methylpropoxy)-5-methyl-2,3-dihydrobenzo[b][1,4]oxazepin-4(5H)-one hydrochloride

The title compound was prepared in a similar fashion to Example 15 (Step B) with tert-butyl (S)-(7-(2-hydroxy-2-methylpropoxy)-5-methyl-4-oxo-2,3,4,5-tetrahydrobenzo[b][1,4]oxazepin-3-yl)carbamate. After concentration in vacuo, the crude product was used for the next reaction without purification. LC-MS: m/z=281.1 [M+H]⁺.

### Step C: (S)-4-(3-fluorobenzyl)-N-(7-(2-hydroxy-2-methylpropoxy)-5-methyl-4-oxo-2,3,4,5-tetrahydrobenzo[b][1,4]oxazepin-3-yl)-1H-pyrazole-1-carboxamide

The title compound was prepared in a similar fashion to Example 15 (Step C) with (S)-3-amino-7-(2-hydroxy-2-methylpropoxy)-5-methyl-2,3-dihydrobenzo[b][1,4]oxazepin-4(5H)-one hydrochloride and 4-(3-fluorobenzyl)-1H-pyrazole hydrochloride (Intermediate 6). The crude product was purified by column chromatography on SiO₂ (Hexanes:EtOAc=3:1 to EtOAc only) to give (S)-4-(3-fluorobenzyl)-N-(7-(2-hydroxy-2-methylpropoxy)-5-methyl-4-oxo-2,3,4,5-tetrahydrobenzo[b][1,4]oxazepin-3-yl)-1H-pyrazole-1-carboxamide (63% for 2 steps) as a colorless oil. ¹H-NMR (400 MHz, CDCl₃): δ 7.96 (1H, d, J=7.2 Hz), 7.86 (1H, s), 7.46 (1H, s), 7.25-7.23 (1H, m), 7.11-7.10 (1H, m), 6.95-6.82 (3H, m), 6.78-6.75 (2H, m), 4.92-4.85 (1H, m), 4.65 (1H, dd, J=9.6, 7.2 Hz), 4.24 (1H, dd, J=11.6, 10.4 Hz), 3.80 (2H, s), 3.78 (2H, s), 3.41 (3H, s), 1.35 (6H, s). LC-MS: m/z=483.1 [M+H]⁺.

### Example 21: (S)-4-(3-fluorobenzyl)-N-(7-(3-hydroxy-3-methylbutoxy)-5-methyl-4-oxo-2,3,4,5-tetrahydrobenzo[b][1,4]oxazepin-3-yl)-1H-pyrazole-1-carboxamide

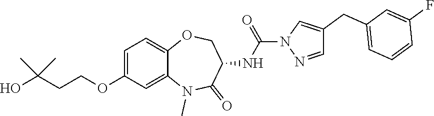 [SMILES: CN1C(=O)[C@@H](NC(=O)n2cc(Cc3cccc(F)c3)cn2)COc2ccc(OCCC(C)(C)O)cc21]

### Step A: tert-butyl (S)-(7-(3-hydroxy-3-methylbutoxy)-5-methyl-4-oxo-2,3,4,5-tetrahydrobenzo[b][1,4]oxazepin-3-yl)carbamate

The title compound was prepared in a similar fashion to Example 15 (Step A) with Intermediate 2 and 3-hydroxy-3-methylbutyl-4-methylbenzenesulfonate. The crude product was purified by column chromatography on SiO₂ (Hexanes:EtOAc=5:1 to 1:1) to afford tert-butyl (S)-(7-(3-hydroxy-3-methylbutoxy)-5-methyl-4-oxo-2,3,4,5-tetrahydrobenzo[b][1,4]oxazepin-3-yl)carbamate (93%) as a white solid. ¹H-NMR (400 MHz, CDCl₃): δ 7.07-7.04 (1H, m), 6.74-6.71 (2H, m), 5.47 (1H, d, J=7.2 Hz), 4.65 (1H, td, J=11.6, 5.6 Hz), 4.52 (1H, dd, J=9.6, 8.0 Hz), 4.15 (2H, t, J=6.4 Hz), 4.12-4.07 (1H, m), 3.38 (3H, s), 2.00 (1H, t, J=6.4 Hz), 1.39 (9H, s), 1.33 (6H, s).

### Step B: (S)-3-amino-7-(3-hydroxy-3-methylbutoxy)-5-methyl-2,3-dihydrobenzo[b][1,4]oxazepin-4(5H)-one hydrochloride

The title compound was prepared in a similar fashion to Example 15 (Step B) with tert-butyl (S)-(7-(3-hydroxy-3-methylbutoxy)-5-methyl-4-oxo-2,3,4,5-tetrahydrobenzo[b][1,4]oxazepin-3-yl)carbamate. After concentration in vacuo, the crude product was used for the next reaction without purification. LC-MS: m/z=295.1 [M+H]⁺.

### Step C: (S)-4-(3-fluorobenzyl)-N-(7-(3-hydroxy-3-methylbutoxy)-5-methyl-4-oxo-2,3,4,5-tetrahydrobenzo[b][1,4]oxazepin-3-yl)-1H-pyrazole-1-carboxamide

The title compound was prepared in a similar fashion to Example 15 (Step C) with (S)-3-amino-N-(2-hydroxy-2-methylpropoxy)-5-methyl-4-oxo-2,3,4,5-tetrahydrobenzo[b][1,4]oxazepine-7-carboxamide hydrochloride and 4-(3-fluorobenzyl)-1H-pyrazole hydrochloride (Intermediate 6). The crude product was purified by column chromatography on SiO₂ (Hexanes:EtOAc=1:4) to give (S)-4-(3-fluorobenzyl)-N-(7-(3-hydroxy-3-methylbutoxy)-5-methyl-4-oxo-2,3,4,5-tetrahydrobenzo[b][1,4]oxazepin-3-yl)-1H-pyrazole-1-carboxamide (60% for 2 steps) as a white foam. ¹H-NMR (400 MHz, CDCl₃): δ 7.97 (1H, d, J=8.0 Hz), 7.88 (1H, s), 7.46 (1H, s), 7.27-7.22 (1H, m), 7.10 (1H, d, J=8.0 Hz), 6.96-6.85 (3H, m), 6.78-6.75 (2H, m), 4.90 (1H, td, J=10.8, 5.6 Hz), 4.65 (1H, dd, J=10.0, 7.6 Hz), 4.24 (1H, dd, J=11.2, 10.0 Hz), 4.17 (2H, t, J=6.4 Hz), 3.80 (2H, s), 3.41 (3H, s), 2.00 (2H, t, J=6.4 Hz), 1.32 (6H, s). 496.54; LC-MS: m/z=497.1 [M+H]⁺.

### Example 22: (S)-4-(3-fluorobenzyl)-N-(7-((4-hydroxy-4-methylpent-2-yn-1-yl)oxy)-5-methyl-4-oxo-2,3,4,5-tetrahydrobenzo[b][1,4]oxazepin-3-yl)-1H-pyrazole-1-carboxamide

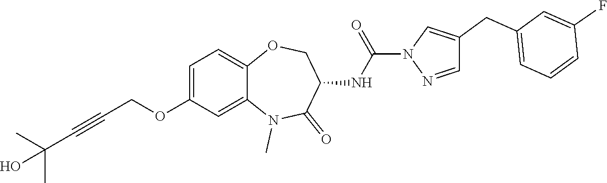 [SMILES: CN1C(=O)[C@@H](NC(=O)n2cc(Cc3cccc(F)c3)cn2)COc2ccc(OCC#CC(C)(C)O)cc21]

### Step A: tert-butyl (S)-(7-((4-hydroxy-4-methylpent-2-yn-1-yl)oxy)-5-methyl-4-oxo-2,3,4,5-tetrahydrobenzo[b][1,4]oxazepin-3-yl)carbamate

The title compound was prepared in a similar fashion to Example 15 (Step A) with Intermediate 2 and 5-chloro-2-methylpent-3-yn-2-ol. The crude product was purified by column chromatography on SiO₂ (Hexanes:EtOAc=4:1 to 1:1) to afford tert-butyl (S)-(7-((4-hydroxy-4-methylpent-2-yn-1-yl)oxy)-5-methyl-4-oxo-2,3,4,5-tetrahydrobenzo[b][1,4]oxazepin-3-yl)carbamate (93%) as a yellow solid. ¹H-NMR (400 MHz, CDCl₃): δ 7.08-7.05 (1H, m), 6.79-6.77 (2H, m), 5.48 (1H, d, J=7.2 Hz), 4.72-4.61 (3H, m), 4.55-4.50 (1H, m), 4.16-4.07 (2H, m), 3.80 (3H, s), 1.51 (6H, s), 1.39 (9H, s).

### Step B: (S)-3-amino-7-((4-hydroxy-4-methylpent-2-yn-1-yl)oxy)-5-methyl-2,3-dihydrobenzo[b][1,4]oxazepin-4(5H)-one hydrochloride

The title compound was prepared in a similar fashion to Example 15 (Step B) with tert-butyl (S)-(7-((4-hydroxy-4-methylpent-2-yn-1-yl)oxy)-5-methyl-4-oxo-2,3,4,5-tetrahydrobenzo[b][1,4]oxazepin-3-yl)carbamate. After concentration in vacuo, the crude product was used for the next reaction without purification. LC-MS: m/z=305.1 [M+H]⁺.

### Step C: (S)-4-(3-fluorobenzyl)-N-(7-((4-hydroxy-4-methylpent-2-yn-1-yl)oxy)-5-methyl-4-oxo-2,3,4,5-tetrahydrobenzo[b][1,4]oxazepin-3-yl)-1H-pyrazole-1-carboxamide

The title compound was prepared in a similar fashion to Example 15 (Step C) with (S)-3-amino-7-((4-hydroxy-4-methylpent-2-yn-1-yl)oxy)-5-methyl-2,3-dihydrobenzo[b][1,4]oxazepin-4(5H)-one hydrochloride and 4-(3-fluorobenzyl)-1H-pyrazole hydrochloride (Intermediate 6). The residue was purified by column chromatography on SiO₂ (Hexanes:EtOAc=1:4) to give (S)-4-(3-fluorobenzyl)-N-(7-((4-hydroxy-4-methylpent-2-yn-1-yl)oxy)-5-methyl-4-oxo-2,3,4,5-tetrahydrobenzo[b][1,4]oxazepin-3-yl)-1H-pyrazole-1-carboxamide (67% for 2 steps) as a white foam. ¹H-NMR (400 MHz, CDCl₃): δ 7.97 (1H, d, J=7.2 Hz), 7.87 (1H, d, J=0.8 Hz), 7.46 (1H, s), 7.29-7.21 (1H, m), 7.13-7.10 (1H, m), 6.95-6.81 (5H, m), 4.90 (1H, td, J=10.8, 6.0 Hz), 4.70 (2H, d, J=2.0 Hz), 4.64 (1H, dd, J=10.0, 8.0 Hz), 4.24 (1H, dd, J=11.2, 10.0 Hz), 3.80 (2H, s), 3.41 (3H, s), 1.52 (6H, s). LC-MS: m/z=507.1 [M+H]⁺.

### Example 23: (S)-4-(3-fluorobenzyl)-N-(7-((3-(1-hydroxycyclohexyl)prop-2-yn-1-yl)oxy)-5-methyl-4-oxo-2,3,4,5-tetrahydrobenzo[b][1,4]oxazepin-3-yl)-1H-pyrazole-1-carboxamide

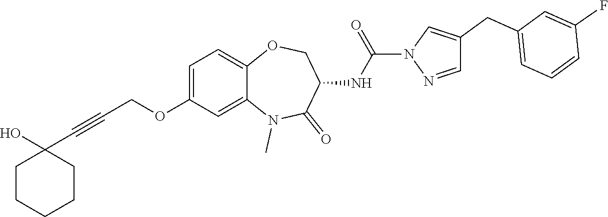 [SMILES: CN1C(=O)[C@@H](NC(=O)n2cc(Cc3cccc(F)c3)cn2)COc2ccc(OCC#CC3(O)CCCCC3)cc21]

### Step A: tert-butyl (S)-(7-((3-(1-hydroxycyclohexyl)prop-2-yn-1-yl)oxy)-5-methyl-4-oxo-2,3,4,5-tetrahydrobenzo[b][1,4]oxazepin-3-yl)carbamate

The title compound was prepared in a similar fashion to Example 15 (Step A) with Intermediate 2 and 1-(3-chloroprop-1-yn-1-yl)cyclohexan-1-ol. The crude product was purified by column chromatography on SiO₂ (Hexanes:EtOAc=1:1) to afford tert-butyl (S)-(7-((3-(1-hydroxycyclohexyl)prop-2-yn-1-yl)oxy)-5-methyl-4-oxo-2,3,4,5-tetrahydrobenzo[b][1,4]oxazepin-3-yl)carbamate (80%) as a white foam. ¹H-NMR (400 MHz, CDCl₃): δ 7.07 (1H, dd, J=6.9, 2.3 Hz), 6.80 (2H, dd, J=7.5, 2.5 Hz), 5.47 (1H, d, J=7.3 Hz), 4.72 (2H, d, J=2.3 Hz), 4.68-4.61 (1H, m), 4.53 (1H, dd, J=9.6, 7.3 Hz), 4.10 (1H, dd, J=11.4, 9.6 Hz), 3.38 (3H, s), 1.89-1.86 (2H, m), 1.70-1.65 (2H, m), 1.60-1.59 (2H, m), 1.52-1.43 (3H, m), 1.40 (9H, s), 1.27 (1H, m).

### Step B: (S)-3-amino-7-((3-(1-hydroxycyclohexyl)prop-2-yn-1-yl)oxy)-5-methyl-2,3-dihydrobenzo[b][1,4]oxazepin-4(5H)-one hydrochloride

The title compound was prepared in a similar fashion to Example 15 (Step B) with tert-butyl (S)-(7-((3-(1-hydroxycyclohexyl)prop-2-yn-1-yl)oxy)-5-methyl-4-oxo-2,3,4,5-tetrahydrobenzo[b][1,4]oxazepin-3-yl)carbamate. After concentration in vacuo, the crude product was used for the next reaction without purification. ¹H-NMR (400 MHz, DMSO-d₆): δ 8.43 (3H, s), 7.22-7.15 (2H, m), 6.92 (1H, dd, J=8.9, 3.0 Hz), 4.87 (2H, s), 4.51 (1H, dd, J=10.1, 7.8 Hz), 4.37 (1H, t, J=10.5 Hz), 4.22 (1H, dd, J=11.0, 7.8 Hz), 3.35 (3H, s), 1.71-1.69 (2H, m), 1.56-1.53 (2H, m), 1.46-1.30 (5H, m), 1.20-1.16 (1H, m).

### Step C: (S)-4-(3-fluorobenzyl)-N-(7-((3-(1-hydroxycyclohexyl)prop-2-yn-1-yl)oxy)-5-methyl-4-oxo-2,3,4,5-tetrahydrobenzo[b][1,4]oxazepin-3-yl)-1H-pyrazole-1-carboxamide

The title compound was prepared in a similar fashion to Example 15 (Step C) with (S)-3-amino-7-((3-(1-hydroxycyclohexyl)prop-2-yn-1-yl)oxy)-5-methyl-2,3-dihydrobenzo[b][1,4]oxazepin-4(5H)-one hydrochloride and 4-(3-fluorobenzyl)-1H-pyrazole hydrochloride (Intermediate 6). The crude product was purified by column chromatography on SiO₂ (Hexanes:EtOAc=1:2) to give (S)-4-(3-fluorobenzyl)-N-(7-((3-(1-hydroxycyclohexyl)prop-2-yn-1-yl)oxy)-5-methyl-4-oxo-2,3,4,5-tetrahydrobenzo[b][1,4] oxazepin-3-yl)-1H-pyrazole-1-carboxamide (55% for 2 steps) as a yellow solid. ¹H-NMR (400 MHz, CDCl₃): δ 7.98 (1H, d, J=7.3 Hz), 7.88 (1H, d, J=0.9 Hz), 7.47 (1H, s), 7.24-7.22 (1H, m), 7.13 (1H, dd, J=6.6, 2.5 Hz), 6.96-6.83 (5H, m), 4.93-4.86 (1H, m), 4.75 (2H, d, J=1.4 Hz), 4.67 (1H, dd, J=9.8, 7.5 Hz), 4.25 (1H, dd, J=11.2, 9.8 Hz), 3.81 (2H, s), 3.42 (3H, s), 1.90-1.87 (2H, m), 1.70-1.63 (2H, m), 1.60-1.56 (2H, m), 1.51-1.42 (4H, m). LC-MS: m/z=547.10 [M+H]⁺.

### Example 24: (S)-4-(3-fluorobenzyl)-N-(7-((3-(1-hydroxycyclobutyl)prop-2-yn-1-yl)oxy)-5-methyl-4-oxo-2,3,4,5-tetrahydrobenzo[b][1,4]oxazepin-3-yl)-1H-pyrazole-1-carboxamide

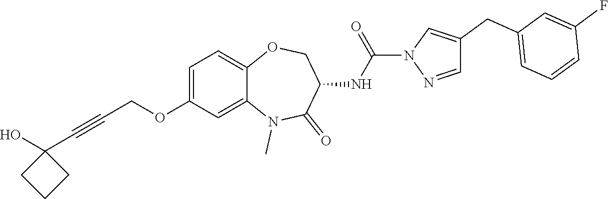 [SMILES: CN1C(=O)[C@@H](NC(=O)n2cc(Cc3cccc(F)c3)cn2)COc2ccc(OCC#CC3(O)CCC3)cc21]

### Step A: tert-butyl (S)-(7-((3-(1-hydroxycyclobutyl)prop-2-yn-1-yl)oxy)-5-methyl-4-oxo-2,3,4,5-tetrahydrobenzo[b][1,4]oxazepin-3-yl)carbamate

The title compound was prepared in a similar fashion to Example 15 (Step A) with Intermediate 2 and 1-(3-chloroprop-1-yn-1-yl)cyclobutan-1-ol (117 mg, 0.811 mmol). The crude product was purified by column chromatography on SiO₂ (Hexanes:EtOAc=2:1) to afford tert-butyl (S)-(7-((3-(1-hydroxycyclobutyl)prop-2-yn-1-yl)oxy)-5-methyl-4-oxo-2,3,4,5-tetrahydrobenzo[b][1,4]oxazepin-3-yl)carbamate (24%) as a white foam. ¹H-NMR (400 MHz, CDCl₃): δ 7.07 (1H, q, J=3.2 Hz), 6.82-6.79 (2H, m), 5.48 (1H, d, J=7.3 Hz), 4.73 (2H, d, J=1.8 Hz), 4.69-4.62 (1H, m), 4.53 (1H, dd, J=9.6, 7.3 Hz), 4.11 (1H, dd, J=11.2, 9.8 Hz), 3.38 (3H, s), 2.45-2.39 (2H, m), 2.30-2.21 (3H, m), 1.89-1.75 (2H, m), 1.40 (9H, s).

### Step B: (S)-3-amino-7-((3-(1-hydroxycyclobutyl)prop-2-yn-1-yl)oxy)-5-methyl-2,3-dihydrobenzo[b][1,4]oxazepin-4(5H)-one hydrochloride

The title compound was prepared in a similar fashion to Example 15 (Step B) with tert-butyl (S)-(7-((3-(1-hydroxycyclobutyl)prop-2-yn-1-yl)oxy)-5-methyl-4-oxo-2,3,4,5-tetrahydrobenzo[b][1,4]oxazepin-3-yl)carbamate. After concentration in vacuo, the crude product was used for the next reaction without purification. ¹H-NMR (400 MHz, DMSO-d₆): δ 8.38 (3H, s), 7.21 (1H, d, J=8.7 Hz), 7.15 (1H, d, J=2.7 Hz), 6.93 (1H, dd, J=8.7, 2.7 Hz), 4.88 (2H, s), 4.50 (1H, dd, J=9.6, 7.8 Hz), 4.36 (1H, t, J=10.5 Hz), 4.26 (1H, dd, J=11.0, 7.8 Hz), 3.35 (3H, s), 2.25-2.09 (4H, m), 1.77-1.61 (2H, m).

### Step C: (S)-4-(3-fluorobenzyl)-N-(7-((3-(1-hydroxycyclobutyl)prop-2-yn-1-yl)oxy)-5-methyl-4-oxo-2,3,4,5-tetrahydrobenzo[b][1,4]oxazepin-3-yl)-1H-pyrazole-1-carboxamide

The title compound was prepared in a similar fashion to Example 15 (Step C) with (S)-3-amino-7-((3-(1-hydroxycyclohexyl)prop-2-yn-1-yl)oxy)-5-methyl-2,3-dihydrobenzo[b][1,4]oxazepin-4(5H)-one hydrochloride and 4-(3-fluorobenzyl)-1H-pyrazole hydrochloride (Intermediate 6). The crude product was purified by column chromatography on SiO₂ (Hexanes:EtOAc=1:2) to give (S)-4-(3-fluorobenzyl)-N-(7-((3-(1-hydroxycyclobutyl)prop-2-yn-1-yl)oxy)-5-methyl-4-oxo-2,3,4,5-tetrahydrobenzo[b][1,4]oxazepin-3-yl)-1H-pyrazole-1-carboxamide (48% for 2 steps) as a white foam. ¹H-NMR (400 MHz, CDCl₃): δ 7.98 (1H, d, J=7.3 Hz), 7.88 (1H, d, J=0.9 Hz), 7.47 (1H, s), 7.24-7.22 (1H, m), 7.13 (1H, dt, J=8.8, 1.4 Hz), 6.96-6.84 (5H, m), 4.94-4.87 (1H, m), 4.75 (2H, d, J=1.4 Hz), 4.67 (1H, dd, J=9.6, 7.3 Hz), 4.26 (1H, dd, J=11.0, 10.1 Hz), 3.81 (2H, s), 3.42 (3H, s), 2.45-2.39 (2H, m), 2.2-2.22 (2H, m), 1.89-1.75 (2H, m). LC-MS: m/z=519.10 [M+H]⁺.

### Example 25: (S)-4-(3-fluorobenzyl)-N-(5-methyl-4-oxo-7-((3-(pyridin-3-yl)prop-2-yn-1-yl)oxy)-2,3,4,5-tetrahydrobenzo[b][1,4]oxazepin-3-yl)-1H-pyrazole-1-carboxamide

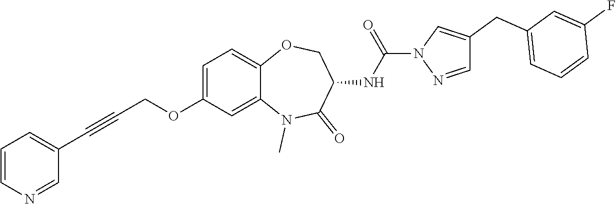 [SMILES: CN1C(=O)[C@@H](NC(=O)n2cc(Cc3cccc(F)c3)cn2)COc2ccc(OCC#Cc3cccnc3)cc21]

### Step A: tert-butyl (S)-(5-methyl-4-oxo-7-((3-(pyridin-3-yl)prop-2-yn-1-yl)oxy)-2,3,4,5-tetrahydrobenzo[b][1,4]oxazepin-3-yl)carbamate

The title compound was prepared in a similar fashion to Example 15 (Step A) with Intermediate 2 and 3-(pyridin-3-yl)prop-2-yn-1-yl methanesulfonate in ACN as a solvent. The crude product was purified by column chromatography on SiO₂ (Hexanes:EtOAc=4:1 to 1:1) to afford tert-butyl (S)-(5-methyl-4-oxo-7-((3-(pyridin-3-yl)prop-2-yn-1-yl)oxy)-2,3,4,5-tetrahydrobenzo[b][1,4]oxazepin-3-yl)carbamate (120 mg, 87%) as a yellow solid. ¹H-NMR (400 MHz, CDCl₃): δ 8.69 (1H, d, J=1.2 Hz), 8.56 (1H, dd, J=4.8, 1.6 Hz), 7.74 (1H, dt, J=8.4, 1.6 Hz), 7.30-7.25 (1H, m), 7.10 (1H, dd, J=7.2, 2.0 Hz), 6.88-6.84 (2H, m), 5.48 (1H, d, J=7.6 Hz), 4.92 (2H, s), 4.70-4.63 (1H, m), 4.54 (1H, dd, J=9.6, 7.6 Hz), 4.15-4.08 (1H, m), 3.39 (3H, s), 1.39 (9H, s).

### Step B: (S)-3-amino-5-methyl-7-((3-(pyridin-3-yl)prop-2-yn-1-yl)oxy)-2,3-dihydrobenzo[b][1,4]oxazepin-4(5H)-one dihydrochloride

The title compound was prepared in a similar fashion to Example 15 (Step B) with tert-butyl (S)-(5-methyl-4-oxo-7-((3-(pyridin-3-yl)prop-2-yn-1-yl)oxy)-2,3,4,5-tetrahydrobenzo[b][1,4]oxazepin-3-yl)carbamate. After concentration in vacuo, the crude product was used for the next reaction without purification. ¹H-NMR (400 MHz, DMSO-d₆): δ 8.74 (1H, d, J=1.2 Hz), 8.65 (1H, dd, J=4.8, 1.6 Hz), 8.55 (3H, d, J=3.6 Hz), 8.02 (1H, d, J=8.0 Hz), 7.55 (1H, dd, J=8.0, 4.8 Hz), 7.25-7.21 (2H, m), 7.00 (1H, dd, J=8.8, 3.2 Hz), 5.14 (2H, s), 4.55 (1H, dd, J=10.0, 8.0 Hz), 4.38 (1H, t, J=10.4 Hz), 4.25-4.23 (1H, m), 3.36 (3H, s).

### Step C: (S)-4-(3-fluorobenzyl)-N-(5-methyl-4-oxo-7-((3-(pyridin-3-yl)prop-2-yn-1-yl)oxy)-2,3,4,5-tetrahydrobenzo[b][1,4]oxazepin-3-yl)-1H-pyrazole-1-carboxamide

The title compound was prepared in a similar fashion to Example 15 (Step C) with (S)-3-amino-5-methyl-7-((3-(pyridin-3-yl)prop-2-yn-1-yl)oxy)-2,3-dihydrobenzo[b][1,4]oxazepin-4(5H)-one dihydrochloride and 4-(3-fluorobenzyl)-1H-pyrazole hydrochloride (Intermediate 6). The crude product was purified by column chromatography on SiO₂ (Hexanes:EtOAc=1:1) to give (S)-4-(3-fluorobenzyl)-N-(5-methyl-4-oxo-7-((3-(pyridin-3-yl)prop-2-yn-1-yl)oxy)-2,3,4,5-tetrahydrobenzo[b][1,4]oxazepin-3-yl)-1H-pyrazole-1-carboxamide (49% for 2 steps) as a white foam. ¹H-NMR (400 MHz, CDCl₃): δ 8.69 (1H, t, J=1.2 Hz), 8.56 (1H, dd, J=4.8, 1.6 Hz), 7.99 (1H, d, J=7.2 Hz), 7.88 (1H, d, J=0.8 Hz), 7.74 (1H, dt, J=7.6, 2.0 Hz), 7.47 (1H, s), 7.30-7.22 (2H, m), 7.15 (1H, d, J=8.8 Hz), 6.96-6.84 (5H, m), 4.94 (2H, s), 4.93-4.88 (1H, m), 4.67 (1H, dd, J=9.6, 7.2 Hz), 4.26 (1H, dd, J=10.8, 10.0 Hz), 3.81 (2H, s), 3.42 (3H, s). LC-MS: m/z=526.00 [M+H]⁺.

### Example 26: (S)-4-(3-fluorobenzyl)-N-(7-((4-hydroxy-4-methylpentyl)oxy)-5-methyl-4-oxo-2,3,4,5-tetrahydrobenzo[b][1,4]oxazepin-3-yl)-1H-pyrazole-1-carboxamide

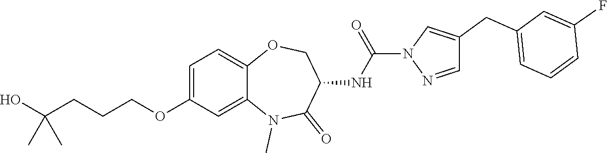 [SMILES: CN1C(=O)[C@@H](NC(=O)n2cc(Cc3cccc(F)c3)cn2)COc2ccc(OCCCC(C)(C)O)cc21]

A suspension of (S)-4-(3-fluorobenzyl)-N-(7-((4-hydroxy-4-methylpent-2-yn-1-yl)oxy)-5-methyl-4-oxo-2,3,4,5-tetrahydrobenzo[b][1,4]oxazepin-3-yl)-1H-pyrazole-1-carboxamide (Example 22, 15.0 mg, 0.0300 mmol) and Pd/C (10 wt %, 1.58 mg) in EtOAc (0.30 mL) was stirred at room temperature for 1 hour under H₂ atmosphere (1 atm). The reaction mixture was filtered through a Celite pad, washed with EtOAc and concentrated in vacuo to afford (S)-4-(3-fluorobenzyl)-N-(7-((4-hydroxy-4-methylpentyl)oxy)-5-methyl-4-oxo-2,3,4,5-tetrahydrobenzo[b][1,4]oxazepin-3-yl)-1H-pyrazole-1-carboxamide (14.0 mg, 93%). ¹H-NMR (400 MHz, CDCl₃): δ 7.98 (1H, d, J=7.2 Hz), 7.88 (1H, s), 7.47 (1H, s), 7.27-7.22 (1H, m), 7.11-7.09 (1H, m), 6.96-6.85 (3H, m), 6.76-6.74 (2H, m), 4.90 (1H, td, J=11.2, 5.6 Hz), 4.66 (1H, dd, J=9.6, 3.6 Hz), 4.24 (1H, dd, J=11.2, 9.6 Hz), 3.98 (1H, t, J=6.0 Hz), 3.81 (2H, m), 3.41 (3H, s), 1.94-1.86 (2H, m), 1.67-1.63 (2H, m), 1.28 (6H, s). LC-MS: m/z=511.10 [M+H]⁺.

### Example 27: (S)-4-(3-fluorobenzyl)-N-(7-(3-(1-hydroxycyclohexyl)propoxy)-5-methyl-4-oxo-2,3,4,5-tetrahydrobenzo[b][1,4]oxazepin-3-yl)-1H-pyrazole-1-carboxamide

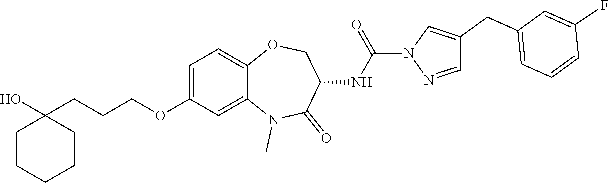 [SMILES: CN1C(=O)[C@@H](NC(=O)n2cc(Cc3cccc(F)c3)cn2)COc2ccc(OCCCC3(O)CCCCC3)cc21]

The title compound was prepared in a similar fashion to Example 26 with (S)-4-(3-fluorobenzyl)-N-(7-((3-(1-hydroxycyclohexyl)prop-2-yn-1-yl)oxy)-5-methyl-4-oxo-2,3,4,5-tetrahydrobenzo[b][1,4]oxazepin-3-yl)-1H-pyrazole-1-carboxamide (Example 23). ¹H-NMR (400 MHz, CDCl₃): δ 7.98 (1H, d, J=7.3 Hz), 7.88 (1H, d, J=0.9 Hz), 7.47 (1H, s), 7.24-7.22 (1H, m), 7.10 (1H, q, J=3.2 Hz), 6.96-6.85 (3H, m), 6.75 (2H, dd, J=7.1, 2.5 Hz), 4.93-4.86 (1H, m), 4.66 (1H, dd, J=9.8, 7.5 Hz), 4.24 (1H, dd, J=11.0, 10.1 Hz), 3.98 (2H, t, J=6.4 Hz), 3.81 (2H, s), 3.41 (3H, s), 1.94-1.86 (2H, m), 1.65-1.59 (5H, m), 1.51-1.43 (5H, m), 1.28-1.25 (2H, m). LC-MS: m/z=551.20 [M+H]⁺.

### Example 28: (S)-4-(3-fluorobenzyl)-N-(7-(3-(1-hydroxycyclobutyl)propoxy)-5-methyl-4-oxo-2,3,4,5-tetrahydrobenzo[b][1,4]oxazepin-3-yl)-1H-pyrazole-1-carboxamide

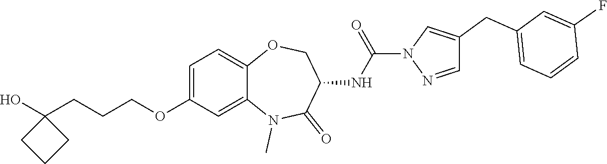 [SMILES: CN1C(=O)[C@@H](NC(=O)n2cc(Cc3cccc(F)c3)cn2)COc2ccc(OCCCC3(O)CCC3)cc21]

The title compound was prepared in a similar fashion to Example 26 with (S)-4-(3-fluorobenzyl)-N-(7-((3-(1-hydroxycyclobutyl)prop-2-yn-1-yl)oxy)-5-methyl-4-oxo-2,3,4,5-tetrahydrobenzo[b][1,4]oxazepin-3-yl)-1H-pyrazole-1-carboxamide (Example 24). ¹H-NMR (400 MHz, CDCl₃): δ 7.98 (1H, d, J=7.3 Hz), 7.88 (1H, d, J=0.9 Hz), 7.47 (1H, s), 7.24-7.22 (1H, m), 7.10 (1H, dd, J=6.9, 2.3 Hz), 6.96-6.85 (3H, m), 6.76 (2H, dd, J=7.5, 2.5 Hz), 4.93-4.87 (1H, m), 4.66 (1H, dd, J=9.8, 7.5 Hz), 4.24 (1H, dd, J=11.2, 9.8 Hz), 4.01 (2H, t, J=6.2 Hz), 3.81 (2H, s), 3.42 (3H, s), 2.14-1.99 (4H, m), 1.94-1.88 (2H, m), 1.83-1.75 (4H, m). LC-MS: m/z=523.20 [M+H]⁺.

### Example 29: (S)-4-(3-fluorobenzyl)-N-(5-methyl-4-oxo-7-(3-(pyridin-3-yl)propoxy)-2,3,4,5-tetrahydrobenzo[b][1,4]oxazepin-3-yl)-1H-pyrazole-1-carboxamide

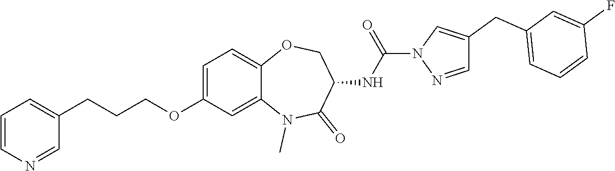 [SMILES: CN1C(=O)[C@@H](NC(=O)n2cc(Cc3cccc(F)c3)cn2)COc2ccc(OCCCc3cccnc3)cc21]

The title compound was prepared in a similar fashion to Example 26 with (S)-4-(3-fluorobenzyl)-N-(5-methyl-4-oxo-7-((3-(pyridin-3-yl)prop-2-yn-1-yl)oxy)-2,3,4,5-tetrahydrobenzo[b][1,4]oxazepin-3-yl)-1H-pyrazole-1-carboxamide (Example 25). ¹H-NMR (400 MHz, CDCl₃): δ 8.51 (1H, s), 8.48-8.46 (1H, m), 7.98 (1H, d, J=7.2 Hz), 7.88 (1H, s), 7.55 (1H, d, J=8.0 Hz), 7.46 (1H, s), 7.26-7.22 (2H, m), 7.11-7.09 (1H, m), 6.96-6.85 (3H, m), 6.74-6.73 (2H, m), 5.13 (1H, t, J=4.2 Hz), 4.91-4.84 (1H, m), 4.65 (1H, dd, J=7.2, 9.6 Hz), 4.26-4.19 (3H, m), 3.801 (2H, s), 3.41 (3H, d, J=2.4 Hz). LC-MS: m/z=530.20 [M+H]⁺.

### Example 30: (S)—N-(7-(cyclohexyloxy)-5-methyl-4-oxo-2,3,4,5-tetrahydrobenzo[b][1,4]oxazepin-3-yl)-4-(3-fluorobenzyl)-1H-pyrazole-1-carboxamide

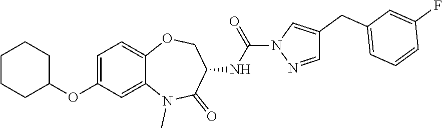 [SMILES: CN1C(=O)[C@@H](NC(=O)n2cc(Cc3cccc(F)c3)cn2)COc2ccc(OC3CCCCC3)cc21]

### Step A: tert-butyl (S)-(7-(cyclohexyloxy)-5-methyl-4-oxo-2,3,4,5-tetrahydrobenzo[b][1,4]oxazepin-3-yl)carbamate

The title compound was prepared in a similar fashion to Example 15 (Step A) with Intermediate 2 and cyclohexyl methanesulfonate. The crude product was purified by column chromatography on SiO₂ (Hexanes:EtOAc=2:1) to afford tert-butyl (S)-(7-(cyclohexyloxy)-5-methyl-4-oxo-2,3,4,5-tetrahydrobenzo[b][1,4]oxazepin-3-yl)carbamate (32%) as a white foam. ¹H-NMR (400 MHz, CDCl₃): δ 7.03-7.00 (1H, m), 6.70 (2H, q, J=3.2 Hz), 5.48 (1H, d, J=7.3 Hz), 4.73-4.63 (1H, m), 4.53 (1H, dd, J=9.6, 7.8 Hz), 4.17 (1H, td, J=8.6, 4.1 Hz), 4.08 (1H, dd, J=11.4, 9.6 Hz), 3.37 (3H, s), 2.01-1.96 (2H, m), 1.82-1.76 (2H, m), 1.70-1.61 (2H, m), 1.53-1.48 (1H, m), 1.40 (9H, s), 1.38-1.25 (3H, m).

### Step B: (S)-3-amino-7-(cyclohexyloxy)-5-methyl-2,3-dihydrobenzo[b][1,4]oxazepin-4(5H)-one hydrochloride

The title compound was prepared in a similar fashion to Example 15 (Step B) with tert-butyl (S)-(7-(cyclohexyloxy)-5-methyl-4-oxo-2,3,4,5-tetrahydrobenzo[b][1,4]oxazepin-3-yl)carbamate. After concentration in vacuo, the crude product was used for the next reaction without purification. LC-MS: m/z=291.10 [M+H]⁺.

### Step C: (S)—N-(7-(cyclohexyloxy)-5-methyl-4-oxo-2,3,4,5-tetrahydrobenzo[b][1,4]oxazepin-3-yl)-4-(3-fluorobenzyl)-1H-pyrazole-1-carboxamide

The title compound was prepared in a similar fashion to Example 15 (Step C) (S)-3-amino-7-(cyclohexyloxy)-5-methyl-2,3-dihydrobenzo[b][1,4]oxazepin-4(5H)-one hydrochloride and 4-(3-fluorobenzyl)-1H-pyrazole hydrochloride (Intermediate 6) The crude product was purified by column chromatography on SiO₂ (Hexanes:EtOAc=3:1) to give (S)—N-(7-(cyclohexyloxy)-5-methyl-4-oxo-2,3,4,5-tetrahydrobenzo[b][1,4]oxazepin-3-yl)-4-(3-fluorobenzyl)-1H-pyrazole-1-carboxamide (15% for 2 steps) as a white foam. ¹H-NMR (400 MHz, CDCl₃): δ 7.99 (1H, d, J=7.3 Hz), 7.88 (1H, d, J=0.9 Hz), 7.47 (1H, s), 7.24-7.22 (1H, m), 7.09-7.07 (1H, m), 7.00-6.85 (3H, m), 6.75 (2H, dd, J=8.5, 2.1 Hz), 4.95-4.88 (1H, m), 4.66 (1H, dd, J=9.8, 7.5 Hz), 4.26-4.18 (2H, m), 3.81 (2H, s), 3.41 (3H, s), 2.00-1.95 (2H, m), 1.8-1.78 (2H, m), 1.61-157 (1H, m), 1.54-1.49 (1H, m), 1.43-1.28 (4H, m). LC-MS: m/z=493.20 [M+H]⁺.

### Example 31: (S)—N-(7-((4,4-dimethylcyclohexyl)oxy)-5-methyl-4-oxo-2,3,4,5-tetrahydrobenzo[b][1,4]oxazepin-3-yl)-4-(3-fluorobenzyl)-1H-pyrazole-1-carboxamide

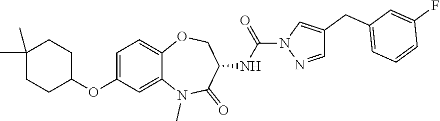 [SMILES: CN1C(=O)[C@@H](NC(=O)n2cc(Cc3cccc(F)c3)cn2)COc2ccc(OC3CCC(C)(C)CC3)cc21]

### Step A: tert-butyl-(S)-(7-((4,4-dimethylcyclohexyl)oxy)-5-methyl-4-oxo-2,3,4,5-tetrahydrobenzo[b][1,4]oxazepin-3-yl)carbamate

The title compound was prepared in a similar fashion to Example 15 (Step A) with Intermediate 2 and 4,4-dimethylcyclohexyl methanesulfonate. The crude product was purified by column chromatography on SiO₂ (Hexanes:EtOAc=1:1) to afford tert-butyl(S)-(7-((4,4-dimethylcyclohexyl)oxy)-5-methyl-4-oxo-2,3,4,5-tetrahydrobenzo[b][1,4]oxazepin-3-yl)carbamate (68%) as a white foam. ¹H-NMR (400 MHz, CDCl₃): δ 7.00 (1H, dd, J=6.6, 2.5 Hz), 6.68 (2H, dd, J=7.3, 2.7 Hz), 5.47 (1H, d, J=6.9 Hz), 4.71-4.63 (2H, m), 4.51 (1H, dd, J=9.6, 7.3 Hz), 4.11-4.04 (1H, m), 3.35 (3H, s), 1.90-1.73 (2H, m), 1.70-1.61 (1H, m), 1.54-1.45 (2H, m), 1.38 (9H, s), 1.29-1.22 (3H, m), 0.92 (6H, d, J=12.3 Hz).

### Step B: (S)-3-amino-7-((4,4-dimethylcyclohexyl)oxy)-5-methyl-2,3-dihydrobenzo[b][1,4]oxazepin-4(5H)-one hydrochloride

The title compound was prepared in a similar fashion to Example 15 (Step B) with tert-butyl(S)-(7-((4,4-dimethylcyclohexyl)oxy)-5-methyl-4-oxo-2,3,4,5-tetrahydrobenzo[b][1,4]oxazepin-3-yl)carbamate. After concentration in vacuo, the crude product was used for the next reaction without purification. LC-MS: m/z=319.10 [M+H]⁺.

### Step C: (S)—N-(7-((4,4-dimethylcyclohexyl)oxy)-5-methyl-4-oxo-2,3,4,5-tetrahydrobenzo[b][1,4]oxazepin-3-yl)-4-(3-fluorobenzyl)-1H-pyrazole-1-carboxamide

The title compound was prepared in a similar fashion to Example 15 (Step C) with (S)-3-amino-7-((4,4-dimethylcyclohexyl)oxy)-5-methyl-2,3-dihydrobenzo[b][1,4]oxazepin-4(5H)-one hydrochloride and 4-(3-fluorobenzyl)-1H-pyrazole hydrochloride (Intermediate 6). The crude product was purified by column chromatography on SiO₂ (Hexanes:EtOAc=5:1) to give (S)—N-(7-((4,4-dimethylcyclohexyl)oxy)-5-methyl-4-oxo-2,3,4,5-tetrahydrobenzo[b][1,4]oxazepin-3-yl)-4-(3-fluorobenzyl)-1H-pyrazole-1-carboxamide (55% for 2 steps) as a white foam. ¹H-NMR (400 MHz, CDCl₃): δ 7.99 (1H, d, J=7.3 Hz), 7.88 (1H, d, J=0.9 Hz), 7.47 (1H, s), 7.24-7.22 (1H, m), 7.09-7.07 (1H, m), 7.00-6.85 (3H, m), 6.75 (2H, dd, J=8.0, 2.5 Hz), 4.95-4.88 (1H, m), 4.66 (1H, dd, J=9.8, 7.5 Hz), 4.26-4.16 (1H, m), 3.81 (2H, s), 3.41 (3H, s), 1.90-1.84 (2H, m), 1.73-1.64 (2H, m), 1.53-1.49 (1H, m), 1.31-1.24 (3H, m), 0.97 (6H, d, J=10.5 Hz). LC-MS: m/z=521.10 [M+H]⁺.

### Example 32: 4-(3-fluorobenzyl)-N—((S)-5-methyl-4-oxo-7-(((R)-tetrahydrofuran-3-yl)oxy)-2,3,4,5-tetrahydrobenzo[b][1,4]oxazepin-3-yl)-1H-pyrazole-1-carboxamide

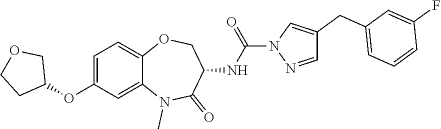

### Step A: tert-butyl ((S)-5-methyl-4-oxo-7-(((R)-tetrahydrofuran-3-yl)oxy)-2,3,4,5-tetrahydrobenzo[b][1,4]oxazepin-3-yl)carbamate

To a solution of tert-butyl (S)-(7-hydroxy-5-methyl-4-oxo-2,3,4,5-tetrahydrobenzo[b][1,4]oxazepin-3-yl)carbamate (Intermediate 2, 100 mg, 0.324 mmol), (S)-tetrahydrofuran-3-ol (0.0370 mg, 0.442 mmol) in THF (3.0 mL) was added PPh₃ (170 mg, 0.649 mmol) followed by DIAD (0.126 mL, 0.649 mmol) at 0° C. The reaction mixture was stirred at 40° C. for 5 hours. After concentration in vacuo, the residue was purified by column chromatography on SiO₂ (Hexanes:EtOAc=2:1) followed by column chromatography on NH₂—SiO₂ (Hexanes:EtOAc=1:1) to give tert-butyl ((S)-5-methyl-4-oxo-7-(((R)-tetrahydrofuran-3-yl)oxy)-2,3,4,5-tetrahydrobenzo[b][1,4]oxazepin-3-yl)carbamate (96.0 mg, 78%) as a white solid. ¹H-NMR (400 MHz, CDCl₃): δ 7.04 (1H, d, J=8.8 Hz), 6.69 (1H, d, J=2.8 Hz), 6.64 (1H, dd, J=8.8, 3.2 Hz), 5.47 (1H, d, J=7.2 Hz), 4.86-4.87 (1H, m), 4.63-4.66 (1H, m), 4.51 (1H, dd, J=10.0, 7.6 Hz), 4.08-4.11 (1H, m), 3.96-3.99 (3H, m), 3.90-3.92 (1H, m), 3.36 (3H, s), 2.22-2.13 (2H, m), 1.39 (9H, s).

### Step B: (S)-3-amino-5-methyl-7-(((R)-tetrahydrofuran-3-yl)oxy)-2,3-dihydrobenzo[b][1,4]oxazepin-4(5H)-one hydrochloride

The title compound was prepared in a similar fashion to Example 15 (Step B) with tert-butyl ((S)-5-methyl-4-oxo-7-(((R)-tetrahydrofuran-3-yl)oxy)-2,3,4,5-tetrahydrobenzo[b][1,4]oxazepin-3-yl)carbamate. After concentration in vacuo, the crude product was used for the next reaction without purification. LC-MS: m/z=279.1 [M+H]⁺.

### Step C: 4-(3-fluorobenzyl)-N—((S)-5-methyl-4-oxo-7-(((R)-tetrahydrofuran-3-yl)oxy)-2,3,4,5-tetrahydrobenzo[b][1,4]oxazepin-3-yl)-1H-pyrazole-1-carboxamide

The title compound was prepared in a similar fashion to Example 15 (Step C) with (S)-3-amino-5-methyl-7-(((R)-tetrahydrofuran-3-yl)oxy)-2,3-dihydrobenzo[b][1,4]oxazepin-4(5H)-one hydrochloride and 4-(3-fluorobenzyl)-1H-pyrazole hydrochloride (Intermediate 6). The crude product was purified by column chromatography on SiO₂ (Hexanes:EtOAc=3:1 to 1:1) followed by column chromatography on NH₂—SiO₂ (Hexanes:EtOAc=1:1) to give 4-(3-fluorobenzyl)-N—((S)-5-methyl-4-oxo-7-(((R)-tetrahydrofuran-3-yl)oxy)-2,3,4,5-tetrahydrobenzo[b][1,4]oxazepin-3-yl)-1H-pyrazole-1-carboxamide (30% for 2 steps) as a white foam. ¹H-NMR (400 MHz, CDCl₃): δ7.97 (1H, d, J=7.2 Hz), 7.86 (1H, s), 7.45 (1H, s), 7.29-7.21 (1H, m), 7.10 (1H, d, J=8.8 Hz), 6.95-8.84 (3H, m), 6.73 (1H, d, J=3.5 Hz), 6.69 (1H, dd, J=8.6, 2.6 Hz), 4.93-4.87 (2H, m), 4.65 (1H, dd, J=9.6, 7.6 Hz), 4.24 (1H, dd, J=11.0, 9.8 Hz), 3.97-4.00 (3H, m), 3.91-3.93 (1H, m), 3.80 (2H, s), 3.39 (3H, s), 2.17-2.21 (2H, m). LC-MS: m/z=481.1 [M+H]⁺.

### Example 33: 4-(3-fluorobenzyl)-N—((S)-5-methyl-7-(((R)-1-methylpyrrolidin-3-yl)oxy)-4-oxo-2,3,4,5-tetrahydrobenzo[b][1,4]oxazepin-3-yl)-1H-pyrazole-1-carboxamide

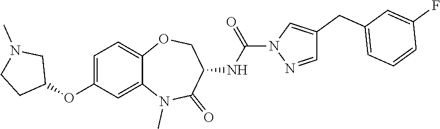

### Step A: tert-butyl ((S)-5-methyl-7-(((R)-1-methylpyrrolidin-3-yl)oxy)-4-oxo-2,3,4,5-tetrahydrobenzo[b][1,4]oxazepin-3-yl)carbamate

The title compound was prepared in a similar fashion to Example 15 (Step A) with Intermediate 2 and (S)-1-methylpyrrolidin-3-yl methanesulfonate. The crude product was purified by column chromatography on SiO₂ (DCM:MeOH=10:1 to 7:1) to afford tert-butyl ((S)-5-methyl-7-(((R)-1-methylpyrrolidin-3-yl)oxy)-4-oxo-2,3,4,5-tetrahydrobenzo[b][1,4]oxazepin-3-yl)carbamate (31%) as a white solid. ¹H-NMR (400 MHz, CDCl₃): δ 7.02 (1H, d, J=8.8 Hz), 6.67 (1H, d, J=2.8 Hz), 6.62 (1H, dd, J=8.6, 2.6 Hz), 5.47 (1H, d, J=7.2 Hz), 4.78-4.75 (1H, m), 4.65-4.60 (1H, m), 4.51 (1H, dd, J=9.6, 7.2 Hz), 4.07 (1H, dd, J=11.2, 9.6 Hz), 3.35 (3H, s), 2.85-2.78 (3H, m), 2.45-2.40 (1H, m), 2.40 (3H, s), 2.31-2.28 (1H, m), 2.02-1.95 (1H, m), 1.38 (9H, s).

### Step B: (S)-3-amino-5-methyl-7-(((R)-1-methylpyrrolidin-3-yl)oxy)-2,3-dihydrobenzo[b][1,4]oxazepin-4(5H)-one hydrochloride

The title compound was prepared in a similar fashion to Example 15 (Step B) with tert-butyl ((S)-5-methyl-7-(((R)-1-methylpyrrolidin-3-yl)oxy)-4-oxo-2,3,4,5-tetrahydrobenzo[b][1,4]oxazepin-3-yl)carbamate. After concentration in vacuo, the crude product was used for the next reaction without purification. LC-MS: m/z=292.1 [M+H]⁺.

### Step C: 4-(3-fluorobenzyl)-N—((S)-5-methyl-7-(((R)-1-methylpyrrolidin-3-yl)oxy)-4-oxo-2,3,4,5-tetrahydrobenzo[b][1,4]oxazepin-3-yl)-1H-pyrazole-1-carboxamide

The title compound was prepared in a similar fashion to Example 15 (Step C) with (S)-3-amino-5-methyl-7-(((R)-1-methylpyrrolidin-3-yl)oxy)-2,3-dihydrobenzo[b][1,4]oxazepin-4(5H)-one and 4-(3-fluorobenzyl)-1H-pyrazole hydrochloride (Intermediate 6). The crude product was purified by column chromatography on SiO₂ (Hexanes:EtOAc=3:1 to EtOAc only) to give 4-(3-fluorobenzyl)-N—((S)-5-methyl-7-(((R)-1-methylpyrrolidin-3-yl)oxy)-4-oxo-2,3,4,5-tetrahydrobenzo[b][1,4]oxazepin-3-yl)-1H-pyrazole-1-carboxamide (18% for 2 steps) as a colorless oil. ¹H-NMR (400 MHz, CDCl₃): δ 7.97 (1H, d, J=7.6 Hz), 7.97 (1H, d, J=7.6 Hz), 7.87 (1H, s), 7.46 (1H, s), 7.24-7.22 (1H, m), 7.08 (1H, d, J=8.8 Hz), 6.95-6.86 (3H, m), 6.70 (1H, d, J=2.8 Hz), 6.67 (1H, dd, J=9.2, 2.8 Hz), 4.92-4.86 (1H, m), 4.83-4.76 (1H, m), 4.64 (1H, dd, J=10.0, 7.6 Hz), 4.23 (1H, dd, J=11.2, 10.0 Hz), 3.79 (2H, s), 3.39 (3H, s), 2.50-2.44 (3H, m), 2.41 (3H, s), 2.37-2.30 (1H, m), 2.05-1.99 (1H, m). LC-MS: m/z=494.1 [M+H]⁺.

### Example 34: 4-(3-fluorobenzyl)-N—((S)-7-(((1s,4R)-4-hydroxycyclohexyl)oxy)-5-methyl-4-oxo-2,3,4,5-tetrahydrobenzo[b][1,4]oxazepin-3-yl)-1H-pyrazole-1-carboxamide

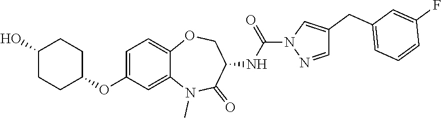 [SMILES: CN1C(=O)[C@@H](NC(=O)n2cc(Cc3cccc(F)c3)cn2)COc2ccc(O[C@H]3CC[C@@H](O)CC3)cc21]

### Step A: tert-butyl ((S)-7-(((1s,4R)-4-((tert-butyldimethylsilyl)oxy)cyclohexyl)oxy)-5-methyl-4-oxo-2,3,4,5-tetrahydrobenzo[b][1,4]oxazepin-3-yl)carbamate

The title compound was prepared in a similar fashion to Example 32 (Step A) with Intermediate 2 and (1r,4r)-4-((tert-butyldimethylsilyl)oxy)cyclohexan-1-ol. The crude product was purified by column chromatography on SiO₂ (Hexanes:EtOAc=3:1) to afford tert-butyl ((S)-7-(((1s,4R)-4-((tert-butyldimethylsilyl)oxy)cyclohexyl)oxy)-5-methyl-4-oxo-2,3,4,5-tetrahydrobenzo[b][1,4]oxazepin-3-yl)carbamate (54%) as colorless oil. ¹H-NMR (400 MHz, CDCl₃) δ 7.03 (1H, d, J=8.4 Hz), 6.74-6.71 (2H, m), 5.49 (1H, d, J=7.2 Hz), 4.67 (1H, dt, J=10.8, 7.2 Hz), 4.54 (1H, dd, J=9.6, 7.2 Hz), 4.25-4.21 (1H, m), 4.10-4.07 (1H, m), 3.84-3.80 (1H, m), 3.38 (3H, s), 1.89-1.82 (4H, m), 1.52-1.45 (4H, m), 0.91 (9H, s), 0.07 (6H, s).

### Step B: (S)-3-amino-7-(((1s,4R)-4-hydroxycyclohexyl)oxy)-5-methyl-2,3-dihydrobenzo[b][1,4]oxazepin-4(5H)-one hydrochloride

The title compound was prepared in a similar fashion to Example 15 (Step B) with tert-butyl ((S)-7-(((1s,4R)-4-((tert-butyldimethylsilyl)oxy)cyclohexyl)oxy)-5-methyl-4-oxo-2,3,4,5-tetrahydrobenzo[b][1,4]oxazepin-3-yl)carbamate. After concentration in vacuo, the crude product was used for the next reaction without purification. LC-MS: m/z=307.10 [M+H]⁺.

### Step C: 4-(3-fluorobenzyl)-N—((S)-7-(((1s,4R)-4-hydroxycyclohexyl)oxy)-5-methyl-4-oxo-2,3,4,5-tetrahydrobenzo[b][1,4]oxazepin-3-yl)-1H-pyrazole-1-carboxamide

The title compound was prepared in a similar fashion to Example 15 (Step C) with (S)-3-amino-7-(((1s,4R)-4-hydroxycyclohexyl)oxy)-5-methyl-2,3-dihydrobenzo[b][1,4]oxazepin-4(5H)-one hydrochloride and 4-(3-fluorobenzyl)-1H-pyrazole hydrochloride (Intermediate 6).

The crude product was purified by column chromatography on SiO₂ (Hexanes:EtOAc=1:1 to 1:3) to afford 4-(3-fluorobenzyl)-N—((S)-7-(((1s,4R)-4-hydroxycyclohexyl)oxy)-5-methyl-4-oxo-2,3,4,5-tetrahydrobenzo[b][1,4]oxazepin-3-yl)-1H-pyrazole-1-carboxamide (21% for 2 steps) as white foam. ¹H-NMR (400 MHz, CDCl₃) δ 7.99 (1H, d, J=7.2 Hz), 7.88 (1H, d, J=0.8 Hz), 7.47 (1H, s), 7.28-7.23 (1H, m), 7.10 (1H, d, J=9.6 Hz), 6.97-6.85 (3H, m), 6.78-6.75 (2H, m), 4.92 (2H, dt, J=11.6, 7.6 Hz), 4.66 (1H, dd, J=9.6, 7.6 Hz), 4.38-4.34 (1H, m), 4.24 (1H, dd, J=11.6, 9.6 Hz), 3.84-3.78 (3H, m), 3.41 (3H, s), 2.07-1.98 (2H, m), 1.80-1.75 (4H, m), 1.73-1.65 (2H, m). LC-MS: m/z=509.10 [M+H]⁺.

### Example 35: 4-(3-fluorobenzyl)-N—((S)-7-(((1r,4S)-4-hydroxycyclohexyl)oxy)-5-methyl-4-oxo-2,3,4,5-tetrahydrobenzo[b][1,4]oxazepin-3-yl)-1H-pyrazole-1-carboxamide

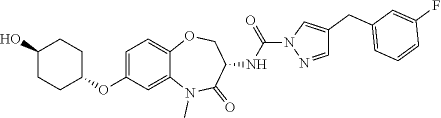 [SMILES: CN1C(=O)[C@@H](NC(=O)n2cc(Cc3cccc(F)c3)cn2)COc2ccc(O[C@H]3CC[C@H](O)CC3)cc21]

### Step A: of tert-butyl-((S)-7-(((1r,4S)-4-((tert-butyldimethylsilyl)oxy)cyclohexyl)oxy)-5-methyl-4-oxo-2,3,4,5-tetrahydrobenzo[b][1,4]oxazepin-3-yl)carbamate

The title compound was prepared in a similar fashion to Example 32 (Step A) with Intermediate 2 and (1s,4s)-4-((tert-butyldimethylsilyl)oxy)cyclohexan-1-ol. The crude product was purified by column chromatography on SiO₂ (Hexanes:EtOAc=1:1) to give tert-butyl ((S)-7-(((1r,4S)-4-((tert-butyldimethylsilyl)oxy)cyclohexyl)oxy)-5-methyl-4-oxo-2,3,4,5-tetrahydrobenzo[b][1,4]oxazepin-3-yl)carbamate (60.0 mg, 12%) as a white foam. ¹H-NMR (400 MHz, CDCl₃): δ 7.03 (1H, dd, J=5.5, 3.7 Hz), 6.69 (2H, q, J=2.7 Hz), 5.48 (1H, d, J=7.3 Hz), 4.69-4.63 (1H, m), 4.53 (1H, dd, J=9.6, 7.8 Hz), 4.24-4.20 (1H, m), 4.09 (1H, dd, J=11.2, 9.8 Hz), 3.81-3.77 (1H, m), 3.37 (3H, s), 2.10-2.04 (2H, m), 1.92-1.87 (2H, m), 1.40 (9H, s), 1.27-1.22 (4H. m), 0.87 (9H, s), 0.04 (6H, s).

### Step B: (S)-3-amino-7-(((1r,4S)-4-hydroxycyclohexyl)oxy)-5-methyl-2,3-dihydrobenzo[b][1,4]oxazepin-4(5H)-one hydrochloride

The title compound was prepared in a similar fashion to Example 15 (Step B) with tert-butyl-((S)-7-(((1r,4S)-4-((tert-butyldimethylsilyl)oxy)cyclohexyl)oxy)-5-methyl-4-oxo-2,3,4,5-tetrahydrobenzo[b][1,4]oxazepin-3-yl)carbamate. After concentration in vacuo, the crude product was used for the next reaction without purification. LC-MS: m/z=307.10 [M+H]⁺.

### Step C: 4-(3-fluorobenzyl)-N—((S)-7-(((1r,4S)-4-hydroxycyclohexyl)oxy)-5-methyl-4-oxo-2,3,4,5-tetrahydrobenzo[b][1,4]oxazepin-3-yl)-1H-pyrazole-1-carboxamide

The title compound was prepared in a similar fashion to Example 15 (Step C) with (S)-3-amino-7-(((1r,4S)-4-hydroxycyclohexyl)oxy)-5-methyl-2,3-dihydrobenzo[b][1,4]oxazepin-4(5H)-one hydrochloride and 4-(3-fluorobenzyl)-1H-pyrazole hydrochloride (Intermediate 6). The crude product was purified by column chromatography on SiO₂ (Hexanes:EtOAc=2:1) to give 4-(3-fluorobenzyl)-N—((S)-7-(((1r,4S)-4-hydroxycyclohexyl)oxy)-5-methyl-4-oxo-2,3,4,5-tetrahydrobenzo[b][1,4]oxazepin-3-yl)-1H-pyrazole-1-carboxamide (24% for 2 steps) as a white foam. ¹H-NMR (400 MHz, CDCl₃): δ 7.98 (1H, d, J=7.3 Hz), 7.88 (1H, s), 7.47 (1H, s), 7.24-7.22 (1H, m), 7.09 (1H, d, J=9.1 Hz), 7.00-6.85 (3H, m), 6.75 (2H, dd, J=11.2, 2.5 Hz), 4.95-4.88 (1H, m), 4.66 (1H, dd, J=9.6, 7.8 Hz), 4.27-4.21 (2H, m), 3.85-3.81 (3H, m), 3.41 (3H, s), 2.15-2.10 (2H, m), 2.05 (2H, m), 1.51-1.44 (4H, m). LC-MS: m/z=509.10 [M+H]⁺.

### General Synthetic Scheme for 2-Oxyacetamide Analogues

### Route 1

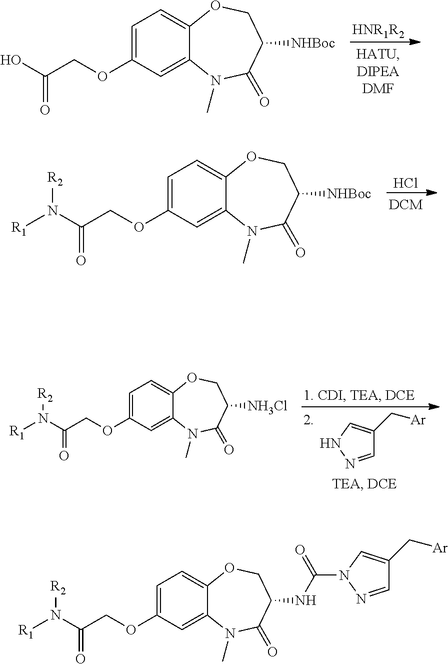

### Route 2

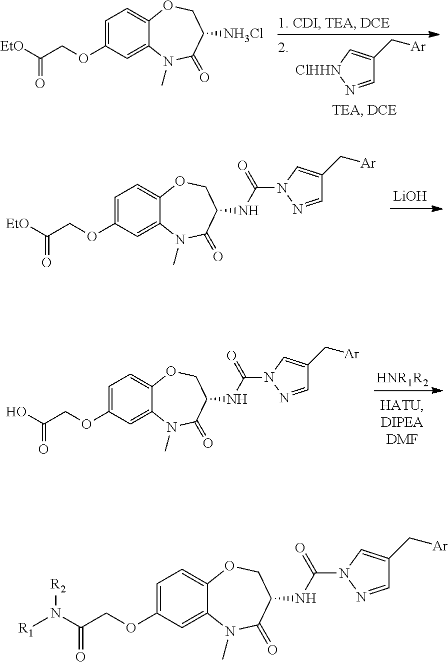

### Example 36: (S)-4-(3-fluorobenzyl)-N-(5-methyl-4-oxo-7-(2-oxo-2-(piperidin-1-yl)ethoxy)-2,3,4,5-tetrahydrobenzo[b][1,4]oxazepin-3-yl)-1H-pyrazole-1-carboxamide

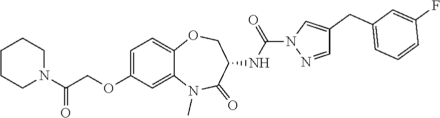 [SMILES: CN1C(=O)[C@@H](NC(=O)n2cc(Cc3cccc(F)c3)cn2)COc2ccc(OCC(=O)N3CCCCC3)cc21]

To a solution of(S)-2-((3-(4-(3-fluorobenzyl)-1H-pyrazole-1-carboxamido)-5-methyl-4-oxo-2,3,4,5-tetrahydrobenzo[b][1,4]oxazepin-7-yl)oxy)acetic acid (Intermediate 4, 50.0 mg, 0.110 mmol) and piperidine (13.0 μL, 0.130 mmol) in DMF (1.0 mL) was added DIPEA (28.0 μL, 0.0 mmol) followed by HATU (60.0 mg, 0.160 mmol) at 0° C. The reaction mixture was stirred at 0° C. for 10 min. After quenched with water, the mixture was extracted with EtOAc twice. The combined organic layers were washed with water and brine, dried over Na₂SO₄, filtered, and concentrated in vacuo. The residue was purified by column chromatography on SiO₂ (Hexanes:EtOAc=1:1) to give (S)-4-(3-fluorobenzyl)-N-(5-methyl-4-oxo-7-(2-oxo-2-(piperidin-1-yl)ethoxy)-2,3,4,5-tetrahydrobenzo[b][1,4]oxazepin-3-yl)-1H-pyrazole-1-carboxamide (35.0 mg, 61%) as a white foam. ¹H-NMR (400 MHz, CDCl₃): δ 7.98 (1H, d, J=7.3 Hz), 7.88 (1H, d, J=0.9 Hz), 7.47 (1H, s), 7.24-7.22 (1H, m), 7.11 (1H, d, J=8.7 Hz), 7.00-6.85 (4H, m), 6.80 (1H, dd, J=8.7, 3.2 Hz), 4.93-4.87 (1H, m), 4.69 (2H, s), 4.66-4.64 (1H, m), 4.25 (1H, dd, J=11.2, 9.8 Hz), 3.81 (2H, s), 3.59-3.56 (2H, m), 3.50-3.48 (2H, m), 3.41 (3H, s), 1.67-1.61 (6H, m). LC-MS: m/z=536.10 [M+H]⁺.

### Example 37: (S)—N-(7-(2-(4,4-dimethylpiperidin-1-yl)-2-oxoethoxy)-5-methyl-4-oxo-2,3,4,5-tetrahydrobenzo[b][1,4]oxazepin-3-yl)-4-(3-fluorobenzyl)-1H-pyrazole-1-carboxamide

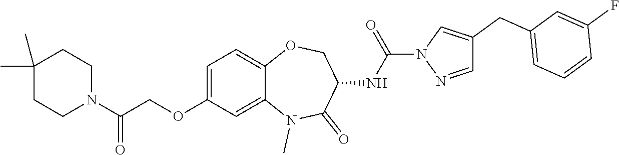 [SMILES: CN1C(=O)[C@@H](NC(=O)n2cc(Cc3cccc(F)c3)cn2)COc2ccc(OCC(=O)N3CCC(C)(C)CC3)cc21]

The title compound was prepared in a similar fashion to Example 36 with Intermediate 4 and 4,4-dimethylpiperidine hydrochloride in DMSO as a solvent. The crude product was purified by column chromatography on SiO₂ (Hexanes:EtOAc=1:1) to give (S)—N-(7-(2-(4,4-dimethylpiperidin-1-yl)-2-oxoethoxy)-5-methyl-4-oxo-2,3,4,5-tetrahydrobenzo[b][1,4]oxazepin-3-yl)-4-(3-fluorobenzyl)-1H-pyrazole-1-carboxamide (50%) as a white solid. ¹H-NMR (400 MHz, CDCl₃): δ 7.98 (1H, d, J=7.3 Hz), 7.88 (1H, d, J=0.9 Hz), 7.47 (1H, s), 7.24-7.22 (1H, m), 7.11 (1H, d, J=8.7 Hz), 7.00-6.85 (4H, m), 6.80 (1H, dd, J=8.9, 3.0 Hz), 4.93-4.87 (1H, m), 4.68 (2H, s), 4.66-4.64 (1H, m), 4.25 (1H, dd, J=11.0, 10.1 Hz), 3.81 (2H, s), 3.63-3.54 (2H, m), 3.49 (2H, t, J=5.7 Hz), 3.41 (3H, s), 1.42-1.37 (4H, m), 0.99 (6H, s). LC-MS: m/z=564.20 [M+H]⁺.

### Example 38: (S)—N-(7-(2-(4,4-difluoropiperidin-1-yl)-2-oxoethoxy)-5-methyl-4-oxo-2,3,4,5-tetrahydrobenzo[b][1,4]oxazepin-3-yl)-4-(3-fluorobenzyl)-1H-pyrazole-1-carboxamide

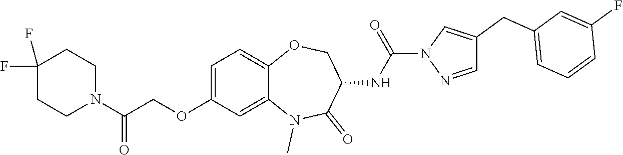 [SMILES: CN1C(=O)[C@@H](NC(=O)n2cc(Cc3cccc(F)c3)cn2)COc2ccc(OCC(=O)N3CCC(F)(F)CC3)cc21]

The title compound was prepared in a similar fashion to Example 36 with Intermediate 4 and 4,4-difluoropiperidine hydrochloride in DMSO as a solvent. The crude product was purified by column chromatography on SiO₂ (Hexanes:EtOAc=1:1) to give (S)—N-(7-(2-(4,4-difluoropiperidin-1-yl)-2-oxoethoxy)-5-methyl-4-oxo-2,3,4,5-tetrahydrobenzo[b][1,4]oxazepin-3-yl)-4-(3-fluorobenzyl)-1H-pyrazole-1-carboxamide (74%) as a white solid. ¹H-NMR (400 MHz, CDCl₃): δ 7.97 (1H, d, J=7.3 Hz), 7.88 (1H, s), 7.47 (1H, s), 7.25-7.22 (1H, m), 7.13 (1H, d, J=8.7 Hz), 7.00-6.78 (5H, m), 4.94-4.87 (1H, m), 4.71 (2H, s), 4.66 (1H, dd, J=9.8, 7.5 Hz), 4.26 (1H, dd, J=11.2, 9.8 Hz), 3.81 (2H, s), 3.79-3.70 (4H, m), 3.41 (3H, s), 2.08-1.96 (4H, m). LC-MS: m/z=572.10 [M+H]⁺.

### Example 39: (S)-4-(3-fluorobenzyl)-N-(5-methyl-7-(2-morpholino-2-oxoethoxy)-4-oxo-2,3,4,5-tetrahydrobenzo[b][1,4]oxazepin-3-yl)-1H-pyrazole-1-carboxamide

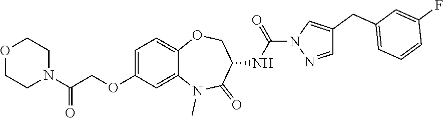 [SMILES: CN1C(=O)[C@@H](NC(=O)n2cc(Cc3cccc(F)c3)cn2)COc2ccc(OCC(=O)N3CCOCC3)cc21]

The title compound was prepared in a similar fashion to Example 36 with Intermediate 4 and morpholine in DMSO as a solvent. The crude product was purified by column chromatography on SiO₂ (DCM:EtOAc=1:1) to afford (S)-4-(3-fluorobenzyl)-N-(5-methyl-7-(2-morpholino-2-oxoethoxy)-4-oxo-2,3,4,5-tetrahydrobenzo[b][1,4]oxazepin-3-yl)-1H-pyrazole-1-carboxamide (71%) as a white foam. ¹H-NMR (400 MHz, CDCl₃) δ 7.98 (1H, d, J=7.2 Hz), 7.88 (1H, d, J=1.2 Hz), 7.47 (1H, s), 7.29-7.23 (1H, m), 7.13 (1H, d, J=9.2 Hz), 6.96-6.85 (4H, m), 6.80 (1H, dd, J=9.2, 2.8 Hz), 4.90 (1H, dt, J=11.2, 7.6 Hz), 4.70 (2H, s), 4.66 (1H, dd, J=9.6, 7.6 Hz), 4.26 (1H, dd, J=11.2, 9.6 Hz), 3.81 (2H, s), 3.70-3.70 (4H, m), 3.67-3.65 (2H, m), 3.62-3.59 (2H, m), 3.41 (3H, s). LC-MS: m/z=538.20 [M+H]⁺.

### Example 40: (S)-4-(3-fluorobenzyl)-N-(7-(2-(4-hydroxypiperidin-1-yl)-2-oxoethoxy)-5-methyl-4-oxo-2,3,4,5-tetrahydrobenzo[b][1,4]oxazepin-3-yl)-1H-pyrazole-1-carboxamide

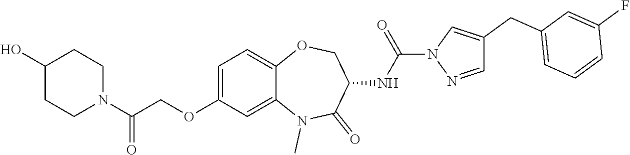 [SMILES: CN1C(=O)[C@@H](NC(=O)n2cc(Cc3cccc(F)c3)cn2)COc2ccc(OCC(=O)N3CCC(O)CC3)cc21]

The title compound was prepared in a similar fashion to Example 36 with Intermediate 4 and piperidin-4-ol. The crude product was purified by column chromatography on SiO₂ (EtOAc only) to afford (S)-4-(3-fluorobenzyl)-N-(7-(2-(4-hydroxypiperidin-1-yl)-2-oxoethoxy)-5-methyl-4-oxo-2,3,4,5-tetrahydrobenzo[b][1,4]oxazepin-3-yl)-1H-pyrazole-1-carboxamide (65%) as a white foam. ¹H-NMR (400 MHz, CDCl₃): δ 7.97 (1H, d, J=7.2 Hz), 7.87 (1H, s), 7.47 (1H, s), 7.26-7.22 (1H, m), 7.11 (1H, d, J=8.8 Hz), 6.96-6.85 (4H, m), 6.80 (1H, dd, J=8.8, 2.8 Hz), 4.90 (1H, dt, J=11.6, 5.6 Hz), 4.71 (2H, s), 4.64 (1H, t, J=9.2 Hz), 4.25 (1H, dd, J=11.8, 10.0 Hz), 4.09-4.00 (1H, m), 4.00-3.94 (1H, m), 3.89-3.81 (1H, m), 3.81 (2H, s), 3.41 (3H, s), 3.37-3.23 (2H, m), 1.97-1.85 (2H, m), 1.58-1.45 (2H, m). LC-MS: m/z=552.1 [M+H]⁺.

### Example 41: 4-(3-fluorobenzyl)-N-((3S)-7-(3-hydroxypyrrolidine-1-carbonyl)-5-methyl-4-oxo-2,3,4,5-tetrahydrobenzo[b][1,4]oxazepin-3-yl)-1H-pyrazole-1-carboxamide

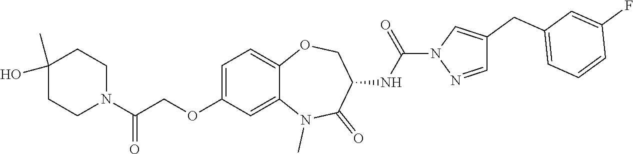 [SMILES: CN1C(=O)[C@@H](NC(=O)n2cc(Cc3cccc(F)c3)cn2)COc2ccc(OCC(=O)N3CCC(C)(O)CC3)cc21]

### Step A: tert-butyl (S)-(7-(2-(4-hydroxy-4-methylpiperidin-1-yl)-2-oxoethoxy)-5-methyl-4-oxo-2,3,4,5-tetrahydrobenzo[b][1,4]oxazepin-3-yl)carbamate

To a solution of (S)-2-((3-((tert-butoxycarbonyl)amino)-5-methyl-4-oxo-2,3,4,5-tetrahydrobenzo[b][1,4]oxazepin-7-yl)oxy)acetic acid (Intermediate 3, 0.100 g, 0.273 mmol) and 4-methylpiperidin-4-ol (31.0 mg, 0.273 mmol) in DMF (2.7 mL) was added DIPEA (0.143 mL, 0.819 mmol) followed by HATU (0.156 g, 0.409 mmol) at 0° C. The reaction mixture was stirred at room temperature for 1 hour. After quenched with water, the mixture was extracted with EtOAc twice. The combined organic layers were washed with brine, dried over Na₂SO₄, filtered, and concentrated in vacuo. The residue was purified by column chromatography on SiO₂ (DCM:MeOH=15:1) to afford tert-butyl (S)-(7-(2-(4-hydroxy-4-methylpiperidin-1-yl)-2-oxoethoxy)-5-methyl-4-oxo-2,3,4,5-tetrahydrobenzo[b][1,4]oxazepin-3-yl)carbamate (86.0 mg, 68%) as white foam. ¹H-NMR (400 MHz, CDCl₃) δ 7.06 (1H, d, J=8.8 Hz), 6.84 (1H, d, J=2.8 Hz), 6.78-6.74 (1H, m), 5.56 (1H, d, J=7.2 Hz), 4.72-4.61 (3H, m), 4.52 (1H, dd, J=9.6, 7.6 Hz), 4.20 (1H, m), 4.15-4.09 (1H, m), 3.72-3.60 (1H, m), 3.58-3.45 (1H, m), 3.38 (3H, s), 3.25-3.11 (1H, m), 1.68-1.50 (4H, m), 1.40 (9H, s), 1.28 (3H, s).

### Step B: (S)-3-amino-7-(4-hydroxy-4-methylpiperidine-1-carbonyl)-5-methyl-2,3-dihydrobenzo[b][1,4]oxazepin-4(5H)-one hydrochloride

To a solution of tert-butyl (S)-(7-(2-(4-hydroxy-4-methylpiperidin-1-yl)-2-oxoethoxy)-5-methyl-4-oxo-2,3,4,5-tetrahydrobenzo[b][1,4]oxazepin-3-yl)carbamate (86.0 mg, 0.186 mmol) in DCM (0.93 mL) was added HCl (4 M solution in dioxane, 1.39 mL, 5.57 mmol) at 0° C. The reaction mixture was stirred at room temperature for 20 hours. A precipitated solid was collected by filtration, washed with DCM and IPE and dried under vacuum to afford (S)-3-amino-7-(2-(4-hydroxy-4-methylpiperidin-1-yl)-2-oxoethoxy)-5-methyl-2,3-dihydrobenzo[b][1,4]oxazepin-4(5H)-one hydrochloride (77.0 mg, 99%) as yellow-green solid. ¹H-NMR (400 MHz, MeOH-d₄) δ 7.17 (1H, d, J=8.8 Hz), 7.04 (1H, t, J=2.8 Hz), 6.89 (1H, dd, J=8.8, 2.8 Hz), 4.87 (2H, s), 4.58 (1H, dd, J=9.6, 7.6 Hz), 4.41 (1H, dd, J=10.8, 9.6 Hz), 4.32 (1H, dd, J=10.8, 7.2 Hz), 4.15-4.06 (1H, m), 3.71-3.60 (1H, m), 3.55-3.45 (1H, m), 3.41 (3H, s), 3.23-3.15 (1H, m), 1.66-1.48 (4H, m), 1.25 (3H, s).

### Step C: 4-(3-fluorobenzyl)-N-((3S)-7-(3-hydroxypyrrolidine-1-carbonyl)-5-methyl-4-oxo-2,3,4,5-tetrahydrobenzo[b][1,4]oxazepin-3-yl)-1H-pyrazole-1-carboxamide

To a solution of (S)-3-amino-7-(2-(4-hydroxy-4-methylpiperidin-1-yl)-2-oxoethoxy)-5-methyl-2,3-dihydrobenzo[b][1,4]oxazepin-4(5H)-one hydrochloride (77.0 mg, 0.193 mmol) in DCE (2.0 mL) was added CDI (62.0 mg, 0.385 mmol) followed by TEA (0.0670 mL, 0.481 mmol) at 0° C. The mixture was stirred at room temperature for 2.5 hours. After quenched with water, the mixture was extracted with DCM twice. The combined organic layers were washed with brine, dried over Na₂SO₄, filtered, and concentrated in vacuo.

To a solution of the residue in DCE (2.0 mL) was added 4-(3-fluorobenzyl)-1H-pyrazole hydrochloride (Intermediate 6, 0.0330 g, 0.154 mmol) followed by TEA (0.0670 mL, 0.481 mmol) at 0° C., the reaction mixture was stirred at room temperature for 18 hours. After quenched with water, the mixture was extracted with DCM twice. The combined organic layers were washed with brine, dried over Na₂SO₄, filtered, and concentrated in vacuo. The residue was purified by column chromatography on SiO₂ (DCM:MeOH=15:1) to afford (S)-4-(3-fluorobenzyl)-N-(7-(2-(4-hydroxy-4-methylpiperidin-1-yl)-2-oxoethoxy)-5-methyl-4-oxo-2,3,4,5-tetrahydrobenzo[b][1,4]oxazepin-3-yl)-1H-pyrazole-1-carboxamide (46.0 mg, 42%) as a white foam. ¹H-NMR (400 MHz, CDCl₃) δ 7.99 (1H, d, J=7.2 Hz), 7.88 (1H, s), 7.47 (1H, s), 7.28-7.22 (1H, m), 7.12 (1H, d, J=8.8 Hz), 6.97-6.85 (4H, m), 6.80 (1H, dt, J=8.8, 2.8 Hz), 4.90 (1H, dt, J=10.8, 7.6 Hz), 4.70 (2H, d, J=5.2 Hz), 4.65 (1H, dd, J=9.6, 7.2 Hz), 4.26 (1H, dd, J=10.8, 9.6 Hz), 4.25-4.17 (1H, m), 3.81 (2H, s), 3.73-3.62 (1H, m), 3.59-3.48 (1H, m), 3.41 (3H, s), 3.24-3.16 (1H, m), 1.72-1.52 (4H, m), 1.29 (3H, d, J=2.0 Hz). LC-MS: m/z=566.1 [M+H]⁺.

### Example 42: (S)-4-(3-fluorobenzyl)-N-(7-((1-(4-hydroxy-4-methylpiperidin-1-yl)-2-methyl-1-oxopropan-2-yl)oxy)-5-methyl-4-oxo-2,3,4,5-tetrahydrobenzo[b][1,4]oxazepin-3-yl)-1H-pyrazole-1-carboxamide

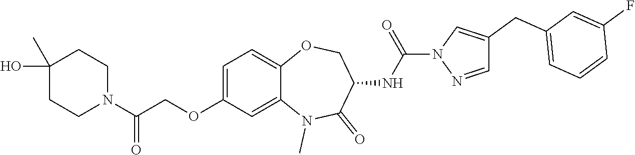 [SMILES: CN1C(=O)[C@@H](NC(=O)n2cc(Cc3cccc(F)c3)cn2)COc2ccc(OCC(=O)N3CCC(C)(O)CC3)cc21]

### Step A: (S)-2-((3-((tert-butoxycarbonyl)amino)-5-methyl-4-oxo-2,3,4,5-tetrahydrobenzo[b][1,4]oxazepin-7-yl)oxy)-2-methylpropanoic acid

To a solution of (S)-(7-hydroxy-5-methyl-4-oxo-2,3,4,5-tetrahydrobenzo[b][1,4]oxazepin-3-yl)carbamate (Intermediate 2, 0.500 g, 1.62 mmol) in DMF (13 mL) was portionwise added NaH (60 wt %, 0.259 mg, 6.49 mmol) at 0° C. The mixture was stirred at 0° C. for 1 hour. After addition of 2-bromo-2-methylpropanoic acid (0.425 mL, 8.25 mmol) at 0° C., the reaction mixture was stirred at 0° C. for 20 hours and cooled to −10° C. After quenched with 0.5 N aq. HCl until pH 3-4, the mixture was extracted with DCM twice. The combined organic layers were washed with brine, dried over Na₂SO₄, filtered, and concentrated in vacuo. The residue was purified by column chromatography on SiO₂ (EtOAc only) to afford (S)-2-((3-((tert-butoxycarbonyl)amino)-5-methyl-4-oxo-2,3,4,5-tetrahydrobenzo[b][1,4]oxazepin-7-yl)oxy)-2-methylpropanoic acid (0.420 g, 66%) as a yellow solid. LC-MS: m/z=395.10 [M+H]⁺.

### Step B: tert-butyl (S)-(7-((1-(4-hydroxy-4-methylpiperidin-1-yl)-2-methyl-1-oxopropan-2-yl)oxy)-5-methyl-4-oxo-2,3,4,5-tetrahydrobenzo[b][1,4]oxazepin-3-yl)carbamate

The title compound was prepared in a similar fashion to Example 41 (Step A) with (S)-2-((3-((tert-butoxycarbonyl)amino)-5-methyl-4-oxo-2,3,4,5-tetrahydrobenzo[b][1,4]oxazepin-7-yl)oxy)-2-methylpropanoic acid and 4-methylpiperidin-4-ol. The crude product was purified by column chromatography on SiO₂ (Hexanes:EtOAc=1:1) to afford tert-butyl (S)-(7-((1-(4-hydroxy-4-methylpiperidin-1-yl)-2-methyl-1-oxopropan-2-yl)oxy)-5-methyl-4-oxo-2,3,4,5-tetrahydrobenzo[b][1,4]oxazepin-3-yl)carbamate (89%) as a white solid. ¹H-NMR (400 MHz, CDCl₃): δ 7.00 (1H, d, J=8.8 Hz), 6.69 (1H, brs), 6.65 (1H, dd, J=8.8, 3.2 Hz), 5.46 (1H, d, J=7.2 Hz), 4.66-4.60 (1H, m), 4.50 (1H, t, J=8.8 Hz), 4.11-3.96 (1H, m), 3.64-3.50 (1H, m), 3.40-3.67 (1H, m), 3.34 (3H, s), 1.66-1.64 (6H, m), 1.57-1.46 (4H, m), 1.39 (9H, s), 1.19 (3H, d, J=6.4 Hz).

### Step C: (S)-3-amino-7-((1-(4-hydroxy-4-methylpiperidin-1-yl)-2-methyl-1-oxopropan-2-yl)oxy)-5-methyl-2,3-dihydrobenzo[b][1,4]oxazepin-4(5H)-one hydrochloride

The title compound was prepared in a similar fashion to Example 41 (Step B) with tert-butyl (S)-(7-((1-(4-hydroxy-4-methylpiperidin-1-yl)-2-methyl-1-oxopropan-2-yl)oxy)-5-methyl-4-oxo-2,3,4,5-tetrahydrobenzo[b][1,4]oxazepin-3-yl)carbamate. After concentration in vacuo, the crude product was used for the next reaction without purification. ¹H-NMR (400 MHz, CDCl₃): δ 8.41 (2H, brs), 7.18 (1H, d, J=9.2 Hz), 6.91 (1H, brs), 6.67 (1H, dd, J=8.8, 2.8 Hz), 4.52-4.48 (1H, m), 4.40-4.33 (2H, m), 4.28-4.19 (1H, m), 4.11-3.95 (1H, m), 3.28 (3H, s), 3.09-2.99 (2H, m), 1.55 (6H, s), 1.47-1.39 (1H, m), 1.35-1.20 (1H, m), 1.02 (3H, s).

### Step D: (S)-4-(3-fluorobenzyl)-N-(7-((1-(4-hydroxy-4-methylpiperidin-1-yl)-2-methyl-1-oxopropan-2-yl)oxy)-5-methyl-4-oxo-2,3,4,5-tetrahydrobenzo[b][1,4]oxazepin-3-yl)-1H-pyrazole-1-carboxamide

The title compound was prepared in a similar fashion to Example 41 (Step C) with (S)-3-amino-7-((1-(4-hydroxy-4-methylpiperidin-1-yl)-2-methyl-1-oxopropan-2-yl)oxy)-5-methyl-2,3-dihydrobenzo[b][1,4]oxazepin-4(5H)-one hydrochloride and 4-(3-fluorobenzyl)-1H-pyrazole hydrochloride (Intermediate 6). The crude product was purified by column chromatography on SiO₂ (Hexanes:EtOAc=1:1) to give (S)-4-(3-fluorobenzyl)-N-(7-((1-(4-hydroxy-4-methylpiperidin-1-yl)-2-methyl-1-oxopropan-2-yl)oxy)-5-methyl-4-oxo-2,3,4,5-tetrahydrobenzo[b][1,4]oxazepin-3-yl)-1H-pyrazole-1-carboxamide (90%) as a white foam. ¹H-NMR (400 MHz, CDCl₃): δ 7.97 (1H, d, J=6.0 Hz), 7.87 (1H, d, J=0.8 Hz), 7.46 (1H, s), 7.27-7.22 (1H, m), 7.04 (1H, d, J=8.8 Hz), 6.96-6.84 (3H, m), 6.72 (1H, d, J=2.8 Hz), 6.69 (1H, dd, J=8.8, 3.2 Hz), 4.89 (1H, dt, J=11.2, 7.6 Hz), 4.64 (1H, dd, J=10.0, 7.2 Hz), 4.27-4.21 (1H, m), 4.19-4.02 (2H, m), 3.80 (2H, s), 3.64-3.51 (1H, m), 3.37 (3H, s), 3.37-3.31 (1H, m), 1.65 (6H, s), 1.57-1.46 (2H, m), 1.42-1.37 (1H, m), 1.30-1.23 (1H, m), 1.20 (1H, d, J=4.4 Hz). LC-MS: m/z=594.1 [M+H]⁺.

### Example 43: (S)-4-(3-fluorobenzyl)-N-(7-(2-(4-(2-hydroxypropan-2-yl)piperidin-1-yl)-2-oxoethoxy)-5-methyl-4-oxo-2,3,4,5-tetrahydrobenzo[b][1,4]oxazepin-3-yl)-1H-pyrazole-1-carboxamide

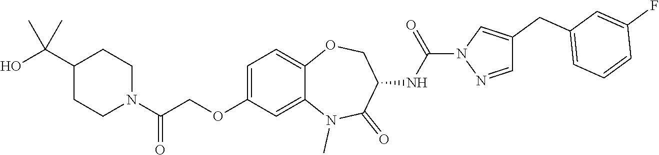 [SMILES: CN1C(=O)[C@@H](NC(=O)n2cc(Cc3cccc(F)c3)cn2)COc2ccc(OCC(=O)N3CCC(C(C)(C)O)CC3)cc21]

The title compound was prepared in a similar fashion to Example 36 with Intermediate 4 and 2-(piperidin-4-yl)propan-2-ol. The crude product was purified by column chromatography on SiO₂ (EtOAc only) to afford (S)-4-(3-fluorobenzyl)-N-(7-(2-(4-(2-hydroxypropan-2-yl)piperidin-1-yl)-2-oxoethoxy)-5-methyl-4-oxo-2,3,4,5-tetrahydrobenzo[b][1,4]oxazepin-3-yl)-1H-pyrazole-1-carboxamide (80%) as a white foam. ¹H-NMR (400 MHz, CDCl₃): δ 7.97 (1H, dd, J=3.2, 2.0 Hz), 7.87 (1H, s), 7.47 (1H, s), 7.28-7.22 (1H, m), 7.11 (1H, d, J=8.8 Hz), 6.96-6.85 (4H, m), 6.80 (1H, dd, J=8.8, 3.2 Hz), 4.89 (1H, td, J=11.2, 5.6 Hz), 4.71-4.62 (3H, m), 4.24 (1H, dd, J=11.2, 10.0 Hz), 4.05 (1H, d, J=13.6 Hz), 3.81 (2H, s), 3.41 (3H, s), 2.99-3.08 (1H, m), 2.59-2.53 (1H, m), 1.89-1.79 (2H, m), 1.58-1.49 (2H, m), 1.16 (6H, s). LC-MS: m/z=594.1 [M+H]⁺.

### Example 44: (S)-4-(3-fluorobenzyl)-N-(5-methyl-4-oxo-7-(2-oxo-2-(pyrrolidin-1-yl)ethoxy)-2,3,4,5-tetrahydrobenzo[b][1,4]oxazepin-3-yl)-1H-pyrazole-1-carboxamide

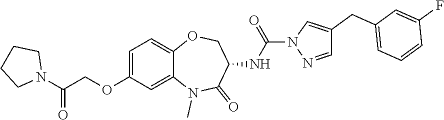 [SMILES: CN1C(=O)[C@@H](NC(=O)n2cc(Cc3cccc(F)c3)cn2)COc2ccc(OCC(=O)N3CCCC3)cc21]

The title compound was prepared in a similar fashion to Example 36 with Intermediate 4 and pyrrolidine. The crude product was purified by column chromatography on SiO₂ (DCM:MeOH=20:1) to give (S)-4-(3-fluorobenzyl)-N-(5-methyl-4-oxo-7-(2-oxo-2-(pyrrolidin-1-yl)ethoxy)-2,3,4,5-tetrahydrobenzo[b][1,4]oxazepin-3-yl)-1H-pyrazole-1-carboxamide (45%) as a white foam. ¹H-NMR (400 MHz, CDCl₃): δ 7.98 (1H, d, J=7.3 Hz), 7.88 (1H, s), 7.47 (1H, s), 7.24-7.22 (1H, m), 7.11 (1H, d, J=8.7 Hz), 7.00-6.85 (4H, m), 6.81-6.78 (1H, m), 4.93-4.87 (1H, m), 4.68-4.66 (1H, m), 4.63 (2H, s), 4.25 (1H, dd, J=11.2, 9.8 Hz), 3.81 (2H, s), 3.56-3.50 (4H, m), 3.41 (3H, s), 2.04-1.97 (2H, m), 1.92-1.85 (2H, m). LC-MS: m/z=522.10 [M+H]⁺.

### Example 45: (S)—N-(7-(2-(3,3-difluoropyrrolidin-1-yl)-2-oxoethoxy)-5-methyl-4-oxo-2,3,4,5-tetrahydrobenzo[b][1,4]oxazepin-3-yl)-4-(3-fluorobenzyl)-1H-pyrazole-1-carboxamide

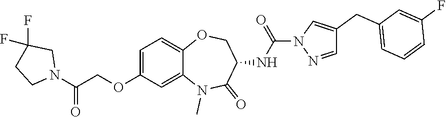 [SMILES: CN1C(=O)[C@@H](NC(=O)n2cc(Cc3cccc(F)c3)cn2)COc2ccc(OCC(=O)N3CCC(F)(F)C3)cc21]

The title compound was prepared in a similar fashion to Example 36 with Intermediate 4 and 3,3-difluoropyrrolidine. The crude product was purified by column chromatography on SiO₂ (Hexanes:EtOAc=1:1) to give (S)—N-(7-(2-(3,3-difluoropyrrolidin-1-yl)-2-oxoethoxy)-5-methyl-4-oxo-2,3,4,5-tetrahydrobenzo[b][1,4]oxazepin-3-yl)-4-(3-fluorobenzyl)-1H-pyrazole-1-carboxamide (34%) as a white foam. ¹H-NMR (400 MHz, CDCl₃): δ 7.98 (1H, d, J=5.0 Hz), 7.88 (1H, s), 7.47 (1H, s), 7.25-7.22 (1H, m), 7.14-7.12 (1H, m), 7.00-6.77 (5H, m), 4.94-4.87 (1H, m), 4.68-4.63 (3H, m), 4.26 (1H, dd, J=11.0, 10.1 Hz), 3.96-3.76 (6H, m), 3.41 (3H, d, J=1.8 Hz), 2.52-2.32 (2H, m). LC-MS: m/z=558.10 [M+H]⁺.

### Example 46: 4-(3-fluorobenzyl)-N—((S)-7-(2-((S)-3-hydroxypyrrolidin-1-yl)-2-oxoethoxy)-5-methyl-4-oxo-2,3,4,5-tetrahydrobenzo[b][1,4]oxazepin-3-yl)-1H-pyrazole-1-carboxamide

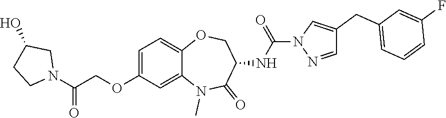 [SMILES: CN1C(=O)[C@@H](NC(=O)n2cc(Cc3cccc(F)c3)cn2)COc2ccc(OCC(=O)N3CC[C@H](O)C3)cc21]

The title compound was prepared in a similar fashion to Example 36 with Intermediate 4 and (S)-pyrrolidin-3-ol. The crude product was purified by column chromatography on SiO₂ (DCM:MeOH=20:1) to afford 4-(3-fluorobenzyl)-N—((S)-7-(2-((S)-3-hydroxypyrrolidin-1-yl)-2-oxoethoxy)-5-methyl-4-oxo-2,3,4,5-tetrahydrobenzo[b][1,4]oxazepin-3-yl)-1H-pyrazole-1-carboxamide (79%) as pale-yellow foam. ¹H-NMR (400 MHz, CDCl₃) δ 7.99 (1H, dd, J=7.6, 2.8 Hz), 7.88 (1H, s), 7.47 (1H, s), 7.28-7.23 (1H, m), 7.11 (1H, d, J=8.8 Hz), 6.97-6.85 (4H, m), 6.79 (1H, m), 4.89 (1H, dt, J=11.6, 7.2 Hz), 4.67-4.63 (3H, m), 4.58 and 4.50 (1H, m), 4.25 (1H, dd, J=10.8, 9.6 Hz), 3.81 (2H, s), 3.74-3.63 (3H, m), 3.57-3.52 (1H, m), 3.41 (3H, s), 2.12-2.04 (1H, m), 2.02-1.91 (1H, m). LC-MS: m/z=538.1 [M+H]⁺.

### Example 47: 4-(3-fluorobenzyl)-N—((S)-7-(2-((R)-3-hydroxypyrrolidin-1-yl)-2-oxoethoxy)-5-methyl-4-oxo-2,3,4,5-tetrahydrobenzo[b][1,4]oxazepin-3-yl)-1H-pyrazole-1-carboxamide

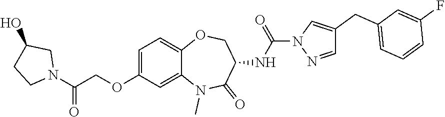 [SMILES: CN1C(=O)[C@@H](NC(=O)n2cc(Cc3cccc(F)c3)cn2)COc2ccc(OCC(=O)N3CC[C@@H](O)C3)cc21]

The title compound was prepared in a similar fashion to Example 36 with Intermediate 4 and (R)-pyrrolidin-3-ol. The crude product was purified by column chromatography on SiO₂ (DCM:MeOH=20:1) to afford 4-(3-fluorobenzyl)-N—((S)-7-(2-((R)-3-hydroxypyrrolidin-1-yl)-2-oxoethoxy)-5-methyl-4-oxo-2,3,4,5-tetrahydrobenzo[b][1,4]oxazepin-3-yl)-1H-pyrazole-1-carboxamide (81%) as a white foam. ¹H-NMR (400 MHz, CDCl₃) δ 7.99 (1H, d, J=7.2 Hz), 7.88 (1H, s), 7.47 (1H, s), 7.27-7.22 (1H, m), 7.11 (1H, dd, J=8.8, 1.2 Hz), 6.96-6.85 (4H, m), 6.79 (1H, dd, J=8.8, 2.4 Hz), 4.93-4.86 (1H, m), 4.67-4.62 (3H, m), 4.55 and 4.50 (1H, m), 4.25 (1H, t, J=10.8 Hz), 3.81 (2H, s), 3.73-3.52 (4H, m), 3.40 (3H, d, J=1.6 Hz), 2.10-2.05 (1H, m), 2.03-1.93 (1H, m). LC-MS: m/z=538.0 [M+H]⁺.

### Example 48: 4-(3-fluorobenzyl)-N-((3S)-7-(2-(3-(2-hydroxypropan-2-yl)pyrrolidin-1-yl)-2-oxoethoxy)-5-methyl-4-oxo-2,3,4,5-tetrahydrobenzo[b][1,4]oxazepin-3-yl)-1H-pyrazole-1-carboxamide

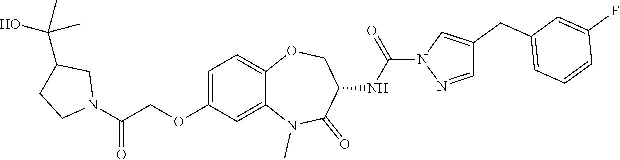 [SMILES: CN1C(=O)[C@@H](NC(=O)n2cc(Cc3cccc(F)c3)cn2)COc2ccc(OCC(=O)N3CCC(C(C)(C)O)C3)cc21]

The title compound was prepared in a similar fashion to Example 36 with Intermediate 4 and 2-(pyrrolidin-3-yl)propan-2-ol. The residue was purified by column chromatography on SiO₂ (DCM:MeOH=20:1) to give 4-(3-fluorobenzyl)-N-((3S)-7-(2-(3-(2-hydroxypropan-2-yl)pyrrolidin-1-yl)-2-oxoethoxy)-5-methyl-4-oxo-2,3,4,5-tetrahydrobenzo[b][1,4]oxazepin-3-yl)-1H-pyrazole-1-carboxamide (47%) as a white foam. ¹H-NMR (400 MHz, CDCl₃): δ 7.98 (1H, d, J=7.3 Hz), 7.88 (1H, s), 7.47 (1H, s), 7.24-7.22 (1H, m), 7.11 (1H, d, J=8.7 Hz), 6.96-6.77 (5H, m), 4.93-4.87 (1H, m), 4.68-4.59 (3H, m), 4.25 (1H, t, J=10.5 Hz), 3.81 (2H, s), 3.77-3.64 (1H, m), 3.53-3.44 (2H, m), 3.41 (3H, s), 3.38-3.31 (1H, m), 2.38-2.17 (1H, m), 2.06-1.73 (2H, m), 1.28 (3H, s), 1.26 (3H, s). LC-MS: m/z=580.10 [M+H]⁺.

### Example 49: (S)-4-(3-fluorobenzyl)-N-(7-(2-(3-hydroxy-3-methylazetidin-1-yl)-2-oxoethoxy)-5-methyl-4-oxo-2,3,4,5-tetrahydrobenzo[b][1,4]oxazepin-3-yl)-1H-pyrazole-1-carboxamide

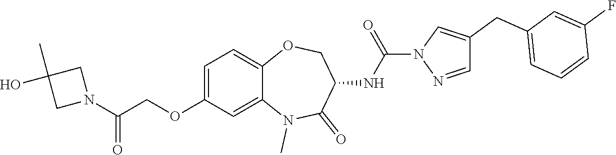 [SMILES: CN1C(=O)[C@@H](NC(=O)n2cc(Cc3cccc(F)c3)cn2)COc2ccc(OCC(=O)N3CC(C)(O)C3)cc21]

The title compound was prepared in a similar fashion to Example 36 with Intermediate 4 and 3-methylazetidin-3-ol TFA. The crude product was purified by column chromatography on SiO₂ (DCM:MeOH=20:1) to give (S)-4-(3-fluorobenzyl)-N-(7-(2-(3-hydroxy-3-methylazetidin-1-yl)-2-oxoethoxy)-5-methyl-4-oxo-2,3,4,5 tetrahydrobenzo[b][1,4]oxazepin-3-yl)-1H-pyrazole-1-carboxamide (26%) as a white foam. ¹H-NMR (400 MHz, CDCl₃): δ 7.98 (1H, d, J=7.8 Hz), 7.87 (1H, d, J=0.9 Hz), 7.47 (1H, s), 7.24-7.22 (1H, m), 7.14 (1H, d, J=8.7 Hz), 6.96-6.85 (3H, m), 6.79-6.74 (2H, m), 4.93-4.86 (1H, m), 4.66 (1H, t, J=8.7 Hz), 4.29-4.26 (1H, m), 4.23 (2H, d, J=2.3 Hz), 4.03-3.99 (2H, m), 3.81 (2H, s), 3.41 (3H, s), 1.56 (3H, s). LC-MS: m/z=538.10 [M+H]⁺.

### Example 50: (S)-4-(3-fluorobenzyl)-N-(7-(2-(3-hydroxyazetidin-1-yl)-2-oxoethoxy)-5-methyl-4-oxo-2,3,4,5-tetrahydrobenzo[b][1,4]oxazepin-3-yl)-1H-pyrazole-1-carboxamide

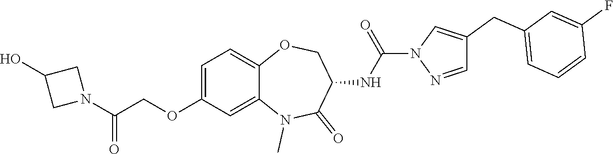 [SMILES: CN1C(=O)[C@@H](NC(=O)n2cc(Cc3cccc(F)c3)cn2)COc2ccc(OCC(=O)N3CC(O)C3)cc21]

The title compound was prepared in a similar fashion to Example 36 with Intermediate 4 and azetidin-3-ol hydrochloride. The crude product was purified by column chromatography on SiO₂ (EtOAc:MeOH=20:1) to (S)-4-(3-fluorobenzyl)-N-(7-(2-(3-hydroxyazetidin-1-yl)-2-oxoethoxy)-5-methyl-4-oxo-2,3,4,5-tetrahydrobenzo[b][1,4]oxazepin-3-yl)-1H-pyrazole-1-carboxamide (83%) as white foam. ¹H-NMR (400 MHz, CDCl₃) δ 7.99 (1H, d, J=7.2 Hz), 7.88 (1H, s), 7.47 (1H, s), 7.30-7.22 (1H, m), 7.13 (1H, d, J=8.4 Hz), 6.96-6.85 (3H, m), 6.79 (1H, t, J=2.8 Hz), 6.75 (1H, dt, J=8.8, 2.8 Hz), 4.90 (1H, dt, J=10.8, 7.6 Hz), 4.72-4.63 (2H, m), 4.56 (2H, s), 4.55-4.50 (1H, m), 4.34-4.31 (1H, m), 4.26 (1H, dd, J=11.2, 10.0 Hz), 4.22-4.13 (1H, m), 3.99-3.90 (1H, m), 3.81 (2H, s), 3.40 (3H, s). LC-MS: m/z=524.1 [M+H]⁺.

### Example 51: (S)—N-(7-(2-(cyclopropylamino)-2-oxoethoxy)-5-methyl-4-oxo-2,3,4,5-tetrahydrobenzo[b][1,4]oxazepin-3-yl)-4-(3-fluorobenzyl)-1H-pyrazole-1-carboxamide

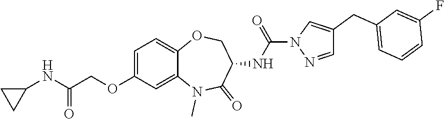 [SMILES: CN1C(=O)[C@@H](NC(=O)n2cc(Cc3cccc(F)c3)cn2)COc2ccc(OCC(=O)NC3CC3)cc21]

### Step A: tert-butyl (S)-(7-(2-(cyclopropylamino)-2-oxoethoxy)-5-methyl-4-oxo-2,3,4,5-tetrahydrobenzo[b][1,4]oxazepin-3-yl)carbamate

The title compound was prepared in a similar fashion to Example 41 (Step A) with Intermediate 3 and cyclopropanamine. The crude product was purified by column chromatography on SiO₂ (Hexanes:EtOAc=1:1 to EtOAc only) to afford tert-butyl (S)-(7-(2-(cyclopropylamino)-2-oxoethoxy)-5-methyl-4-oxo-2,3,4,5-tetrahydrobenzo[b][1,4]oxazepin-3-yl)carbamate (76%) as a yellow oil. ¹H-NMR (400 MHz, CDCl₃): 7.73 (1H, d, J=8.4 Hz), 6.73-6.69 (2H, m), 6.57 (1H, s), 5.46 (1H, d, J=7.2 Hz), 4.66-4.60 (1H, m), 4.51 (1H, dd, J=10.6, 7.6 Hz), 4.43 (2H, s), 4.10 (1H, dd, J=11.2, 10.0 Hz), 3.36 (3H, s), 2.81-2.76 (1H, m), 1.38 (9H, s), 0.87-0.82 (2H, m), 0.62-0.58 (2H, m).

### Step B: (S)-2-((3-amino-5-methyl-4-oxo-2,3,4,5-tetrahydrobenzo[b][1,4]oxazepin-7-yl)oxy)-N-cyclopropylacetamide hydrochloride

The title compound was prepared in a similar fashion to Example 41 (Step B) with tert-butyl (S)-(7-(2-(cyclopropylamino)-2-oxoethoxy)-5-methyl-4-oxo-2,3,4,5-tetrahydrobenzo[b][1,4]oxazepin-3-yl)carbamate. After concentration in vacuo, the crude product was used for the next reaction without purification. LC-MS: m/z=306.1 [M+H]⁺.

### Step C: (S)—N-(7-(2-(cyclopropylamino)-2-oxoethoxy)-5-methyl-4-oxo-2,3,4,5-tetrahydrobenzo[b][1,4]oxazepin-3-yl)-4-(3-fluorobenzyl)-1H-pyrazole-1-carboxamide

The title compound was prepared in a similar fashion to Example 41 (Step C) with (S)-2-((3-amino-5-methyl-4-oxo-2,3,4,5-tetrahydrobenzo[b][1,4]oxazepin-7-yl)oxy)-N-cyclopropylacetamide hydrochloride and 4-(3-fluorobenzyl)-1H-pyrazole hydrochloride (Intermediate 6). The crude product was purified by column chromatography on SiO₂ (Hexanes:EtOAc=3:1 to EtOAc only) to give (S)—N-(7-(2-(cyclopropylamino)-2-oxoethoxy)-5-methyl-4-oxo-2,3,4,5-tetrahydrobenzo[b][1,4]oxazepin-3-yl)-4-(3-fluorobenzyl)-1H-pyrazole-1-carboxamide (40% for 2 steps) as a colorless oil. ¹H-NMR (400 MHz, CDCl₃): δ7.96 (1H, d, J=7.2 Hz), 7.87 (1H, s), 7.46 (1H, s), 7.28-7.21 (1H, m), 7.14 (1H, d, J=8.0 Hz), 6.95-6.84 (3H, m), 6.76-6.73 (2H, m), 6.58 (1H, s), 4.90-4.85 (1H, m), 4.65 (1H, dd, J=9.2, 8.8 Hz), 4.45 (2H, s), 4.25 (1H, dd, J=10.4, 9.6 Hz), 3.80 (2H, s), 3.40 (3H, s), 2.82-2.77 (1H, m), 0.86-0.82 (2H, m), 0.60-0.59 (2H, m). LC-MS: m/z=508.1 [M+H]⁺.

### Example 52: (S)-4-(3-fluorobenzyl)-N-(7-(2-((2-hydroxy-2-methylpropyl)amino)-2-oxoethoxy)-5-methyl-4-oxo-2,3,4,5-tetrahydrobenzo[b][1,4]oxazepin-3-yl)-1H-pyrazole-1-carboxamide

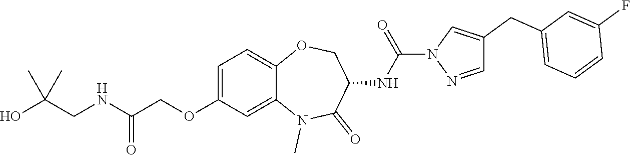 [SMILES: CN1C(=O)[C@@H](NC(=O)n2cc(Cc3cccc(F)c3)cn2)COc2ccc(OCC(=O)NCC(C)(C)O)cc21]

### Step A: tert-butyl (S)-(7-(2-((2-hydroxy-2-methylpropyl)amino)-2-oxoethoxy)-5-methyl-4-oxo-2,3,4,5-tetrahydrobenzo[b][1,4]oxazepin-3-yl)carbamate

The title compound was prepared in a similar fashion to Example 41 with Intermediate 3 and 1-amino-2-methylpropan-2-ol. The crude product was purified by column chromatography on SiO₂ (DCM:MeOH=20:1) to afford tert-butyl (S)-(7-(2-((2-hydroxy-2-methylpropyl)amino)-2-oxoethoxy)-5-methyl-4-oxo-2,3,4,5-tetrahydrobenzo[b][1,4]oxazepin-3-yl)carbamate (79%) as orange oil. ¹H-NMR (400 MHz, CDCl₃) δ 7.10 (1H, d, J=8.7 Hz), 6.96 (1H, t, J=5.2 Hz), 6.81 (1H, d, J=2.8 Hz), 6.77 (1H, dd, J=8.8, 2.8 Hz), 5.53 (1H, d, J=7.6 Hz), 4.62 (1H, dt, J=11.2, 7.6 Hz), 4.56 (2H, d, J=2.8 Hz), 4.47 (1H, dd, J=9.6, 7.2 Hz), 4.12 (1H, dd, J=11.2, 9.6 Hz), 3.38 (3H, s), 3.34 (2H, t, J=5.2 Hz), 1.40 (9H, s), 1.21 (3H, s), 1.17 (3H, s).

### Step B: (3S)-3-amino-7-(2-(3-hydroxypyrrolidin-1-yl)-2-oxoethoxy)-5-methyl-2,3-dihydrobenzo[b][1,4]oxazepin-4(5H)-one hydrochloride

The title compound was prepared in a similar fashion to Example 41 with tert-butyl (S)-(7-(2-((2-hydroxy-2-methylpropyl)amino)-2-oxoethoxy)-5-methyl-4-oxo-2,3,4,5-tetrahydrobenzo[b][1,4]oxazepin-3-yl)carbamate and HCl in dioxane as a solvent.

After concentration in vacuo, the crude product was used for the next reaction without purification. LC-MS: m/z=338.1 [M+H]⁺.

### Step C: (S)-4-(3-fluorobenzyl)-N-(7-(2-((2-hydroxy-2-methylpropyl)amino)-2-oxoethoxy)-5-methyl-4-oxo-2,3,4,5-tetrahydrobenzo[b][1,4]oxazepin-3-yl)-1H-pyrazole-1-carboxamide

The title compound was prepared in a similar fashion to Example 41 (Step C) with (S)-2-((3-amino-5-methyl-4-oxo-2,3,4,5-tetrahydrobenzo[b][1,4]oxazepin-7-yl)oxy)-N-(2-hydroxy-2-methylpropyl)acetamide hydrochloride and 4-(3-fluorobenzyl)-1H-pyrazole hydrochloride (Intermediate 6). The crude product was purified by column chromatography on SiO₂ (Hexanes:EtOAc=3:1) to afford (S)-4-(3-fluorobenzyl)-N-(7-(2-((2-hydroxy-2-methylpropyl)amino)-2-oxoethoxy)-5-methyl-4-oxo-2,3,4,5-tetrahydrobenzo[b][1,4]oxazepin-3-yl)-1H-pyrazole-1-carboxamide (13.0 mg, 10% for 2 steps) as oil. ¹H-NMR (400 MHz, CDCl₃) δ 8.00 (1H, d, J=6.8 Hz), 7.88 (1H, s), 7.48 (1H, s), 7.27-7.22 (1H, m), 7.13 (1H, d, J=8.8 Hz), 6.97-6.82 (5H, m), 4.90 (1H, dt, J=10.8, 7.2 Hz), 4.69 (2H, s), 4.68-4.65 (1H, m), 4.26 (1H, t, J=10.8 Hz), 3.81 (2H, s), 3.65 (2H, s), 3.41 (3H, s), 1.42 (6H, s). LC-MS: m/z=540.1 [M+H]⁺.

### Example 53: (S)—N-(7-(2-(ethyl(2-hydroxy-2-methylpropyl)amino)-2-oxoethoxy)-5-methyl-4-oxo-2,3,4,5-tetrahydrobenzo[b][1,4]oxazepin-3-yl)-4-(3-fluorobenzyl)-1H-pyrazole-1-carboxamide

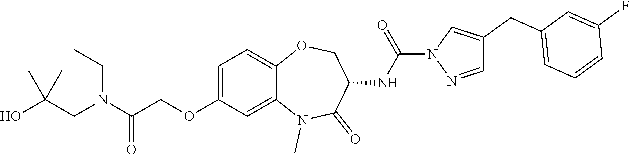 [SMILES: CCN(CC(C)(C)O)C(=O)COc1ccc2c(c1)N(C)C(=O)[C@@H](NC(=O)n1cc(Cc3cccc(F)c3)cn1)CO2]

The title compound was prepared in a similar fashion to Example 36 with Intermediate 4 and 1-(ethylamino)-2-methylpropan-2-ol. The crude product was purified by column chromatography on SiO₂ (Hexanes:EtOAc=1:3) to afford (S)—N-(7-(2-(ethyl(2-hydroxy-2-methylpropyl)amino)-2-oxoethoxy)-5-methyl-4-oxo-2,3,4,5-tetrahydrobenzo[b][1,4]oxazepin-3-yl)-4-(3-fluorobenzyl)-1H-pyrazole-1-carboxamide (93%) as white foam. ¹H-NMR (400 MHz, CDCl₃) δ 7.98 (1H, d, J=7.6 Hz), 7.88 (1H, d, J=1.2 Hz), 7.47 (1H, s), 7.30-7.23 (1H, m), 7.12 (1H, d, J=9.2 Hz), 6.97-6.85 (4H, m), 6.79 (1H, dd, J=8.8, 2.8 Hz), 4.90 (1H, m), 4.78 (2H, s), 4.66 (1H, dd, J=9.6, 7.6 Hz), 4.25 (1H, dd, J=10.8, 10.0 Hz), 3.89 (1H, s), 3.81 (2H, s), 3.53 (2H, q, J=7.2 Hz), 3.43 (2H, s), 3.41 (3H, s), 1.26 (4H, t, J=7.2 Hz), 1.22 (6H, s). LC-MS: m/z=568.3 [M+H]⁺.

### Example 54: (S)—N-(7-(2-(4-((tert-butyldimethylsilyl)oxy)-1H-pyrazol-1-yl)-2-oxoethoxy)-5-methyl-4-oxo-2,3,4,5-tetrahydrobenzo[b][1,4]oxazepin-3-yl)-4-(3-fluorobenzyl)-1H-pyrazole-1-carboxamide

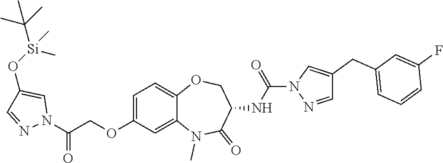 [SMILES: CN1C(=O)[C@@H](NC(=O)n2cc(Cc3cccc(F)c3)cn2)COc2ccc(OCC(=O)n3cc(O[Si](C)(C)C(C)(C)C)cn3)cc21]

The title compound was prepared in a similar fashion to Example 36 with Intermediate 4 and 4-((tert-butyldimethylsilyl)oxy)-1H-pyrazole. The crude product was purified by column chromatography on SiO₂ (EtOAc:MeOH=20:1) to (S)—N-(7-(2-(4-((tert-butyldimethylsilyl)oxy)-1H-pyrazol-1-yl)-2-oxoethoxy)-5-methyl-4-oxo-2,3,4,5-tetrahydrobenzo[b][1,4]oxazepin-3-yl)-4-(3-fluorobenzyl)-1H-pyrazole-1-carboxamide (90%) as white foam. ¹H-NMR (400 MHz, CDCl₃) δ 7.99 (1H, d, J=7.2 Hz), 7.88 (1H, d, J=0.8 Hz), 7.76 (1H, s), 7.48 (1H, s), 7.47 (1H, s), 7.27-7.22 (1H, m), 7.13 (1H, d, J=8.8 Hz), 6.96-6.85 (4H, m), 6.82 (1H, dd, J=8.8, 2.8 Hz), 5.44 (2H, s), 4.90 (1H, dt, J=10.8, 7.2 Hz), 4.67 (1H, dd, J=9.6, 7.2 Hz), 4.26 (1H, dd, J=10.8, 9.6 Hz), 3.81 (2H, s), 3.41 (3H, s), 0.97 (9H, s), 0.22 (6H, s). LC-MS: m/z=649.1 [M+H]⁺.

### Example 55: (S)-4-(3-fluorobenzyl)-N-(5-methyl-4-oxo-7-((tetrahydro-2H-pyran-4-yl)methoxy)-2,3,4,5-tetrahydrobenzo[b][1,4]oxazepin-3-yl)-1H-pyrazole-1-carboxamide

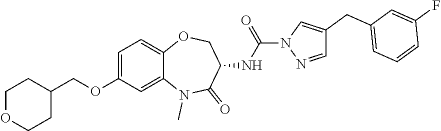 [SMILES: CN1C(=O)[C@@H](NC(=O)n2cc(Cc3cccc(F)c3)cn2)COc2ccc(OCC3CCOCC3)cc21]

### Step A: tert-butyl (S)-(5-methyl-4-oxo-7-((tetrahydro-2H-pyran-4-yl)methoxy)-2,3,4,5-tetrahydrobenzo[b][1,4]oxazepin-3-yl)carbamate

The title compound was prepared in a similar fashion to Example 15 (Step A) with Intermediate 2 and (tetrahydro-2H-pyran-4-yl)methyl methanesulfonate. The crude product was purified by column chromatography on SiO₂ (Hexanes:EtOAc=2:1) to afford tert-butyl (S)-(5-methyl-4-oxo-7-((tetrahydro-2H-pyran-4-yl)methoxy)-2,3,4,5-tetrahydrobenzo[b][1,4]oxazepin-3-yl)carbamate (57%) as a white foam. ¹H-NMR (400 MHz, CDCl₃): δ 7.04 (1H, dd, J=7.4, 2.2 Hz), 6.69 (2H, J=7.6 2.4 Hz), 5.47 (1H, d, J=7.2 Hz), 4.68-4.61 (1H, m), 4.52 (1H, dd, J=9.6, 7.6 Hz), 4.12-4.07 (1H, m), 4.03 (2H, dd, J=11.0, 3.4 Hz), 3.78 (2H, d, J=6.4 Hz), 3.49-3.42 (2H, m), 3.38 (3H, s), 2.09-2.03 (1H, m), 1.76 (2H, d, J=12.8 Hz), 1.51-1.44 (2H, m), 1.40 (9H, s).

### Step B: (S)-3-amino-5-methyl-7-((tetrahydro-2H-pyran-4-yl)methoxy)-2,3-dihydrobenzo[b][1,4]oxazepin-4(5H)-one hydrochloride

The title compound was prepared in a similar fashion to Example 15 (Step B) with tert-butyl (S)-(5-methyl-4-oxo-7-((tetrahydro-2H-pyran-4-yl)methoxy)-2,3,4,5-tetrahydrobenzo[b][1,4]oxazepin-3-yl)carbamate. After concentration in vacuo, the crude product was used for the next reaction without purification. ¹H-NMR (400 MHz, DMSO-d₆): δ 8.45 (3H, s), 7.17 (1H, d, J=8.8 Hz), 7.08 (1H, d, J=2.4 Hz), 6.86 (1H, dd, J=8.8, 2.8 Hz), 4.50 (1H, dd, J=9.8, 7.5 Hz), 4.35 (1H, t, J=10.5 Hz), 4.26 (1H, dd, J=11.0, 7.8 Hz), 3.90-3.84 (4H, m), 3.57-3.57 (3H, s), 2.02-1.96 (1H, m), 1.68 (2H, d, J=12.8 Hz), 1.38-1.32 (2H, m), 0.90-0.73 (2H, m).

### Step C: (S)-4-(3-fluorobenzyl)-N-(5-methyl-4-oxo-7-((tetrahydro-2H-pyran-4-yl)methoxy)-2,3,4,5-tetrahydrobenzo[b][1,4]oxazepin-3-yl)-1H-pyrazole-1-carboxamide

The title compound was prepared in a similar fashion to Example 15 (Step C) with (S)-3-amino-5-methyl-7-((tetrahydro-2H-pyran-4-yl)methoxy)-2,3-dihydrobenzo[b][1,4]oxazepin-4(5H)-one hydrochloride and 4-(3-fluorobenzyl)-1H-pyrazole hydrochloride (Intermediate 6). The crude product was purified by column chromatography on SiO₂ (DCM:EtOAc=15:1) to give (S)-4-(3-fluorobenzyl)-N-(5-methyl-4-oxo-7-((tetrahydro-2H-pyran-4-yl)methoxy)-2,3,4,5-tetrahydrobenzo[b][1,4]oxazepin-3-yl)-1H-pyrazole-1-carboxamide (34% for 2 steps) as a white foam. ¹H-NMR (400 MHz, CDCl₃): δ 7.97 (1H, d, J=7.3 Hz), 7.88 (1H, d, J=0.9 Hz), 7.47 (1H, s), 7.24-7.22 (1H, m), 7.10 (1H, dd, J=7.1, 2.1 Hz), 7.00-6.85 (3H, m), 6.74 (2H, dd, J=7.5, 2.5 Hz), 4.93-4.87 (1H, m), 4.65 (1H, dd, J=9.8, 7.4 Hz), 4.24 (1H, dd, J=11.0, 10.1 Hz), 4.03 (2H, dd, J=11.4, 3.2 Hz), 3.80 (4H, d, J=6.9 Hz), 3.49-3.43 (2H, m), 3.42 (3H, s), 2.07 (1H, q, J=5.9 Hz), 1.76 (2H, d, J=11.0 Hz), 1.47 (2H, dd, J=12.8, 4.6 Hz). LC-MS: m/z=509.10 [M+H]⁺.

### Example 56: (S)—N-(7-(benzyloxy)-5-methyl-4-oxo-2,3,4,5-tetrahydrobenzo[b][1,4]oxazepin-3-yl)-4-(3-fluorobenzyl)-1H-pyrazole-1-carboxamide

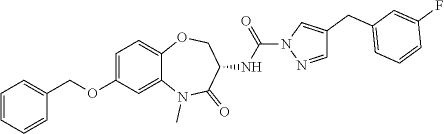 [SMILES: CN1C(=O)[C@@H](NC(=O)n2cc(Cc3cccc(F)c3)cn2)COc2ccc(OCc3ccccc3)cc21]

### Step A: tert-butyl (S)-(7-(benzyloxy)-5-methyl-4-oxo-2,3,4,5-tetrahydrobenzo[b][1,4]oxazepin-3-yl)carbamate

The title compound was prepared in a similar fashion to Example 15 (Step A) with Intermediate 2 and benzyl bromide. The crude product was purified by column chromatography on SiO₂ (Hexanes:EtOAc=3:1) to afford tert-butyl (S)-(7-(benzyloxy)-5-methyl-4-oxo-2,3,4,5-tetrahydrobenzo[b][1,4]oxazepin-3-yl)carbamate (94%) as white solid. ¹H-NMR (400 MHz, CDCl₃): δ 7.44-7.33 (5H, m), 7.05 (1H, dd, J=7.8, 1.4 Hz), 6.79-6.76 (2H, m), 5.48 (1H, d, J=7.2 Hz), 5.04 (2H, s), 4.66 (1H, dd, J=10.8, 7.2 Hz), 4.53 (1H, dd, J=9.6, 7.2 Hz), 4.10 (1H, dd, J=11.2, 9.6 Hz), 3.36 (3H, s), 1.40 (9H, s).

### Step B: (S)-3-amino-7-(benzyloxy)-5-methyl-2,3-dihydrobenzo[b][1,4]oxazepin-4(5H)-one hydrochloride

The title compound was prepared in a similar fashion to Example 15 (Step B) with tert-butyl (S)-(7-(benzyloxy)-5-methyl-4-oxo-2,3,4,5-tetrahydrobenzo[b][1,4]oxazepin-3-yl)carbamate. After concentration in vacuo, the crude product was used for the next reaction without purification. ¹H-NMR (400 MHz, MeOH-d₄): δ 7.45-7.43 (2H, m), 7.39-7.35 (2H, m), 7.33-7.29 (1H, m), 7.16 (1H, d, J=9.2 Hz), 7.04 (1H, d, J=3.2 Hz), 6.92 (1H, dd, J=8.8, 2.8 Hz), 5.11 (2H, s), 4.61 (1H, dd, J=10.0, 8.0 Hz), 4.42 (1H, dd, J=10.8, 10.0 Hz), 4.31 (1H, dd, J=10.8, 8.0 Hz), 3.39 (3H, s).

### Step C: (S)—N-(7-(benzyloxy)-5-methyl-4-oxo-2,3,4,5-tetrahydrobenzo[b][1,4]oxazepin-3-yl)-4-(3-fluorobenzyl)-1H-pyrazole-1-carboxamide

The title compound was prepared in a similar fashion to Example 15 (Step C) with (S)-3-amino-7-(benzyloxy)-5-methyl-2,3-dihydrobenzo[b][1,4]oxazepin-4(5H)-one hydrochloride and 4-(3-fluorobenzyl)-1H-pyrazole hydrochloride (Intermediate 6). The crude product was purified by column chromatography on SiO₂ (DCM:EtOAc=15:1) to afford (S)—N-(7-(benzyloxy)-5-methyl-4-oxo-2,3,4,5-tetrahydrobenzo[b][1,4]oxazepin-3-yl)-4-(3-fluorobenzyl)-1H-pyrazole-1-carboxamide (67% for 2 steps) as a white foam. ¹H-NMR (400 MHz, CDCl₃): δ 7.98 (1H, d, J=7.6 Hz), 7.88 (1H, d, J=0.8 Hz), 7.47 (1H, s), 7.45-7.33 (5H, m), 7.28-7.22 (1H, m), 7.11 (1H, d, J=9.2 Hz), 6.96-6.85 (3H, m), 6.84-6.81 (2H, m), 5.07 (2H, s), 4.91 (1H, dt, J=11.2, 7.6 Hz), 4.66 (1H, dd, J=9.6, 7.6 Hz), 4.25 (1H, dd, J=10.8, 9.6 Hz), 3.81 (2H, s), 3.39 (3H, s). LC-MS: m/z=501.10 [M+H]⁺.

### Example 57: (S)-4-(3-fluorobenzyl)-N-(7-((4-fluorobenzyl)oxy)-5-methyl-4-oxo-2,3,4,5-tetrahydrobenzo[b][1,4]oxazepin-3-yl)-1H-pyrazole-1-carboxamide

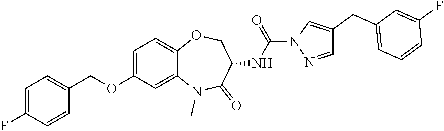 [SMILES: CN1C(=O)[C@@H](NC(=O)n2cc(Cc3cccc(F)c3)cn2)COc2ccc(OCc3ccc(F)cc3)cc21]

### Step A: tert-butyl (S)-(7-((4-fluorobenzyl)oxy)-5-methyl-4-oxo-2,3,4,5-tetrahydrobenzo[b][1,4]oxazepin-3-yl)carbamate

The title compound was prepared in a similar fashion to Example 15 (Step A) with Intermediate 2 and 1-(chloromethyl)-4-fluorobenzene. The crude product was purified by column chromatography on SiO₂ (Hexanes:EtOAc=2:1) to afford tert-butyl (S)-(7-((4-fluorobenzyl)oxy)-5-methyl-4-oxo-2,3,4,5-tetrahydrobenzo[b][1,4]oxazepin-3-yl)carbamate (60%) as white solid. ¹H-NMR (400 MHz, CDCl₃): δ 7.42-7.38 (2H, m), 7.12-7.04 (3H, m), 6.77-6.71 (2H, m), 5.47 (1H, d, J=7.6 Hz), 5.00 (2H, s), 4.65 (1H, dt, J=11.2, 7.2 Hz), 4.53 (1H, dd, J=9.6, 7.2 Hz), 4.10 (1H, dd, J=11.2, 9.6 Hz), 3.37 (3H, s), 1.40 (9H, s).

### Step B: (S)-3-amino-7-((5-fluoropyridin-2-yl)methoxy)-5-methyl-2,3-dihydrobenzo[b][1,4]oxazepin-4(5H)-one hydrochloride

The title compound was prepared in a similar fashion to Example 15 (Step B) with tert-butyl (S)-(7-((4-fluorobenzyl)oxy)-5-methyl-4-oxo-2,3,4,5-tetrahydrobenzo[b][1,4]oxazepin-3-yl)carbamate. After concentration in vacuo, the crude product was used for the next reaction without purification. ¹H-NMR (400 MHz, MeOH-d₄): δ 7.47 (2H, m), 7.18-7.08 (3H, m), 7.05 (1H, d, J=2.8 Hz), 6.93 (1H, dd, J=8.8, 2.8 Hz), 5.09 (2H, s), 4.58 (1H, dd, J=9.6, 7.2 Hz), 4.40 (1H, dd, J=10.8, 9.6 Hz), 4.30 (1H, dd, J=11.2, 7.2 Hz), 3.41 (3H, s).

### Step C: (S)-4-(3-fluorobenzyl)-N-(7-((4-fluorobenzyl)oxy)-5-methyl-4-oxo-2,3,4,5-tetrahydrobenzo[b][1,4]oxazepin-3-yl)-1H-pyrazole-1-carboxamide

The title compound was prepared in a similar fashion to Example 15 (Step C) with (S)-3-amino-7-((4-fluorobenzyl)oxy)-5-methyl-2,3-dihydrobenzo[b][1,4]oxazepin-4(5H)-one hydrochloride and 4-(3-fluorobenzyl)-1H-pyrazole hydrochloride (Intermediate 6). The crude product was purified by column chromatography on SiO₂ (Hexanes:EtOAc=2:1) to afford (S)-4-(3-fluorobenzyl)-N-(7-((4-fluorobenzyl)oxy)-5-methyl-4-oxo-2,3,4,5-tetrahydrobenzo[b][1,4]-oxazepin-3-yl)-1H-pyrazole-1-carboxamide (70% for 2 steps) as white foam. ¹H-NMR (400 MHz, CDCl₃): δ 7.99 (1H, d, J=7.6 Hz), 7.88 (1H, d, J=0.8 Hz), 7.46 (1H, s), 7.43-7.39 (2H, m), 7.25 (1H, td, J=8.0, 6.0 Hz), 7.12-7.06 (3H, m), 6.96-6.85 (3H, m), 6.82-6.79 (2H, m), 5.02 (2H, s), 4.90 (1H, dt, J=11.2, 7.2 Hz), 4.66 (1H, dd, J=9.6, 7.2 Hz), 4.25 (1H, dd, J=11.2, 9.6 Hz), 3.80 (2H, s), 3.39 (3H, s). LC-MS: m/z=519.10 [M+H]⁺.

### Example 58: (S)-4-(3-fluorobenzyl)-N-(7-((5-fluoropyridin-2-yl)methoxy)-5-methyl-4-oxo-2,3,4,5-tetrahydrobenzo[b][1,4]oxazepin-3-yl)-1H-pyrazole-1-carboxamide

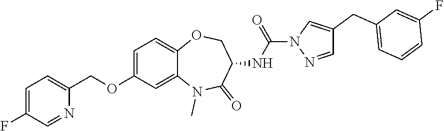 [SMILES: CN1C(=O)[C@@H](NC(=O)n2cc(Cc3cccc(F)c3)cn2)COc2ccc(OCc3ccc(F)cn3)cc21]

### Step A: tert-butyl (S)-(7-((5-fluoropyridin-2-yl)methoxy)-5-methyl-4-oxo-2,3,4,5-tetrahydrobenzo[b][1,4]oxazepin-3-yl)carbamate

The title compound was prepared in a similar fashion to Example 15 (Step A) with Intermediate 2 and (5-fluoropyridin-2-yl)methyl methanesulfonate using K₂CO₃ as a base. The crude product was purified by column chromatography on SiO₂ (Hexanes:EtOAc=1:1) afford tert-butyl (S)-(7-((5-fluoropyridin-2-yl)methoxy)-5-methyl-4-oxo-2,3,4,5-tetrahydrobenzo[b][1,4]oxazepin-3-yl)carbamate (68%) as a white foam. ¹H-NMR (400 MHz, CDCl₃): δ 8.47 (1H, d, J=2.8 Hz), 7.53 (1H, dd, J=8.8, 4.4 Hz), 7.46 (1H, td, J=8.8, 2.8 Hz), 7.05 (1H, d, J=8.4 Hz), 6.81 (1H, d, J=3.2 Hz), 6.77 (1H, dd, J=8.8, 3.2 Hz), 5.48 (1H, d, J=7.2 Hz), 5.16 (2H, s), 4.65 (1H, dt, J=11.2, 7.2 Hz), 4.53 (1H, dd, J=9.6, 7.6 Hz), 4.10 (1H, dd, J=11.2, 9.6 Hz), 3.37 (3H, s), 1.40 (9H, s).

### Step B: (S)-3-amino-7-((5-fluoropyridin-2-yl)methoxy)-5-methyl-2,3-dihydrobenzo[b][1,4]oxazepin-4(5H)-one hydrochloride

The title compound was prepared in a similar fashion to Example 15 (Step B) with tert-butyl (S)-(7-((5-fluoropyridin-2-yl)methoxy)-5-methyl-4-oxo-2,3,4,5-tetrahydrobenzo[b][1,4]oxazepin-3-yl)carbamate. After concentration in vacuo, the crude product was used for the next reaction without purification. ¹H-NMR (400 MHz, MeOH-d₄): δ 8.90 (1H, s), 8.31 (1H, td, J=8.8, 2.8 Hz), 8.10 (1H, dd, J=8.8, 4.4 Hz), 7.25-7.22 (2H, m), 7.04 (1H, dd, J=8.8, 2.8 Hz), 5.45 (2H, s), 4.63 (1H, dd, J=9.6, 7.2 Hz), 4.44 (1H, dd, J=10.8, 9.6 Hz), 4.35 (1H, dd, J=10.8, 7.2 Hz), 3.44 (3H, s).

### Step C: (S)-4-(3-fluorobenzyl)-N-(7-((5-fluoropyridin-2-yl)methoxy)-5-methyl-4-oxo-2,3,4,5-tetrahydrobenzo[b][1,4]oxazepin-3-yl)-1H-pyrazole-1-carboxamide

The title compound was prepared in a similar fashion to Example 15 (Step C) with (S)-3-amino-7-((5-fluoropyridin-2-yl)methoxy)-5-methyl-2,3-dihydrobenzo[b][1,4]oxazepin-4(5H)-one hydrochloride and 4-(3-fluorobenzyl)-1H-pyrazole hydrochloride (Intermediate 6). The crude product was purified by column chromatography on SiO₂ (DCM:EtOAc=10:1) to afford (S)-4-(3-fluorobenzyl)-N-(7-((5-fluoropyridin-2-yl)methoxy)-5-methyl-4-oxo-2,3,4,5-tetrahydro-benzo[b][1,4]oxazepin-3-yl)-1H-pyrazole-1-carboxamide (68% for 2 steps) as a white foam. ¹H-NMR (400 MHz, CDCl₃): δ 8.47 (1H, d, J=3.2 Hz), 8.00 (1H, d, J=7.2 Hz), 7.88 (1H, s), 7.54 (1H, dd, J=8.8, 4.4 Hz), 7.49-7.44 (2H, m), 7.25 (1H, td, J=8.0, 6.0 Hz), 7.11 (1H, d, J=8.8 Hz), 6.96-6.85 (4H, m), 6.82 (1H, dd, J=8.8, 3.2 Hz), 5.19 (2H, s), 4.90 (1H, dt, J=10.8, 7.6 Hz), 4.66 (1H, dd, J=9.6, 7.6 Hz), 4.25 (1H, dd, J=10.8, 9.6 Hz), 3.81 (2H, s), 3.40 (3H, s). LC-MS: m/z=520.10 [M+H]⁺.

### Example 59: (S)-4-(3-fluorobenzyl)-N-(7-((6-fluoropyridin-3-yl)methoxy)-5-methyl-4-oxo-2,3,4,5-tetrahydrobenzo[b][1,4]oxazepin-3-yl)-1H-pyrazole-1-carboxamide

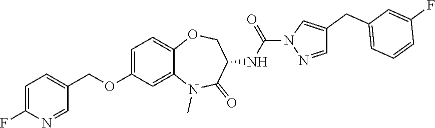 [SMILES: CN1C(=O)[C@@H](NC(=O)n2cc(Cc3cccc(F)c3)cn2)COc2ccc(OCc3ccc(F)nc3)cc21]

### Step A: tert-butyl-(S)-(7-((6-fluoropyridin-3-yl)methoxy)-5-methyl-4-oxo-2,3,4,5-tetrahydrobenzo[b][1,4]oxazepin-3-yl)carbamate

The title compound was prepared in a similar fashion to Example 15 (Step A) with Intermediate 2 and (6-fluoropyridin-3-yl)methyl methanesulfonate. The crude product was purified by column chromatography on SiO₂ (DCM:MeOH=20:1) to afford tert-butyl-(S)-(7-((6-fluoropyridin-3-yl)methoxy)-5-methyl-4-oxo-2,3,4,5-tetrahydrobenzo[b][1,4]oxazepin-3-yl)carbamate (59%) as a white foam. ¹H-NMR (400 MHz, CDCl₃): δ 8.30 (1H, d, J=1.8 Hz), 7.89 (1H, td, J=8.0, 2.6 Hz), 7.10-7.07 (1H, m), 7.00 (1H, dd, J=8.2, 2.7 Hz), 6.77 (2H, dd, J=11.0, 2.7 Hz), 5.47 (1H, d, J=6.9 Hz), 5.03 (2H, s), 4.69-4.62 (1H, m), 4.53 (1H, dd, J=9.6, 7.3 Hz), 4.15-4.08 (1H, m), 3.38 (3H, s), 1.40 (9H, s).

### Step B: (S)-3-amino-7-((6-fluoropyridin-3-yl)methoxy)-5-methyl-2,3 dihydrobenzo[b][1,4]oxazepin-4(5H)-one hydrochloride

The title compound was prepared in a similar fashion to Example 15 (Step B) with tert-butyl-(S)-(7-((6-fluoropyridin-3-yl)methoxy)-5-methyl-4-oxo-2,3,4,5-tetrahydrobenzo[b][1,4]oxazepin-3-yl)carbamate. After concentration in vacuo, the crude product was used for the next reaction without purification. LC-MS: m/z=318.10 [M+H]⁺.

### Step C: (S)-4-(3-fluorobenzyl)-N-(7-((6-fluoropyridin-3-yl)methoxy)-5-methyl-4-oxo-2,3,4,5-tetrahydrobenzo[b][1,4]oxazepin-3-yl)-1H-pyrazole-1-carboxamide

The title compound was prepared in a similar fashion to Example 15 (Step C) with (S)-3-amino-7-((6-fluoropyridin-3-yl)methoxy)-5-methyl-2,3-dihydrobenzo[b][1,4]oxazepin-4(5H)-one hydrochloride and 4-(3-fluorobenzyl)-1H-pyrazole hydrochloride (Intermediate 6). The crude product was purified by column chromatography on SiO₂ (DCM:EtOAc=10:1) to give (S)-4-(3-fluorobenzyl)-N-(7-((6-fluoropyridin-3-yl)methoxy)-5-methyl-4-oxo-2,3,4,5-tetrahydrobenzo[b][1,4]oxazepin-3-yl)-1H-pyrazole-1-carboxamide (21% for 2 steps) as a white foam. ¹H-NMR (400 MHz, CDCl₃): δ 8.31 (1H, d, J=2.3 Hz), 7.98 (1H, d, J=7.3 Hz), 7.91 (1H, dd, J=8.0, 2.5 Hz), 7.88 (1H, d, J=1.1 Hz), 7.47 (1H, s), 7.24-7.22 (1H, m), 7.14 (1H, t, J=4.8 Hz), 7.02-6.81 (6H, m), 5.06 (2H, s), 4.94-4.87 (1H, m), 4.67 (1H, dd, J=9.8, 7.5 Hz), 4.26 (1H, dd, J=11.2, 9.8 Hz), 3.81 (2H, s), 3.42 (3H, s). LC-MS: m/z=520.10 [M+H]⁺.

### Example 60: (S)-4-(3-fluorobenzyl)-N-(7-((2-fluoropyridin-4-yl)methoxy)-5-methyl-4-oxo-2,3,4,5-tetrahydrobenzo[b][1,4]oxazepin-3-yl)-1H-pyrazole-1-carboxamide

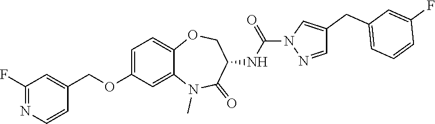 [SMILES: CN1C(=O)[C@@H](NC(=O)n2cc(Cc3cccc(F)c3)cn2)COc2ccc(OCc3ccnc(F)c3)cc21]

### Step A: tert-butyl (S)-(7-((2-fluoropyridin-4-yl)methoxy)-5-methyl-4-oxo-2,3,4,5-tetrahydrobenzo[b][1,4]oxazepin-3-yl)carbamate

The title compound was prepared in a similar fashion to Example 15 (Step A) with Intermediate 2 and (2-fluoropyridin-4-yl)methyl methanesulfonate using K₂CO₃ as a base. The crude product was purified by column chromatography on SiO₂ (Hexanes:EtOAc=1:1) to afford tert-butyl (S)-(7-((2-fluoropyridin-4-yl)methoxy)-5-methyl-4-oxo-2,3,4,5-tetrahydrobenzo[b][1,4]oxazepin-3-yl)carbamate (77%) as a white solid. ¹H-NMR (400 MHz, CDCl₃): δ 8.21 (1H, d, J=4.8 Hz), 7.96 (1H, ddd, J=9.6, 7.2, 1.6 Hz), 7.29-7.25 (1H, m), 7.08 (1H, d, J=8.8 Hz), 6.82 (1H, d, J=3.2 Hz), 6.79 (1H, dd, J=8.8, 2.8 Hz), 5.52 (1H, d, J=7.6 Hz), 5.11 (2H, s), 4.66 (1H, dt, J=10.8, 7.2 Hz), 4.53 (1H, dd, J=9.6, 7.6 Hz), 4.11 (1H, dd, J=11.2, 9.6 Hz), 3.39 (3H, s), 1.40 (9H, s).

### Step B: (S)-3-amino-7-((2-fluoropyridin-4-yl)methoxy)-5-methyl-2,3-dihydrobenzo[b][1,4]oxazepin-4(5H)-one hydrochloride

The title compound was prepared in a similar fashion to Example 15 (Step B) with tert-butyl (S)-(7-((2-fluoropyridin-4-yl)methoxy)-5-methyl-4-oxo-2,3,4,5-tetrahydrobenzo[b][1,4]oxazepin-3-yl)carbamate. After concentration in vacuo, the crude product was used for the next reaction without purification. ¹H-NMR (400 MHz, MeOH-d₄): δ 8.20 (1H, d, J=4.8 Hz), 8.09 (1H, ddd, J=9.6, 7.2, 2.0 Hz), 7.36 (1H, ddd, J=7.2, 4.8, 1.6 Hz), 7.20 (1H, d, J=8.8 Hz), 7.11 (1H, d, J=2.8 Hz), 6.98 (1H, dd, J=8.8, 2.8 Hz), 5.18 (2H, s), 4.58 (1H, dd, J=9.6, 7.6 Hz), 4.41 (1H, dd, 11.2, 10.0 Hz), 4.31 (1H, dd, J=11.2, 7.6 Hz), 3.42 (3H, s).

### Step C: (S)-4-(3-fluorobenzyl)-N-(7-((2-fluoropyridin-4-yl)methoxy)-5-methyl-4-oxo-2,3,4,5-tetrahydrobenzo[b][1,4]oxazepin-3-yl)-1H-pyrazole-1-carboxamide

The title compound was prepared in a similar fashion to Example 15 (Step C) with (S)-3-amino-7-((2-fluoropyridin-4-yl)methoxy)-5-methyl-2,3-dihydrobenzo[b][1,4]oxazepin-4(5H)-one hydrochloride and 4-(3-fluorobenzyl)-1H-pyrazole hydrochloride (Intermediate 6). The crude product was purified by column chromatography on SiO₂ (DCM:EtOAc=15:1) to afford (S)-4-(3-fluorobenzyl)-N-(7-((2-fluoropyridin-4-yl)methoxy)-5-methyl-4-oxo-2,3,4,5-tetrahydrobenzo[b][1,4]oxazepin-3-yl)-1H-pyrazole-1-carboxamide (78% for 2 steps) as a white solid. ¹H-NMR (400 MHz, CDCl₃): δ 8.22 (1H, d, J=5.2 Hz), 7.99-7.95 (2H, m), 7.88 (1H, d, J=0.8 Hz), 7.47 (1H, s), 7.28-7.22 (2H, m), 7.14 (1H, dd, J=8.4, 0.8 Hz), 6.97-6.82 (5H, m), 5.13 (2H, s), 4.91 (1H, dt, J=10.8, 7.6 Hz), 4.67 (1H, dd, J=9.6, 7.6 Hz), 4.26 (1H, dd, J=11.2, 9.6 Hz), 3.81 (2H, s), 3.42 (3H, s). LC-MS: m/z=520.10 [M+H]⁺.

### Example 61: (S)-4-(3-fluorobenzyl)-N-(7-((2-methoxypyridin-4-yl)methoxy)-5-methyl-4-oxo-2,3,4,5-tetrahydrobenzo[b][1,4]oxazepin-3-yl)-1H-pyrazole-1-carboxamide

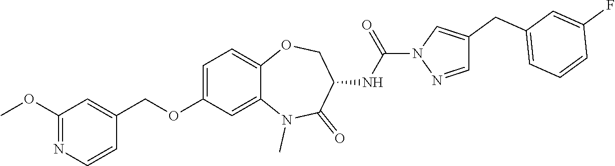 [SMILES: COc1cc(COc2ccc3c(c2)N(C)C(=O)[C@@H](NC(=O)n2cc(Cc4cccc(F)c4)cn2)CO3)ccn1]

### Step A: tert-butyl (S)-(7-((2-methoxypyridin-4-yl)methoxy)-5-methyl-4-oxo-2,3,4,5-tetrahydrobenzo[b][1,4]oxazepin-3-yl)carbamate

The title compound was prepared in a similar fashion to Example 15 (Step A) with Intermediate 2 and (2-methoxypyridin-4-yl)methyl methanesulfonate. The crude product was purified by column chromatography on SiO₂ (Hexanes:EtOAc=1:1) to afford tert-butyl (S)-(7-((2-methoxypyridin-4-yl)methoxy)-5-methyl-4-oxo-2,3,4,5-tetrahydrobenzo[b][1,4]oxazepin-3-yl)carbamate (80%) as a white foam. ¹H-NMR (400 MHz, CDCl₃) δ 8.18 (1H, d, J=4.8 Hz), 7.06 (1H, d, J=8.8 Hz), 6.92 (1H, dd, J=5.2, 1.2 Hz), 6.81 (1H, s), 6.78 (1H, d, J=3.2 Hz), 6.73 (1H, dd, J=9.2, 3.2 Hz), 5.48 (1H, d, J=6.8 Hz), 5.01 (2H, s), 4.65 (1H, dt, J=11.6, 7.2 Hz), 4.53 (1H, dd, J=9.6, 7.2 Hz), 4.10 (1H, dd, J=11.6, 9.6 Hz), 3.96 (3H, s), 3.37 (3H, s), 1.40 (9H, s).

### Step B: (S)-3-amino-7-((2-methoxypyridin-4-yl)methoxy)-5-methyl-2,3-dihydrobenzo[b][1,4]oxazepin-4(5H)-one hydrochloride

The title compound was prepared in a similar fashion to Example 15 (Step B) with tert-butyl (S)-(7-((2-methoxypyridin-4-yl)methoxy)-5-methyl-4-oxo-2,3,4,5-tetrahydrobenzo[b][1,4]oxazepin-3-yl)carbamate. After concentration in vacuo, the crude product was used for the next reaction without purification. ¹H-NMR (400 MHz, MeOH-d₄): δ 8.36 (1H, d, J=6.0 Hz), 7.68 (1H, s), 7.60 (1H, d, J=6.0 Hz), 7.25-7.22 (2H, m), 7.03 (1H, dd, J=8.8, 2.8 Hz), 5.43 (2H, s), 4.63 (1H, dd, J=9.6, 7.2 Hz), 4.44 (1H, dd, J=11.2, 9.6 Hz), 4.35 (1H, dd, J=11.2, 7.2 Hz), 4.27 (3H, s), 3.45 (3H, s).

### Step C: (S)-4-(3-fluorobenzyl)-N-(7-((2-methoxypyridin-4-yl)methoxy)-5-methyl-4-oxo-2,3,4,5-tetrahydrobenzo[b][1,4]oxazepin-3-yl)-1H-pyrazole-1-carboxamide

The title compound was prepared in a similar fashion to Example 15 (Step C) with (S)-3-amino-7-((2-methoxypyridin-4-yl)methoxy)-5-methyl-2,3-dihydrobenzo[b][1,4]oxazepin-4(5H)-one hydrochloride and 4-(3-fluorobenzyl)-1H-pyrazole hydrochloride (Intermediate 6). The crude product was purified by column chromatography on SiO₂ (Hexanes:EtOAc=3:1) to afford (S)-4-(3-fluorobenzyl)-N-(7-((2-methoxypyridin-4-yl)methoxy)-5-methyl-4-oxo-2,3,4,5-tetrahydrobenzo[b][1,4]oxazepin-3-yl)-1H-pyrazole-1-carboxamide (60% for 2 steps) as pale-yellow foam. ¹H-NMR (400 MHz, CDCl₃): δ 8.18 (1H, d, J=4.8 Hz), 8.00 (1H, d, J=7.6 Hz), 7.88 (1H, d, J=0.8 Hz), 7.47 (1H, s), 7.24 (1H, td, J=7.6, 6.0 Hz), 7.11 (1H, d, J=9.2 Hz), 6.96-6.84 (4H, m), 6.82 (2H, d, J=2.3 Hz), 6.77 (1H, dd, J=8.8, 3.2 Hz), 5.04 (2H, s), 4.90 (1H, dt, J=12.0, 7.2 Hz), 4.66 (1H, dd, J=9.6, 7.2 Hz), 4.26 (1H, dd, J=10.8, 10.0 Hz), 3.95 (3H, s), 3.80 (2H, s), 3.40 (3H, s). LC-MS: m/z=532.1 [M+H]⁺.

### Example 62: (S)—N-(7-((2-chloropyrimidin-5-yl)methoxy)-5-methyl-4-oxo-2,3,4,5-tetrahydrobenzo[b][1,4]oxazepin-3-yl)-4-(3-fluorobenzyl)-1H-pyrazole-1-carboxamide

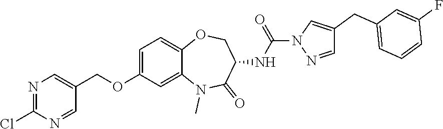 [SMILES: CN1C(=O)[C@@H](NC(=O)n2cc(Cc3cccc(F)c3)cn2)COc2ccc(OCc3cnc(Cl)nc3)cc21]

### Step A: tert-butyl-(S)-(7-((2-chloropyrimidin-5-yl)methoxy)-5-methyl-4-oxo-2,3,4,5-tetrahydrobenzo[b][1,4]oxazepin-3-yl)carbamate

The title compound was prepared in a similar fashion to Example 15 (Step A) with Intermediate 2 and (2-chloropyrimidin-5-yl)methyl methanesulfonate. The crude product was purified by column chromatography on SiO₂ (Hexanes:EtOAc=1:1) to afford tert-butyl-(S)-(7-((2-chloropyrimidin-5-yl)methoxy)-5-methyl-4-oxo-2,3,4,5-tetrahydrobenzo[b][1,4]oxazepin-3-yl)carbamate (80%) as a white foam. ¹H-NMR (400 MHz, CDCl₃): δ 8.73 (2H, s), 7.11 (1H, d, J=8.7 Hz), 6.81-6.76 (2H, m), 5.47 (1H, d, J=7.3 Hz), 5.05 (2H, s), 4.69-4.62 (1H, m), 4.54 (1H, dd, J=9.6, 7.3 Hz), 4.15-4.09 (2H, m), 3.39 (3H, s), 1.40 (9H, s).

### Step B: (S)-3-amino-7-((2-chloropyrimidin-5-yl)methoxy)-5-methyl-2,3-dihydrobenzo[b][1,4]oxazepin-4(5H)-one hydrochloride

The title compound was prepared in a similar fashion to Example 15 (Step B) with tert-butyl-(S)-(7-((2-chloropyrimidin-5-yl)methoxy)-5-methyl-4-oxo-2,3,4,5-tetrahydrobenzo[b][1,4]oxazepin-3-yl)carbamate. After concentration in vacuo, the crude product was used for the next reaction without purification. LC-MS: m/z=335.10 [M+H]⁺.

### Step C: (S)—N-(7-((2-chloropyrimidin-5-yl)methoxy)-5-methyl-4-oxo-2,3,4,5-tetrahydrobenzo[b][1,4]oxazepin-3-yl)-4-(3-fluorobenzyl)-1H-pyrazole-1-carboxamide

The title compound was prepared in a similar fashion to Example 15 (Step C) with (S)-3-amino-7-((2-chloropyrimidin-5-yl)methoxy)-5-methyl-2,3-dihydrobenzo[b][1,4]oxazepin-4(5H)-one hydrochloride and 4-(3-fluorobenzyl)-1H-pyrazole hydrochloride (Intermediate 6). The crude product was purified by column chromatography on SiO₂ (DCM:EtOAc=10:1) to give (S)—N-(7-((2-chloropyrimidin-5-yl)methoxy)-5-methyl-4-oxo-2,3,4,5-tetrahydrobenzo[b][1,4]oxazepin-3-yl)-4-(3-fluorobenzyl)-1H-pyrazole-1-carboxamide (40% for 2 steps) as a white foam. ¹H-NMR (400 MHz, CDCl₃): δ 8.74 (2H, s), 7.98 (1H, d, J=7.3 Hz), 7.88 (1H, s), 7.47 (1H, s), 7.23 (1H, d, J=8.2 Hz), 7.17 (1H, d, J=9.1 Hz), 7.00-6.81 (5H, m), 5.07 (2H, s), 4.94-4.88 (1H, m), 4.67 (1H, dd, J=9.8, 7.5 Hz), 4.27 (1H, t, J=10.5 Hz), 3.81 (2H, s), 3.43 (3H, s). LC-MS: m/z=537.10 [M+H]⁺.

### Example 63: (S)-4-(3-fluorobenzyl)-N-(5-methyl-7-((1-methyl-1H-imidazol-2-yl)methoxy)-4-oxo-2,3,4,5-tetrahydrobenzo[b][1,4]oxazepin-3-yl)-1H-pyrazole-1-carboxamide

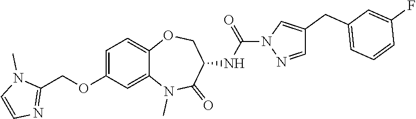 [SMILES: CN1C(=O)[C@@H](NC(=O)n2cc(Cc3cccc(F)c3)cn2)COc2ccc(OCc3nccn3C)cc21]

### Step A: tert-butyl (S)-(5-methyl-7-((1-methyl-1H-imidazol-2-yl)methoxy)-4-oxo-2,3,4,5-tetrahydrobenzo[b][1,4]oxazepin-3-yl)carbamate

The title compound was prepared in a similar fashion to Example 15 (Step A) with Intermediate 2 and 2-(chloromethyl)-1-methyl-1H-imidazole. The crude product was purified by column chromatography on SiO₂ (DCM:MeOH=20:1) to afford tert-butyl (S)-(5-methyl-7-((1-methyl-1H-imidazol-2-yl)methoxy)-4-oxo-2,3,4,5-tetrahydrobenzo[b][1,4]oxazepin-3-yl)carbamate (61%) as a yellow solid. ¹H-NMR (400 MHz, CDCl₃): δ 7.03 (1H, d, J=8.8 Hz), 7.00 (1H, d, J=1.2 Hz), 6.90-6.84 (3H, m), 5.47 (1H, d, J=7.2 Hz), 5.14 (2H, s), 4.68-4.57 (1H, m), 4.50 (1H, dd, J=9.6, 8.0 Hz), 4.08 (1H, dd, J=11.2, 9.6 Hz), 3.73 (3H, s), 3.35 (3H, s), 1.38 (9H, s).

### Step B: (S)-3-amino-5-methyl-7-((1-methyl-1H-imidazol-2-yl)methoxy)-2,3-dihydrobenzo[b][1,4]oxazepin-4(5H)-one hydrochloride

The title compound was prepared in a similar fashion to Example 15 (Step B) with tert-butyl (S)-(5-methyl-7-((1-methyl-1H-imidazol-2-yl)methoxy)-4-oxo-2,3,4,5-tetrahydrobenzo[b][1,4]oxazepin-3-yl)carbamate. After concentration in vacuo, the crude product was used for the next reaction without purification. LC-MS: m/z=303.10 [M+H]⁺.

### Step C: (S)-4-(3-fluorobenzyl)-N-(5-methyl-7-((1-methyl-1H-imidazol-2-yl)methoxy)-4-oxo-2,3,4,5-tetrahydrobenzo[b][1,4]oxazepin-3-yl)-1H-pyrazole-1-carboxamide

The title compound was prepared in a similar fashion to Example 15 (Step C) with (S)-3-amino-5-methyl-7-((1-methyl-1H-imidazol-2-yl)methoxy)-2,3-dihydrobenzo[b][1,4]oxazepin-4(5H)-one hydrochloride and 4-(3-fluorobenzyl)-1H-pyrazole hydrochloride (Intermediate 6). The crude product was purified by column chromatography on SiO₂ (Hexanes:EtOAc=1:1) to give (S)-4-(3-fluorobenzyl)-N-(5-methyl-7-((1-methyl-1H-imidazol-2-yl)methoxy)-4-oxo-2,3,4,5-tetrahydrobenzo[b][1,4]oxazepin-3-yl)-1H-pyrazole-1-carboxamide (54% for 2 steps) as a white foam. ¹H-NMR (400 MHz, CDCl₃): δ 7.97 (1H, d, J=7.2 Hz), 7.86 (1H, d, J=1.2 Hz), 7.45 (1H, m), 7.26-7.21 (1H, m), 7.09 (1H, d, J=8.0 Hz), 7.00 (1H, d, J=1.6 Hz), 6.95-6.83 (6H, m), 5.16 (2H, s), 4.88 (1H, td, J=10.8, 7.2 Hz), 4.63 (1H, dd, J=10.0, 7.2 Hz), 4.23 (1H, dd, J=11.2, 10.0 Hz), 3.80 (2H, s), 3.74 (3H, s), 3.38 (3H, s). LC-MS: m/z=505.10 [M+H]⁺.

### Example 64: (S)-4-(3-fluorobenzyl)-N-(5-methyl-7-((5-methyl-1,3,4-oxadiazol-2-yl)methoxy)-4-oxo-2,3,4,5-tetrahydrobenzo[b][1,4]oxazepin-3-yl)-1H-pyrazole-1-carboxamide

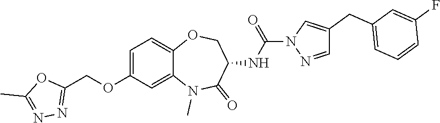 [SMILES: Cc1nnc(COc2ccc3c(c2)N(C)C(=O)[C@@H](NC(=O)n2cc(Cc4cccc(F)c4)cn2)CO3)o1]

### Step A: tert-butyl-(S)-(5-methyl-7-((5-methyl-1,3,4-oxadiazol-2-yl)methoxy)-4-oxo-2,3,4,5-tetrahydrobenzo[b][1,4]oxazepin-3-yl)carbamate

The title compound was prepared in a similar fashion to Example 15 with Intermediate 2 and 2-(chloromethyl)-5-methyl-1,3,4-oxadiazole. The crude product was purified by column chromatography on SiO₂ (Hexanes:EtOAc=2:1) to afford tert-butyl-(S)-(5-methyl-7-((5-methyl-1,3,4-oxadiazol-2-yl)methoxy)-4-oxo-2,3,4,5-tetrahydrobenzo[b][1,4]oxazepin-3-yl)carbamate (66%) as a white solid. ¹H-NMR (400 MHz, CDCl₃): δ 7.08 (1H, d, J=8.7 Hz), 6.84 (2H, td, J=8.0, 2.7 Hz), 5.47 (1H, d, J=7.3 Hz), 4.67-4.60 (1H, m), 4.52 (1H, dd, J=9.6, 7.3 Hz), 4.11 (1H, dd, J=11.0, 9.6 Hz), 3.38 (3H, s), 2.59 (3H, s), 1.40 (9H, s).

### Step B: (S)-3-amino-5-methyl-7-((5-methyl-1,3,4-oxadiazol-2-yl)methoxy)-2,3-dihydrobenzo[b][1,4]oxazepin-4(5H)-one hydrochloride

The title compound was prepared in a similar fashion to Example 15 (Step B) with tert-butyl-(S)-(5-methyl-7-((5-methyl-1,3,4-oxadiazol-2-yl)methoxy)-4-oxo-2,3,4,5-tetrahydrobenzo[b][1,4]oxazepin-3-yl)carbamate. After concentration in vacuo, the crude product was used for the next reaction without purification. ¹H-NMR (400 MHz, DMSO-d₆): δ 8.50 (3H, s), 7.19 (1H, d, J=8.7 Hz), 7.13 (1H, dd, J=11.4, 3.2 Hz), 6.85 (1H, td, J=9.4, 2.9 Hz), 4.84 (2H, s), 4.52 (1H, dd, J=9.6, 7.8 Hz), 4.37 (1H, t, J=10.5 Hz), 4.23 (1H, s), 3.71 (3H, s), 3.34 (3H, s).

### Step C: (S)-4-(3-fluorobenzyl)-N-(5-methyl-7-((5-methyl-1,3,4-oxadiazol-2-yl)methoxy)-4-oxo-2,3,4,5-tetrahydrobenzo[b][1,4]oxazepin-3-yl)-1H-pyrazole-1-carboxamide

The title compound was prepared in a similar fashion to Example 15 (Step C) with (S)-3-amino-5-methyl-7-((5-methyl-1,3,4-oxadiazol-2-yl)methoxy)-2,3-dihydrobenzo[b][1,4]oxazepin-4(5H)-one hydrochloride and 4-(3-fluorobenzyl)-1H-pyrazole hydrochloride (Intermediate 6). The crude product was purified by column chromatography on SiO₂ (DCM:EtOAc=10:1) to give (S)-4-(3-fluorobenzyl)-N-(5-methyl-7-((5-methyl-1,3,4-oxadiazol-2-yl)methoxy)-4-oxo-2,3,4,5-tetrahydrobenzo[b][1,4]oxazepin-3-yl)-1H-pyrazole-1-carboxamide (13% for 2 steps) as a white foam. ¹H-NMR (400 MHz, CDCl₃): δ 7.98 (1H, d, J=7.3 Hz), 7.88 (1H, d, J=0.9 Hz), 7.47 (1H, s), 7.24-7.22 (1H, m), 7.12 (1H, d, J=8.7 Hz), 7.00-6.85 (3H, m), 6.83 (1H, d, J=2.7 Hz), 6.73 (1H, dd, J=8.7, 2.7 Hz), 4.93-4.87 (1H, m), 4.69-4.67 (1H, m), 4.64 (2H, s), 4.25 (1H, dd, J=11.2, 9.8 Hz), 3.83 (3H, s), 3.81 (2H, s), 3.41 (3H, s). LC-MS: m/z=507.10 [M+H]⁺.

### Example 65: Methyl (S)-2-(((3-(4-(3-fluorobenzyl)-1H-pyrazole-1-carboxamido)-5-methyl-4-oxo-2,3,4,5-tetrahydrobenzo[b][1,4]oxazepin-7-yl)oxy)methyl)thiazole-4-carboxylate

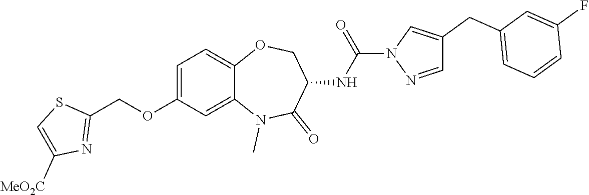 [SMILES: COC(=O)c1csc(COc2ccc3c(c2)N(C)C(=O)[C@@H](NC(=O)n2cc(Cc4cccc(F)c4)cn2)CO3)n1]

### Step A: Methyl (S)-2-(((3-((tert-butoxycarbonyl)amino)-5-methyl-4-oxo-2,3,4,5-tetrahydrobenzo[b][1,4]oxazepin-7-yl)oxy)methyl)thiazole-4-carboxylate

The title compound was prepared in a similar fashion to Example 15 (Step A) with Intermediate 2 and methyl 2-(chloromethyl)thiazole-4-carboxylate. The crude product was purified by column chromatography on SiO₂ (Hexanes:EtOAc=2:1) to afford methyl (S)-2-(((3-((tert-butoxycarbonyl)amino)-5-methyl-4-oxo-2,3,4,5-tetrahydrobenzo[b][1,4]oxazepin-7-yl)oxy)methyl)thiazole-4-carboxylate (47%) as a yellow solid. ¹H-NMR (400 MHz, CDCl₃): δ 8.25 (1H, s), 7.07 (1H, d, J=8.7 Hz), 6.84-6.78 (2H, m), 5.47 (1H, d, J=7.3 Hz), 5.39 (2H, s), 4.67-4.61 (1H, m), 4.53 (1H, dd, J=9.6, 7.3 Hz), 4.16-4.08 (1H, m), 3.98 (3H, s), 3.38 (3H, s), 1.39 (9H, s).

### Step B: Methyl (S)-2-(((3-amino-5-methyl-4-oxo-2,3,4,5-tetrahydrobenzo[b][1,4]oxazepin-7-yl)oxy)methyl)thiazole-4-carboxylate hydrochloride

The title compound was prepared in a similar fashion to Example 15 (Step B) with methyl (S)-2-(((3-((tert-butoxycarbonyl)amino)-5-methyl-4-oxo-2,3,4,5-tetrahydrobenzo[b][1,4]oxazepin-7-yl)oxy)methyl)thiazole-4-carboxylate. After concentration in vacuo, the crude product was used for the next reaction without purification. ¹H-NMR (400 MHz, DMSO-d₆): δ 8.62 (1H, s), 8.40 (3H, s), 7.30 (1H, d, J=2.7 Hz), 7.23 (1H, d, J=8.7 Hz), 7.01 (1H, dd, J=8.9, 3.0 Hz), 5.52 (2H, d, J=2.3 Hz), 4.51 (1H, dd, J=9.6, 7.8 Hz), 4.37 (1H, t, J=10.3 Hz), 4.28 (1H, dd, J=11.0, 7.8 Hz), 3.84 (3H, s), 3.36 (3H, s).

### Step C: Methyl (S)-2-(((3-(4-(3-fluorobenzyl)-1H-pyrazole-1-carboxamido)-5-methyl-4-oxo-2,3,4,5-tetrahydrobenzo[b][1,4]oxazepin-7-yl)oxy)methyl)thiazole-4-carboxylate

The title compound was prepared in a similar fashion to Example 15 (Step C) with methyl (S)-2-(((3-amino-5-methyl-4-oxo-2,3,4,5-tetrahydrobenzo[b][1,4]oxazepin-7-yl)oxy)methyl)thiazole-4-carboxylate hydrochloride and 4-(3-fluorobenzyl)-1H-pyrazole hydrochloride (Intermediate 6). The crude product was purified by column chromatography on SiO₂ (Hexanes:EtOAc=1:1) to give methyl (S)-2-(((3-(4-(3-fluorobenzyl)-1H-pyrazole-1-carboxamido)-5-methyl-4-oxo-2,3,4,5-tetrahydrobenzo[b][1,4]oxazepin-7-yl)oxy)methyl)thiazole-4-carboxylate (46% for 2 steps) as a yellow solid. ¹H-NMR (400 MHz, CDCl₃): δ 8.25 (1H, s), 7.97 (1H, d, J=7.3 Hz), 7.87 (1H, d, J=0.9 Hz), 7.47 (1H, s), 7.24-7.22 (1H, m), 7.14 (1H, d, J=9.1 Hz), 6.96-6.83 (5H, m), 5.42 (2H, s), 4.92-4.86 (1H, m), 4.66 (1H, dd, J=9.8, 7.5 Hz), 4.26 (1H, dd, J=11.2, 9.8 Hz), 3.98 (3H, s), 3.81 (2H, s), 3.41 (3H, s). LC-MS: m/z=566.00 [M+H]⁺.

### Example 66: (S)-4-(3-fluorobenzyl)-N-(7-((4-(2-hydroxypropan-2-yl)thiazol-2-yl)methoxy)-5-methyl-4-oxo-2,3,4,5-tetrahydrobenzo[b][1,4]oxazepin-3-yl)-1H-pyrazole-1-carboxamide

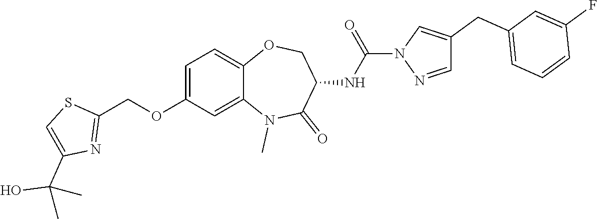 [SMILES: CN1C(=O)[C@@H](NC(=O)n2cc(Cc3cccc(F)c3)cn2)COc2ccc(OCc3nc(C(C)(C)O)cs3)cc21]

To a solution of methyl (S)-2-(((3-(4-(3-fluorobenzyl)-1H-pyrazole-1-carboxamido)-5-methyl-4-oxo-2,3,4,5-tetrahydrobenzo[b][1,4]oxazepin-7-yl)oxy)methyl)thiazole-4-carboxylate (Example 65, 20.0 mg, 0.035 mmol) in THF (0.20 mL) was added MeMgCl (3 M solution in THF, 58.0 μL, 0.177 mmol) at 0° C. The reaction mixture was stirred at room temperature for 5 hours. After quenched with saturated aq. NH₄Cl solution at 0° C., the mixture was extracted with DCM twice. The combined organic layers were washed with water and brine, dried over Na₂SO₄, filtered, and concentrated in vacuo. The residue was purified by column chromatography on SiO₂ (Hexanes:EtOAc=1:1) to give (S)-4-(3-fluorobenzyl)-N-(7-((4-(2-hydroxypropan-2-yl)thiazol-2-yl)methoxy)-5-methyl-4-oxo-2,3,4,5-tetrahydrobenzo[b][1,4]oxazepin-3-yl)-1H-pyrazole-1-carboxamide (10.0 mg, 50%) as a white foam. ¹H-NMR (400 MHz, CDCl₃): δ 7.98 (1H, d, J=7.3 Hz), 7.88 (1H, d, J=0.9 Hz), 7.47 (1H, s), 7.24-7.22 (1H, m), 7.17 (1H, s), 7.13 (1H, dd, J=8.0, 1.1 Hz), 6.96-6.85 (5H, m), 5.34 (2H, s), 4.92-4.86 (1H, m), 4.66 (1H, dd, J=9.6, 7.3 Hz), 4.25 (1H, dd, J=11.0, 9.6 Hz), 3.81 (2H, s), 3.41 (3H, s), 1.63 (6H, s). LC-MS: m/z=566.20 [M+H]⁺.

### Example 67: (S)—N-(7-((3,5-dimethylisoxazol-4-yl)methoxy)-5-methyl-4-oxo-2,3,4,5-tetrahydrobenzo[b][1,4]oxazepin-3-yl)-4-(3-fluorobenzyl)-1H-pyrazole-1-carboxamide

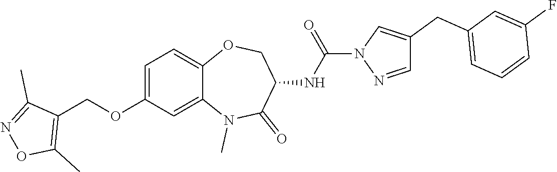 [SMILES: Cc1noc(C)c1COc1ccc2c(c1)N(C)C(=O)[C@@H](NC(=O)n1cc(Cc3cccc(F)c3)cn1)CO2]

### Step A: tert-butyl (S)-(7-((3,5-dimethylisoxazol-4-yl)methoxy)-5-methyl-4-oxo-2,3,4,5-tetrahydrobenzo[b][1,4]oxazepin-3-yl)carbamate

The title compound was prepared in a similar fashion to Example 15 (Step A) with Intermediate 2 and 4-(chloromethyl)-3,5-dimethylisoxazole. The residue was purified by column chromatography on SiO₂ (Hexanes:EtOAc=1:1) to afford tert-butyl (S)-(7-((3,5-dimethylisoxazol-4-yl)methoxy)-5-methyl-4-oxo-2,3,4,5-tetrahydrobenzo[b][1,4]oxazepin-3-yl)carbamate (59%) as a white foam. ¹H-NMR (400 MHz, CDCl₃): δ 7.09 (1H, dd, J=7.8, 1.0 Hz), 6.76 (2H, dd, J=11.0, 2.3 Hz), 5.46 (1H, d, J=7.3 Hz), 4.75 (2H, s), 4.70-4.63 (1H, m), 4.53 (1H, dd, J=9.6, 7.3 Hz), 4.11 (1H, dd, J=11.2, 9.8 Hz), 3.38 (3H, s), 2.43 (3H, s), 2.31 (3H, s), 1.40 (9H, s).

### Step B: (S)-3-amino-7-((3,5-dimethylisoxazol-4-yl)methoxy)-5-methyl-2,3-dihydrobenzo[b][1,4]oxazepin-4(5H)-one hydrochloride

The title compound was prepared in a similar fashion to Example 15 (Step B) with tert-butyl (S)-(7-((3,5-dimethylisoxazol-4-yl)methoxy)-5-methyl-4-oxo-2,3,4,5-tetrahydrobenzo[b][1,4]oxazepin-3-yl)carbamate. After concentration in vacuo, the crude product was used for the next reaction without purification. ¹H-NMR (400 MHz, DMSO-d₆): δ 8.41 (3H, s), 7.22-7.20 (2H, m), 6.95 (1H, dd, J=8.7, 2.7 Hz), 4.95 (2H, s), 3.36 (3H, s), 2.41 (3H, s), 2.23 (3H, s).

### Step C: (S)—N-(7-((3,5-dimethylisoxazol-4-yl)methoxy)-5-methyl-4-oxo-2,3,4,5-tetrahydrobenzo[b][1,4]oxazepin-3-yl)-4-(3-fluorobenzyl)-1H-pyrazole-1-carboxamide

The title compound was prepared in a similar fashion to Example 15 (Step C) with (S)-3-amino-7-((3,5-dimethylisoxazol-4-yl)methoxy)-5-methyl-2,3-dihydrobenzo[b][1,4]oxazepin-4(5H)-one hydrochloride and 4-(3-fluorobenzyl)-1H-pyrazole hydrochloride (Intermediate 6). The crude product was purified by column chromatography on SiO₂ (DCM:EtOAc=15:1) to give (S)—N-(7-((3,5-dimethylisoxazol-4-yl)methoxy)-5-methyl-4-oxo-2,3,4,5-tetrahydrobenzo[b][1,4]oxazepin-3-yl)-4-(3-fluorobenzyl)-1H-pyrazole-1-carboxamide (18% for 2 steps) as a white foam. ¹H-NMR (400 MHz, CDCl₃): δ 7.97 (1H, d, J=7.6 Hz), 7.88 (1H, d, J=0.8 Hz), 7.47 (1H, s), 7.25-7.23 (1H, m), 7.15 (1H, d, J=8.4 Hz), 7.00-6.78 (5H, m), 4.95-4.89 (1H, m), 4.78 (2H, s), 4.66 (1H, dd, J=9.8, 7.4 Hz), 4.27 (1H, dd, J=11.2, 10 Hz), 3.81 (2H, s), 3.42 (3H, s), 2.43 (3H, s), 2.32 (3H, s). LC-MS: m/z=520.10 [M+H]⁺.

### Example 68: (S)-4-(3-fluorobenzyl)-N-(7-((1-(2-hydroxy-2-methylpropyl)-1H-1,2,3-triazol-4-yl)methoxy)-5-methyl-4-oxo-2,3,4,5-tetrahydrobenzo[b][1,4]oxazepin-3-yl)-1H-pyrazole-1-carboxamide

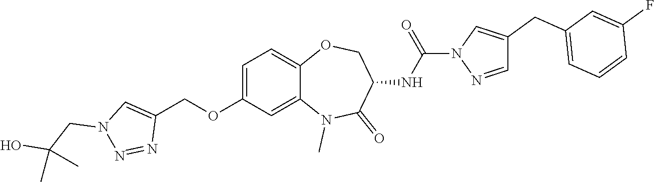 [SMILES: CN1C(=O)[C@@H](NC(=O)n2cc(Cc3cccc(F)c3)cn2)COc2ccc(OCc3cn(CC(C)(C)O)nn3)cc21]

### Step A: tert-butyl (S)-(5-methyl-4-oxo-7-(prop-2-yn-1-yloxy)-2,3,4,5-tetrahydrobenzo[b][1,4]oxazepin-3-yl)carbamate

A mixture of tert-butyl (S)-(7-hydroxy-5-methyl-4-oxo-2,3,4,5-tetrahydrobenzo[b][1,4]oxazepin-3-yl)carbamate (Intermediate 2, 250 mg, 0.811 mmol), propargyl bromide (73.0 μL, 0.973 mmol) and Cs₂CO₃ (528 mg, 1.62 mmol) in CH₃CN (8.0 mL) was stirred at room temperature for 2 hours. After quenched with water, the mixture was extracted with DCM twice. The combined organic layers were washed with water and brine, dried over Na₂SO₄, filtered, and concentrated in vacuo. The residue was purified by column chromatography on SiO₂ (Hexanes:EtOAc=2:1) to afford tert-butyl (S)-(5-methyl-4-oxo-7-(prop-2-yn-1-yloxy)-2,3,4,5-tetrahydrobenzo[b][1,4]oxazepin-3-yl)carbamate (230 mg, 80%) as a white foam. ¹H-NMR (400 MHz, CDCl₃): δ 7.08 (1H, dd, J=7.2, 2.0 Hz), 6.81 (1H, s), 6.80 (1H, t, J=4.2 Hz), 5.48 (1H, d, J=7.6 Hz), 4.68 (2H, d, J=2.0 Hz), 4.67-4.62 (1H, m), 4.54 (1H, dd, J=9.6, 7.6 Hz, 1H), 4.11 (1H, dd, J=11.2, 9.6 Hz), 3.38 (3H, s), 2.55 (1H, t, J=2.6 Hz), 1.40 (9H, s).

### Step B: tert-butyl (S)-(7-((1-(2-hydroxy-2-methylpropyl)-1H-1,2,3-triazol-4-yl)methoxy)-5-methyl-4-oxo-2,3,4,5-tetrahydrobenzo[b][1,4]oxazepin-3-yl)carbamate

A mixture of tert-butyl (S)-(5-methyl-4-oxo-7-(prop-2-yn-1-yloxy)-2,3,4,5-tetrahydrobenzo[b][1,4]oxazepin-3-yl)carbamate (200 mg, 0.577 mmol), 1-azido-2-methylpropan-2-ol (80.0 mg, 0.693 mmol), CuI (2.20 mg, 0.012 mmol) and DIPEA (0.100 mL, 0.577 mmol) in DCM (6.0 mL) was stirred at room temperature for 1 hour. After quenched with water, the mixture was extracted with DCM twice. The combined organic layers were washed with water and brine, dried over Na₂SO₄, filtered, and concentrated in vacuo. The residue was purified by column chromatography on SiO₂ (DCM:MeOH=20:1) to afford tert-butyl (S)-(7-((1-(2-hydroxy-2-methylpropyl)-1H-1,2,3-triazol-4-yl)methoxy)-5-methyl-4-oxo-2,3,4,5-tetrahydrobenzo[b][1,4]oxazepin-3-yl)carbamate (130 mg, 49%) as a white foam. ¹H-NMR (400 MHz, CDCl₃): δ 7.64 (1H, s), 7.02 (1H, d, J=8.0 Hz), 6.80-6.78 (2H, m), 5.47 (1H, d, J=8.0 Hz), 5.28 (2H, d, J=4.4 Hz), 4.59-4.53 (1H, m), 4.45 (1H, dd, J=9.6, 8.0 Hz), 4.30 (2H, s), 4.08 (1H, dd, J=11.4, 9.8 Hz), 3.36 (3H, s), 1.40 (9H, s), 1.20 (3H, s), 1.14 (3H, s).

### Step C: (S)-3-amino-7-((1-(2-hydroxy-2-methylpropyl)-1H-1,2,3-triazol-4-yl)methoxy)-5-methyl-2,3-dihydrobenzo[b][1,4]oxazepin-4(5H)-one hydrochloride

To a solution of tert-butyl (S)-(7-((1-(2-hydroxy-2-methylpropyl)-1H-1,2,3-triazol-4-yl)methoxy)-5-methyl-4-oxo-2,3,4,5-tetrahydrobenzo[b][1,4]oxazepin-3-yl)carbamate (130 mg, 0.82 mmol) in DCM (2.8 mL) was added HCl (4 M solution in dioxane, 0.700 mL, 2.82 mmol) at 0° C. The reaction mixture was stirred at room temperature for 18 hours and concentrated in vacuo to give (S)-3-amino-7-((1-(2-hydroxy-2-methylpropyl)-1H-1,2,3-triazol-4-yl)methoxy)-5-methyl-2,3-dihydrobenzo[b][1,4]oxazepin-4(5H)-one hydrochloride (90.0 mg, 80%) as a white foam. ¹H-NMR (400 MHz, DMSO-d₆): δ 8.48 (3H, s), 8.11 (1H, s), 7.21 (2H, dd, J=12.2, 5.8 Hz), 6.99 (1H, dd, J=9.0, 2.6 Hz), 5.18 (2H, d, J=2.0 Hz), 4.52 (1H, t, J=8.6 Hz), 4.37 (1H, t, J=10.4 Hz), 4.29 (2H, s), 4.27-4.20 (1H, m), 3.35 (3H, s), 1.07 (6H, s).

### Step D: (S)-4-(3-fluorobenzyl)-N-(7-((1-(2-hydroxy-2-methylpropyl)-1H-1,2,3-triazol-4-yl)methoxy)-5-methyl-4-oxo-2,3,4,5-tetrahydrobenzo[b][1,4]oxazepin-3-yl)-1H-pyrazole-1-carboxamide

To a solution of (S)-3-amino-7-((1-(2-hydroxy-2-methylpropyl)-1H-1,2,3-triazol-4-yl)methoxy)-5-methyl-2,3-dihydrobenzo[b][1,4]oxazepin-4(5H)-one hydrochloride (90.0 mg, 0.226 mmol) in DCE (2.0 mL) was added CDI (55.0 mg, 0.339 mmol) followed by TEA (79.0 μL, 0.566 mmol) at 0° C. The mixture was stirred at 0° C. for 2 hours. After quenched with water, the mixture was extracted with DCM twice. The combined organic layers were washed with water and brine, dried over Na₂SO₄, filtered, and concentrated in vacuo.

To a solution of the residue in DCE (2.0 mL) was added 4-(3-fluorobenzyl)-1H-pyrazole hydrochloride (Intermediate 6, 52.9 mg, 0.249 mmol) and TEA (79.0 μL, 0.566 mmol) at 0° C. The reaction mixture was stirred at room temperature for 18 hours. After quenched with water, the mixture was extracted with DCM twice. The combined organic layers were washed with water and brine, dried over Na₂SO₄, filtered, and concentrated in vacuo. The residue was purified by column chromatography on SiO₂ (Hexanes:EtOAc=1:2) to give (S)-4-(3-fluorobenzyl)-N-(7-((1-(2-hydroxy-2-methylpropyl)-1H-1,2,3-triazol-4-yl)methoxy)-5-methyl-4-oxo-2,3,4,5-tetrahydrobenzo[b][1,4]oxazepin-3-yl)-1H-pyrazole-1-carboxamide (45.0 mg, 35%) as a white foam. ¹H-NMR (400 MHz, CDCl₃): δ 7.98 (1H, d, J=7.2 Hz), 7.87 (1H, d, J=0.8 Hz), 7.72 (1H, s), 7.47 (1H, s), 7.25-7.22 (1H, m), 7.10 (1H, q, J=3.1 Hz), 7.00-6.84 (5H, m), 5.26 (2H, s), 4.89-4.83 (1H, m), 4.62 (1H, dd, J=9.8, 7.6 Hz), 4.33 (2H, s), 4.24 (1H, dd, J=11.0, 9.8 Hz), 3.81 (2H, s), 3.40 (3H, s), 1.21 (6H, d, J=3.2 Hz). LC-MS: m/z=564.20 [M+H]⁺.

### General Synthetic Scheme for Hydroxy Ether Analogues.

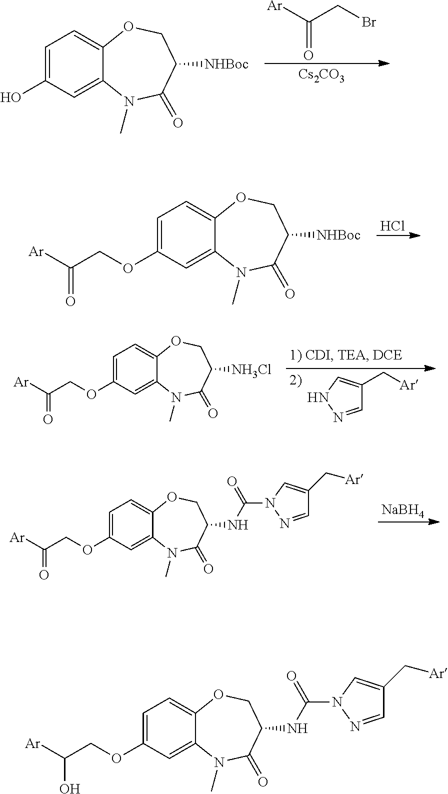

### Example 69: 4-(3-fluorobenzyl)-N-((3S)-7-(2-hydroxy-2-(pyridin-2-yl)ethoxy)-5-methyl-4-oxo-2,3,4,5-tetrahydrobenzo[b][1,4]oxazepin-3-yl)-1H-pyrazole-1-carboxamide

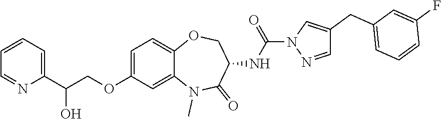 [SMILES: CN1C(=O)[C@@H](NC(=O)n2cc(Cc3cccc(F)c3)cn2)COc2ccc(OCC(O)c3ccccn3)cc21]

### Step A: tert-butyl (S)-(5-methyl-4-oxo-7-(2-oxo-2-(pyridin-2-yl)ethoxy)-2,3,4,5-tetrahydrobenzo[b][1,4]oxazepin-3-yl)carbamate

To a solution of tert-butyl (S)-(7-hydroxy-5-methyl-4-oxo-2,3,4,5-tetrahydrobenzo[b][1,4]oxazepin-3-yl)carbamate (Intermediate 2, 200 mg, 0.649 mmol) and 2-bromo-1-(pyridin-2-yl)ethan-1-one hydrobromide (273 mg, 0.973 mmol) in MeCN (2.2 mL) was added Cs₂CO₃ (0.528 mg, 1.62 mmol) at room temperature. The reaction mixture was stirred at 60° C. for 3 hours and cooled to room temperature. After quenched with water, the mixture was extracted with EtOAc twice. The combined organic layers were washed with brine, dried over Na₂SO₄, filtered, and concentrated in vacuo. The residue was purified by column chromatography on SiO₂ (Hexanes:EtOAc=2:1 to 1:1) to afford tert-butyl (S)-(5-methyl-4-oxo-7-(2-oxo-2-(pyridin-2-yl)ethoxy)-2,3,4,5-tetrahydrobenzo[b][1,4]oxazepin-3-yl)carbamate (242 mg, 87%) as a white solid. ¹H-NMR (400 MHz, CDCl₃): δ 7.03 (1H, d, J=8.8 Hz), 6.75-6.70 (2H, m), 5.46 (1H, d, J=7.2 Hz), 4.67-4.60 (1H, m), 4.51 (1H, dd, J=9.6, 7.2 Hz), 4.10-4.00 (3H, m), 3.36 (3H, s), 2.77-2.67 (2H, m), 2.37 (6H, s), 1.38 (9H, s).

### Step B: (S)-3-amino-5-methyl-7-(2-oxo-2-(pyridin-2-yl)ethoxy)-2,3-dihydrobenzo[b][1,4]oxazepin-4(5H)-one dihydrochloride

To a solution of tert-butyl (S)-(5-methyl-4-oxo-7-(2-oxo-2-(pyridin-2-yl)ethoxy)-2,3,4,5-tetrahydrobenzo[b][1,4]oxazepin-3-yl)carbamate (242 mg, 0.566 mmol) in DCM (2.8 mL) was added HCl (4 M in dioxane, 0.708 mL, 2.83 mmol) at 0° C. The reaction mixture was stirred at room temperature overnight and concentrated in vacuo to afford (S)-3-amino-5-methyl-7-(2-oxo-2-(pyridin-2-yl)ethoxy)-2,3-dihydrobenzo[b][1,4]oxazepin-4(5H)-one dihydrochloride as a yellow solid, which was used for next step without further purification. LC-MS: m/z=280.1 [M+H]⁺.

### Step C: (S)-4-(3-fluorobenzyl)-N-(5-methyl-4-oxo-7-(2-oxo-2-(pyridin-2-yl)ethoxy)-2,3,4,5-tetrahydrobenzo[b][1,4]oxazepin-3-yl)-1H-pyrazole-1-carboxamide

To a solution of (S)-3-amino-5-methyl-7-(2-oxo-2-(pyridin-2-yl)ethoxy)-2,3-dihydrobenzo[b][1,4]oxazepin-4(5H)-one dihydrochloride (230 mg, 0.575 mmol) in DCE (2.9 mL) was added CDI (107 mg, 0.661 mmol) followed by TEA (0.200 mL, 1.44 mmol) at 0° C. The mixture was stirred at 0° C. for 1 hour. After quenched with water, the mixture was extracted with DCM, washed with brine, dried over Na₂SO₄, filtered, and concentrated in vacuo.

To a solution of the residue in DCE (2.9 mL) was added 4-(3-fluorobenzyl)-1H-pyrazole hydrochloride (Intermediate 6, 134 mg, 0.632 mmol) followed by TEA (0.200 mL, 1.44 mmol) at 0° C. The reaction mixture was stirred at 40° C. for 2 hours and cooled to room temperature. After quenched with water, the mixture was extracted with DCM twice. The combined organic layers were washed with brine, dried over Na₂SO₄, filtered, and concentrated in vacuo. The residue was purified by column chromatography on SiO₂ (Hexanes:EtOAc=4:1 to 2:1) to afford (S)-4-(3-fluorobenzyl)-N-(5-methyl-4-oxo-7-(2-oxo-2-(pyridin-2-yl)ethoxy)-2,3,4,5-tetrahydrobenzo[b][1,4]oxazepin-3-yl)-1H-pyrazole-1-carboxamide (215 mg, 71% for 2 steps) as a yellow solid. ¹H-NMR (400 MHz, CDCl₃): δ7.97 (1H, d, J=7.2 Hz), 7.86 (1H, s), 7.45 (1H, s), 7.29-7.21 (1H, m), 7.09 (1H, d, J=8.8 Hz), 6.98-8.84 (3H, m), 6.78-6.74 (2H, m), 4.92-4.85 (1H, m), 4.65 (1H, dd, J=9.6, 7.6 Hz), 4.23 (1H, dd, J=10.8, 10.0 Hz), 4.05 (2H, t, J=5.8 Hz), 3.80 (2H, s), 3.39 (3H, s), 2.79-2.69 (2H, m), 2.35 (6H, s). LC-MS: m/z=482.1 [M+H]⁺.

### Step D: 4-(3-fluorobenzyl)-N-((3S)-7-(2-hydroxy-2-(pyridin-2-yl)ethoxy)-5-methyl-4-oxo-2,3,4,5-tetrahydrobenzo[b][1,4]oxazepin-3-yl)-1H-pyrazole-1-carboxamide

To a solution of (S)-4-(3-fluorobenzyl)-N-(5-methyl-4-oxo-7-(2-oxo-2-(pyridin-2-yl)ethoxy)-2,3,4,5-tetrahydrobenzo[b][1,4]oxazepin-3-yl)-1H-pyrazole-1-carboxamide (16.0 mg, 0.0300 mmol) in MeOH (0.15 mL) was added NaBH₄ (1.14 mg, 0.0300 mmol) at 0° C. The reaction mixture was stirred at 0° C. for 30 min. After quenched with water, the mixture was extracted with EtOAc twice. The combined organic layers were washed with water and brine, dried over Na₂SO₄, filtered, and concentrated in vacuo. The residue was purified by column chromatography on SiO₂(Hexanes:EtOAc=1:1 to 1:3) to afford 4-(3-fluorobenzyl)-N-((3S)-7-(2-hydroxy-2-(pyridin-4-yl)ethoxy)-5-methyl-4-oxo-2,3,4,5-tetrahydrobenzo[b][1,4]oxazepin-2-yl)-1H-pyrazole-1-carboxamide (16 mg, 100%) as a white foam. ¹H-NMR (400 MHz, CDCl₃): δ 8.60 (1H, d, J=4.4 Hz), 7.97 (1H, d, J=6.8 Hz), 7.87 (1H, s), 7.77-7.73 (1H, m), 7.52-7.46 (2H, m), 7.29-7.22 (2H, m), 7.09 (1H, dd, J=8.8, 2.4 Hz), 7.00-6.85 (3H, m), 6.80-6.76 (2H, m), 5.13 (1H, t, J=4.2 Hz), 4.91-4.84 (1H, m), 4.65 (1H, dd, J=7.2, 9.6 Hz), 4.26-4.19 (3H, m), 3.801 (2H, s), 3.41 (3H, d, J=2.4 Hz). LC-MS: m/z=532.10 [M+H]⁺.

### Example 70: 4-(3-fluorobenzyl)-N-((3S)-7-(2-hydroxy-2-(pyridin-3-yl)ethoxy)-5-methyl-4-oxo-2,3,4,5-tetrahydrobenzo[b][1,4]oxazepin-3-yl)-1H-pyrazole-1-carboxamide

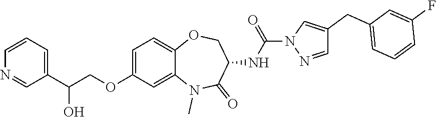 [SMILES: CN1C(=O)[C@@H](NC(=O)n2cc(Cc3cccc(F)c3)cn2)COc2ccc(OCC(O)c3cccnc3)cc21]

### Step A: tert-butyl (S)-(5-methyl-4-oxo-7-(2-oxo-2-(pyridin-3-yl)ethoxy)-2,3,4,5-tetrahydrobenzo[b][1,4]oxazepin-3-yl)carbamate

To a solution of tert-butyl (S)-(7-hydroxy-5-methyl-4-oxo-2,3,4,5-tetrahydrobenzo[b][1,4]oxazepin-3-yl)carbamate (Intermediate 2, 200 mg, 0.649 mmol) and Cs₂CO₃ (0.528 mg, 1.62 mmol) in MeCN (6.5 mL) was portionwise added 2-bromo-1-(pyridin-3-yl)ethan-1-one hydrobromide (364 mg, 1.30 mmol) at room temperature. The reaction mixture was stirred at room temperature for 30 min. After quenched with water, the mixture was extracted with EtOAc twice. The combined organic layers were washed with brine, dried over Na₂SO₄, filtered, and concentrated in vacuo. The residue was purified by column chromatography on SiO₂ (Hexanes:EtOAc=2:1 to 1:1) to afford tert-butyl (S)-(5-methyl-4-oxo-7-(2-oxo-2-(pyridin-3-yl)ethoxy)-2,3,4,5-tetrahydrobenzo[b][1,4]oxazepin-3-yl)carbamate (125 mg, 45%) as a white solid. ¹H-NMR (400 MHz, CDCl₃): δ 9.22 (1H, dd, J=2.8, 1.2 Hz), 8.83 (1H, dd, J=4.8, 1.6 Hz), 8.28 (1H, dt, J=8.0, 2.0 Hz), 7.49-7.46 (1H, m), 7.05 (1H, d, J=8.8 Hz), 6.81 (1H, d, J=3.2 Hz), 6.72 (1H, dd, J=8.4, 3.2 Hz), 5.46 (1H, d, J=6.8 Hz), 5.21 (2H, s), 4.66-4.59 (1H, m), 4.51 (1H, dd, J=9.6, 3.2 Hz), 4.13-4.06 (1H, m), 3.36 (3H, s), 1.38 (9H, s).

### Step B: (S)-3-amino-5-methyl-7-(2-oxo-2-(pyridin-3-yl)ethoxy)-2,3-dihydrobenzo[b][1,4]oxazepin-4(5H)-one dihydrochloride

The title compound was prepared in a similar fashion to Example 69 (Step B) with tert-butyl (S)-(5-methyl-4-oxo-7-(2-oxo-2-(pyridin-3-yl)ethoxy)-2,3,4,5-tetrahydrobenzo[b][1,4]oxazepin-3-yl)carbamate. After concentration in vacuo, the crude product was used for the next reaction without purification. LC-MS: m/z=328.1 [M+H]⁺.

### Step C: (S)-4-(3-fluorobenzyl)-N-(5-methyl-4-oxo-7-(2-oxo-2-(pyridin-3-yl)ethoxy)-2,3,4,5-tetrahydrobenzo[b][1,4]oxazepin-3-yl)-1H-pyrazole-1-carboxamide (1300-164)

The title compound was prepared in a similar fashion to Example 69 (Step C) with (S)-3-amino-5-methyl-7-(2-oxo-2-(pyridin-3-yl)ethoxy)-2,3-dihydrobenzo[b][1,4]oxazepin-4(5H)-one dihydrochloride and 4-(3-fluorobenzyl)-1H-pyrazole hydrochloride (Intermediate 6). The crude product was purified by column chromatography on SiO₂ (Hexanes:EtOAc=1:4) to give (S)-4-(3-fluorobenzyl)-N-(5-methyl-4-oxo-7-(2-oxo-2-(pyridin-3-yl)ethoxy)-2,3,4,5-tetrahydrobenzo[b][1,4]oxazepin-3-yl)-1H-pyrazole-1-carboxamide (77% for 2 steps) as a white foam. ¹H-NMR (400 MHz, CDCl₃): δ 9.24 (1H, dd, J=2.0, 1.2 Hz), 8.85 (1H, dd, J=4.4, 2.0 Hz), 8.30 (1H, dt, J=8.4, 1.6 Hz), 7.98 (1H, d, J=7.2 Hz), 7.87 (1H, s), 7.49 (1H, td, J=8.4, 0.8 Hz), 7.47 (1H, s), 7.27-7.22 (1H, m), 7.12 (1H, d, J=7.2 Hz), 6.96-6.90 (2H, m), 6.89-6.84 (2H, m), 6.76 (1H, dd, J=8.4, 3.2 Hz), 5.25 (2H, s), 4.92-4.86 (1H, m), 4.66 (1H, dd, J=9.6, 7.2 Hz), 4.25 (1H, dd, J=10.8, 9.6 Hz), 3.81 (2H, s), 3.41 (3H, s). LC-MS: m/z=530.1 [M+H]⁺.

### Step D: 4-(3-fluorobenzyl)-N-((3S)-7-(2-hydroxy-2-(pyridin-3-yl)ethoxy)-5-methyl-4-oxo-2,3,4,5-tetrahydrobenzo[b][1,4]oxazepin-3-yl)-1H-pyrazole-1-carboxamide

The title compound was prepared in a similar fashion to Example 69 (Step D) with (S)-4-(3-fluorobenzyl)-N-(5-methyl-4-oxo-7-(2-oxo-2-(pyridin-3-yl)ethoxy)-2,3,4,5-tetrahydrobenzo[b][1,4]oxazepin-3-yl)-1H-pyrazole-1-carboxamide. The crude product was purified by column chromatography on SiO₂ (EtOAc only) to afford 4-(3-fluorobenzyl)-N-((3S)-7-(2-hydroxy-2-(pyridin-3-yl)ethoxy)-5-methyl-4-oxo-2,3,4,5-tetrahydrobenzo[b][1,4]oxazepin-3-yl)-1H-pyrazole-1-carboxamide (80%) as a white foam. ¹H-NMR (400 MHz, CDCl₃): δ 8.71 (1H, d, J=2.4 Hz), 8.61 (1H, dd, J=4.4, 0.8 Hz), 7.97 (1H, d, J=7.6 Hz), 7.88 (1H, d, J=1.2 Hz), 7.83 (1H, dt, J=8.0, 2.0 Hz), 7.47 (1H, s), 7.36 (1H, dd, J=8.0, 4.8 Hz), 7.28-7.22 (1H, m), 7.13-7.11 (1H, m), 6.96-6.85 (3H, m), 6.79-6.75 (2H, m), 5.20 (1H, dd, J=8.4, 3.2 Hz), 4.90 (1H, dt, J=10.8, 7.6 Hz), 4.66 (1H, dd, J=10.0, 8.0 Hz), 4.25 (1H, dd, J=10.8, 10.0 Hz), 4.15-4.11 (2H, m), 3.81 (2H, s), 3.41 (3H, s). LC-MS: m/z=532.1 [M+H]⁺.

### Example 71: 4-(3-fluorobenzyl)-N-((3S)-7-(2-hydroxy-2-(pyridin-4-yl)ethoxy)-5-methyl-4-oxo-2,3,4,5-tetrahydrobenzo[b][1,4]oxazepin-3-yl)-1H-pyrazole-1-carboxamide

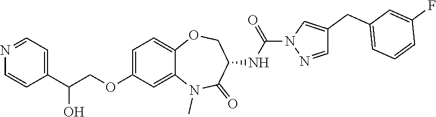 [SMILES: CN1C(=O)[C@@H](NC(=O)n2cc(Cc3cccc(F)c3)cn2)COc2ccc(OCC(O)c3ccncc3)cc21]

### Step A: tert-butyl (S)-(5-methyl-4-oxo-7-(2-oxo-2-(pyridin-4-yl)ethoxy)-2,3,4,5-tetrahydrobenzo[b][1,4]oxazepin-3-yl)carbamate (4)

The title compound was prepared in a similar fashion to Example 70 (Step A) with Intermediate 2 and 2-bromo-1-(pyridin-4-yl)ethan-1-one hydrobromide. The crude product was purified by column chromatography on SiO₂ (Hexanes:EtOAc=1:1 to 1:3) to afford tert-butyl (S)-(5-methyl-4-oxo-7-(2-oxo-2-(pyridin-4-yl)ethoxy)-2,3,4,5-tetrahydrobenzo[b][1,4]oxazepin-3-yl)carbamate (40%) as a white solid. ¹H-NMR (400 MHz, CDCl₃): δ 8.87 (2H, dd, J=4.4, 1.6 Hz), 7.77 (2H, dd, J=4.4, 1.6 Hz), 7.06 (1H, d, J=8.8 Hz), 6.82 (1H, d, J=2.8 Hz), 6.70 (1H, dd, J=8.8, 2.8 Hz), 5.47 (1H, d, J=7.2 Hz), 5.21 (2H, s), 4.67-4.60 (1H, m), 4.51 (1H, dd, J=9.6, 7.2 Hz), 4.10-4.00 (1H, m), 3.37 (3H, s), 1.39 (9H, s).

### Step B: (S)-3-amino-5-methyl-7-(2-oxo-2-(pyridin-4-yl)ethoxy)-2,3-dihydrobenzo[b][1,4]oxazepin-4(5H)-one dihydrochloride

The title compound was prepared in a similar fashion to Example 69 (Step B) with tert-butyl (S)-(5-methyl-4-oxo-7-(2-oxo-2-(pyridin-4-yl)ethoxy)-2,3,4,5-tetrahydrobenzo[b][1,4]oxazepin-3-yl)carbamate. After concentration in vacuo, the crude product was used for the next reaction without purification. LC-MS: m/z=328.0 [M+H]⁺.

### Step C: (S)-4-(3-fluorobenzyl)-N-(5-methyl-4-oxo-7-(2-oxo-2-(pyridin-4-yl)ethoxy)-2,3,4,5-tetrahydrobenzo[b][1,4]oxazepin-3-yl)-1H-pyrazole-1-carboxamide

The title compound was prepared in a similar fashion to Example 69 (Step C) with (S)-3-amino-5-methyl-7-(2-oxo-2-(pyridin-4-yl)ethoxy)-2,3-dihydrobenzo[b][1,4]oxazepin-4(5H)-one dihydrochloride and 4-(3-fluorobenzyl)-1H-pyrazole hydrochloride (Intermediate 6). The crude product was purified by column chromatography on SiO₂ (Hexanes:EtOAc=1:1) to give methyl (S)-2-(((3-(4-(3-fluorobenzyl)-1H-pyrazole-1-carboxamido)-5-methyl-4-oxo-2,3,4,5-tetrahydrobenzo[b][1,4]oxazepin-7-yl)oxy)methyl)thiazole-4-carboxylate (70% for 2 steps) as a white foam. ¹H-NMR (400 MHz, CDCl₃): δ 8.87 (2H, dd, J=4.4, 1.6 Hz), 7.96 (1H, d, J=7.2 Hz), 7.86 (1H, d, J=0.8 Hz), 7.77 (2H, dd, J=4.4, 0.8 Hz), 7.46 (1H, s), 7.26-7.21 (1H, m), 7.12 (1H, d, J=8.8 Hz), 6.98-6.84 (4H, m), 6.75 (1H, dd, J=8.8, 3.2 Hz), 5.24 (2H, s), 4.92-4.85 (1H, m), 4.65 (1H, dd, J=9.6, 7.6 Hz), 4.25 (1H, dd, J=10.0, 9.6 Hz), 3.80 (2H, s), 3.39 (3H, s). LC-MS: m/z=530.1 [M+H]⁺.

### Step D: 4-(3-fluorobenzyl)-N-((3S)-7-(2-hydroxy-2-(pyridin-4-yl)ethoxy)-5-methyl-4-oxo-2,3,4,5-tetrahydrobenzo[b][1,4]oxazepin-3-yl)-1H-pyrazole-1-carboxamide

The title compound was prepared in a similar fashion to Example 69 (Step D) with (S)-4-(3-fluorobenzyl)-N-(5-methyl-4-oxo-7-(2-oxo-2-(pyridin-4-yl)ethoxy)-2,3,4,5-tetrahydrobenzo[b][1,4]oxazepin-3-yl)-1H-pyrazole-1-carboxamide. The crude product was purified by column chromatography on SiO₂ (Hexanes:EtOAc=1:1 to 1:3) to afford 4-(3-fluorobenzyl)-N-((3S)-7-(2-hydroxy-2-(pyridin-4-yl)ethoxy)-5-methyl-4-oxo-2,3,4,5-tetrahydrobenzo[b][1,4]oxazepin-3-yl)-1H-pyrazole-1-carboxamide (100%) as a white foam. ¹H-NMR (400 MHz, CDCl₃): δ 8.65 (2H, d, J=5.6 Hz), 7.97 (1H, d, J=7.6 Hz), 7.87 (1H, d, J=1.2 Hz), 7.47 (1H, s), 7.40-7.41 (2H, m), 7.28-7.22 (1H, m), 7.13-7.11 (1H, m), 6.96-6.85 (3H, m), 6.79-6.74 (2H, m), 5.15 (1H, dd, J=8.0, 3.2 Hz), 4.92-4.86 (1H, m) 4.65 (1H, dd, J=9.6, 7.2 Hz), 4.25 (1H, dd, J=11.2, 9.6 Hz), 4.16-4.09 (1H, m), 4.01 (1H, dd, J=8.4, 9.6 Hz), 3.80 (2H, s), 3.41 (3H, s). LC-MS: m/z=532.10 [M+H]⁺.

### Example 72: (S)-4-(2-fluorobenzyl)-N-(7-((4-hydroxy-4-methylpentyl)oxy)-5-methyl-4-oxo-2,3,4,5-tetrahydrobenzo[b][1,4]oxazepin-3-yl)-1H-pyrazole-1-carboxamide

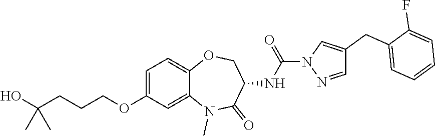 [SMILES: CN1C(=O)[C@@H](NC(=O)n2cc(Cc3ccccc3F)cn2)COc2ccc(OCCCC(C)(C)O)cc21]

### Step A: (S)-4-(2-fluorobenzyl)-N-(7-((4-hydroxy-4-methylpent-2-yn-1-yl)oxy)-5-methyl-4-oxo-2,3,4,5-tetrahydrobenzo[b][1,4]oxazepin-3-yl)-1H-pyrazole-1-carboxamide

To a solution of (S)-3-amino-7-((4-hydroxy-4-methylpent-2-yn-1-yl)oxy)-5-methyl-2,3-dihydrobenzo[b][1,4]oxazepin-4(5H)-one hydrochloride (Step B in Example 22, 50.0 mg, 0.147 mmol) in DCE (1.5 mL) was added CDI (36.0 mg, 0.240 mmol) followed by TEA (0.0500 mL, 0.367 mmol) at 0° C. The mixture was stirred at room temperature for 1 hour. After quenched with water, the mixture was extracted with DCM, washed with water and brine, dried over Na₂SO₄, filtered, and concentrated in vacuo.

To a solution of the residue in DCE (1.5 mL) was added 4-(2-fluorobenzyl)-1H-pyrazole hydrochloride (Intermediate 5, 34.0 mg, 0.161 mmol) followed by TEA (0.0500 mL, 0.367 mmol) at 0° C. The reaction mixture was stirred at room temperature for 2.5 hours and cooled to 0° C. After quenched with water, the mixture was extracted with DCM twice. The combined organic layers were washed with brine, dried over Na₂SO₄, filtered, and concentrated in vacuo. The residue was purified by column chromatography on SiO₂ (DCM:EtOAc=10:1) to afford (S)-4-(2-fluorobenzyl)-N-(7-((4-hydroxy-4-methylpent-2-yn-1-yl)oxy)-5-methyl-4-oxo-2,3,4,5-tetrahydrobenzo[b][1,4]oxazepin-3-yl)-1H-pyrazole-1-carboxamide (18.0 mg, 24%) as a white foam. ¹H-NMR (400 MHz, CDCl₃): δ 7.97 (1H, d, J=7.3 Hz), 7.89 (1H, s), 7.51 (1H, s), 7.24-7.11 (3H, m), 7.08-7.00 (2H, m), 6.83 (2H, dd, J=7.1, 2.5 Hz), 4.93-4.87 (1H, m), 4.71 (2H, d, J=2.3 Hz), 4.68-4.64 (1H, m), 4.25 (1H, dd, J=11.2, 9.8 Hz), 3.83 (2H, s), 3.42 (3H, s), 1.53 (6H, s).

### Step B: (S)-4-(3-fluorobenzyl)-N-(7-((4-hydroxy-4-methylpentyl)oxy)-5-methyl-4-oxo-2,3,4,5-tetrahydrobenzo[b][1,4]oxazepin-3-yl)-1H-pyrazole-1-carboxamide

A suspension of (S)-4-(2-fluorobenzyl)-N-(7-((4-hydroxy-4-methylpent-2-yn-1-yl)oxy)-5-methyl-4-oxo-2,3,4,5-tetrahydrobenzo[b][1,4]oxazepin-3-yl)-1H-pyrazole-1-carboxamide (15.0 mg, 0.0300 mmol) and Pd/C (10 wt %, 0.400 mg, 2.96 μmol) in EtOAc (0.30 mL) was stirred at room temperature for 1 hour under H₂ atmosphere (1 atm). The reaction mixture was filtered through a Celite pad and washed with EtOAc. The filtrate was concentrated in vacuo to afford (S)-4-(3-fluorobenzyl)-N-(7-((4-hydroxy-4-methylpentyl)oxy)-5-methyl-4-oxo-2,3,4,5-tetrahydrobenzo[b][1,4]oxazepin-3-yl)-1H-pyrazole-1-carboxamide (15.0 mg, 99%) as a white foam. ¹H-NMR (400 MHz, CDCl₃): δ 7.97 (1H, d, J=7.3 Hz), 7.89 (1H, s), 7.50 (1H, s), 7.24-7.13 (2H, m), 7.11-7.01 (3H, m), 6.75 (2H, dd, J=7.1, 2.5 Hz), 4.92-4.86 (1H, m), 4.65 (1H, dd, J=9.8, 7.5 Hz), 4.23 (1H, dd, J=11.2, 9.8 Hz), 3.98 (2H, t, J=6.2 Hz), 3.83 (2H, s), 3.41 (3H, s), 1.94-1.87 (2H, m), 1.68-1.64 (2H, m), 1.28 (6H, s). LC-MS: m/z=511.10 [M+H]⁺.

### Example 73: (S)-4-(4-fluorobenzyl)-N-(7-((4-hydroxy-4-methylpentyl)oxy)-5-methyl-4-oxo-2,3,4,5-tetrahydrobenzo[b][1,4]oxazepin-3-yl)-1H-pyrazole-1-carboxamide

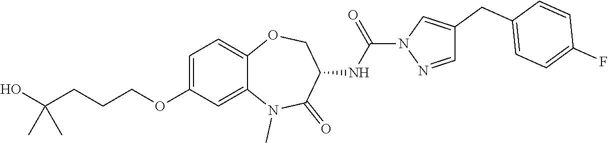 [SMILES: CN1C(=O)[C@@H](NC(=O)n2cc(Cc3ccc(F)cc3)cn2)COc2ccc(OCCCC(C)(C)O)cc21]

The title compound was prepared in a similar fashion to Example 72 with (S)-3-amino-7-((4-hydroxy-4-methylpent-2-yn-1-yl)oxy)-5-methyl-2,3-dihydrobenzo[b][1,4]oxazepin-4(5H)-one hydrochloride (Step B in Example 22) and 4-(4-fluorobenzyl)-1H-pyrazole hydrochloride (Intermediate 7) in 2 steps (8.5%). ¹H-NMR (400 MHz, CDCl₃): δ 7.97 (1H, d, J=7.3 Hz), 7.85 (1H, s), 7.38 (1H, s), 7.18-7.09 (3H, m), 7.00-6.98 (2H, m), 6.76-6.73 (2H, m), 4.93-4.86 (1H, m), 4.65 (1H, dd, J=9.6, 7.3 Hz), 4.24 (1H, dd, J=11.2, 9.8 Hz), 3.98 (2H, t, J=6.4 Hz), 3.84 (2H, s), 3.41 (3H, s), 1.94-1.87 (2H, m), 1.68-1.64 (2H, m), 1.28 (6H, s). LC-MS: m/z=511.20 [M+H]⁺.

### Example 74: (S)-4-(2,3-difluorobenzyl)-N-(7-((4-hydroxy-4-methylpentyl)oxy)-5-methyl-4-oxo-2,3,4,5-tetrahydrobenzo[b][1,4]oxazepin-3-yl)-1H-pyrazole-1-carboxamide

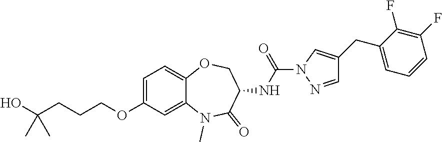 [SMILES: CN1C(=O)[C@@H](NC(=O)n2cc(Cc3cccc(F)c3F)cn2)COc2ccc(OCCCC(C)(C)O)cc21]

The title compound was prepared in a similar fashion to Example 72 with (S)-3-amino-7-((4-hydroxy-4-methylpent-2-yn-1-yl)oxy)-5-methyl-2,3-dihydrobenzo[b][1,4]oxazepin-4(5H)-one hydrochloride (Step B in Example 22) and 4-(2,3-difluorobenzyl)-1H-pyrazole hydrochloride (Intermediate 8) in 2 steps (21%). ¹H-NMR (400 MHz, CDCl₃): δ 7.97 (1H, d, J=7.2 Hz), 7.90 (1H, s), 7.50 (1H, s), 7.11-6.96 (3H, m), 6.91-6.88 (1H, m), 6.76-6.74 (2H, m), 4.89 (1H, dt, J=10.9, 7.5 Hz), 4.65 (1H, dd, J=9.6, 7.6 Hz), 4.23 (1H, dd, J=11.4, 9.8 Hz), 3.98 (2H, t, J=6.4 Hz), 3.85 (2H, s), 3.41 (3H, s), 1.94-1.86 (2H, m), 1.68-1.63 (2H, m), 1.28 (6H, s). LC-MS: m/z=529.2 [M+H]⁺.

### Example 75: (S)-4-(3,4-difluorobenzyl)-N-(7-((4-hydroxy-4-methylpentyl)oxy)-5-methyl-4-oxo-2,3,4,5-tetrahydrobenzo[b][1,4]oxazepin-3-yl)-1H-pyrazole-1-carboxamide

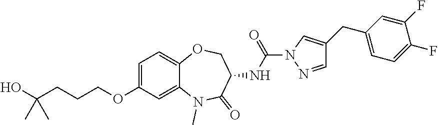 [SMILES: CN1C(=O)[C@@H](NC(=O)n2cc(Cc3ccc(F)c(F)c3)cn2)COc2ccc(OCCCC(C)(C)O)cc21]

The title compound was prepared in a similar fashion to Example 72 with (S)-3-amino-7-((4-hydroxy-4-methylpent-2-yn-1-yl)oxy)-5-methyl-2,3-dihydrobenzo[b][1,4]oxazepin-4(5H)-one hydrochloride (Step B in Example 22) and 4-(3,4-difluorobenzyl)-1H-pyrazole hydrochloride (Intermediate 9) in 2 steps (8%). ¹H-NMR (400 MHz, CDCl₃): 7.98 δ (1H, d, J=7.2 Hz), 7.87 (1H, s), 7.45 (1H, s), 7.11-7.04 (2H, m), 6.98-6.93 (1H, m), 6.89-6.86 (1H, m), 6.76-6.74 (2H, m), 4.89 (1H, dt, J=11.1, 7.5 Hz), 4.65 (1H, dd, J=9.8, 7.4 Hz), 4.24 (1H, dd, J=11.0, 10.2 Hz), 3.98 (2H, t, J=6.4 Hz), 3.77 (2H, s), 3.41 (3H, s), 1.94-1.86 (2H, m), 1.68-1.63 (2H, m), 1.28 (6H, s). LC-MS: m/z=529.2 [M+H]⁺.

### Example 76: (S)-4-(3,5-difluorobenzyl)-N-(7-((4-hydroxy-4-methylpentyl)oxy)-5-methyl-4-oxo-2,3,4,5-tetrahydrobenzo[b][1,4]oxazepin-3-yl)-1H-pyrazole-1-carboxamide

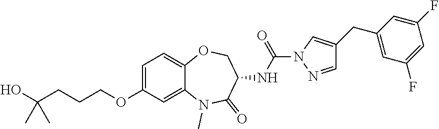 [SMILES: CN1C(=O)[C@@H](NC(=O)n2cc(Cc3cc(F)cc(F)c3)cn2)COc2ccc(OCCCC(C)(C)O)cc21]

The title compound was prepared in a similar fashion to Example 72 with (S)-3-amino-7-((4-hydroxy-4-methylpent-2-yn-1-yl)oxy)-5-methyl-2,3-dihydrobenzo[b][1,4]oxazepin-4(5H)-one hydrochloride (Step B in Example 22) and 4-(3,5-difluorobenzyl)-1H-pyrazole hydrochloride (Intermediate 10) in 2 steps (2%). ¹H-NMR (400 MHz, CDCl₃): 7.99 (1H, d, J=7.2 Hz), 7.90 (1H, s), 7.47 (1H, s), 7.12-7.09 (1H, m), 6.76-6.63 (5H, m), 4.90 (1H, dt, J=11.2, 7.5 Hz), 4.66 (1H, dd, J=9.8, 7.4 Hz), 4.25 (1H, dd, J=11.0, 10.2 Hz), 3.98 (2H, t, J=6.4 Hz), 3.79 (2H, s), 3.41 (3H, s), 1.94-1.87 (2H, m), 1.68-1.64 (2H, m), 1.28 (6H, s). LC-MS: m/z=529.2 [M+H]⁺.

### Example 77: (S)-4-(2,4-difluorobenzyl)-N-(7-((4-hydroxy-4-methylpentyl)oxy)-5-methyl-4-oxo-2,3,4,5-tetrahydrobenzo[b][1,4]oxazepin-3-yl)-1H-pyrazole-1-carboxamide

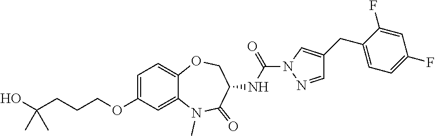 [SMILES: CN1C(=O)[C@@H](NC(=O)n2cc(Cc3ccc(F)cc3F)cn2)COc2ccc(OCCCC(C)(C)O)cc21]

The title compound was prepared in a similar fashion to Example 72 with (S)-3-amino-7-((4-hydroxy-4-methylpent-2-yn-1-yl)oxy)-5-methyl-2,3-dihydrobenzo[b][1,4]oxazepin-4(5H)-one hydrochloride (Step B in Example 22) and 4-(2,4-difluorobenzyl)-1H-pyrazole hydrochloride (Intermediate 11) in 2 steps (1%). ¹H-NMR (400 MHz, CDCl₃): δ 7.97 (1H, d, J=7.3 Hz), 7.88 (1H, s), 7.48 (1H, s), 7.13-7.08 (3H, m), 6.82-6.74 (3H, m), 4.92-4.86 (1H, m), 4.65 (1H, dd, J=9.8, 7.5 Hz), 4.23 (1H, t, J=10.5 Hz), 3.98 (2H, t, J=6.4 Hz), 3.79 (2H, s), 3.41 (3H, s), 1.94-1.88 (2H, m), 1.68-1.62 (2H, m), 1.28 (6H, s). LC-MS: m/z=529.2 [M+H]⁺.

### Example 78: (S)-4-(2,6-difluorobenzyl)-N-(7-((4-hydroxy-4-methylpentyl)oxy)-5-methyl-4-oxo-2,3,4,5-tetrahydrobenzo[b][1,4]oxazepin-3-yl)-1H-pyrazole-1-carboxamide

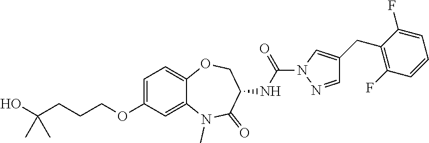 [SMILES: CN1C(=O)[C@@H](NC(=O)n2cc(Cc3c(F)cccc3F)cn2)COc2ccc(OCCCC(C)(C)O)cc21]

The title compound was prepared in a similar fashion to Example 72 with (S)-3-amino-7-((4-hydroxy-4-methylpent-2-yn-1-yl)oxy)-5-methyl-2,3-dihydrobenzo[b][1,4]oxazepin-4(5H)-one hydrochloride (Step B in Example 22) and 4-(2,6-difluorobenzyl)-1H-pyrazole hydrochloride (Intermediate 12) in 2 steps (2%). ¹H-NMR (400 MHz, CDCl₃): δ 7.96 (1H, d, J=7.3 Hz), 7.91 (1H, s), 7.53 (1H, s), 7.21-7.14 (1H, m), 7.12-7.09 (1H, m), 6.89-6.86 (2H, m), 6.75 (2H, dd, J=7.8, 2.3 Hz), 4.91-4.85 (1H, m), 4.64 (1H, dd, J=9.6, 7.3 Hz), 4.22 (1H, dd, J=11.0, 10.1 Hz), 3.98 (2H, t, J=6.2 Hz), 3.83 (2H, s), 3.41 (3H, s), 1.94-1.87 (2H, m), 1.68-1.64 (2H, m), 1.28 (6H, s). LC-MS: m/z=529.2 [M+H]⁺.

### Example 79: (S)—N-(7-((4-hydroxy-4-methylpent-2-yn-1-yl)oxy)-5-methyl-4-oxo-2,3,4,5-tetrahydrobenzo[b][1,4]oxazepin-3-yl)-4-(3-(trifluoromethyl)benzyl)-1H-pyrazole-1-carboxamide

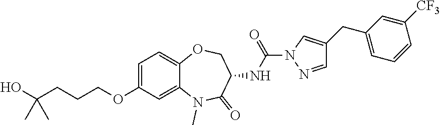 [SMILES: CN1C(=O)[C@@H](NC(=O)n2cc(Cc3cccc(C(F)(F)F)c3)cn2)COc2ccc(OCCCC(C)(C)O)cc21]

The title compound was prepared in a similar fashion to Example 72 with (S)-3-amino-7-((4-hydroxy-4-methylpent-2-yn-1-yl)oxy)-5-methyl-2,3-dihydrobenzo[b][1,4]oxazepin-4(5H)-one hydrochloride (Step B in Example 22) and 4-(3-(trifluoromethyl)benzyl)-1H-pyrazole hydrochloride (Intermediate 13) in 2 steps (1%). ¹H-NMR (400 MHz, CDCl₃): δ 7.97 (1H, d, J=7.2 Hz), 7.87 (1H, s), 7.48-7.33 (5H, m), 7.09-7.07 (1H, m), 6.75-6.72 (2H, m), 4.92-4.85 (1H, m), 4.64 (1H, dd, J=10.0, 7.6 Hz), 4.23 (1H, dd, J=11.0, 10.2 Hz), 3.97 (2H, t, J=6.4 Hz), 3.86 (2H, s), 3.40 (3H, s), 1.93-1.85 (2H, m), 1.66-1.62 (2H, m), 1.26 (6H, s). LC-MS: m/z=560.57 [M+H]⁺.

### Example 80: (S)-4-(3-chlorobenzyl)-N-(7-((4-hydroxy-4-methylpentyl)oxy)-5-methyl-4-oxo-2,3,4,5-tetrahydrobenzo[b][1,4]oxazepin-3-yl)-1H-pyrazole-1-carboxamide

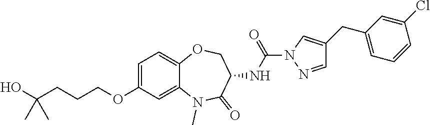 [SMILES: CN1C(=O)[C@@H](NC(=O)n2cc(Cc3cccc(Cl)c3)cn2)COc2ccc(OCCCC(C)(C)O)cc21]

### Step A: (S)-3-amino-7-((4-hydroxy-4-methylpentyl)oxy)-5-methyl-2,3-dihydrobenzo[b][1,4]oxazepin-4(5H)-one hydrochloride

A suspension of (S)-3-amino-7-((4-hydroxy-4-methylpent-2-yn-1-yl)oxy)-5-methyl-2,3-dihydrobenzo[b][1,4]oxazepin-4(5H)-one hydrochloride (Step B in Example 22, 40.0 mg, 0.117 mmol) and Pd/C (10 wt %, 6.25 mg, 5.87 μmol) and TEA (16.0 μL, 0.117 mmol) in EtOAc (1.2 mL) and MeOH (0.10 mL) was stirred at room temperature for 10 min under H₂ atmosphere (1 atm). The reaction mixture was filtered through a Celite pad, washed with EtOAc, and concentrated in vacuo to afford (S)-3-amino-7-((4-hydroxy-4-methylpentyl)oxy)-5-methyl-2,3-dihydrobenzo[b][1,4]oxazepin-4(5H)-one hydrochloride (36.0 mg, 99%) as a colorless oil, which was used for next step without further purification. LC-MS: m/z=309.1 [M+H]⁺.

### Step B: (S)-4-(3-chlorobenzyl)-N-(7-((4-hydroxy-4-methylpentyl)oxy)-5-methyl-4-oxo-2,3,4,5-tetrahydrobenzo[b][1,4]oxazepin-3-yl)-1H-pyrazole-1-carboxamide

The title compound was prepared in a similar fashion to Example 72 (Step A) with (S)-3-amino-7-((4-hydroxy-4-methylpentyl)oxy)-5-methyl-2,3-dihydrobenzo[b][1,4]oxazepin-4(5H)-one hydrochloride and 4-(3-chlorobenzyl)-1H-pyrazole hydrochloride (Intermediate 14). LC-MS: m/z=527.1 [M+H]⁺.

### Example 81: (S)-4-(3-chlorobenzyl)-N-(7-((4-hydroxy-4-methylpentyl)oxy)-5-methyl-4-oxo-2,3,4,5-tetrahydrobenzo[b][1,4]oxazepin-3-yl)-1H-pyrazole-1-carboxamide

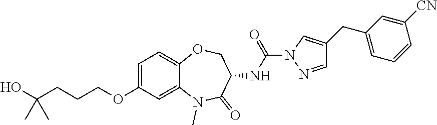 [SMILES: CN1C(=O)[C@@H](NC(=O)n2cc(Cc3cccc(C#N)c3)cn2)COc2ccc(OCCCC(C)(C)O)cc21]

The title compound was prepared in a similar fashion to Example 80 with (S)-3-amino-7-((4-hydroxy-4-methylpent-2-yn-1-yl)oxy)-5-methyl-2,3-dihydrobenzo[b][1,4]oxazepin-4(5H)-one hydrochloride (Step B in Example 22) and 3-((1H-pyrazol-4-yl)methyl)benzonitrile hydrochloride (Intermediate 15) in 2 steps (1%). LC-MS: m/z=517.87 [M+H]⁺.

### Example 82: 4-(3-fluorobenzyl)-N-((3S)-5-methyl-7-((1-methyl-5-oxopyrrolidin-2-yl)methoxy)-4-oxo-2,3,4,5-tetrahydrobenzo[b][1,4]oxazepin-3-yl)-1H-pyrazole-1-carboxamide

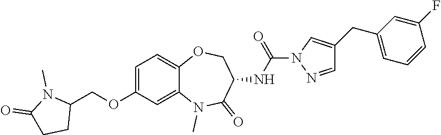 [SMILES: CN1C(=O)[C@@H](NC(=O)n2cc(Cc3cccc(F)c3)cn2)COc2ccc(OCC3CCC(=O)N3C)cc21]

### Step A: tert-butyl ((3S)-5-methyl-4-oxo-7-((5-oxopyrrolidin-2-yl)methoxy)-2,3,4,5-tetrahydrobenzo[b][1,4]oxazepin-3-yl)carbamate

The title compound was prepared in a similar fashion to Example 15 (Step A) with Intermediate 2 and (5-oxopyrrolidin-2-yl)methyl methanesulfonate. The crude product was purified by column chromatography on SiO₂ (Hexanes:EtOAc=2:1) to afford tert-butyl ((3S)-5-methyl-4-oxo-7-((5-oxopyrrolidin-2-yl)methoxy)-2,3,4,5-tetrahydrobenzo[b][1,4]oxazepin-3-yl)carbamate (60%) as a white foam. ¹H-NMR (400 MHz, CDCl₃): δ 7.06 (1H, d, J=8.7 Hz), 6.71-6.67 (2H, m), 5.87 (1H, d, J=5.0 Hz), 5.47 (1H, d, J=7.3 Hz), 4.67-4.61 (1H, m), 4.52 (1H, dd, J=9.6, 7.8 Hz), 4.13-4.05 (2H, m), 3.97 (1H, td, J=5.9, 2.9 Hz), 3.83-3.78 (1H, m), 3.38 (3H, s), 2.46-2.33 (3H, m), 1.97-1.87 (1H, m), 1.40 (9H, s).

### Step B: tert-butyl ((3S)-5-methyl-7-((1-methyl-5-oxopyrrolidin-2-yl)methoxy)-4-oxo-2,3,4,5-tetrahydrobenzo[b][1,4]oxazepin-3-yl)carbamate

A mixture of tert-butyl ((3S)-5-methyl-4-oxo-7-((5-oxopyrrolidin-2-yl)methoxy)-2,3,4,5-tetrahydrobenzo[b][1,4]oxazepin-3-yl)carbamate (100 mg, 0.247 mmol), Mel (0.0170 mL, 0.271 mmol) and Cs₂CO₃ (161 mg, 0.493 mmol) in DMF (3.0 mL) was stirred at room temperature for 18 hours. After quenched with water, the mixture was extracted with DCM twice. The combined organic layers were washed with water and brine, dried over Na₂SO₄, filtered, and concentrated in vacuo. The residue was purified by column chromatography on SiO₂ (DCM:MeOH=20:1) to afford tert-butyl ((3S)-5-methyl-7-((1-methyl-5-oxopyrrolidin-2-yl)methoxy)-4-oxo-2,3,4,5-tetrahydrobenzo[b][1,4]oxazepin-3-yl)carbamate (30.0 mg, 29%) as a white foam. ¹H-NMR (400 MHz, CDCl₃): δ 7.06 (1H, d, J=8.7 Hz), 6.71-6.68 (2H, m), 5.47 (1H, d, J=4.1 Hz), 4.64 (1H, dd, J=16.7, 7.5 Hz), 4.54-4.49 (1H, m), 4.13-4.03 (2H, m), 3.99-3.87 (2H, m), 3.38 (3H, s), 2.92 (3H, s), 2.60-2.51 (1H, m), 2.44-2.36 (1H, m), 2.31-2.24 (1H, m), 2.01-1.93 (1H, m), 1.40 (9H, s).

### Step C: (3S)-3-amino-5-methyl-7-((1-methyl-5-oxopyrrolidin-2-yl)methoxy)-2,3-dihydrobenzo[b][1,4]oxazepin-4(5H)-one hydrochloride

The title compound was prepared in a similar fashion to Example 15 (Step B) with tert-butyl ((3S)-5-methyl-7-((1-methyl-5-oxopyrrolidin-2-yl)methoxy)-4-oxo-2,3,4,5-tetrahydrobenzo[b][1,4]oxazepin-3-yl)carbamate. After concentration in vacuo, the crude product was used for the next reaction without purification. ¹H-NMR (400 MHz, DMSO-d₆): δ 8.46 (3H, d, J=4.6 Hz), 7.20 (1H, d, J=9.1 Hz), 7.13 (1H, d, J=2.7 Hz), 6.90 (1H, dd, J=8.9, 3.0 Hz), 4.50 (1H, dd, J=9.6, 7.8 Hz), 4.40-4.33 (1H, m), 4.28 (1H, t, J=6.2 Hz), 4.20-4.17 (2H, m), 3.88 (1H, t, J=4.1 Hz), 3.35 (3H, s), 3.17 (3H, s), 2.43-2.33 (1H, m), 2.26-2.20 (1H, m), 2.18-2.13 (1H, m), 1.86-1.81 (1H, m).

### Step D: 4-(3-fluorobenzyl)-N-((3S)-5-methyl-7-((1-methyl-5-oxopyrrolidin-2-yl)methoxy)-4-oxo-2,3,4,5-tetrahydrobenzo[b][1,4]oxazepin-3-yl)-1H-pyrazole-1-carboxamide

The title compound was prepared in a similar fashion to Example 15 (Step C) with (3S)-3-amino-5-methyl-7-((1-methyl-5-oxopyrrolidin-2-yl)methoxy)-2,3-dihydrobenzo[b][1,4]oxazepin-4(5H)-one hydrochloride and 4-(3-fluorobenzyl)-1H-pyrazole hydrochloride (Intermediate 6). The crude product was purified by column chromatography on SiO₂ (DCM:EtOAc=15:1) to give 4-(3-fluorobenzyl)-N-((3S)-5-methyl-7-((1-methyl-5-oxopyrrolidin-2-yl)methoxy)-4-oxo-2,3,4,5-tetrahydrobenzo[b][1,4]oxazepin-3-yl)-1H-pyrazole-1-carboxamide (22% for 2 steps) as a white foam. ¹H-NMR (400 MHz, CDCl₃): δ 7.98-7.97 (1H, m), 7.88 (1H, s), 7.47 (1H, s), 7.25-7.23 (1H, m), 7.13 (1H, t, J=4.6 Hz), 7.00-6.85 (3H, m), 6.77-6.74 (2H, m), 4.90 (1H, dd, J=18.8, 7.3 Hz), 4.68-4.63 (1H, m), 4.26 (1H, dd, J=11.2, 9.8 Hz), 4.11-4.06 (1H, m), 4.01-3.97 (1H, m), 3.90 (1H, td, J=8.6, 4.4 Hz), 3.81 (2H, s), 3.42 (3H, s), 2.93 (3H, s), 2.59-2.53 (1H, m), 2.45-2.37 (1H, m), 2.32-2.22 (1H, m), 2.02-1.94 (1H, m). LC-MS: m/z=522.20 [M+H]⁺.

### Biological Activity

### Cell Culture:

Human colon carcinoma cell HT-29 (KCLB 30038), BV2 mouse microglial cell (cell was a kind gift from Dr. Nak-Yun Sung, Senior researcher at Korea Prime Pharmacy CO., LTD.) and human microglial cell HMC3 (ATCC® CRL-3304™). HT-29 cell was grown in Roswell Park Memorial Institute (RPMI) 1640, BV2 cell was grown in Dulbecco's Modified Eagle's Medium (DMEM) and HMC3 cell was grown in Minimum Essential Media Eagle (MEM) supplemented with 10% fetal bovine serum and 1% mixture of penicillin and streptomycin (Gibco). Cells were maintained at 37° C. in a humidified 5% CO2 atmosphere.

### Cell-Based Necroptosis Assay for RIPK1 Activity:

To measure the activity of RIPK1 inhibitor in necroptotic cells, HT-29 cells were treated by control DMSO, human TNFα (Peprotech, Rocky Hill, USA), SM-164 (Biovision, California, USA) and a pan-caspase inhibitor Z-VAD-FMK (Invivogen, San Diego, USA). Cells were pretreated with Z-VAD-FMK 20 μM. After 30 min, human TNFα 10 ng/ml, SM-164 100 nM and RIPK1 Inhibitor (0.0001, 0.001, 0.01, 0.02, 0.05, 0.1, 1, 10 uM) were treated for 24 h. Cell viability was measured by Cell Counting Kit 8 (CCK-8) (Dong-in, Seoul, Korea).

### Immunoblotting:

Biological activity of the compounds of RIPK1 inhibitor was determined by measuring their ability to inhibitor TNFα induced phospho-RIPK1 (ser 166) levels, phospho-RIPK3 levels, phospho-MLKL levels in HMC3 cells. Cells were pretreated with Z-VAD-FMK 20 PM. After 30 min, human TNFα 20 ng/ml, SM-164 100 nM and RIPK1 inhibitor (0.1, 1, 10 nM) were treated for 7 h under serum free media. Cells were lysed with cold lysis buffer containing 25 mM HEPES pH 7.6, 150 nM NaCl, 1% NP40, 1% sodium deoxycholate, 0.1% SDS, and protease inhibitor mixture (Bimake, Houston, USA) using sonicators. The cells were centrifuged at 15,000 rpm, 4° C. for 5 min. After protein concentration of the lysates (supernatants) was quantified using BCA assay (Thermo Fisher Scientific, Waltham, USA), lysates were mixed with LDS sample buffer and heating at 70 for 10 min. (Invitrogen, California, USA). Extracts were separated by sodium dodecyl sulfate polyacrylamide gel electrophoresis (SDS-PAGE) followed by electro-transfer to polyvinylidene difluoride (PVDF) membranes and probed with an anti-phospho-RIPK1 antibody, anti-phospho-RIPK3 antibody and anti-phospho-MLKL antibody (Cell Signaling technology, Danvers, USA) and 3-actin (Proteintech, Rosement, USA), followed by horseradish peroxidase conjugated anti-rabbit (Cell Signaling technology, Danvers, USA), anti-mouse IgG and revealed with Super Signal West dura kit (Pierce). The membranes are placed in an image analyzer (Imagequant, LAS 500, GE Healthcare), connected to a computer which allows the image generation (software Image reader LAS 500).

### Inflammation Cytokine:

Total RNA was extracted and purified from PureLink™ RNA mini kit (Thermo Fisher Scientific, Waltham, USA) according to the manufacture's protocol. Reverse transcription reactions were performed with AccuPower CycleScript RT PreMix (dT20) (Bioneer, Daejeon, Korea). Synthesis of cDNA was carried out using SimpliAmp Thermal Cycler (Applied Biosystems, Carlsbad, CA) and RT-PCR conditions were 15° C. for 30 sec, 42° C. for 4 min, 55° C. for 30 sec in 12 cycles, and heat inactivation was performed 95° C. for 5 min. For qPCR, SYBR Green PCR Master Mix (Thermo Fisher Scientific, Waltham, USA) was used in QuantStudio 3 (Applied Biosystems, Carlsbad, CA) and the PCR conditions were 95° C. for 10 min, 40 cycles of 95° C. for 15 s, and 60° C. for 30 s. The relative mRNA levels were calculated using cycle threshold (Ct) method. GAPDH was used as the endogenous control. PCR primers used in this study are listed in Table 1.

TABLE 1
PCR primers used in this study.

| Primer | Species | SEQ ID NO | | Sequence |
|---|---|---|---|---|
| TNF-α | mouse | Forward | 1 | TGTAGCCCACGTCGTAGCAA |
| | | Reverse | 2 | AGGTACAACCCATCGGCTGG |
| IL-1β | mouse | Forward | 3 | TGTGCAAGTGTCTGAAGCAGC |
| | | Reverse | 4 | TGGAAGCAGCCCTTCATCTT |
| IL-6 | mouse | Forward | 5 | CCACTTCACAAGTCGGAGGC |
| | | Reverse | 6 | GCCATTGCACAACTCTTTTCTC |
| GAPDH | mouse | Forward | 7 | TCACCACCATGGAGAAGGC |
| | | Reverse | 8 | GCTAAGCAGTTGGTGGTGCA |

Cell-base RIPK1 activity
A: below 10 nM, B: 10~50 nM, C: above 50 nM

| Example | Necroptosis | phspho-RIPK1 (Ser166) |
|---|---|---|
| 1 | B | B |
| 2 | A | A |
| 3 | B | B |
| 4 | B | A |
| 5 | A | A |
| 6 | A | A |
| 7 | B | — |
| 8 | C | — |
| 9 | B | — |
| 10 | A | — |
| 11 | A | A |
| 12 | B | B |
| 13 | B | — |
| 14 | B | B |
| 15 | A | A |
| 16 | A | A |
| 17 | B | — |
| 18 | A | A |
| 19 | A | A |
| 20 | A | A |
| 21 | A | A |
| 22 | A | A |
| 23 | A | A |
| 24 | A | A |
| 25 | A | A |
| 26 | A | A |
| 27 | A | A |
| 28 | A | A |
| 29 | A | A |
| 30 | A | A |
| 31 | A | A |
| 32 | A | — |
| 33 | B | — |
| 34 | A | A |
| 35 | A | A |
| 36 | A | A |
| 37 | A | A |
| 38 | A | A |
| 39 | A | A |
| 40 | A | A |
| 41 | A | A |
| 42 | B | B |
| 43 | A | A |
| 44 | A | A |
| 45 | A | A |
| 46 | B | A |
| 47 | A | A |
| 48 | A | A |
| 49 | A | A |
| 50 | A | A |
| 51 | A | A |
| 52 | A | — |
| 53 | A | A |
| 54 | B | B |
| 55 | A | A |
| 56 | A | A |
| 57 | A | A |
| 58 | A | A |
| 59 | A | A |
| 60 | A | A |
| 61 | A | A |
| 62 | A | A |
| 63 | A | A |
| 64 | B | B |
| 65 | A | A |
| 66 | A | A |
| 67 | A | A |
| 68 | A | A |
| 69 | A | A |
| 70 | A | A |
| 71 | A | A |

What is claimed is:

1. A compound, according to formula I 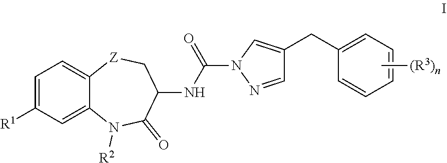
or a pharmaceutically acceptable salt, solvate, polymorph, ester, tautomer, stereoisomer or prodrug thereof, wherein
R¹ is —O[(C₁-C₆)alkyl]NR⁴R⁵, —O(C₁-C₆)alkyl, —O[gem-dimethylhydroxy(C₁-C₆)alkyl], —O(C₁-C₆)alkyl[(C₁-C₆)alkoxy], —O(C₁-C₆)alkyl[(C₃-C₆)cycloalkyl], —O(C₁-C₆)alkyl[(C₃-C₆)cycloalkyl]hydroxy, —O(C₃-C₆)cycloalkyl, —O(C₃-C₆)hydroxycycloalkyl, —O(C₁-C₆)alkyl-(hetAr² or hetAr³), —O(C₁-C₆)alkyl-(hetCyc¹ or hetCyc²), —C(O)NR⁴R⁵, —OC(O)NR⁴R⁵, —O[(C₁-C₃)alkyl]C(O)NR⁴R⁵, or 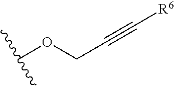
R² is H, CD₃, or optionally substituted by C₁-C₆ alkyl;
each R³ is independently H, methyl, CF₃, halogen, or cyano;
n is 1, 2 or 3;
Z is CH₂, NR², O, or S;
R⁴ is H or C₁-C₆ alkyl;
R⁵ is H, —(C₁-C₆)alkyl, —(C₁-C₆)fluoroalkyl, —(C₁-C₆)difluoroalkyl, —(C₁-C₆)trifluoroalkyl, -gem-dimethyl(C₁-C₆)hydroxy, —(C₁-C₆)hydroxyalkyl, —(C₂-C₆)dihydroxyalkyl, [(C₁-C₆)alkoxy](C₁-C₆)alkyl-, [(C₁-C₆)alkoxy]-[(C₁-C₆)alkoxy]-(C₁-C₆)alkyl-, —O(C₁-C₆)alkyl, —O(C₁-C₆)hydroxyalkyl, —O(C₁-C₆)alkyl[(C₁-C₆)alkoxy], —O(C₁-C₃)alkyl[(C₃-C₆)cycloalkyl], Cyc¹, Ar¹, —CH₂Ar¹, hetCyc¹, hetAr², hetAr³, hetCyc²(C₁-C₂)alkyl- or hetCyc³(C₁-C₂)alkyl-;
or NR⁴R⁵ forms a 4-6 membered heterocyclic ring having a ring nitrogen atom and optionally having a second ring heteroatom selected from N and O, wherein said ring is optionally substituted with one or more substituents independently selected from (C₁-C₆)alkyl, OH, alkoxy, and (C₁-C₆)hydroxyalkyl;
R⁶ is H, —(C₁-C₆)alkyl, —(C₃-C₆)cycloalkyl, -gem-dimethylhydroxy(C₁-C₆)alkyl, —(C₃-C₆)hydroxycycloalkyl, hetAr²;
hetAr¹ is a 5-membered heteroaryl ring having 2-3 ring heteroatoms, wherein at least 1 of said ring heteroatoms is N and said ring is optionally substituted with a substituent selected from (C₁-C₆)alkyl, NH₂, (C₁-C₆ hydroxyalkyl)NH—, (HO)₂P(═O)OCH₂—, (C₁-C₆)hydroxyalkyl, Cyc¹, and (C₁-C₆ alkyl)COOH;
Cyc¹ is a 3-6 membered cycloalkyl ring which is optionally substituted with one or more substituents independently selected from —(C₁-C₄ alkyl), OH, OCH₃, COOH, —(C₁-C₄ alkyl)OH, halogen and CF₃;
hetCyc¹ is a carbon-linked 4-6 membered heterocyclic ring optionally substituted with a substituent selected from (C₁-C₆)alkyl;
hetCyc² is a 5-6 membered heterocyclic ring having a ring nitrogen atom and optionally having a second ring heteroatom selected from N and O, wherein said ring is optionally substituted with a substituent selected from (C₁-C₆)alkyl, OH, (C₁-C₆)alkoxy, halogen and oxo;
hetCyc³ is a bridged 8-membered heterocyclic ring having a ring nitrogen atom and optionally having a ring oxygen atom;
Ar¹ is phenyl optionally substituted with one or more substituents independently selected from (C₁-C₆)alkoxy, halogen, (C₁-C₆)alkyl and CF₃;
hetAr² is pyridyl optionally substituted with one or more substituents independently selected from halogen, CF₃, (C₁-C₆)alkyl and (C₁-C₆)alkoxy;
hetAr³ is a 5-membered heteroaryl having 2-3 ring heteroatoms independently selected from N, O and S and optionally substituted with (C₁-C₆)alkyl and OH;

2. The compound of claim 1, wherein formula I further includes the absolute configuration compounds of Formula IIa and IIb: 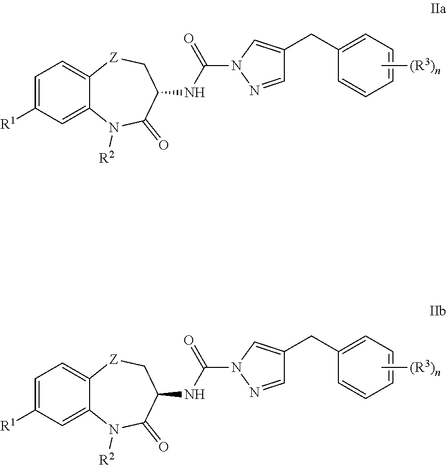
or a pharmaceutically acceptable salt, thereof, wherein
R¹ is —O[(C₁-C₆)alkyl]NR⁴R⁵, —O(C₁-C₆)alkyl, —O[gem-dimethylhydroxy(C₁-C₆)alkyl], —O(C₁-C₆)alkyl[(C₁-C₆)alkoxy], —O(C₁-C₆)alkyl[(C₃-C₆)cycloalkyl], —O(C₁-C₆)alkyl[(C₃-C₆)cycloalkyl]hydroxy, —O(C₃-C₆)cycloalkyl, —O(C₃-C₆)hydroxycycloalkyl, —O(C₁-C₆)alkyl-(hetAr² or hetAr³), —O(C₁-C₆)alkyl-(hetCyc¹ or hetCyc²), —C(O)NR⁴R⁵, —OC(O)NR⁴R⁵, —O[(C₁-C₃)alkyl]C(O)NR⁴R⁵, or 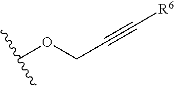
R² is H, CD₃, or optionally substituted by C₁-C₆ alkyl;
each R³ is independently H, methyl, CF₃, halogen, or cyano;
n is 1, 2 or 3;
Z is CH₂, NR², O, or S;
R⁴ is H or C₁-C₆ alkyl;
R⁵ is H, —(C₁-C₆)alkyl, —(C₁-C₆)fluoroalkyl, —(C₁-C₆)difluoroalkyl, —(C₁-C₆)trifluoroalkyl, -gem-dimethyl(C₁-C₆)hydroxy, —(C₁-C₆)hydroxyalkyl, —(C₂-C₆)dihydroxyalkyl, [(C₁-C₆)alkoxy](C₁-C₆)alkyl-, [(C₁-C₆)alkoxy]-[(C₁-C₆)alkoxy]-(C₁-C₆)alkyl-, —O(C₁-C₆)alkyl, —O(C₁-C₆)hydroxyalkyl, —O(C₁-C₆)alkyl[(C₁-C₆)alkoxy], —O(C₁-C₃)alkyl[(C₃-C₆)cycloalkyl], Cyc¹, Ar¹, —CH₂Ar¹, hetCyc¹, hetAr², hetAr³, hetCyc²(C₁-C₂)alkyl- or hetCyc³(C₁-C₂)alkyl-;
or NR⁴R⁵ forms a 4-6 membered heterocyclic ring having a ring nitrogen atom and optionally having a second ring heteroatom selected from N and O, wherein said ring is optionally substituted with one or more substituents independently selected from (C₁-C₆)alkyl, OH, alkoxy, and (C₁-C₆)hydroxyalkyl;
R⁶ is H, —(C₁-C₆)alkyl, —(C₃-C₆)cycloalkyl, -gem-dimethylhydroxy(C₁-C₆)alkyl, —(C₃-C₆)hydroxycycloalkyl, hetAr²;

3. The compound of claim 2, wherein formula IIa further includes the compounds of Formula III: 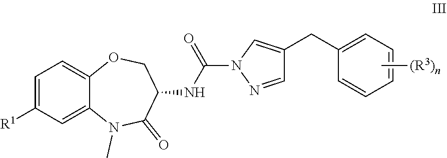
or a pharmaceutically acceptable salt, thereof, wherein
R¹ is —O[(C₁-C₆)alkyl]NR⁴R⁵, —O(C₁-C₆)alkyl, —O[gem-dimethylhydroxy(C₁-C₆)alkyl], —O(C₁-C₆)alkyl[(C₁-C₆)alkoxy], —O(C₁-C₆)alkyl[(C₃-C₆)cycloalkyl], —O(C₁-C₆)alkyl[(C₃-C₆)cycloalkyl]hydroxy, —O(C₃-C₆)cycloalkyl, —O(C₃-C₆)hydroxycycloalkyl, —O(C₁-C₆)alkyl-(hetAr² or hetAr³), —O(C₁-C₆)alkyl-(hetCyc¹ or hetCyc²), —C(O)NR⁴R⁵, —OC(O)NR⁴R⁵, —O[(C₁-C₃)alkyl]C(O)NR⁴R⁵, or 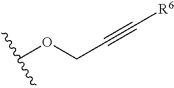
each R³ is independently H, methyl, CF₃, halogen, or cyano;
n is 1, 2 or 3;
R⁴ is H or C₁-C₆ alkyl;
R⁵ is H, —(C₁-C₆)alkyl, —(C₁-C₆)fluoroalkyl, —(C₁-C₆)difluoroalkyl, —(C₁-C₆)trifluoroalkyl, -gem-dimethyl(C₁-C₆)hydroxy, —(C₁-C₆)hydroxyalkyl, —(C₂-C₆)dihydroxyalkyl, [(C₁-C₆)alkoxy](C₁-C₆)alkyl-, [(C₁-C₆)alkoxy]-[(C₁-C₆)alkoxy]-(C₁-C₆)alkyl-, —O(C₁-C₆)alkyl, —O(C₁-C₆)hydroxyalkyl, —O(C₁-C₆)alkyl[(C₁-C₆)alkoxy], —O(C₁-C₃)alkyl[(C₃-C₆)cycloalkyl], Cyc¹, Ar¹, —CH₂Ar¹, hetCyc¹, hetAr², hetAr³, hetCyc²(C₁-C₂)alkyl- or hetCyc³(C₁-C₂)alkyl-;
or NR⁴R⁵ forms a 4-6 membered heterocyclic ring having a ring nitrogen atom and optionally having a second ring heteroatom selected from N and O, wherein said ring is optionally substituted with one or more substituents independently selected from (C₁-C₆)alkyl, OH, alkoxy, and (C₁-C₆)hydroxyalkyl;
R⁶ is H, —(C₁-C₆)alkyl, —(C₃-C₆)cycloalkyl, -gem-dimethylhydroxy(C₁-C₆)alkyl, —(C₃-C₆)hydroxycycloalkyl, hetAr²;

4. A pharmaceutical composition comprising a pharmaceutically effective amount of the compound of claim 1 or a pharmaceutically acceptable salt, solvate, polymorph, ester, tautomer or prodrug thereof, and a pharmaceutically acceptable carrier.

5. Use of the compound of claim 1, wherein the disease is involving both inflammation and necroptosis.

6. Use of the compound of claim 1, wherein the disease is inflammatory bowel disease (including Crohn's disease and ulcerative colitis), psoriasis, systemic lupus erythematosus (SLE), Sjogren's syndrome, systemic scleroderma, anti-phospholipid syndrome (APS), vasculitis, retinal detachment, retinitis pigmentosa, arthritis (including rheumatoid arthritis, spondylarthritis, gout, osteoarthritis, and systemic onset juvenile idiopathic arthritis (SoJIA)), liver damage/diseases (non-alcohol steatohepatitis, alcohol steatohepatitis, autoimmune hepatitis, autoimmune hepatobiliary disease, primary sclerosing cholangitis (PSC), acetaminophen toxicity, hepatotoxicity), kidney damage/injury (nephritis, renal transplant, surgery, administration of nephrotoxic drug), acute kidney injury (AKI), Celiac disease, autoimmune idiopathic thrombocytopenic purpura, systemic inflammatory response syndrome (SIRS), atherosclerosis, or cerebrovascular accident (CVA, stoke).

7. Use of the compound of claim 1, wherein the disease is Parkinson's Disease, Lewy body dementia, multiple system atrophy, Parkinson-plus syndromes, tauopathies, Alzheimer's Disease, Frontotemporal dementia, amyotrophic lateral sclerosis (ALS), spinal muscular atrophy, primary lateral sclerosis, Huntington's disease, ischemia, stroke, intracranial hemorrhage, cerebral hemorrhage, muscular dystrophy, progressive muscular atrophy, progressive muscular atrophy, pseudobulbar palsy, spinal muscular atrophy, inherited muscular atrophy, peripheral neuropathies, progressive supranuclear palsy, corticobasal degeneration, multiple sclerosis, or demyelinating disease.

8. Use of the compound of claim 1, wherein the disease is pancreatic cancer, metastatic adenocarcinoma of the pancreas, pancreatic ductal adenocarcinoma, mesothelioma, melanoma, colorectal cancer, acute myeloid leukemia, metastasis, glioblastoma, breast cancer, gallbladder cancer, clear cell renal carcinoma, or non-small cell lung carcinoma.

9. A method for inhibiting an RIPK1 enzyme comprising the step of contacting the RIPK1 enzyme with an amount sufficient to inhibit said enzyme of the compound of claim 1 or a pharmaceutically acceptable salt, solvate, polymorph, ester, tautomer or prodrug thereof.

10. A method for treating a RIPK1 mediated disease or disorder comprising administering to an individual in need thereof an effective amount of a composition comprising the compound of claim 1 or a pharmaceutically acceptable salt, solvate, polymorph, ester, tautomer or pro-drug thereof, wherein the disease or disorder is selected from inflammatory diseases and neurodegenerative diseases.

11. The use of claim 6, wherein the disease is selected from the group consisting of inflammatory diseases.

12. The use of claim 7, wherein the disease is selected from the group consisting of neurodegenerative disease.

13. The use of claim 8, wherein the disease is selected from the group consisting of cancers.


SEQUENCE LISTING (8 sequences)

SEQ ID NO: 1 - DNA, 20 bp, synthetic construct
  misc_feature 1..20: note = Synthetic primer
  source 1..20: mol_type = other DNA
tgtagcccacgtcgtagcaa

SEQ ID NO: 2 - DNA, 20 bp, synthetic construct
  misc_feature 1..20: note = Synthetic primer
  source 1..20: mol_type = other DNA
aggtacaacccatcggctgg

SEQ ID NO: 3 - DNA, 21 bp, synthetic construct
  misc_feature 1..21: note = Synthetic primer
  source 1..21: mol_type = other DNA
tgtgcaagtgtctgaagcagc

SEQ ID NO: 4 - DNA, 20 bp, synthetic construct
  misc_feature 1..20: note = Synthetic primer
  source 1..20: mol_type = other DNA
tggaagcagcccttcatctt

SEQ ID NO: 5 - DNA, 20 bp, synthetic construct
  misc_feature 1..20: note = Synthetic primer
  source 1..20: mol_type = other DNA
ccacttcacaagtcggaggc

SEQ ID NO: 6 - DNA, 22 bp, synthetic construct
  misc_feature 1..22: note = Synthetic primer
  source 1..22: mol_type = other DNA
gccattgcacaactcttttctc

SEQ ID NO: 7 - DNA, 19 bp, synthetic construct
  misc_feature 1..19: note = Synthetic primer
  source 1..19: mol_type = other DNA
tcaccaccatggagaaggc

SEQ ID NO: 8 - DNA, 20 bp, synthetic construct
  misc_feature 1..20: note = Synthetic primer
  source 1..20: mol_type = other DNA
gctaagcagttggtggtgca